(19) United States
(12) Patent Application Publication
(10) Pub. No.: US 2011/0046108 A1
(43) Pub. Date: Feb. 24, 2011

(54) PYRIMIDINE DERIVATIVES

(75) Inventors: Jason Grant Kettle, Macclesfield (GB); Jon Read, Macclesfield (GB); Andrew Leach, Macclesfield (GB); Bernard Christophe Barlaam, Cedex (FR); Richard Ducray, Cedex (FR); Christine Marie Paul Lambert-Van Der Brempt, Cedex (FR)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/161,766

(22) PCT Filed: Jan. 25, 2007
(86) PCT No.: PCT/GB07/00251

§ 371 Date: Jul. 22, 2008

Related U.S. Application Data
(60) Provisional application No. 60/762,387, filed on Jan. 26, 2006.

(51) Int. Cl. A61K 31/506; C07D 405/12; C07D 413/14; C07D 405/14; C07D 403/12; A61K 31/5377; A61K 31/553; A61K 31/551; A61P 35/00; A61P 9/00

(52) U.S. Cl. 514/210.18; 544/324; 544/122; 544/295; 544/79; 540/544; 514/275; 514/233.8; 514/252.2; 514/249; 514/232.5; 514/211.15; 514/218; 540/492

(57) ABSTRACT

The invention concerns benzamide compounds of Formula (I), or a pharmaceutically acceptable salt thereof, where R¹, ring A, n, R³, and R⁴ are as defined in the description. The present invention also relates to processes for the preparation of such compounds, pharmaceutical compositions containing them and their use in the manufacture of a medicament for use as an antiproliferative agent in the prevention or treatment of tumours or other proliferative conditions which are sensitive to the inhibition of EphB4, and/or EphA2 and/or Src kinases.

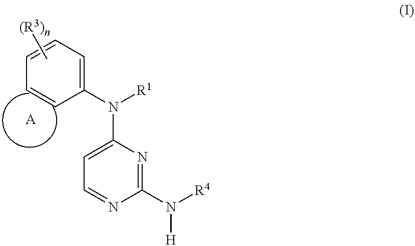

The present invention relates to novel pyrimidine derivatives, to pharmaceutical compositions containing these derivatives and to their use in therapy, in particular in the prevention and treatment of solid tumour disease in a warm blooded animal such as man.

Many of the current treatment regimes for cell proliferation diseases such as psoriasis and cancer utilise compounds which inhibit DNA synthesis. Such compounds are toxic to cells generally but their toxic effect on rapidly dividing cells such as tumour cells can be beneficial. Alternative approaches to target tumours using agents that act on mechanisms other than the inhibition of DNA synthesis have the potential to display enhanced selectivity of action.

In recent years it has been discovered that a cell may become cancerous by virtue of the transformation of a portion of its DNA into an oncogene i.e. a gene which, on activation, leads to the formation of malignant tumour cells (Bradshaw, Mutagenesis 1986, 1, 91). Several such oncogenes give rise to the production of peptides which are receptors for growth factors. Activation of the growth factor receptor complex subsequently leads to an increase in cell proliferation. It is known, for example, that several oncogenes encode tyrosine kinase enzymes and that certain growth factor receptors are also tyrosine kinase enzymes (Yarden et al., Ann. Rev. Biochem., 1988, 57, 443; Larsen et al., Ann. Reports in Med. Chem. 1989, Chpt. 13).

The first group of tyrosine kinases to be identified arose from such viral oncogenes, for example pp60^{v-Src} tyrosine kinase (otherwise known as v-Src), and the corresponding tyrosine kinases in normal cells, for example pp60^{c-Src} tyrosine kinase (otherwise known as c-Src).

Receptor tyrosine kinases are important in the transmission of biochemical signals which initiate a variety of cell responses including proliferation, survival and migration. They are large enzymes which span the cell membrane and possess an extracellular binding domain for growth factors such as epidermal growth factor (EGF) and an intracellular portion which functions as a kinase to phosphorylate tyrosine amino acids in proteins and hence to influence cell proliferation. Various classes of receptor tyrosine kinases are known (Wilks, Advances in Cancer Research, 1993, 60 43-73) and are classified on the basis of the growth factor family to which they bind. This classification includes Class I receptor tyrosine kinases comprising the EGF family of receptor tyrosine kinases such as the EGF, TGFα, Neu and erbB receptors, Class II receptor tyrosine kinases comprising the insulin family of receptor tyrosine kinases such as the insulin and IGF1 receptors and insulin-related receptor (IRR) and Class III receptor tyrosine kinases comprising the platelet-derived growth factor (PDGF) family of receptor tyrosine kinases such as the PDGFα, PDGβ and colony-stimulating factor 1 (CSF1) receptors.

The Eph family is the largest known family of receptor tyrosine kinases, with 14 receptors and 8 cognate ephrin ligands identified in mammals (Reviewed in Kullander and Klein, Nature Reviews Molecular Cell Biology, 2002, 3, 475-486). The receptor family is further sub-divided into two sub-families, which are defined largely by the homology of the extracellular domains and their affinity towards a particular ligand type. In general, all Ephs contain an intracellular tyrosine kinase domain and an extracellular Ig-like domain with a cysteine-rich region with 19 conserved cysteines and two fibronectin type III domains. The A-class of Ephs consists of 8 receptors, termed EphA1-8, which generally bind to their cognate ephrinA class of ligands, termed ephrinA1-5. The B-class consists of 6 receptors, termed EphB1-6, which bind to their cognate ephrinB ligands, termed ephrinB1-3. Eph receptor ligands are unusual and different to most other receptor tyrosine kinase ligands in that they are also tethered to cells, via a glycosylphosphatidylinositol linker in ephrinA ligands or an integral transmembrane region in ephrinB ligands. Binding of ephrin ligand to the Eph partner induces a conformational change within the Eph intracellular domain that enables phosphorylation of tyrosine residues within an auto-inhibitory juxtamembrane region, which relieves this inhibition of catalytic site and enables additional phosphorylation to stabilise the active conformation and generate more docking sites for downstream signalling effectors.

Furthermore, evidence indicates that Eph/ephrin signalling can regulate other cell responses such as proliferation and survival.

There is growing evidence that Eph receptor signalling may contribute to tumourigenesis in a wide variety of human cancers, either on tumour cells directly or indirectly via modulation of vascularisation. For instance, many Eph receptors are over-expressed in various tumour types (Reviewed in Surawska et al., Cytokine & Growth Factor Reviews, 2004, 15, 419-433, Nakamoto and Bergemann, Microscopy Res and Technique, 2002, 59, 58-67); EphA2 and other EphA receptor levels are elevated in diverse tumours such as leukemias, breast, liver, lung, ovarian and prostate. Similarly expression of EphB receptors including EphB4 is up-regulated in tumours such as neuroblastomas, leukemias, breast, liver, lung and colon. Moreover, various in vitro and in vivo studies, particularly relating to EphA2 and EphB4, have indicated that over-expression of Eph receptors on cancer cells is able to confer tumourigenic phenotypes such as proliferation and invasion, consistent with the speculated role in oncogenesis.

For instance, inhibition of EphB4 expression using interfering-RNA or antisense oligodeoxynucleotides inhibited proliferation, survival and invasion of PC3 prostate cancer cells in vitro and in vivo xenograft model (Xia et al., Cancer Res., 2005, 65, 4623-4632). EphA2 over-expression in MCF-10A mammary epithelial cells is sufficient to cause tumourigenesis (Zelinski et al., Cancer Res., 2001, 61, 2301-2306). Inhibition of EphA2 function with therapeutic antibodies (Coffman et al., Cancer Res., 2003, 63, 7907-7912) or interfering-RNA (Landen et al., Cancer Res., 2005, 15, 6910-6918) has is been demonstrated to inhibit tumour growth in in vivo xenograft models. Expression of kinase-dead EphA2 mutant receptors in breast cancer cell lines inhibited growth and metastasis of xenograft tumours in vivo, consistent with an essential role of the kinase domain (Fang et al., Oncogene, 2005, 24, 7859-7868).

In addition to compelling role of Eph receptors on tumour cells, there is good evidence that both EphA2 and EphB4 may contribute to tumour vascularisation (Reviewed in Brantley-Sieders et al., Current Pharmaceutical Design, 2004, 10, 3431-3442, Cheng et al., Cytokine and Growth Factor Reviews, 2002, 13, 75-85). Members of Eph family including both EphA2 and EphB4 are expressed on endothelial cells. Transgenic studies have shown that disruption of EphB4 (Gerety et al., Molecular Cell, 1999, 4, 403-414) or its ligand ephrinB2 (Wang et al., Cell, 1998, 93, 741-753) causes embryonic lethality associated with vascular modelling defects consistent with a critical role in vessel development. EphB4 activation stimulates endothelial cell proliferation and migration in vitro (Steinle et al., J. Biol. Chem., 2002, 277, 43830-43835).

Moreover, inhibition of EphB4 signalling using soluble extracellular-domains of EphB4 have been shown to inhibit tumour growth and angiogenesis in in vivo xenograft studies (Martiny-Baron et al., Neoplasia, 2004, 6, 248-257, Kertesz et al., Blood, 2005, Pre-published online). Similarly, soluble EphA2 inhibited tumour vascularisation in a variety of in vivo models (Brantley et al., Oncogene, 2002, 21, 7011-7026, Cheng et al., Neoplasia, 2003, 5, 445-456).

Accordingly it has been recognised that an inhibitor of Eph receptors, particularly EphB4 or EphA2, should be of value as a selective inhibitor of the proliferation and survival of tumour cells by either targeting tumour cells directly or via effects on tumour vascularisation. Thus, such inhibitors should be valuable therapeutic agents for the containment and/or treatment of tumour disease.

It is also known that certain tyrosine kinases belong to the class of non-receptor tyrosine kinases which are located intracellularly and are involved in the transmission of biochemical signals such as those that influence tumour cell motility, dissemination and invasiveness and subsequently metastatic tumour growth (Ullrich et al., Cell 1990, 61, 203-212, Bolen et al., FASEB J., 1992, 6, 3403-3409, Brickell et al., Critical Reviews in Oncogenesis, 1992, 3, 401-406, Bohlen et al., Oncogene, 1993, 8, 2025-2031, Courtneidge et al., Semin. Cancer Biol., 1994, 5, 239-246, Lauffenburger et al., Cell, 1996, 84, 359-369, Hanks et al., BioEssays, 1996, 19, 137-145, Parsons et al., Current Opinion in Cell Biology, 1997, 9, 187-192, Brown et al., Biochimica et Biophysica Acta., 1996, 1287, 121-149 and Schlaepfer et al., Progress in Biophysics and Molecular Biology, 1999, 71 435-478). Various classes of non-receptor tyrosine kinases are known including the Src family such as the Src, Lyn and Yes tyrosine kinases, the Abl family such as Abl and Arg and the Jak family such as Jak 1 and Tyk 2.

It is known that the Src family of non-receptor tyrosine kinases are highly regulated in normal cells and in the absence of extracellular stimuli are maintained in an inactive conformation. However, some Src family members, for example c-Src tyrosine kinase, are frequently significantly activated (when compared to normal cell levels) in common human cancers such as gastrointestinal cancer, for example colon, rectal and stomach cancer (Cartwright et al., Proc. Natl. Acad. Sci. USA., 1990, 87, 558-562 and Mao et al., Oncogene, 1997, 15, 3083-3090), and breast cancer (Muthuswamy et al., Oncogene, 1995, 11, 1801-1810). The Src family of non-receptor tyrosine kinases has also been located in other common human cancers such as non-small cell lung cancers (NSCLCs) including adenocarcinomas and squamous cell cancer of the lung (Mazurenko et al. European Journal of Cancer, 1992, 28, 372-7), bladder cancer (Fanning et al., Cancer Research, 1992, 52, 1457-62), oesophageal cancer (Jankowski et al., Gut, 1992, 33, 1033-8), cancer of the prostate, ovarian cancer (Wiener et al., Clin. Cancer Research, 1999, 5, 2164-70) and pancreatic cancer (Lutz et al., Biochem. and Biophys. Res. Comm., 1998, 243, 503-8). As further human tumour tissues are tested for the Src family of non-receptor tyrosine kinases it is expected that its widespread prevalence will be established.

It is further known that the predominant role of c-Src non-receptor tyrosine kinase is to regulate the assembly of focal adhesion complexes through interaction with a number of cytoplasmic proteins including, for example, focal adhesion kinase and paxillin. In addition c-Src is coupled to signalling pathways that regulate the actin cytoskeleton which facilitates cell motility. Likewise, important roles are played by the c-Src, c-Yes and c-Fyn non-receptor tyrosine kinases in integrin mediated signalling and in disrupting cadherin-dependent cell-cell junctions (Owens et al., Molecular Biology of the Cell, 2000, 11, 51-64 and Klinghoffer et al., EMBO Journal, 1999, 18, 2459-2471). Cellular motility is necessarily required for a localised tumour to progress through the stages of dissemination into the blood stream, invasion of other tissues and initiation of metastatic tumour growth. For example, colon tumour progression from localised to disseminated, invasive metastatic disease has been correlated with c-Src non-receptor tyrosine kinase activity (Brunton et al., Oncogene, 1997, 14, 283-293, Fincham et al., EMBO J, 1998, 17, 81-92 and Verbeek et al., Exp. Cell Research, 1999, 248, 531-537).

Accordingly it has been recognised that an inhibitor of such non-receptor tyrosine kinases should be of value as a selective inhibitor of the motility of tumour cells and as a selective inhibitor of the dissemination and invasiveness of mammalian cancer cells leading to inhibition of metastatic tumour growth. In particular an inhibitor of such non-receptor tyrosine kinases should be of value as an anti-invasive agent for use in the containment and/or treatment of solid tumour disease.

The applicants have found that certain pyrimidines are useful in the inhibition of EphB4 and, in some cases, EphA2 and Src kinase as well. Such pyrimidines are therefore are useful in therapy, where such enzymes are implicated.

According to a first aspect of the invention, there is provided a compound of formula (I)

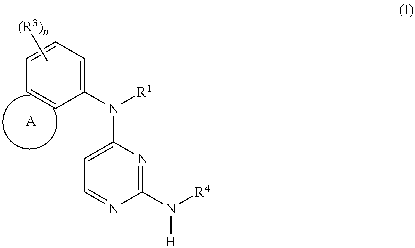

where R¹ is selected from hydrogen, C₁₋₆alkyl, C₂₋₆alkenyl, or C₂₋₆alkynyl, wherein the alkyl, alkenyl and alkynyl groups are optionally substituted by one or more substituents selected from cyano, nitro, —OR², —NR^{2a}R^{2b}, —C(O)NR^{2a}R^{2b}, —N(R^{2a})C(O)R², halo or haloC₁₋₄alkyl (such as trifluoromethyl), where R², R^{2a} and R^{2b} are selected from hydrogen or C₁₋₆alkyl such as methyl, or R^{2a} and R^{2b} together with the nitrogen atom to which they are attached may form a 5 or 6-membered heterocyclic ring, which optionally contains an additional heteroatom selected from N, O or S;
ring A is fused 5 or 6-membered carbocyclic or heterocyclic ring, which is saturated or unsaturated, and is optionally substituted on any available carbon atom by one or more substituent groups selected from halo, cyano, hydroxy, C₁₋₆alkyl, C₁₋₆alkoxy, —S(O)_{z}—C₁₋₆alkyl (where z is 0, 1 or 2), or —NR^{a}R^{b} (where R^{a} and R^{b} are each independently selected from hydrogen, C₁₋₄alkyl, or C₁₋₄alkylcarbonyl), and where any nitrogen atoms in the ring are optionally substituted by a C₁₋₆alkyl or C₁₋₆alkylcarbonyl;
n is 0, 1, 2 or 3
and each group R³ is independently selected from halo, trifluoromethyl, cyano, nitro or a group of sub-formula (i):

—X¹—R¹¹ (i)
where X¹ is selected from a direct bond or O, S, SO, SO₂, OSO₂, NR¹³, CO, CH(OR¹³), CONR¹³, N(R¹³)CO, SO₂N(R¹³), N(R¹³)SO₂, C(R¹³)₂O, C(R¹³)₂S, C(R¹³)₂N(R¹³) and N(R¹³)C(R¹³)₂, wherein R¹³ is hydrogen or C₁₋₆alkyl and R¹¹ is selected from hydrogen, C₁₋₆ alkyl, C₂₋₈alkenyl, C₂₋₈alkynyl, C₃₋₈cycloalkyl, aryl or heterocyclyl, C₃₋₈cycloalkylC₁₋₆ alkyl, arylC₁₋₆ alkyl or heterocyclylC₁₋₆ alkyl, any of which may be optionally substituted with one or more groups selected from halo, trifluoromethyl, cyano, nitro, hydroxy, amino, carboxy, carbamoyl, C₁₋₆alkoxy, C₂₋₆alkenyoxyl, C₂₋₆alkynyloxy, C₁₋₆alkylthio, C₁₋₆alkylsulphinyl, C₁₋₆alkylsulphonyl, C₁₋₆alkylamino, di-(C₁₋₆alkyl)amino, C₁₋₆alkoxycarbonyl, N—C₁₋₆alkylcarbamoyl, N,N-di-(C₁₋₆alkyl)carbamoyl, C₂₋₆alkanoyl, C₂₋₆alkanoyloxy, C₂₋₆alkanoylamino, N—C₁₋₆alkyl-C₂₋₆alkanoylamino, C₃₋₆alkenoylamino, N—C₁₋₆alkyl-C₃₋₆alkenoylamino, C₃₋₆alkynoylamino, N—C₁₋₆alkyl-C₃₋₆alkynoylamino, N—C₁₋₆alkylsulphamoyl, N,N-di-(C₁₋₆alkyl)sulphamoyl, C₁₋₆alkanesulphonylamino and N—C₁₋₆alkyl-C₁₋₆alkanesulphonylamino, and any heterocyclyl group within R¹¹ optionally bears 1 or 2 oxo or thioxo substituents; and
R⁴ is a group of sub-formula (iii)

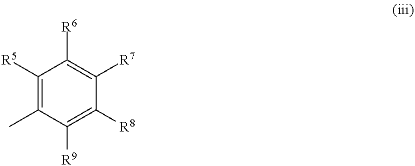

where R⁵, R⁶, R⁷, R⁸ and R⁹ are each independently selected from:
(a) hydrogen, halo, trifluoromethyl, trifluoromethoxy, cyano, nitro, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by halo, hydroxy, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino, and any nitrogen atoms present in a heterocyclyl group may, depending upon valency considerations, be substituted by a group selected from hydrogen, C₁₋₆alkyl or C₁₋₆alkylcarbonyl, and where any sulphur atoms may be optionally oxidised to a sulphur oxide;
(b) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, —N(R¹⁶)C(O)N(R¹⁶)—, —N(R¹⁶)C(O)O—, SON(R¹⁶), N(R¹⁶)SO, SO₂N(R¹⁶), N(R¹⁶)SO₂, C(R¹⁶)₂O, C(R¹⁶)₂S and N(R¹⁶)C(R¹⁶)₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, aryl-C₁₋₆alkyl, or a 4- to 8-membered mono or bicyclic heterocyclyl ring (including 5 or 6 membered heteroaryl rings) or 4- to 8-membered mono or bicyclic heterocyclyl-C₁₋₆alkyl groups (including 5 or 6 membered heteroaryl-C₁₋₆alkyl groups) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v):

—X³—R¹⁵—Z (v)
where X³ is a direct bond or is selected from O, NR¹⁷, S, SO, SO₂, OSO₂, CO, C(O)O, OC(O), CH(OR¹⁷), CON(R¹⁷), N(R¹⁷)CO, —N(R¹⁷)C(O)N(R¹⁷)—, —N(R¹⁷)C(O)O—, SO₂N(R¹⁷), N(R¹⁷)SO₂, C(R¹⁷)₂O, C(R¹⁷)₂S and N(R¹⁷)C(R¹⁷)₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, C₃₋₁₂ carbocyclyl or heterocyclyl (including heteroaryl) which optionally bears 1 or 2 substituents, which may be the same or different, selected from halo, C₁₋₆alkyl, C₂₋₈alkenyl, C₂₋₈alkynyl and C₁₋₆alkoxy and wherein any heterocyclyl group within Z optionally bears 1 or 2 oxo substituents,
or Z is a group of sub-formula (vi)

—X⁴—R¹⁸ (vi)
where X⁴ is selected from O, NR¹⁹, S, SO, SO₂, OSO₂, CO, C(O)O, OC(O), CH(OR¹⁹), CON(R¹⁹), N(R¹⁹)CO, SO₂N(R¹⁹), —N(R¹⁹)C(O)N(R¹⁹)—, —N(R¹⁹)C(O)O—N(R¹⁹)SO₂, C(R¹⁹)₂O, C(R¹⁹)₂S and N(R¹⁹)C(R¹⁹)₂, where each R¹⁹ is independently selected from hydrogen or C₁₋₆alkyl; and R¹⁸ is selected from hydrogen, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl) which optionally bears 1 or 2 substituents, which may be the same or different, selected from halo, C₁₋₆alkyl, C₂₋₈alkenyl, C₂₋₈alkynyl and C₁₋₆alkoxy, and wherein any heterocyclyl group within R¹⁸ optionally bears 1 or 2 oxo substituents;
or
(d) R⁵ and R⁶, R⁶ and R⁷, R⁷ and R⁸ or R⁸ and R⁹ are joined together to form a fused 5, 6 or 7-membered ring, wherein said ring is unsaturated or partially or fully saturated and is optionally substituted on any available carbon atom by halo, C₁₋₆alkyl, hydroxyC₁₋₆alkyl, amino, N—C₁₋₆alkylamino, or N,N-diC₁₋₆alkylamino, and said ring may contain one or more heteroatoms selected from oxygen, sulphur or nitrogen, where sulphur atoms may be optionally oxidised to a sulphur oxide, where any CH₂ groups may be substituted by a C(O) group, and where nitrogen atoms, depending upon valency considerations, may be substituted by a group R²¹, where R²¹ is selected from hydrogen, C ₁₋₆alkyl or C₁₋₆alkylcarbonyl;
or a pharmaceutically acceptable salt thereof,
   with the proviso that if Ring A, together with the phenyl ring to which it is attached, forms an indazol-4-yl group, then R¹ is not hydrogen.

According to a second aspect of the present invention, there is provided a compound of formula (I)

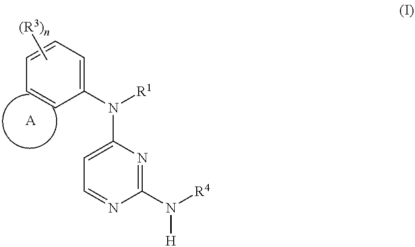

where R¹ is selected from hydrogen or optionally substituted C₁₋₆alkyl, optionally substituted C₂₋₆alkenyl or optionally substituted C₂₋₆alkynyl;
ring A is fused 5 or 6-membered carbocyclic or heterocyclic ring which is optionally substituted on a carbon atom by one or more halo groups or C₁₋₆alkyl groups, and where any nitrogen atoms in the ring are optionally substituted by a C₁₋₆alkyl or C-₁₋₆alkylcarbonyl;
n is 0, 1, 2 or 3
and each group R³ is independently selected from halo, trifluoromethyl, cyano, nitro or a group of sub-formula (i):

—X¹—R¹¹ (i)
where X¹ is selected from a direct bond or O, S, SO, SO₂, OSO₂, NR¹³, CO, CH(OR¹³), CONR¹³, N(R¹³)CO, SO₂N(R¹³), N(R¹³)SO₂, C(R¹³)₂O, C(R¹³)₂S, C(R¹³)₂N(R¹³) and N(R¹³)C(R¹³)₂, wherein R¹³ is hydrogen or C₁₋₆alkyl and
R¹¹ is selected from hydrogen, C₁₋₆ alkyl, C₂₋₈alkenyl, C₂₋₈alkynyl, C₃₋₈cycloalkyl, aryl or heterocyclyl, C₁₋₆ alkylC₃₋₈cycloalkyl, C₁₋₆ alkylaryl or C₁₋₆ alkylheterocyclyl, any of which may be optionally substituted with one or more groups selected from halo, trifluoromethyl, cyano, nitro, hydroxy, amino, carboxy, carbamoyl, C₁₋₆alkoxy, C₂₋₆alkenyoxyl, C₂₋₆alkynyloxy, C₁₋₆alkylthio, C₁₋₆alkylsulphonyl, C₁₋₆alkylamino, di-(C₁₋₆alkyl)amino, C₁₋₆alkoxycarbonyl, N—C₁₋₆alkylcarbamoyl, N,N-di-(C₁₋₆alkyl)carbamoyl, C₂₋₆alkanoyl, C₂₋₆alkanoyloxy, C₂₋₆alkanoylamino, N—C₁₋₆alkyl-C₂₋₆alkanoylamino, C₃₋₆alkenoylamino, N—C₁₋₆alkyl-C₃₋₆alkenoylamino, C₃₋₆alkynoylamino, N—C₁₋₆alkyl-C₃₋₆alkynoylamino, N—C₁₋₆alkylsulphamoyl, N,N-di-(C₁₋₆alkyl)sulphamoyl, C₁₋₆alkanesulphonylamino and N—C₁₋₆alkyl-C₁₋₆alkanesulphonylamino, and any heterocyclyl group within R¹¹ optionally bears 1 or 2 oxo or thioxo substituents; and

R⁴ is an optionally substituted phenyl ring, wherein one or more adjacent substituents may be joined together to form a fused bicyclic or tricyclic ring; or a pharmaceutically acceptable salt thereof.

It is to be understood that, insofar as certain of the compounds of Formula (I) defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula (I) defined above may exhibit the phenomenon of tautomerism. In particular, tautomerism may affect any heterocyclic groups that bear 1 or 2 oxo substituents. It is also to be understood that the present invention includes in its definition any such tautomeric form, or a mixture thereof, which possesses the above-mentioned activity and is not to be limited merely to any one tautomeric form utilised within the formulae drawings or named in the Examples.

It is to be understood that certain compounds of Formula I above may exist in unsolvated forms as well as solvated forms, such as, for example, hydrated forms. It is also to be understood that the present invention encompasses all such solvated forms that possess anticancer or antitumour activity.

It is also to be understood that certain compounds of the Formula I may exhibit polymorphism, and that the present invention encompasses all such forms which possess anticancer or antitumour activity.

Where optional substituents are selected from “one or more” substituent groups it is to be understood that this definition includes all substituents being chosen from one of the specified groups, or the substituents being chosen from two or more of the specified groups. In this specification the generic term “alkyl” includes both straight-chain and branched-chain alkyl groups such as propyl, isopropyl and tert-butyl. However references to individual alkyl groups such as “propyl” are specific for the straight-chain version only, references to individual branched-chain alkyl groups such as “isopropyl” are specific for the branched-chain version only. An analogous convention applies to other generic terms, for example (1-6C)alkoxy includes methoxy, ethoxy and isopropoxy, (1-6C)alkylamino includes methylamino, isopropylamino and ethylamino, and di-[(1-6Calkyl]amino includes dimethylamino, diethylamino and N-methyl-N-isopropylamino. Similarly alkenyl or alkynyl groups may be straight chain or branched.

The term “aryl” refers to phenyl or naphthyl, particularly phenyl.

The terms “halo” or “halogen” refers to fluoro, chloro, bromo, or iodo.

The term “heterocyclyl” or “heterocyclic ring”, unless otherwise defined herein, refers to saturated, partially saturated or unsaturated, mono, bicyclic or tricyclic rings containing 3-15 atoms, of which at least one atom is chosen from nitrogen, sulphur or oxygen. These groups may, unless otherwise specified, be carbon or nitrogen linked. In addition, or a ring sulphur atom may be optionally oxidised to form the S-oxides. More particularly, a “heterocyclyl” or “heterocyclic ring” is a saturated, partially saturated or unsaturated, mono or bicyclic ring containing 3-12 atoms, and especially 4 to 10 atoms, of which at least one atom is chosen from nitrogen, sulphur or oxygen. Monocyclic “heterocyclyls” or “heterocyclic rings” suitably contain from 3-7 ring atoms, in particular 5 or 6 ring atoms.

Examples and suitable values of the term “heterocyclyl” are thienyl, piperidinyl, morpholinyl, furyl, thiazolyl, pyridyl, imidazolyl, 1,2,4-triazolyl, thiomorpholinyl, coumarinyl, pyrimidinyl, phthalidyl, pyrazolyl, pyrazinyl, pyridazinyl, benzothienyl, benzimidazolyl, tetrahydrofuryl, [1,2,4]triazolo[4,3-a]pyrimidinyl, piperidinyl, indolyl, indazolyl, benzothiazolyl, benzoxazolyl, 1,3-benzodioxolyl, pyrrolidinyl, pyrrolyl, quinolinyl, isoquinolinyl, isoxazolyl, benzofuranyl, 1,2,3-thiadiazolyl, 1,2,5-thiadiazolyl, pyrimidinyl, 2,1-benzisoxazolyl, 4,5,6,7-tetrahydro-2H-indazolyl, imidazo[2,1-b][1,3]thiazolyl, tetrahydrofuranyl, tetrahydropyranyl, piperidinyl, morpholinyl, 2,3-dihydro-1-benzofuryl, 2,3-dihydro-1,4-benzodioxinyl, 1,3-benzothiazolyl, 3,4-dihydro-2H-benzodioxepinyl, 2,3-dihydro-1,4-benzodioxinyl, chromanyl, 2,3-dihydrobenzofuranyl, imidazo[2,1-b][1,3]thiazolyl, isoindolinyl, oxazolyl, pyridazinyl, quinoxalinyl, tetrahydrofuryl, 4,5,6,7-tetrahydro-1-benzofuryl, 4,5,6,7-tetrahydro-2H-indazolyl, 4,5,6,7-tetrahydro-1H-indolyl, tetrahydropyranyl or 1,2,3,4-tetrahydroquinolinyl.

Heterocyclyl groups may be non-aromatic or aromatic in nature. Aromatic heterocyclyl groups are specifically referred to as heteroaryl. Heteroaryl groups are totally unsaturated, mono or bicyclic rings containing 3-12 atoms of which at least one atom is chosen from nitrogen, sulphur or oxygen, which may, unless otherwise specified, be carbon or nitrogen linked. Suitably “heteroaryl” refers to a totally unsaturated, monocyclic ring containing 5 or 6 atoms or a bicyclic ring containing 8-10 atoms of which at least one atom is chosen from nitrogen, sulphur or oxygen, which may, unless otherwise specified, be carbon or nitrogen linked. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyranyl, indolyl, pyrimidyl, pyrazinyl, pyridazinyl, benzothienyl, pyridyl and quinolyl.

As stated above, when R¹ is an optionally substituted C₁₋₆alkyl, optionally substituted C₂₋₆alkenyl or optionally substituted C₂₋₆alkynyl, optional substituents are suitably selected from cyano, —OR², —NR^{2a}R^{2b}, —C(O)NR^{2a}R^{2b}, or —N(R^{2a})C(O)R², halo or haloC₁₋₄alkyl such as trifluoromethyl, where R², R^{2a} and R^{2b} are selected from hydrogen or C₁₋₆alkyl such as methyl, or R^{2a} and R^{2b} together with the nitrogen atom to which they are attached may form a heterocyclic ring which optionally contains an additional heteroatom.

In one embodiment of the invention, R¹ is hydrogen.

In a further embodiment, n is 0, 1, or 2. For instance, n is 0 or 1. In yet a further embodiment, n is 1.

Where n is 1 or more, a substituent R³ is suitably positioned on the available ortho-carbon atom of the ring, forming a compound of formula (IA)

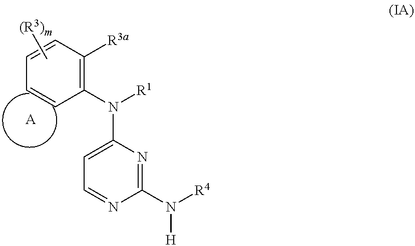

where A, R¹, R³ and R⁴ are as defined herein relation to formula (I), R^{3a} is a group R³ as defined herein, and in particular is halo, and m is 0, 1 or 2. Particular examples of A groups are set out below, and include for example groups A′ as defined below. In particular A is —OCH₂O—, O—CF₂—O—, —OCH═N—, —N═CH—O—, —N═CH—S—, —NH—N═CH—, or —CH═N—NH—.

When n is other than zero, particular examples of R³ or R^{3a} groups are groups selected from halo, trifluoromethyl, cyano, hydroxy, C₁₋₆alkyl, C₂₋₈alkenyl, C₂₋₈alkynyl and C₁₋₆alkoxy

For instance, R³ or R^{3a} may be selected from chloro, fluoro, bromo, trifluoromethyl, cyano, hydroxy, methyl, ethyl, ethynyl, methoxy and ethoxy.

In one embodiment, R³ or R^{3a} is halo, such as bromo, chloro or fluoro, and in particular chloro.

In a particular embodiment, n is 1 and R³ or R^{3a} is halo such as chloro.

Suitably in formula (IA), m is 0.

Examples of ring A include made up of a group of formula —CR²²═CR²²—CR²²═CR²²—, —N═CR²²—CR²²═CR²²—, —CR²²═N—CR²²═CR²²—, —CR²²═CR²²—N═CR²²—, —CR²²═CR²²—CR²²═N—, —N═CR²²—N═CR²²—, —CR²²═N—CR²²═N—, —N═CR²²—CR²²═N—, —N═N—CR²²═CR²², —CR²²═CR²²—N═N—, —CR²²═CR²²—O—, —O—CR²²═CR²²—, —CR²²═CR²²—S—, —S—CR²²═CR²²—, —CR²²H—CR²²H—O—, —O—CR²²H—CR²²H—, —CR²²H—CR²²H—S—, —S—CR²²H—CR²²H—, —O—CR²²H—O—, —O—CF₂—O—, —O—CR²²H—CR²²H—O—, —S—CR²²H—S—, —S—CR²²H—CR²²H—S—, —CR²²═CR²²—NR²⁰—, —NR²⁰—CR²²═CR²²—, —CR²²H—CR²²H—NR²⁰—, —NR²⁰—CR²²H—CR²²H—, —N═CR²²—NR²⁰—, —NR²⁰—CR²²═N—, —NR²⁰—CR²²H—NR²⁰—, —OCR²²═N—, —N═CR²²—O—, —S—CR²²═N—, —N═CR²²—S—, —O—CR²²H—NR²⁰—, —NR²⁰—CR²²H—O—, —S—CR²²H—NR²⁰—, —NR²⁰—CR²²H—S—, —O—N═CR²²—, —CR²²═N—O—, —S—N═CR²²—, —CR²²═N—S—, —O—NR²⁰—CR²²H—, —CR²²H—NR²⁰—O—, —S—NR²⁰—CR²²H—, —CR²²H—NR²⁰—S—, —NR²⁰—N═CR²²—, —CR²²═N—NR²⁰—, —NR²⁰—NR²⁰—CR²²H—, —CR²²H—NR²⁰—NR²⁰—, —N═N—NR²⁰— or —NR²⁰—N═N—, where each R²⁰ is independently selected from hydrogen, C₁₋₆alkyl or C₁₋₆alkylcarbonyl, and where each R²² is independently selected from hydrogen, halo or C₁₋₆alkyl.

In a particular embodiment, where a group A includes more than one group R²⁰ or R²², at least one such group is hydrogen.

Particular examples of groups R²⁰ include hydrogen, methyl, ethyl or methylcarbonyl, in particular hydrogen.

Particular examples of groups R²² include hydrogen, chloro, fluoro, methyl or ethyl, in particular hydrogen.

In a particular embodiment, ring A is a fused five-membered ring. Thus particular examples of A are Ring A is made up of a group of formula —CH═CH—O—, —O—CH═CH—, —CH═CH—S—, —S—CH═CH—, —CH₂—CH₂—O—, —O—CH₂—CH₂—, —CH₂—CH₂—S—, —S—CH₂—CH₂—, —O—CH₂—O—, —O—CH₂—CH₂—O—, —S—CH₂—S—, —S—CH₂—CH₂—S—, —CH═CH—NR²⁰—, —NR²⁰—CH═CH—, —CH₂—CH₂—NR²⁰—, —NR²⁰—CH₂—CH₂—, —N═CH—NR²⁰—, —NR²⁰—CH═N—, —NR²⁰—CH₂—NR²⁰—, —OCH═N—, —N═CH—O—, —S—CH═N—, —N═CH—S—, —O—CH₂—NR²⁰—, —NR²⁰—CH₂—O—, —S—CH₂—NR²⁰—, —NR²⁰—CH₂—S—, —O—N═CH—, —CH═N—O—, —S—N═CH—, —CH═N—S—, —O—NR²⁰—CH₂—, —CH₂—NR²⁰—O—, —S—NR²⁰—-CH₂—, —CH₂—NR²⁰—S—, —NR²⁰—N═CH—, —CH═N—NR²⁰—, —NR²⁰—NR²⁰—CH₂—, —CH₂—NR²⁰—NR²⁰—, —N═N—NR²⁰ — or —NR²⁰—N═N—.

Particular examples of R²⁰ include hydrogen, methyl, and acetyl. For instance, R²⁰ is hydrogen.

In one embodiment, Ring A includes one nitrogen atom. For instance, it is a group of formula —CH═CH—NR²⁰— or —NR²⁰—CH═CH—.

Ring A may also include two nitrogen atoms. For instance, it may be a group of formula —NR²⁰—N═CH—, —CH═N—NR²⁰—, —NR²⁰—NR²⁰—CH₂—, or —CH₂—NR²⁰—NR²⁰ and in particular is a group —NR²⁰—N═CH— or —CH═N—NR²⁰—.

In another embodiment, Ring A includes one nitrogen and one oxygen atom. It is therefore suitably selected from —O—N═CH—, —CH═N—O—, —O—NR²⁰—CH₂— or —CH₂—NR²⁰—O—.

In yet a further embodiment, Ring A is a group of formula —O—CH₂—O— or —O—CF₂—O—, in particular —O—CH₂—O—.

In particular, examples of compounds of formula (I) are compounds of formula (IB)

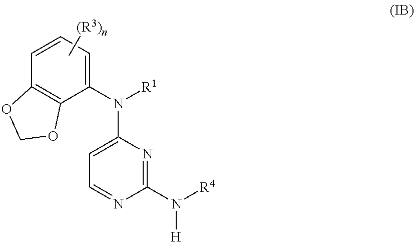

wherein R¹, R³, R⁴ and n are as defined.

Particular examples of optionally substituted phenyl groups R⁴ are groups of sub-formula (iii)

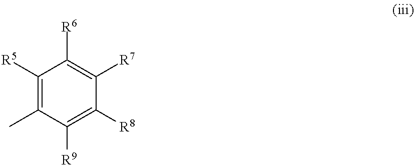

where R⁵, R⁶, R⁷, R⁸ and R⁹ are independently selected from:
(a) hydrogen, halo, trifluoromethyl, trifluoromethoxy, cyano, nitro, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl);
(b) a group of sub-formula (iv):

—X²—R¹⁴ (iv)
where X² is selected from O, NR¹⁶S, SO, SO₂, OSO₂, CO, C(O)O, OC(O), CH(OR¹⁶), CON(R¹⁶), N(R¹⁶)CO, —N(R¹⁶)C(O)N(R¹⁶)—, —N(R¹⁶)C(O)O—SO₂N(R¹⁶), N(R¹⁶)SO₂, C(R¹⁶)₂O, C(R¹⁶)₂S and N(R¹⁶)C(R¹⁶)₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl);
(c) a group of sub-formula (v):

—X³—R¹⁵—Z (v)
where X³ is a direct bond or is selected from O, NR¹⁷ S, SO, SO₂, OSO₂, CO, C(O)O, OC(O), CH(OR¹⁷), CON(R¹⁷), N(R¹⁷)CO, —N(R¹⁷)C(O)N(R¹⁷)—, —N(R¹⁷)C(O)O—, SO₂N(R¹⁷), N(R¹⁷)SO₂, C(R¹⁷)₂O, C(R¹⁷)₂S and N(R¹⁷)C(R¹⁷)₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkoxy, cyano, amino, C₁₋₆alkylamino or di-(C₁₋₆alkyl)amino;
Z is halo, trifluoromethyl, cyano, nitro, aryl, C₃₋₁₂ carbocyclyl or heterocyclyl (including heteroaryl) which optionally bears 1 or 2 substituents, which may be the same or different, selected from halo, C₁₋₆alkyl, C₂₋₈alkenyl, C₂₋₈alkynyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹S, SO, SO₂, OSO₂, CO, C(O)O, OC(O), CH(OR¹⁹), CON(R¹⁹), N(R¹⁹)CO, SO₂N(R¹⁹), —N(R¹⁹)C(O)N(R¹⁹)—, —N(R¹⁹)C(O)O—N(R¹⁹)SO₂, C(R¹⁹)₂O, C(R¹⁹)₂S and N(R¹⁹)C(R¹⁹)₂, where each R¹⁹ is independently selected from hydrogen or C₁₋₆alkyl; and R¹⁸ is selected from hydrogen, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl) which optionally bears 1 or 2 substituents, which may be the same or different, selected from halo, C₁₋₆alkyl, C₂₋₈alkenyl, C₂₋₈alkynyl and C₁₋₆alkoxy, and wherein any heterocyclyl group within R¹⁸ optionally bears 1 or 2 oxo substituents;
or
(d) R⁵ and R⁶, R⁶ and R⁷, R⁷ and R⁸ or R⁸ and R⁹ are joined together to form a fused ring, which is optionally substituted, and which may contain one or more heteroatoms selected from oxygen, sulphur or nitrogen, where sulphur atoms may be optionally oxidised to a sulphur oxide, where any CH₂ groups may be substituted by a C(O) group, and where nitrogen atoms, depending upon valency considerations, may be substituted by a group R²¹, where R²¹ is selected from hydrogen, C₁₋₆alkyl or C₁₋₆alkylcarbonyl.

In particular at least one, for instance at least two, of R⁵, R⁶, R⁷, R⁸ and R⁹ are hydrogen. In one embodiment, at least three of R⁵, R⁶, R⁷, R⁸ and R⁹ are hydrogen.

In one embodiment, at least one of R⁵, R⁶, R⁷, R⁸ and R⁹ is other than hydrogen. In a particular embodiment, at least one of R⁶, R⁷ or R⁸ is other than hydrogen.

Particular examples of R⁵, R⁶, R⁷, R⁸ and R⁹, where these are other than hydrogen include halo, trifluoromethoxy, cyano, C₂₋₈alkynyl, heterocyclyl,

a group of sub-formula (iv)

—X²—R¹⁴ (iv)
where X² is selected from O, NR¹⁶, SO₂, CON(R¹⁶), N(R¹⁶)CO, SO₂N(R¹⁶), N(R¹⁶)SO₂,
where each R¹⁶ is independently selected from hydrogen or C₁₋₆alkyl, and R¹⁴ is hydrogen, C₁₋₆ alkyl or trifluoromethyl,

or a group of sub-formula (v):

—X³—R¹⁵—Z (v)
where X³ is a direct bond or is selected from O, CON(R¹⁷), N(R¹⁷)CO, SO₂N(R¹⁷),
N(R¹⁷)SO₂, where each R¹⁷ is independently selected from hydrogen or C₁₋₆alkyl, and in particular is hydrogen,
R¹⁵ is a C₁₋₆alkylene, and
Z is cyano, or heterocyclyl which optionally bears 1 or 2 substituents, which may be the same or different, selected from halo or C₁₋₆alkyl, or Z is a group of sub-formula (vi)

—X⁴—R¹⁸ (vi)
where X⁴ is selected from O, NR¹⁹CON(R¹⁹), N(R¹⁹)CO, SO₂N(R¹⁹) or N(R¹⁹)SO₂, where each R¹⁹ is independently selected from hydrogen or C₁₋₆alkyl; and R¹⁸ is selected from hydrogen, C₁₋₆ alkyl, or heterocyclyl.

Particular examples of heterocyclic groups for R⁵, R⁶, R⁷, R⁸ and R⁹ as well as Z include saturated five or six membered rings which contain at least one nitrogen atom and optionally also one or more further heteroatoms selected from oxygen, nitrogen and sulphur. These may be linked either to the phenyl ring in the case of R⁵, R⁶, R⁷, R⁸ and R⁹ or to the group R¹⁵ in the case of Z via a carbon or nitrogen atom. In a particular embodiment, at least one of R⁵, R⁶, R⁷, R⁸ and R⁹ or Z is an N-linked heterocyclic group. Particular examples of such groups include pyrrolidine and N-morpholino.

Specific examples of groups R⁵, R⁶, R⁷, R⁸ or R⁹ where these are other than hydrogen include chloro, fluoro, methyl, methoxy, ethoxyethoxy trifluoromethoxy, ethynyl, cyano, hydroxymethyl, hydroxyethyl, cyanomethyl, amido, N-methylamido, N-(2-methoxyethyl)amido, 4-(pyridin-2-ylmethoxy), N-methylmethanesulfonamido, pyrrolidin-1-ylethoxy, morpholino, 2-morpholin-4-ylethoxy, 2-hydroxyethyl)-N-methylsulfonamido, diethylaminoethylamido, 4-methylpiperazin-1-yl)ethoxy, fluorobenzyloxy, sulfonamido, methanesulfonamido, methoxyethylsulfonamido, acetamido, N-methylacetamido, methylacetamidomethyl, methylsulfonyl and dimethylamino.

Where R⁵ and R⁶, R⁶ and R⁷, R⁷ and R⁸ or R⁸ and R⁹ are joined to form a fused ring, the ring suitably includes at least one heteroatom. In particular, a fused ring formed by R⁵ and R⁶, R⁶ and R⁷, R⁷ and R⁸ or R⁸ and R⁹ contains one or two nitrogen atoms or one nitrogen atom and one sulphur atom. Suitably the ring includes 5 ring atoms including the carbon atoms to which R⁵ and R⁶, R⁶ and R⁷, R⁷ and R⁸ or R⁸ and R⁹ are attached.

Fused rings formed by R⁵ and R⁶, R⁶ and R⁷, R⁷ and R⁸ or R⁸ and R⁹ may carry optional substituents which may be selected from those listed above for R³.

Particular examples of fused rings include formed by R⁵ and R⁶, R⁶ and R⁷, R⁷ and R⁸ or R⁸ and R⁹ and the phenyl ring to which they are attached include indolyl, indazolyl, indolone and benzothiazolyl.

In another embodiment, the invention provides a compound of formula (IC)

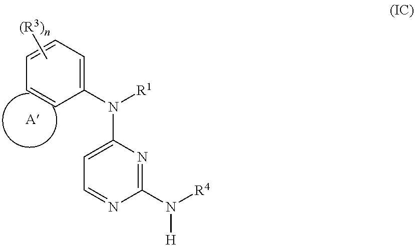

where R¹, R³, R⁴ and n are as defined herein relation to formula (I) and A′ is selected from a group —OCH₂O—, —OCF₂O—, —CH═CH—NR²⁰— or —NR²⁰—CH═CH—, —O—N═CH—, —CH═N—O—, —O—NR²⁰—CH₂—, —CH₂—NR²⁰—O—, —NR²⁰—N═CH—, —CH═N—NR²⁰—, —NR²⁰—NR²⁰—CH₂— or —CH₂—NR²⁰—NR²⁰.

In particular, A′ is a selected from —OCH₂O—, —OCF₂O—, —CH═CH—NR²⁰—, —NR²⁰—CH═CH—, —O—N═CH—, —CH═N—O—, —O—NR²⁰—CH₂—, —CH₂—NR²⁰—O—, —NR²⁰—N═CH— or —CH═N—NR²⁰—.

Particular examples of compounds of formula (IC) are compounds of formula (IB) as set out above, and these form a particular aspect of the invention.

Particular options for R¹, R³, R⁴ n and R²⁰ in formula (IC) are as set out herein in relation to formula (I). In particular, compounds of formula (IB) form a particular aspect of the invention.

Particular novel compounds of the invention include, for example, compounds of Formula (I), or pharmaceutically-acceptable salts thereof, wherein, unless otherwise stated, is each of R¹, R², R³, ring A, n or R⁴ has any of the meanings defined hereinbefore or in paragraphs (1) to (34) hereinafter:
1. Ring A is selected from: —CR²²═CR²²—CR²²═CR²²—, —N═CR²²—CR²²═CR²²—, —CR²²═N—CR²²═CR²²—, —CR²²═CR²²—N═CR²²—, —CR²²═CR²²—CR²²═N—, —N═CR²²—N═CR²²—, —CR²²═N—CR²²═N—, —N═CR²²—CR²²═N—, —N═N—CR²²═CR²²—, —CR²²═CR²²—N═N—, —CR²²═CR²²—O—, —O—CR²²═CR²²—, —CR²²═CR²²—S—, —S—CR²²═CR²²—, —CR²²H—CR²²H—O—, —O—CR²²H—CR²²H—, —CR²²H—CR²²H—S—, —S—CR²²H—CR²²H—, —O—CR²²H—O—, —O—CF₂—O—, —O—CR²²H—CR²²H—O—, —S—CR²²H—S—, —S—CR²²H—CR²²H—S—, —CR²²═CR²²—NR²⁰—, —NR²⁰—CR²²═CR²²—, —CR²²H—CR²²H—NR²⁰—, —NR²⁰—CR²²H—CR²²H—, —N═CR²²—NR²⁰—, —NR²⁰—CR²²═N—, —NR²⁰—CR²²H—NR²⁰—, —OCR²²═N—, —N═CR²²—O—, —S—CR²²═N—, —N═CR²²—S—, —O—CR²²H—NR²⁰—, —NR²⁰—CR²²H—O—, —S—CR²²H—NR²⁰—, —NR²⁰—CR²²H—S—, —O—N═CR²²—, —CR²²═N—O—, —S—N═CR²²—, —CR²²═N—S—, —O—NR²⁰—CR²²H—, —CR²²H—NR²⁰—O—, —S—NR²⁰—CR²²H—, —CR²²H—NR²⁰—S—, —NR²⁰—N═CR²²—, —CR²²═N—NR²⁰—, —NR²⁰—NR²⁰—CR²²H—, —CR²²H—NR²⁰—NR²⁰—, —N═N—NR²⁰—, or —NR²⁰—N═N—, where each R²⁰ is independently selected from hydrogen, C₁₋₄alkyl or C₁₋₄alkylcarbonyl, and where each R²² is independently selected from hydrogen, halo, cyano, hydroxy, C₁₋₄alkyl, C₁₋₄alkoxy, —S(O)_{z}—C₁₋₄alkyl (where z is 0, 1 or 2), or —NR^{a}R^{b} (where R^{a} and R^{b} are each independently selected from hydrogen, C₁₋₂alkyl, or C₁₋₂alkanoyl).
2. Ring A is selected from —N═CR²²—CR²²═CR²²—, —CR²²═N—CR²²═CR²²—, —CR²²═CR²²—N═CR²²—, —CR²²═CR²²—CR²²═N—, —CR²²═CR²²—O—, —O—CR²²═CR²²—, —O—CR²²H—O—, —O—CF₂—O—, —O—CR²²H—CR²²H—O—, —CR²²═CR²²—NR²⁰—, —NR²⁰—CR²²═CR²²—, —CR²²H—CR²²H—NR²⁰—, —NR²⁰—CR²²H—CR²²H—, —OCR²²═N—, —N═CR²²—O—, —S—CR²²═N—, —N═CR²²—S—, —NR²⁰—N═CR²²—, or —CR²²═N—NR²⁰—, where to each R²⁰ is independently selected from hydrogen, C₁₋₂alkyl or C₁₋₂alkylcarbonyl, and where each R²² is independently selected from hydrogen, halo, cyano, hydroxy, C₁₋₂alkyl, C₁₋₂alkoxy, —S(O)_{z}—C₁₋₂alkyl (where z is 0, 1 or 2), or —NR^{a}R^{b} (where R^{a} and R^{b} are each independently selected from hydrogen, C₁₋₂alkyl, or C₁₋₂alkanoyl).
3. Ring A is selected from —O—CR²²H—O—, —O—CF₂—O—, —OCR²²═N—, —N═CR²²—O—, —S—CR²²═N—, —N═CR²²—S—, —NR²⁰—N═CR²²—, or —CR²²═N—NR²⁰—, where each R²⁰ is independently selected from hydrogen, C₁₋₂alkyl or C₁₋₂alkylcarbonyl, and where each R²² is independently selected from hydrogen, halo, cyano, hydroxy, C₁₋₂alkyl, C₁₋₂alkoxy, —S(O)_{z}—C₁₋₂alkyl (where z is 0, 1 or 2), or —NR^{a}R^{b} (where R^{a} and R^{b} are each independently selected from hydrogen, C₁₋₂alkyl, or C₁₋₂alkanoyl).
4. Ring A is selected from —O—CR²²H—O—, —O—CF₂—O—, —OCR²²═N—, —N═CR²²—O—, —S—CR²²═N—, —N═CR²²—S—, —NR²⁰—N═CR²²—, or —CR²²═N—NR²⁰—, where each R²⁰ is independently selected from hydrogen, or C₁₋₂alkyl, and where each R²² is independently selected from hydrogen, halo, or methyl.
5. Ring A is selected from: —CR²²═CR²²—CR²²═CR²²—, —N═CR²²—CR²²═CR²²—, —CR²²═N—CR²²═CR²²—, —CR²²═CR²²—N═CR²²—, —CR²²═CR²²—CR²²═N—, —N═CR²²—N═CR²²—, —CR²²═N—CR²²═N—, —N═CR²²—CR²²═N—, —N═N—CR²²═CR²²—, —CR²²═CR²²—N═N—, —CR²²═CR²²—O—, —O—CR²²═CR²²—, —CR²²═CR²²—S—, —S—CR²²═CR²²—, —CR²²H—CR²²H—O—, —O—CR²²H—CR²²H—, —CR²²H—CR²²H—S—, —S—CR²²H—CR²²H—, —O—CR²²H—O—, —O—CF₂—O—, —O—CR²²H—CR²²H—O—, —S—CR²²H—S—, —S—CR²²H—CR²²H—S—, —CR²²═CR²²—NR²⁰—, —NR²⁰—CR²²═CR²²—, —CR²²H—CR²²H—NR²⁰—, —NR²⁰—CR²²H—CR²²H—, —N═CR²²—NR²⁰—, —NR²⁰—CR²²═N—, —NR²⁰—CR²²H—NR²⁰—, —OCR²²═N—, —N═CR²²—O—, —S—CR²²═N—, —N═CR²²—S—, —O—CR²²H—NR²⁰—, —NR²⁰—CR²²H—O—, —S—CR²²H—NR²⁰—, —NR²⁰—CR²²H—S—, —O—N═CR²²—, —CR²²═N—O—, —S—N═CR²²—, —CR²²═N—S—, —O—NR²⁰—CR²²H—, —CR²²H—NR²⁰—O—, —S—NR²⁰—CR²²H—, —CR²²H—NR²⁰—S—, —NR²⁰—NR²⁰—CR²²H—, —CR²²H—NR²⁰—NR²⁰—, —N═N—NR²⁰—, or —NR²⁰—N═N—, where each R²⁰ is independently selected from hydrogen, C₁₋₄alkyl or C₁₋₄alkylcarbonyl, and where each R²² is independently selected from hydrogen, halo, cyano, hydroxy, C₁₋₄alkyl, C₁₋₄alkoxy, —S(O)_{z}—C₁₋₄alkyl (where z is 0, 1 or 2), or —NR^{a}R^{b} (where R^{a} and R^{b} are each independently selected from hydrogen, C₁₋₂alkyl, or C₁₋₂alkanoyl).
6. Ring A is selected from —N═CR²²—CR²²═CR²²—, —CR²²═N—CR²²═CR²²—, —CR²²═CR²²—N═CR²²—, —CR²²═CR²²—CR²²═N—, —CR²²═CR²²—O—, —O—CR²²═CR²²—, —O—CR²²H—O—, —O—CF₂—O—, —O—CR²²H—CR²²H—O—, —CR²²═CR²²—NR²⁰—, —NR²⁰—CR²²═CR²²—, —CR²²H—CR²²H—NR²⁰—, —NR²⁰—CR²²H—CR²²H—, —OCR²²═N—, —N═CR²²—O—, —S—CR²²═N—, or —N═CR²²—S—, where each R²⁰ is independently selected from hydrogen, C₁₋₂alkyl or C₁₋₂alkylcarbonyl, and where each R²² is independently selected from hydrogen, halo, cyano, hydroxy, C₁₋₂alkyl, C₁₋₂alkoxy, —S(O)_{z}—C₁₋₂alkyl (where z is 0, 1 or 2), or —NR^{a}R^{b} (where R^{a} and R^{b} are each independently selected from hydrogen, C₁₋₂alkyl, or C₁₋₂alkanoyl).
7. Ring A is selected from —O—CR²²H—O—, —O—CF₂—O—, —OCR²²═N—, —N═CR²²—O—, —S—CR²²═N—, or —N═CR²²—S—, where each R²⁰ is independently selected from hydrogen, C₁₋₂alkyl or C₁₋₂alkylcarbonyl, and where each R²² is independently selected from hydrogen, halo, cyano, hydroxy, C₁₋₂alkyl, C₁₋₂alkoxy, —S(O)_{z}—C₁₋₂alkyl (where z is 0, 1 or 2), or —NR^{a}R^{b} (where R^{a} and R^{b} are each independently selected from hydrogen, C₁₋₂alkyl, or C₁₋₂alkanoyl).
8. Ring A is selected from —O—CR²²H—O—, —O—CF₂—O—, —OCR²²═N—, —N═CR²²—O—, —S—CR²²═N—, or —N═CR²²—S—, where each R²⁰ is independently selected from hydrogen, or C₁₋₂alkyl, and where each R²² is independently selected from hydrogen, halo, or methyl.
9. R¹ is hydrogen or a C₁₋₄alkyl group which is optionally substituted with one or more substituents selected from cyano, —OR², —NR^{2a}R^{2b}, —C(O)NR^{2a}R^{2b}, or —N(R^{2a})C(O)R², halo or haloC₁₋₄alkyl (such as trifluoromethyl), where R², R^{2a} and R^{2b} are selected from hydrogen or C₁₋₄alkyl;
10. R¹ is hydrogen or a C₁₋₂alkyl group, which is optionally substituted with one or more substituents selected from cyano, —OR², —NR^{2a}R^{2b}, —C(O)NR^{2a}R^{2b}, or —N(R^{2a})C(O)R², halo or haloC₁₋₄alkyl (such as trifluoromethyl), where R², R^{2a} and R^{2b} are selected from hydrogen or C₁₋₄alkyl;
11. R¹ is hydrogen or a C₁₋₂alkyl group, which is optionally substituted with one or more substituents selected from cyano, —OR², —NR^{2a}R^{2b}, where R², R^{2a} and R^{2b} are selected from hydrogen or C₁₋₂alkyl;
12. R¹ is hydrogen or a C₁₋₂alkyl group;
13. R¹ is hydrogen;
14. R¹ is a C₁₋₂alkyl group, which is optionally substituted with one or more substituents selected from cyano, —OR², —NR^{2a}R^{2b}, where R², R^{2a} and R^{2b} are selected from hydrogen or C₁₋₂alkyl;
15. R¹ is a C₁₋₂alkyl group;
16. R¹ is methyl;
17. n is 0, 1, or 2;
18. n is 0 or 1;
19. n is 0;
20. n is 1;
21. each group R³ present is independently selected from halo, trifluoromethyl, cyano, nitro or a group of sub-formula (i):

—X¹—R¹¹ (i)
where X¹ is selected from a direct bond or O, S, SO, SO₂, OSO₂, NR¹³, CO, CH(OR¹³), CONR¹³, N(R¹³)CO, SO₂N(R¹³), N(R¹³)SO₂, C(R¹³)₂O, C(R¹³)₂S, C(R¹³)₂N(R¹³) and N(R¹³)C(R¹³)₂, wherein R¹³ is hydrogen or C₁₋₆alkyl and R¹¹ is selected from hydrogen, or C₁₋₆ alkyl, which may be optionally substituted with one or more groups selected from halo, trifluoromethyl, cyano, nitro, hydroxy, amino, carboxy, carbamoyl, and C₁₋₆alkoxy;
22. each group R³ present is independently selected from halo, trifluoromethyl, cyano, nitro or a group of sub-formula (i):

—X¹—R¹¹ (i)
where X¹ is selected from a direct bond or O, NR¹³, CO, CONR¹³, N(R¹³)CO, wherein R¹³ is hydrogen or C₁₋₄alkyl and R¹¹ is selected from hydrogen or C₁₋₄alkyl, which may be optionally substituted with one or more groups selected from halo, cyano, or C₁₋₄alkoxy;
23. each group R³ present is independently selected from halo, trifluoromethyl, cyano, nitro or a group of sub-formula (i):

—X¹—R¹¹ (i)
where X¹ is selected from a direct bond or O, CONR¹³, wherein R¹³ is hydrogen or C₁₋₆alkyl and R¹¹ is selected from hydrogen or C₁₋₄alkyl, which may be optionally substituted with one or more C₁₋₂alkoxy groups;
24. each group R³ present is independently selected from halo or a group of sub-formula (i):

—X¹—R¹¹ (i)
where X¹ is selected from a direct bond or O, CONR¹³, wherein R¹³ is hydrogen or C₁₋₆alkyl and R¹¹ is selected from hydrogen, C₁₋₂alkyl, any of which may be optionally substituted with one or more C₁₋₂alkoxy groups;
25. each group R³ present is independently selected from fluoro, chloro, cyano, —CONH₂, or C₁₋₂alkyl optionally substituted by C₁₋₂alkoxy;
26. R⁴ is a group of sub-formula (iii)

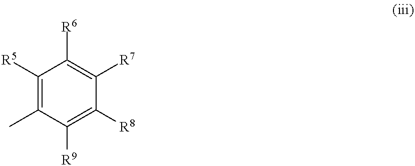

where R⁵, R⁶, R⁷, R⁸ and R⁹ are independently selected from:
(a) hydrogen, halo, trifluoromethyl, trifluoromethoxy, cyano, nitro, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by halo, hydroxy, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(b) a group of sub-formula (iv);

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, —N(R¹⁶)C(O)N(R¹⁶)—, —N(R¹⁶)C(O)O—, SON(R¹⁶), N(R¹⁶)SO, SO₂N(R¹⁶), N(R¹⁶)SO₂, C(R¹⁶)₂O, C(R¹⁶)₂S and N(R¹⁶)C(R¹⁶)₂, where each R¹⁶ is independently selected from to hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, aryl-C₁₋₆alkyl, or a 4- to 8-membered mono or bicyclic heterocyclyl ring (including 5 or 6 membered heteroaryl rings) or 4- to 8-membered mono or bicyclic heterocyclyl-C₁₋₆alkyl groups (including 5 or 6 membered heteroaryl-C₁₋₆alkyl groups) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v):

—X³—R¹⁵—Z (v)
where X³ is a direct bond or is selected from O, NR¹⁷, S, SO, SO₂, OSO₂, CO, C(O)O, OC(O), CH(OR¹⁷), CON(R¹⁷), N(R¹⁷)CO, —N(R¹⁷)C(O)N(R¹⁷)—, —N(R¹⁷)C(O)O—, SO₂N(R¹⁷), N(R¹⁷)SO₂, C(R¹⁷)₂O, C(R¹⁷)₂S and N(R¹⁷)C(R¹⁷)₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, C₃₋₁₂ carbocyclyl or heterocyclyl (including heteroaryl) which optionally bears 1 or 2 substituents, which may be the same or different, selected from halo, C₁₋₆alkyl, C₂₋₈alkenyl, C₂₋₈alkynyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹, S, SO, SO₂, OSO₂, CO, C(O)O, OC(O), CH(OR¹⁹), CON(R¹⁹), N(R¹⁹)CO, SO₂N(R¹⁹), —N(R¹⁹)C(O)N(R¹⁹)—, —N(R¹⁹)C(O)O—N(R¹⁹)SO₂, C(R¹⁹)₂O, C(R¹⁹)₂S and N(R¹⁹)C(R¹⁹)₂, where each R¹⁹ is independently selected from hydrogen or C₁₋₆alkyl; and R¹⁸ is selected from hydrogen, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl) which optionally bears 1 or 2 substituents, which may be the same or different, selected from halo, C₁₋₆alkyl, C₂₋₈alkenyl, C₂₋₈alkynyl and C₁₋₆alkoxy, and wherein any heterocyclyl group within R¹⁸ optionally bears 1 or 2 oxo substituents; or
(d) R⁵ and R⁶, R⁶ and R⁷, R⁷ and R⁸ or R⁸ and R⁹ are joined together to form a fused 5-, 6- or 7-membered saturated or unsaturated ring, which is optionally substituted on any available carbon atom by halo, C₁₋₆alkyl, hydroxyC₁₋₆alkyl, amino, N—C₁₋₆alkylamino, or N,N-diC₁₋₆alkylamino, and which may contain one or more heteroatoms selected from oxygen, sulphur or nitrogen, where sulphur atoms may be optionally oxidised to a sulphur oxide, where any CH₂ groups may be substituted by a C(O) group, and where nitrogen atoms, depending upon valency considerations, may be substituted by a group R²¹, where R²¹ is selected from hydrogen, C₁₋₆alkyl or C₁₋₆alkylcarbonyl;
27. R⁴ is a group of sub-formula (iiia)

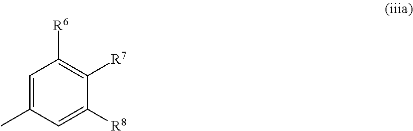

where R⁶, R⁷, and R⁸ are independently selected from:
(a) hydrogen, halo, trifluoromethyl, trifluoromethoxy, cyano, nitro, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by halo, hydroxy, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from is hydrogen, C₁₋₆alkyl or C₁₋₆alkylcarbonyl, and where any sulphur atoms may be optionally oxidised to a sulphur oxide;
(b) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, —N(R¹⁶)C(O)N(R¹⁶)—, —N(R¹⁶)C(O)O—, SON(R¹⁶), N(R¹⁶)SO, SO₂N(R¹⁶), N(R¹⁶)SO₂, C(R¹⁶)₂O, C(R¹⁶)₂S and N(R¹⁶)C(R¹⁶)₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, aryl-C₁₋₆alkyl, or a 4- to 8-membered mono or bicyclic heterocyclyl ring (including 5 or 6 membered heteroaryl rings) or 4- to 8-membered mono or bicyclic heterocyclyl-C₁₋₆alkyl groups (including 5 or 6 membered heteroaryl-C₁₋₆alkyl groups) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by oxo, halo, cyano, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v):

—X³—R¹⁵—Z (v)
where X³ is a direct bond or is selected from O, NR¹⁷, S, SO, SO₂, OSO₂, CO, C(O)O, OC(O), CH(OR¹⁷), CON(R¹⁷), N(R¹⁷)CO, —N(R¹⁷)C(O)N(R¹⁷)—, —N(R¹⁷)C(O)O—, SO₂N(R¹⁷), N(R¹⁷)SO₂, C(R¹⁷)₂O, C(R¹⁷)₂S and N(R¹⁷)C(R¹⁷)₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, C₃₋₁₂ carbocyclyl or heterocyclyl (including heteroaryl) which optionally bears 1 or 2 substituents, which may be the same or different, selected from halo, C₁₋₆alkyl, C₂₋₈alkenyl, C₂₋₈alkynyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹, S, SO, SO₂, OSO₂, CO, C(O)O, OC(O), CH(OR¹⁹), CON(R¹⁹), N(R¹⁹)CO, SO₂N(R¹⁹), —N(R¹⁹)C(O)N(R¹⁹)—, —N(R¹⁹)C(O)O—N(R¹⁹)SO₂, C(R¹⁹)₂O, C(R¹⁹)₂S and N(R¹⁹)C(R¹⁹)₂, where each R¹⁹ is independently selected from hydrogen or C₁₋₆alkyl; and R¹⁸ is selected from hydrogen, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl) which optionally bears 1 or 2 substituents, which may be the same or different, selected from halo, C₁₋₆alkyl, C₂₋₈alkenyl, C₂₋₈alkynyl and C₁₋₆alkoxy, and wherein any heterocyclyl group within R¹⁸ optionally bears 1 or 2 oxo substituents; or
(d) R⁶ and R⁷, or R⁷ and R⁸ are joined together to form a fused 5-, 6- or 7-membered saturated or unsaturated ring, which is optionally substituted on any available carbon atom by halo, C₁₋₆alkyl, hydroxyC₁₋₆alkyl, amino, N—C₁₋₆alkylamino, or N,N-diC₁₋₆alkylamino, and which may contain one or more heteroatoms selected from oxygen, sulphur or nitrogen, where sulphur atoms may be optionally oxidised to a sulphur oxide, where any CH₂ groups may be substituted by a C(O) group, and where nitrogen atoms, depending upon valency considerations, may be substituted by a group R²¹, where R²¹ is selected from hydrogen, C₁₋₆alkyl or C₁₋₆alkylcarbonyl.
28. R⁴ is a group of sub-formula (iiia)

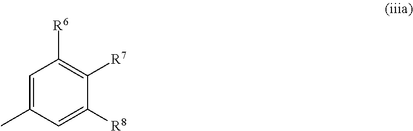

where R⁶, R⁷, and R⁸ are independently selected from:
(a) hydrogen, halo, trifluoromethyl, trifluoromethoxy, cyano, nitro, C₁₋₆ alkyl, C₂₋₈alkenyl, C₂₋₈alkynyl, aryl, heterocyclyl (including heteroaryl), and wherein any aryl or heterocyclyl (including heteroaryl) groups are optionally substituted on any available carbon atoms by halo, hydroxy, cyano, amino, C₁₋₆alkyl, hydroxyC₁₋₆alkyl, C₁₋₆alkoxy, and any nitrogen atoms present in the heterocyclyl moieties may, depending upon valency considerations, be substituted by a group selected from hydrogen, C₁₋₆alkyl or C₁₋₆alkylcarbonyl;
(b) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, SON(R¹⁶), N(R¹⁶)SO, SO₂N(R¹⁶), and N(R¹⁶)SO₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, or a 4- to 8-membered mono or bicyclic heterocyclyl ring (including 5 or 6 membered heteroaryl rings) and wherein any aryl, C₃₋₁₂ carbocyclyl, heterocyclyl (including heteroaryl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v):

—X³—R¹⁵—Z (v)
where X³ is a direct bond or is selected from O, NR¹⁷, S, SO, SO₂, OSO₂, CO, C(O)O, OC(O), CON(R¹⁷), N(R¹⁷)CO, SO₂N(R¹⁷), and N(R¹⁷)SO₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, or heterocyclyl (including heteroaryl) which optionally bears 1 or 2 substituents, which may be the same or different, selected from halo, C₁₋₆alkyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹, S, SO, SO₂, OSO₂, CO, C(O)O, OC(O), CON(R¹⁹), N(R¹⁹)CO, SO₂N(R¹⁹), and N(R¹⁹)SO₂, where each R¹⁹ is independently selected from hydrogen or C₁₋₆alkyl; and R¹⁸ is selected from hydrogen, C₁₋₆ alkyl, aryl, or heterocyclyl (including heteroaryl) which optionally bears 1 or 2 substituents, which may be the same or different, selected from halo, C₁₋₆alkyl, and C₁₋₆alkoxy, and wherein any heterocyclyl group within R¹⁸ optionally bears 1 or 2 oxo substituents;
29. R⁴ is a group of sub-formula (iiib)

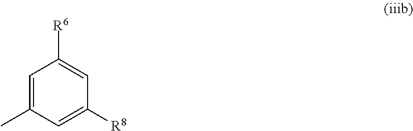

wherein at least one of R⁶ and R⁸ is a 5, 6, or 7-membered heterocyclic ring which is nitrogen-linked and the other is independently selected from:
(a) hydrogen, halo, trifluoromethyl, trifluoromethoxy, cyano, nitro, C₁₋₆ alkyl, C₂₋₈alkenyl, C₂₋₈alkynyl, aryl, heterocyclyl (including heteroaryl), and wherein any aryl or heterocyclyl (including heteroaryl) groups are optionally substituted on any available carbon atoms by halo, hydroxy, cyano, amino, C₁₋₆alkyl, hydroxyC₁₋₆alkyl, C₁₋₆alkoxy, and any nitrogen atoms present in the heterocyclyl moieties may, depending upon valency considerations, be substituted by a group selected from hydrogen, C₁₋₆alkyl or C₁₋₆alkylcarbonyl;
(b) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, SON(R¹⁶), N(R¹⁶)SO, SO₂N(R¹⁶), and N(R¹⁶)SO₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, or a 4- to 8-membered mono or bicyclic heterocyclyl ring (including 5 or 6 membered heteroaryl rings) and wherein any aryl, C₃₋₁₂ carbocyclyl, heterocyclyl (including heteroaryl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v):

—X³—R¹⁵—Z (v)
where X³ is a direct bond or is selected from O, NR¹⁷, S, SO, SO₂, OSO₂, CO, C(O)O, OC(O), CON(R¹⁷), N(R¹⁷)CO, SO₂N(R¹⁷), and N(R¹⁷)SO₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, or heterocyclyl (including heteroaryl) which optionally bears 1 or 2 substituents, which may be the same or different, selected from halo, C₁₋₆alkyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹, S, SO, SO₂, OSO₂, CO, C(O)O, OC(O), CON(R¹⁹), N(R¹⁹)CO, SO₂N(R¹⁹), and N(R¹⁹)SO₂, where each R¹⁹ is independently selected from hydrogen or C₁₋₆alkyl; and R¹⁸ is selected from hydrogen, C₁₋₆ alkyl, aryl, or heterocyclyl (including heteroaryl) which optionally bears 1 or 2 substituents, which may be the same or different, selected from halo, C₁₋₆alkyl, and C₁₋₆alkoxy, and wherein any heterocyclyl group within R¹⁸ optionally bears 1 or 2 oxo substituents;
30. R⁴ is a group of sub-formula (iiib)

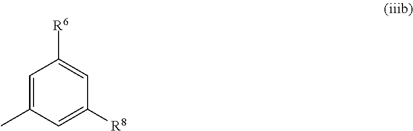

wherein at least one of R⁶ and R⁸ is a 5 or 6-membered nitrogen-linked heterocyclic ring and the other is independently selected from:
(a) hydrogen, halo, trifluoromethyl, trifluoromethoxy, cyano, nitro, C₁₋₆ alkyl, C₂₋₈alkenyl, C₂₋₈alkynyl, aryl, heterocyclyl (including heteroaryl), and wherein any aryl or heterocyclyl (including heteroaryl) groups are optionally substituted on any available carbon atoms by halo, hydroxy, cyano, amino, C₁₋₆alkyl, hydroxyC₁₋₆alkyl, C₁₋₆alkoxy, and any nitrogen atoms present in the heterocyclyl moieties may, depending upon valency considerations, be substituted by a group selected from hydrogen, C₁₋₆alkyl or C₁₋₆alkylcarbonyl;
(b) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, SON(R¹⁶), N(R¹⁶)SO, SO₂N(R¹⁶), and N(R¹⁶)SO₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃-₁₂ carbocyclyl, or a 4- to 8-membered mono or bicyclic heterocyclyl ring (including 5 or 6 membered heteroaryl rings) and wherein any aryl, C₃₋₁₂ carbocyclyl, heterocyclyl (including heteroaryl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v) is

—X³—R¹⁵—Z (v)
where X³ is a direct bond or is selected from O, NR¹⁷, S, SO, SO₂, OSO₂, CO, C(O)O, OC(O), CON(R¹⁷), N(R¹⁷)CO, SO₂N(R¹⁷), and N(R¹⁷)SO₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, or heterocyclyl (including heteroaryl) which optionally bears 1 or 2 substituents, which may be the same or different, selected from halo, C₁₋₆alkyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹, S, SO, SO₂, OSO₂, CO, C(O)O, OC(O), CON(R¹⁹), N(R¹⁹)CO, SO₂N(R¹⁹), and N(R¹⁹)SO₂, where each R¹⁹ is independently selected from hydrogen or C₁₋₆alkyl; and R¹⁸ is selected from hydrogen, C₁₋₆ alkyl, aryl, or heterocyclyl (including heteroaryl) which optionally bears 1 or 2 substituents, which may be the same or different, selected from halo, C₁₋₆alkyl, and C₁₋₆alkoxy, and wherein any heterocyclyl group within R¹⁸ optionally bears 1 or 2 oxo substituents;
31. R⁴ is a group of sub-formula (iiib)

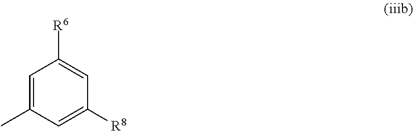

wherein at least one of R⁶ and R⁸ is morpholin-4-yl and the other is independently selected from:
(a) hydrogen, halo, trifluoromethyl, trifluoromethoxy, cyano, nitro, C₁₋₆ alkyl, C₂₋₈alkenyl, C₂₋₈alkynyl, aryl, heterocyclyl (including heteroaryl), and wherein any aryl or heterocyclyl (including heteroaryl) groups are optionally substituted on any available carbon atoms by halo, hydroxy, cyano, amino, C₁₋₆alkyl, hydroxyC₁₋₆alkyl, C₁₋₆alkoxy, and any nitrogen atoms present in the heterocyclyl moieties may, depending upon valency considerations, be substituted by a group selected from hydrogen, C₁₋₆alkyl or C₁₋₆alkylcarbonyl;
(b) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, SON(R¹⁶), N(R¹⁶)SO, SO₂NR¹⁶), and N(R¹⁶)SO₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, or a 4- to 8-membered mono or bicyclic heterocyclyl ring (including 5 or 6 membered heteroaryl rings) and wherein any aryl, C₃₋₁₂ carbocyclyl, heterocyclyl (including heteroaryl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v):

—X³—R¹⁵—Z (v)
where X³ is a direct bond or is selected from O, NR¹⁷, S, SO, SO₂, OSO₂, CO, C(O)O, OC(O), CON(R¹⁷), N(R¹⁷)CO, SO₂N(R¹⁷), and N(R¹⁷)SO₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, or heterocyclyl (including heteroaryl) which optionally bears 1 or 2 substituents, which may be the same or different, selected from halo, C₁₋₆alkyl and C₁₋₆alkoxy and wherein any heterocyclyl group within Z optionally bears 1 or 2 oxo substituents, or Z is a group of sub-formula (vi)

—X⁴—R¹⁸ (vi)
where X⁴ is selected from O, NR¹⁹, S, SO, SO₂, OSO₂, CO, C(O)O, OC(O), CON(R¹⁹), N(R¹⁹)CO, SO₂N(R¹⁹), and N(R¹⁹)SO₂, where each R¹⁹ is independently selected from hydrogen or C₁₋₆alkyl; and R¹⁸ is selected from hydrogen, C₁₋₆ alkyl, aryl, or heterocyclyl (including heteroaryl) which optionally bears 1 or 2 substituents, which may be the same or different, selected from halo, C₁₋₆alkyl, and C₁₋₆alkoxy, and wherein any heterocyclyl group within R¹⁸ optionally bears 1 or 2 oxo substituents;
32. R⁴ is a group of sub-formula (iiib)

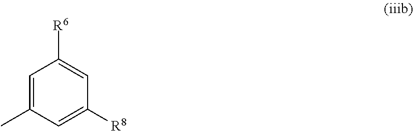

wherein at least one of R⁶ and R⁸ is morpholin-4yl and the other is independently selected from:
(a) hydrogen, halo, trifluoromethyl, cyano, C₁₋₄ alkyl, phenyl, a 5 or 6-membered heterocyclyl (including heteroaryl) comprising one or more heteroatoms selected from N, O or S,
   and wherein any C₁₋₄ alkyl, aryl or heterocyclyl (including heteroaryl) groups are optionally substituted on any available carbon atoms by halo, hydroxy, cyano, amino, C₁₋₄alkyl, hydroxyC₁₋₄alkyl, C₁₋₄alkoxy, and any nitrogen atoms present in the heterocyclyl moieties may, depending upon valency considerations, be substituted by a group selected from hydrogen, C₁₋₄alkyl or C₁₋₄alkylcarbonyl; or
(b) a group of sub-formula (iv):

—X²—R¹⁴ (iv)
where X² is selected from O, NR¹⁶, S, SO, SO₂, OSO₂, CO, CON(R¹⁶), N(R¹⁶)CO, SON(R¹⁶), N(R¹⁶)SO, SO₂N(R¹⁶), and N(R¹⁶)SO₂, where each R¹⁶ is independently selected from hydrogen or C₁₋₄alkyl, R¹⁴ is hydrogen, or C₁₋₄alkyl;
33. R⁴ is a group of sub-formula (iiib)

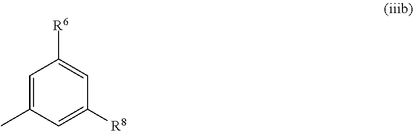

wherein both R⁶ and R⁸ are 5 or 6-membered nitrogen-linked heterocyclylic rings;
34. R⁴ is a group of sub-formula (iiib)

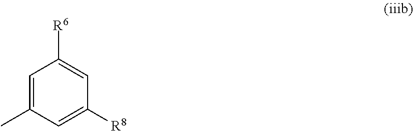

wherein both R⁶ and R⁸ are morpholin-4-yl.

In a particular group of compounds of the invention, R¹ is hydrogen or an alkyl group as defined in any one of paragraphs (9) to (12) above (particularly methyl) and ring A, R³, n, and R⁴ have any one of the definitions set out herein.

In a further particular group of compounds of the invention, R¹ is an alkyl group as defined in any one of paragraphs (14) to (16) above, particularly a methyl group, and ring A, R³, n, and R⁴ have any one of the definitions set out herein.

In a further group of compounds of the invention, R⁴ is a sub-group of formula (iiib) as defined in any one of paragraphs (29) to (34) above, and particularly a sub-group of formula (iiib) as defined in any one of paragraphs (33) to (34) above, and ring A, R¹, R³, and n have any one of the definitions set out herein.

In a further group of compounds of the invention:

R¹ is an alkyl group as defined in any one of paragraphs (14) to (16) above, particularly a methyl group,

R⁴ is a sub-group of formula (iiib) as defined in any one of paragraphs (29) to (34) above, and particularly a sub-group of formula (iiib) as defined in any one of paragraphs (33) to (34) above,

and ring A, R¹, R³, and n have any one of the definitions set out herein.

The compounds of formula I described the first aspect of the invention above are all subject to the proviso that if Ring A, together with the phenyl ring to which it is attached, form an indazol-4-yl group, then R¹ is not hydrogen. Suitably, the compounds of formula (I) defined in the second aspect of the invention are also subject to this proviso.

This proviso excludes compounds of the structural formula shown below:

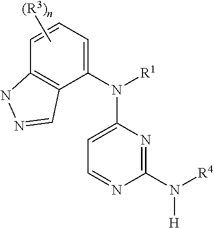

in which R¹ is hydrogen.

A particular group of compounds of formula I are subject to the proviso that if Ring A, together with the phenyl ring to which it is attached, form an indazol-4-yl group, then R¹ is a C₁₋₆alkyl group, particularly a C₁₋₂alkyl group, and most particularly methyl.

A further group of compounds of formula I are subject to the proviso that, if Ring A together with the phenyl ring to which it is attached, form an indazolyl group, then R¹ is a C₁₋₆alkyl group, particularly a C₁₋₂alkyl group and most particularly methyl.

A particular group of compounds of formula I are subject to the proviso that, if Ring A, together with the phenyl ring to which it is attached, form an indazol-4-yl group, to then R¹ is a C₁₋₆alkyl group, particularly a C₁₋₂alkyl group, and most particularly methyl, and R⁴ is a sub-group of formula (iiib) as defined in any one of paragraphs (29) to (34) above, and in particular a sub-group of formula (iiib) as defined in any one of paragraphs (33) to (34) above.

A particular group of compounds of the invention have the general structural formula (ID) shown below

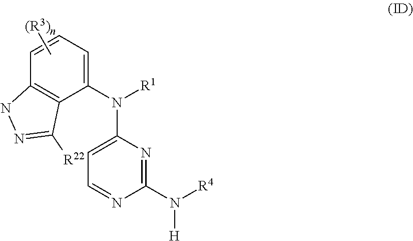

wherein R¹ is a C₁₋₆alkyl group, which is optionally substituted with one or more substituents selected from cyano, —OR², —NR^{2a}R^{2b}, where R², R^{2a} and R^{2b} are selected from hydrogen or C₁₋₂alkyl;

and R³, n, R²², and R⁴ have any one of the definitions set out herein.

In a particular group of compound of formula (ID),
R¹ is as defined in any one of paragraphs (14) to (16) above,
R²² is as defined in any one of paragraphs (1) to (8) above,
R³, if present, is as defined in any one of paragraphs (21) to (25) above,
n is as defined in any one of paragraphs (17) to (20) above, and
R⁴ is as defined in any one of paragraphs (26) to (34) above.

In compounds of formula (ID), R¹ is suitably an alkyl group as defined in any one of paragraphs (14) to (16) above. In particular compounds of formula (ID), R¹ is methyl.

In compounds of formula (ID), n is suitably 0 or 1, particularly 0.

In compounds of formula (ID), R²² is suitably hydrogen, halo, or C₁₋₂alkyl, and is especially hydrogen, methyl or chloro.

In compounds of formula (ID), R⁴ is suitably a phenyl group as defined in any one of paragraphs (26) to (34) above, and particularly a phenyl group as defined in any one of paragraphs (29) to (34) above, and most particularly a phenyl group as defined in either of paragraphs (33) or (34) above.

In a particular sub-group of compounds of formula (ID):
R¹ is an alkyl group as defined in any one of paragraphs (14) to (16) above;
n is 0;
R²² is hydrogen, halo, or C₁₋₂alkyl; and
R⁴ is a phenyl group as defined in any one of paragraphs (29) to (34) above.

In a more particular sub-group of compounds of formula (ID):
R¹ is methyl;
n is 0;
R²² is hydrogen, methyl or chloro; and
R⁴ is a phenyl group as defined in either of paragraphs (33) or (34) above.

A further particular group of compounds of the invention have the general structural formula (IE) shown below

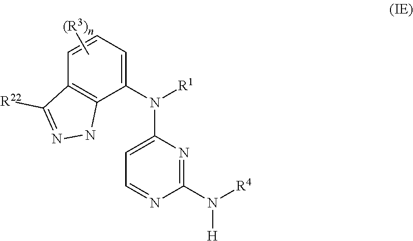

wherein R¹, R²², R³, n, and R⁴ have any of the definitions set out herein.

In a particular group of compound of formula (IE),
R¹ is as defined in any one of paragraphs (9) to (16) above,
R²² is as defined in any one of paragraphs (1) to (8) above,
R³, if present, is as defined in any one of paragraphs (21) to (25) above,
n is as defined in any one of paragraphs (17) to (20) above, and
R⁴ is as defined in any one of paragraphs (26) to (34) above.

In compounds of formula (IE), R¹ is suitably hydrogen or C₁₋₂alkyl, particularly methyl. In a particular group of compounds of formula (IE), R¹ is methyl.

In compounds of formula (IE), n is suitably 0 or 1, particularly 0.

In compounds of formula (IE), R²² is suitably hydrogen, halo, or C₁₋₂alkyl, and is especially hydrogen, methyl or chloro.

In compounds of formula (IE), R⁴ is suitably a phenyl group as defined in any one of paragraphs (26) to (34) above, and particularly a phenyl group as defined in any one of paragraphs (29) to (34) above, and most particularly a phenyl group as defined in either of paragraphs (33) or (34) above.

In a particular sub-group of compounds of formula (IE):
R¹ is hydrogen or an alkyl group as defined in any one of paragraphs (14) to (16) above;
n is 0;
R²² is hydrogen, halo, or C₁₋₂alkyl; and
R⁴ is a phenyl group as defined in any one of paragraphs (29) to (34) above.

In a more particular sub-group of compounds of formula (IE):
R¹ is methyl;
n is 0;
R²² is hydrogen, methyl or chloro; and
R⁴ is a phenyl group as defined in either of paragraphs (33) or (34) above.

A further particular group of compounds of the invention have the general structural formula (IF) shown below

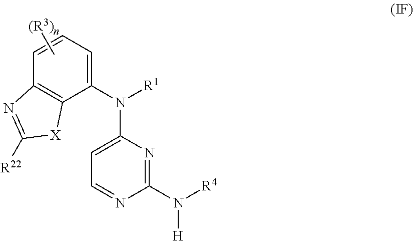

wherein R¹, R²², R³, n, and R⁴ have any of the definitions set out herein.

In a particular group of compound of formula (IF),
R¹ is as defined in any one of paragraphs (9) to (16) above,
R²² is as defined in any one of paragraphs (1) to (8) above,
R³, if present, is as defined in any one of paragraphs (21) to (25) above,
n is as defined in any one of paragraphs (17) to (20) above, and
R⁴ is as defined in any one of paragraphs (26) to (34) above.

In compounds of formula (IF), R¹ is suitably hydrogen or C₁₋₂alkyl, particularly methyl. In a particular group of compounds of formula (IE), R¹ is methyl.

In compounds of formula (IF), n is suitably 0 or 1, particularly 0.

In compounds of formula (IF), R²² is suitably hydrogen, halo, or C₁₋₂alkyl, and is especially hydrogen, methyl or chloro.

In compounds of formula (IF), R⁴ is suitably a phenyl group as defined in any one of paragraphs (26) to (34) above, and particularly a phenyl group as defined in any one of paragraphs (29) to (34) above, and most particularly a phenyl group as defined in either of paragraphs (33) or (34) above.

In a particular sub-group of compounds of formula (IF):
R¹ is hydrogen or an alkyl group as defined in any one of paragraphs (14) to (16) above;
n is 0;
R²² is hydrogen, halo, or C₁₋₂alkyl; and
R⁴ is a phenyl group as defined in any one of paragraphs (29) to (34) above.

In a more particular sub-group of compounds of formula (IF):
R¹ is methyl;
n is 0;
R²² is hydrogen, methyl or chloro; and
R⁴ is a phenyl group as defined in either of paragraphs (33) or (34) above.

A further particular group of compounds of the invention have the general structural formula (IG) shown below

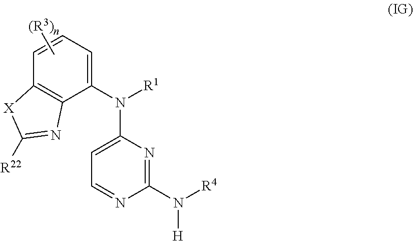

wherein R¹, R²², R³, n, and R⁴ have any of the definitions set out herein.

In a particular group of compound of formula (IG),
R¹ is as defined in any one of paragraphs (9) to (16) above,
R²² as defined in any one of paragraphs (1) to (8) above,
R³, if present, is as defined in any one of paragraphs (21) to (25) above,
n is as defined in any one of paragraphs (17) to (20) above, and
R⁴ is as defined in any one of paragraphs (26) to (34) above.

In compounds of formula (IG), R¹ is suitably hydrogen or C₁₋₂alkyl, particularly methyl. In a particular group of compounds of formula (IE), R¹ is methyl.

In compounds of formula (IG), n is suitably 0 or 1, particularly 0.

In compounds of formula (IG), R²² is suitably hydrogen, halo, or C₁₋₂alkyl, and is especially hydrogen, methyl or chloro.

In compounds of formula (IG), R⁴ is suitably a phenyl group as defined in any one of paragraphs (26) to (34) above, and particularly a phenyl group as defined in any one of paragraphs (29) to (34) above, and most particularly a phenyl group as defined in either of paragraphs (33) or (34) above.

In a particular sub-group of compounds of formula (IG):
R¹ is hydrogen or an alkyl group as defined in any one of paragraphs (14) to (16) above;
n is 0;
R²² is hydrogen, halo, or C₁₋₂alkyl; and
R⁴ is a phenyl group as defined in any one of paragraphs (29) to (34) above.

In a more particular sub-group of compounds of formula (IG):
R¹ is methyl;
n is 0;
R²² is hydrogen, methyl or chloro; and
R⁴ is a phenyl group as defined in either of paragraphs (33) or (34) above.

Particular compounds of the invention include any one of the following:
N˜4˜-(5-Chloro-1,3-benzodioxol-4-yl)-N˜2˜-(3,4,5-trimethoxyphenyl)pyrimidine-2,4-diamine;
N˜4˜-(5-chloro-1,3-benzodioxol-4-yl)-N˜2˜-(2-chlorophenyl)pyrimidine-2,4-diamine;
N˜4˜-(5-chloro-1,3-benzodioxol-4-yl)-N˜2˜-1H-indazol-6-ylpyrimidine-2,4-diamine;
N˜4˜-(5-chloro-1,3-benzodioxol-4-yl)-N˜2˜-phenylpyrimidine-2,4-diamine;
N˜4˜-(5-chloro-1,3-benzodioxol-4-yl)-N˜2˜-(2-fluorophenyl)pyrimidine-2,4-diamine;
N˜4˜-(5-chloro-1,3-benzodioxol-4-yl)-N˜2˜-(3-fluorophenyl)pyrimidine-2,4-diamine;
N˜4˜-(5-chloro-1,3-benzodioxol-4-yl)-N˜2˜-(4-fluorophenyl)pyrimidine-2,4-diamine;
N˜4˜-(5-chloro-1,3-benzodioxol-4-yl)-N˜2˜-(3-ethynylphenyl)pyrimidine-2,4-diamine;
3-({4-[(5-chloro-1,3-benzodioxol-4-yl)amino]pyrimidin-2-yl}amino)benzonitrile;
4-({4-[(5-chloro-1,3-benzodioxol-4-yl)amino]pyrimidin-2-yl}amino)benzonitrile;
[3-({4-[(5-chloro-1,3-benzodioxol-4-yl)amino]pyrimidin-2-yl}amino)phenyl]methanol;
N˜4˜-(5-chloro-1,3-benzodioxol-4-yl)-N˜2˜-(4-methoxyphenyl)pyrimidine-2,4-diamine;
N˜4˜-(5-chloro-1,3-benzodioxol-4-yl)-N˜2˜-(3-chlorophenyl)pyrimidine-2,4-diamine;
N˜4˜-(5-chloro-1,3-benzodioxol-4-yl)-N˜2˜-(4-chlorophenyl)pyrimidine-2,4-diamine;
N˜4˜-(5-chloro-1,3-benzodioxol-4-yl)-N˜2˜-(2,4-difluorophenyl)pyrimidine-2,4-diamine;
N˜4˜-(5-chloro-1,3-benzodioxol-4-yl)-N˜2˜-(3,5-difluorophenyl)pyrimidine-2,4-diamine;
N˜4˜-(5-chloro-1,3-benzodioxol-4-yl)-N˜2˜-1H-indol-5-ylpyrimidine-2,4-diamine;
[4-({4-[(5-chloro-1,3-benzodioxol-4-yl)amino]pyrimidin-2-yl}amino)phenyl]acetonitrile;
N˜4˜-(5-chloro-1,3-benzodioxol-4-yl)-N˜2˜-1H-indol-4-ylpyrimidine-2,4-diamine;
3-({4-[(5-chloro-1,3-benzodioxol-4-yl)amino]pyrimidin-2-yl}amino)benzamide;
4-({4-[(5-chloro-1,3-benzodioxol-4-yl)amino]pyrimidin-2-yl}amino)benzamide;
N˜4˜-(5-chloro-1,3-benzodioxol-4-yl)-N˜2˜-1H-indol-6-ylpyrimidine-2,4-diamine;
3-({4-{(5-chloro-1,3-benzodioxol-4-yl)amino]pyrimidin-2-yl}amino)-N-(2-methoxyethyl)benzamide;
N˜4˜-(5-chloro-1,3-benzodioxol-4-yl)-N˜2˜-[4-(pyridin-2-ylmethoxy)phenyl]pyrimidine-2,4-diamine;
1-[4-({4-[(5-chloro-1,3-benzodioxol-4-yl)amino]pyrimidin-2-yl}amino)phenyl]-N-methylmethanesulfonamide;
N˜4˜-(5-chloro-1,3-benzodioxol-4-yl)-N˜2˜-[3-(2-pyrrolidin-1-ylethoxy)phenyl]pyrimidine-2,4-diamine;
N˜4˜-(5-chloro-1,3-benzodioxol-4-yl)-N˜2˜-(3-chloro-4-morpholin-4-ylphenyl)pyrimidine-2,4-diamine;
N˜4˜-(5-chloro-1,3-benzodioxol-4-yl)-N˜2˜-[4-(2-morpholin-4-ylethoxy)phenyl]pyrimidine-2,4-diamine;
4-({4-[(5-chloro-1,3-benzodioxol-4-yl)amino]pyrimidin-2-yl}amino)-N-(2-hydroxyethyl)-N-methylbenzenesulfonamide;
4-({4-[(5-chloro-1,3-benzodioxol-4-yl)amino]pyrimidin-2-yl}amino)-N-[2-(diethylamino)ethyl]benzamide;
N˜4˜-(5-chloro-1,3-benzodioxol-4-yl)-N˜2˜-{4-[2-(4-methylpiperazin-1-yl)ethoxy]phenyl}pyrimidine-2,4-diamine;
N˜4˜-(5-chloro-1,3-benzodioxol-4-yl)-N˜2˜-[4(3-fluorobenzyl)oxy]-3-methoxyphenyl}pyrimidine-2,4-diamine;
N˜4˜-(5-chloro-1,3-benzodioxol-4-yl)-N˜2˜-{4-[(2-fluorobenzyl)oxy]-3-methoxyphenyl}pyrimidine-2,4-diamine;
4-({4-[(5-chloro-1,3-benzodioxol-4-yl)amino]pyrimidin-2-yl}amino)-N-(2-methoxyethyl)benzenesulfonamide;
N-[4-({4-[(5-chloro-1,3-benzodioxol-4-yl)amino]pyrimidin-2-yl}amino)phenyl]-N-methylacetamide;
N-[5-({4-[(5-chloro-1,3-benzodioxol-4-yl)amino]pyrimidin-2-yl}amino)-2-methylphenyl]acetamide;
N-[4-({4-[(5-chloro-1,3-benzodioxol-4-yl)amino]pyrimidin-2-yl}amino)benzyl]acetamide;
N˜4˜-(5-chloro-1,3-benzodioxol-4-yl)-N˜2˜-[3-(methylsulfonyl)phenyl]pyrimidine-2,4-diamine;
4-({4-[(5-chloro-1,3-benzodioxol-4-yl)amino]pyrimidin-2-yl}amino)benzenesulfonamide;
3-({4-[(5-chloro-1,3-benzodioxol-4-yl)amino]pyrimidin-2-yl}amino)benzenesulfonamide;
N˜4˜-(5-chloro-1,3-benzodioxol-4-yl)-N˜2˜-[4-(trifluoromethoxy)phenyl]pyrimidine-2,4-diamine;
N˜4˜-(5-chloro-1,3-benzodioxol-4-yl)-N[2˜-(4-morpholin-4-ylphenyl)pyrimidine-2,4-diamine;
N˜4˜-(5-chloro-1,3-benzodioxol-4-yl)-N˜2˜-(2-morpholin-4-ylphenyl)pyrimidine-2,4-diamine;
N˜4˜-(5-chloro-1,3-benzodioxol-4-yl)-N˜2˜-(3-morpholin-4-ylphenyl)pyrimidine-2,4-diamine;
N˜4˜-(5-chloro-1,3-benzodioxol-4-yl)-N˜2˜-[4-(2-ethoxyethoxy)phenyl]pyrimidine-2,4-diamine;
N˜4˜-(5-chloro-1,3-benzodioxol-4-yl)-N˜2˜-(2,3,4-trimethoxyphenyl)pyrimidine-2,4-diamine;
N-[3-({4-[(5-chloro-1,3-benzodioxol-4-yl)amino]pyrimidin-2-yl}amino)phenyl]methanesulfonamide;
N˜4˜-(5-chloro-1,3-benzodioxol-4-yl)-N˜2˜-[3-(dimethylamino)phenyl]pyrimidine-2,4-diamine;
2-[4-({4-[(5-chloro-1,3-benzodioxol-4-yl)amino]pyrimidin-2-yl}amino)phenyl]ethanol;
N˜4˜-(5-chloro-1,3-benzodioxol-4-yl)-N˜2˜-(3-chloro-4-fluorophenyl)pyrimidine-2,4-diamine;
N˜4˜-(5-chloro-1,3-benzodioxol-4-yl)-N˜2˜-(4-chloro-2-fluorophenyl)pyrimidine-2,4-diamine;
N˜4˜-(5-chloro-1,3-benzodioxol-4-yl)-N˜2˜-(3-chloro-2-fluorophenyl)pyrimidine-2,4-diamine;
N˜4˜-(5-chloro-1,3-benzodioxol-4-yl)-N˜2˜-(5-chloro-2-fluorophenyl)pyrimidine-2,4-diamine;
N˜4˜-(5-chloro-1,3-benzodioxol-4-yl)-N˜2˜-(4-chloro-3-fluorophenyl)pyrimidine-2,4-diamine;
5-({4-[(5-chloro-1,3-benzodioxol-4-yl)amino]pyrimidin-2-yl}amino)-1,3-dihydro-2H-indol-2-one;
N-[4-({4-[(5-chloro-1,3-benzodioxol-4-yl)amino]pyrimidin-2-yl}amino)phenyl]acetamide;
3-({4-[(5-chloro-1,3-benzodioxol-4-yl)amino]pyrimidin-2-yl}amino)-N-methylbenzamide;
4-({4-[(5-chloro-1,3-benzodioxol-4-yl)amino]pyrimidin-2-yl}amino)-N-methylbenzamide;
N˜2˜-1,3-benzothiazol-6-yl-N˜4˜-(5-chloro-1,3-benzodioxol-4-yl)pyrimidine-2,4-diamine;
N˜4˜-(5-chloro-1,3-benzodioxol-4-yl)-N˜2˜-(2,5-dimethoxyphenyl)pyrimidine-2,4-diamine;
N˜4˜-(5-chloro-1,3-benzodioxol-4-yl)-N˜2˜-(2,4-dimethoxyphenyl)pyrimidine-2,4-diamine;
N˜4˜-(5-chloro-1,3-benzodioxol-4-yl)-N˜2˜-(3,5-dimethoxyphenyl)pyrimidine-2,4-diamine;
N˜4˜-(5-chloro-1,3-benzodioxol-4-yl)-N˜2˜-(3,4-dimethoxyphenyl)pyrimidine-2,4-diamine;
N˜4˜-(5-chloro-1,3-benzodioxol-4-yl)-N˜2˜-(5-chloro-2-methoxyphenyl)pyrimidine-2,4-diamine;
N˜4˜-(5-chloro-1,3-benzodioxol-4-yl)-N˜2˜-(2-chloro-5-methoxyphenyl)pyrimidine-2,4-diamine;
N˜4˜-(5-chloro-1,3-benzodioxol-4-yl)-N˜2˜-(3-chloro-2-methoxyphenyl)pyrimidine-2,4-diamine;
N˜4˜-(5-chloro-1,3-benzodioxol-4-yl)-N˜2˜-[3-(1,3-oxazol-5-yl)phenyl]pyrimidine-2,4-diamine;
N˜4˜-(1H-Indazol-7-yl)-N˜2˜-(3,4,5-trimethoxyphenyl)pyrimidine-2,4-diamine;
N′-(1-methylindol-4-yl)-N-(3,4,5-trimethoxyphenyl)-pyrimidine-2,4-diamine;
N′-(5-bromobenzo[1,3]dioxol-4-yl)-N-(3,4,5-trimethoxyphenyl)-pyrimidine-2,4-diamine;
N′-benzo[1,3]dioxol-4-yl-N-(3,4,5-trimethoxyphenyl)-pyrimidine-2,4-diamine;
N′-(5-fluorobenzo[1,3]dioxol-4-yl)-N-(3,4,5-trimethoxyphenyl)-pyrimidine-2,4-diamine;
N′-(2,2-difluorobenzo[1,3]dioxol-4-yl)-N-(3,4,5-trimethoxyphenyl)-pyrimidine-2,4-diamine;
1-[7-[2-(3,4,5-trimethoxyphenyl)aminopyrimidin-4-yl]amino-2,3-dihydroindol-1-yl]ethanone; N′-(1H-indol-4-yl)-N-(3,4,5-trimethoxyphenyl)-pyrimidine-2,4-diamine;
N′-(6-chlorobenzofuran-7-yl)-N-(3-methylsulfonylphenyl)pyrimidine-2,4-diamine;
N′-(2,3-dihydrobenzofuran-7-yl)-N-(3-methylsulfonylphenyl)pyrimidine-2,4-diamine;
N′-(benzofuran-7-yl)-N-(3-methylsulfonylphenyl)pyrimidine-2,4-diamine;
N′-(1H-benzotriazol-4-yl)-N-(3-methylsulfonylphenyl)pyrimidine-2,4-diamine;
N′-(3-chloro-1H-indol-7-yl)-N-(3-methylsulfonylphenyl)pyrimidine-2,4-diamine;
N′-(6-methoxybenzo[1,3]dioxol-4-yl)-N-(3-methylsulfonylphenyl)pyrimidine-2,4-diamine;
4-[[2-[(3-methylsulfonylphenyl)amino]pyrimidin-4-yl]amino]benzo[1,3]dioxole-5-carboxamide;
N′-isoquinolin-5-yl-N-(3-methylsulfonylphenyl)pyrimidine-2,4-diamine;
N′-benzooxazol-7-yl-N-(3-methylsulfonylphenyl)pyrimidine-2,4-diamine;
N′-benzooxazol-4-yl-N-(3-methylsulfonylphenyl)pyrimidine-2,4-diamine;
3-[4-(1H-indazol-4-yl-methyl-amino)pyrimidin-2-yl]-N,N-dimethyl-benzamide;
N-methyl-N-[2-(3-methylsulfonylphenyl)pyrimidin-4-yl]-1H-indazol-4-amine;
3-[4-(1H-indazol-4-yl-methyl-amino)pyrimidin-2-yl]benzenesulfonamide;
[3-[4-(1H-indazol-4-yl-methyl-amino)pyrimidin-2-yl]phenyl]methanol;
N-[3-[4-(1H-indazol-4-yl-methyl-amino)pyrimidin-2-yl]phenyl]methanesulfonamide
N-(3,5-dimorpholinophenyl)-N′-(1H-indazol-4-yl)-N′-methyl-pyrimidine-2,4-diamine;
[4-[[2-[(3,5-dimorpholin-4-ylphenyl)amino]pyrimidin-4-yl]amino]-1H-indazol-6-yl]methanol;
N-(3,5-dimorpholinophenyl)-N′-(3-methyl-1H-indazol-4-yl)pyrimidine-2,4-diamine;
N′-benzooxazol-7-yl-N-(3,5-dimorpholin-4-ylphenyl)pyrimidine-2,4-diamine;
N′-benzooxazol-7-yl-N-(3,5-dimorpholinophenyl)-N′-methyl-pyrimidine-2,4-diamine;
N-(3,5-dimorpholin-4-ylphenyl)-N′-methyl-N′-(3-methyl-1H-indazol-4-yl)pyrimidine-2,4-diamine;
N′-methyl-N′-(3-methyl-1H-indazol-4-yl)-N-(3-methylsulfonylphenyl)pyrimidine-2,4-diamine;
N-(3,5-dimorpholin-4-ylphenyl)-N′-quinolin-5-yl-pyrimidine-2,4-diamine;
N′-(2,2-difluorobenzo[1,3]dioxol-4-yl)-N-(3,5-dimorpholin-4-ylphenyl)pyrimidine-2,4-diamine;
N-(3,5-dimorpholin-4-ylphenyl)-N′-(1H-indol-4-yl)pyrimidine-2,4-diamine;
N-(3,5-dimorpholin-4-ylphenyl)-N′-(2,5-dioxabicyclo[4.4.0]deca-6,8,10-trien-10-yl)pyrimidine-2,4-diamine;
N′-(1H-benzotriazol-4-yl)-N-(3,5-dimorpholin-4-ylphenyl)pyrimidine-2,4-diamine;
N′-(3-chloro-1H-indol-7-yl)-N-(3,5-dimorpholin-4-ylphenyl)pyrimidine-2,4-diamine;
N-(3,5-dimorpholin-4-ylphenyl)-N′-(1H-indazol-7-yl)pyrimidine-2,4-diamine;
   or a pharmaceutically acceptable salt thereof.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the invention may be administered in the form of a pro-drug that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I) and in vivo cleavable amide derivatives that may be formed at a carboxy group or an amino group in a compound of the Formula (I).

Accordingly, the present invention includes those compounds of the Formula (I) as defined hereinbefore when made available by organic synthesis and when made available is within the human or animal body by way of cleavage of a pro-drug thereof. Accordingly, the present invention includes those compounds of the Formula (I) that are produced by organic synthetic means and also such compounds that are produced in the human or animal body by way of metabolism of a precursor compound, that is a compound of the Formula (I) may be a synthetically-produced compound or a metabolically-produced compound.

A suitable pharmaceutically-acceptable pro-drug of a compound of the Formula (I) is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula (I) that possesses a carboxy group is, for example, an in vivo cleavable ester thereof. An in vivo cleavable ester of a compound of the Formula (I) containing a carboxy group is, for example, a pharmaceutically-acceptable ester, which is cleaved in the human or animal body to produce the parent acid. Suitable pharmaceutically-acceptable esters for carboxy include (1-6C)alkyl esters such as methyl, ethyl and tent-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula (I) that possesses a hydroxy group is, for example, an in vivo cleavable ester or ether thereof. An in vivo cleavable ester or ether of a compound of the Formula (I) containing a hydroxy group is, for example, a pharmaceutically-acceptable ester or ether, which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include a-acyloxyalkyl groups such as acetoxymethyl and pivaloyloxymethyl groups.

A suitable pharmaceutically-acceptable pro-drug of a compound of the Formula (I) that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula (I) may be exerted in part by one or more metabolites that are formed within the human or animal body after administration of a compound of the Formula (I). As stated hereinbefore, the in vivo effects of a compound of the Formula (I) may also be exerted by way of metabolism of a precursor compound (a pro-drug).

### Preparation of Compounds of Formula I

The synthesis of optically active forms may be carried out by standard techniques of organic chemistry well known in the art, for example by synthesis from optically active starting materials or by resolution of a racemic form.

Compounds of formula (I) can be prepared by various conventional methods as would be apparent to a chemist. In particular, compounds of formula (I) may be prepared by reacting a compound of formula (II):

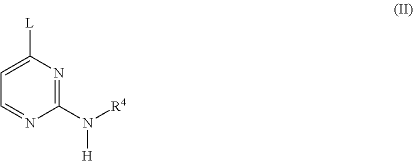

where R⁴ is as defined in relation to formula (I) provided that any functional groups are optionally protected, and L is a leaving group, with a compound of formula (III)

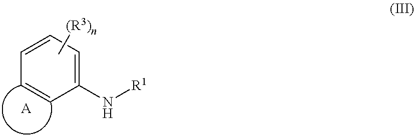

where A, R¹, R³ and n are as defined in relation to formula (I) provided that any functional groups are optionally protected. Thereafter, any protecting groups can be removed using conventional methods, and if required, the compound of formula (I) can be converted to a different compound of formula (I) or a salt, again using conventional chemical methods.

Suitable leaving groups L are halo such as chloro. The reaction is suitably carried out in an organic solvent such as a C₁₋₆alkanol, for instance, n-butanol, dimethylamine (DMA), or N-methylpyrrolidine (NMP) or mixtures thereof. An acid, in particular, and inorganic acid such as hydrochloric acid is suitably added to the reaction mixture. The reaction is suitably conducted at elevated temperatures for example at from 80-150° C., conveniently at the reflux temperature of the solvent.

Compounds of formula (II) may be prepared by various methods including for example, where L is a halogen, by reacting a compound of formula (IV)

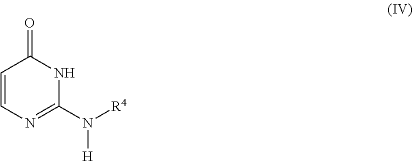

where R⁴ is as defined in relation to formula (I), with a halogenating agent such as phosphorus oxychloride. The reaction is conducted under reactions conditions appropriate to the halogenating agent employed. For instance, it may be conducted at elevated temperatures, for example of from 50-100° C., in an organic solvent such as acetonitrile or dichloromethane (DCM).

Compounds of formula (IV) are suitably prepared by reacting a compound of formula (V)

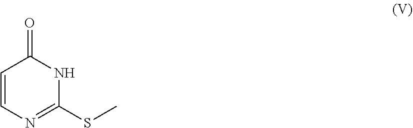

with a compound of formula (VI)

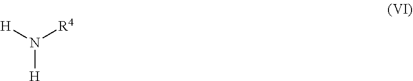

where R⁴ is as defined in relation to formula (I). The reaction is suitably effected in an organic solvent such as diglyme, again at elevated temperatures, for example of from 120-180° C., and conveniently at the reflux temperature of the solvent.

Alternatively, compounds of formula (I) may be prepared by reaction a compound of formula (VII)

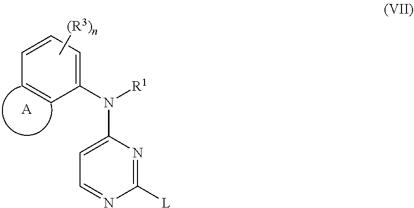

where A, R³ R¹ and n are as defined in relation to formula (I) provided that any functional groups can be optionally protected, and L is a leaving group as defined in relation to formula (II), with a compound of formula (VI) as defined above. Again, any protecting groups can be removed using conventional methods, and if required, the compound of formula (I) can be converted to a different compound of formula (I) or a salt, again using conventional chemical methods.

Conditions for carrying out such a reaction are broadly similar to those required for the reaction between compounds (II) and (III).

Compounds of formula (VII) are suitably prepared by reacting a compound of formula (III) as defined above with a compound of formula (VIII)

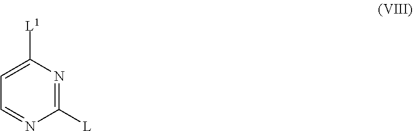

where L and L¹ are leaving groups such as halogen, and in particular chloro. The reaction is suitably effected in the presence of a strong base such as sodium hydride, in an organic solvent such as DMA. Depressed temperatures, for example from −20° C. to 20° C., conveniently at about 0° C. are suitably employed.

Compounds of formula (III) are either known compounds or they can be prepared from known compounds using analogous methods, which would be apparent to the skilled to chemist. For instance, examples of compounds of formula (III) and their preparation are described in WO2001094341.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Compounds of the formula I can be converted into further compounds of the formula I using standard procedures conventional in the art.

Examples of the types of conversion reactions that may be used to convert a compound of formula (I) to a different compound of formula (I) include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of an alkyl group using an alkyl halide and Lewis acid (such as aluminium trichloride) under Friedel Crafts conditions; and the introduction of a halo group. Particular examples of nucleophilic substitution reactions include the introduction of an alkoxy group or of a monoalkylamino group, a dialkyamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating.

The preparation of particular compounds of formula (I), such as compounds of formula (IA), (IB), (IC), (ID), (IE), (IF), and (IG), using the above-described methods form a further aspect of the invention.

According to a further aspect of the invention there is provided a pharmaceutical composition, which comprises a compound of the formula (I) and in particular a compound of formula (IA), (IB), (IC), (ID), (IE), (IF), and (IG), or a pharmaceutically acceptable salt thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the practitioner who is treating any particular patient may determine the optimum dosage.

### Biological Assays

### A) In Vitro EphB4 Enzyme Assay

This assay detects inhibitors of EphB4-mediated phosphorylation of a polypeptide substrate using Alphascreen™ luminescence detection technology. Briefly, recombinant EphB4 was incubated with a biotinylated-polypeptide substrate (biotin-poly-GAT) in presence of magnesium-ATP. The reaction was stopped by addition of EDTA, together with streptavidin-coated donor beads which bind the biotin-substrate containing any phosphorylated tyrosine residues. Anti-phosphotyrosine antibodies present on acceptor beads bind to phosphorylated substrate, thus bringing the donor & acceptor beads into close proximity. Subsequent excitation of the donor beads at 680 nm generated singlet oxygen species that interact with a chemiluminescer on the acceptor beads, leading to light emission at 520-620 nm. The signal intensity is directly proportional to the level of substrate phosphorylation and thus inhibition is measured by a decrease in signal.

Test compounds were prepared as 10 mM stock solutions in DMSO (Sigma-Aldrich Company Ltd, Gillingham, Dorset SP8 4XT Catalogue No.154938) and serially diluted with 5% DMSO to give a range of test concentrations at 6× the required final concentration. A 2 μl aliquot of each compound dilution was transferred to appropriate wells of low volume white 384-well assay plates (Greiner, Stroudwater Business Park, Stonehouse, Gloucestershire, GL10 3SX, Cat No. 784075) in duplicate. Each plate also contained control wells: maximum signal was created using wells containing 2 μl of 5% DMSO, and minimum signal corresponding to 100% inhibition were created using wells containing 2 μl of 0.5M EDTA (Sigma-Aldrich Company Ltd, Catalogue No. E7889).

For the assay, in addition to 2 μl of compound or control, each well of the assay plate contained; 10 μl of assay mix containing final buffer (10 mM Tris, 100 μM EGTA, 10 mM magnesium acetate, 4 μM ATP, 500 μM DTT, 1 mg/ml BSA), 0.25 ng of recombinant active EphB4 (amino acids 563-987; Swiss-Prot Acc. No. P54760) (ProQinase GmbH, Breisacher Str. 117, D-79106 Freiburg, Germany, Catalogue No 0178-0000-3) and 5 nM of the poly-GAT substrate (CisBio International, BP 84175, 30204 Bagnols/Cèze Cedex, France, Catalogue No. 61GATBLB). Assay plates were then incubated at room temperature for 1 hour. The reaction was then stopped by addition of 5 μl/well stop buffer (10 mM Tris, 495 mM EDTA, 1 mg/ml BSA) containing 0.25 ng each of AlphaScreen anti-phosphoTyrosine-100 acceptor beads and streptavidin—coated donor beads (Perkin Elmer, Catalogue No 6760620M). The plates were sealed under natural lighting conditions, wrapped in aluminium foil and incubated in the dark for a further 20 hours.

The resulting assay signal was determined on the Perkin Elmer EnVision plate reader. The minimum value was subtracted from all values, and the signal plotted against compound concentration to generate IC₅₀ data.

### B) In Vitro EphB4 Cell Assay

This assay identifies inhibitors of cellular EphB4 by measuring a decrease in phosphorylation of EphB4 following treatment of cells with compound. The endpoint assay used a sandwich ELISA to detect EphB4 phosphorylation status. Briefly, Myc-tagged EphB4 from treated cell lysate was captured on the ELISA plate via an anti-c-Myc antibody. The phosphorylation status of captured EphB4 was then measured using a generic phosphotyrosine antibody conjugated to HRP via a colourimetric output catalysed by HRP, with level of EphB4 phosphorylation directly proportional to the colour intensity. Absorbance was measured spectrophotometrically at 450 nm.

Full length human EphB4 (Swiss-Prot Acc. No. P54760) was cloned using standard techniques from cDNA prepared from HUVEC using RT-PCR. The cDNA fragment was then sub-cloned into a pcDNA3.1 expression vector containing a Myc-His epitope tag to generate full-length EphB4 containing a Myc-His tag at the C-terminus (Invitrogen Ltd. Paisley, UK). CHO-K1 cells (LGC Promochem, Teddington, Middlesex, UK, Catalogue No. CCL-61) were maintained in HAM's F12 medium (Sigma-Aldrich Company Ltd, Gillingham, Dorset SP8 4XT, Catalogue No. N4888) containing 10% heat-inactivated foetal calf serum (PAA lab GmbH, Pasching, Austria Catalogue No. PAA-A15-043) and 1% glutamax-1 (Invitrogen Ltd., Catalogue No. 35050-038) at 37° C. with 5% CO₂. CHO-K1 cells were engineered to stably express the EphB4-Myc-His construct using standard stable transfection techniques, to generate cells hereafter termed EphB4-CHO.

For each assay, 10,000 EphB4-CHO cells were seeded into each well of Costar 96-well tissue-culture plate (Fisher Scientific UK, Loughborough, Leicestershire, UK., Catalogue No. 3598) and cultured overnight in full media. On day 2, the cells were incubated overnight in 90 μl/well of media containing 0.1% Hyclone stripped-serum (Fisher Scientific UK, Catalogue No. SH30068.02). Test compounds were prepared as 10 mM stock solutions in DMSO (Sigma-Aldrich Company Ltd, Gillingham, Dorset SP8 4XT Catalogue No.154938) and serially diluted with serum-free media to give a range of test concentrations at 10× the required final concentration. A 10 μl aliquot of each compound dilution was transferred to the cell plates in duplicate wells, and the cells incubated for 1 hour at 37° C. Each plate also contained control wells: a maximum signal was created using untreated cells, and minimum signal corresponding to 100% inhibition was created using wells containing a reference compound known to abolish EphB4 activity.

Recombinant ephrin-B2-Fc (R&D Systems, Abingdon Science Park, Abingdon, Oxon OX14 3NB UK, Catalogue No. 496-EB), a Fc-tagged form of the cognate ligand for EphB4, was pre-clustered at a concentration of 3 μg/ml with 0.3 μg/ml anti-human IgG, Fc fragment specific (Jackson ImmunoResearch Labs, Northfield Business Park, Soham, Cambridgeshire, UK CB7 SUE, Catalogue No. 109-005-008) in serum-free media for 30 minutes at 4° C. with occasional mixing. Following compound treatment, cells were is stimulated with clustered ephrin-B2 at a final concentration of 1 μg/ml for 20 minutes at 37° C. to induce EphB4 phosphorylation. Following stimulation, the medium was removed and the cells lysed in 100 μl/well of lysis buffer (25 mM Tris HCl, 3 mM EDTA, 3 mM EGTA, 50 mM NaF, 2 mM orthovanadate, 0.27M Sucrose, 10 mM β-glycerophosphate, 5 mM sodium pyrophosphate, 2% Triton X-100, pH 7.4).

Each well of an ELISA Maxisorp 96-well plate (Nunc; Fisher Scientific UK, Loughborough, Leicestershire, UK., Catalogue No. 456537) was coated overnight at 4° C. with 100 μl of anti-c-Myc antibody in Phosphate Buffered Saline (10 μg/ml; produced at AstraZeneca). Plates were washed twice with PBS containing 0.05% Tween-20 and blocked with 250 μl/well 3% TopBlock (Fluka) (Sigma-Aldrich Company Ltd, Gillingham, Dorset SP8 4XT, Catalogue No. 37766) for a minimum of 2 hours at room temperature. Plates were washed twice with PBS/0.05% Tween-20 and incubated with 100 μl/well cell lysate overnight at 4° C. ELISA plates were washed four times with PBS/0.05% Tween-20 and incubated for 1 hour at room temperature with 100 μl/well HRP-conjugated 4G10 anti-phosphotyrosine antibody (Upstate, Dundee Technology Park, Dundee, UK, DD2 1SW, Catalogue No. 16-105) diluted 1:6000 in 3% Top Block. ELISA plates were washed four times with PBS/0.05% Tween-20 and developed with 100 μl/well TMB substrate (Sigma-Aldrich Company Ltd, Catalogue No. T0440). The reaction was stopped after 15 minutes with the addition of 25 μl/well 2M sulphuric acid. The absorbances were determined at 450 nm using the Tecan SpectraFluor Plus. The minimum value was subtracted from all values, and the signal plotted against compound concentration to generate IC₅₀ data.

### C) Src Assay

### In Vitro Enzyme Assay

The ability of test compounds to inhibit the phosphorylation of a tyrosine containing polypeptide substrate by the enzyme c-Src kinase was assessed using a conventional ELISA assay with a colorimetric endpoint.

Each well of Matrix 384-well plates (Matrix, Brooke Park, Wilmslow, Cheshire, SK9 3LP, UK, Catalogue No. 4311) were coated overnight at 4° C. with 40 μl of 10 ug/ml stock of synthetic polyamino acid pEAY substrate (Sigma-Aldrich Company Ltd, Gillingham, Dorset, SP8 4XT, UK, Catalogue No. P3899) in phosphate buffered saline (PBS). Immediately prior to the assay, the plates were washed with 100 μl/well of PBS containing Tween-20 and then with 50 mM HEPES pH7.4.

Test compounds were prepared as 10 mM stock solutions in DMSO (Sigma-Aldrich Company Ltd, Gillingham, Dorset, SP8 4XT, UK, Catalogue No.154938) and serially diluted with 10% DMSO to give a range of test concentrations at 4× the required final concentration. A 10 μl aliquot of each compound dilution was transferred to the appropriate ELISA wells in duplicate. Each plate also contained control wells: maximum signal was created using wells containing 10 μl of 10% DMSO, and minimum signal corresponding to 100% inhibition were created using wells containing 10 μl of 0.5M EDTA (Sigma-Aldrich Company Ltd, Catalogue No. E7889).

10 μl of a solution containing 8.8 μM ATP and 80 mM MnCl2 was added to each well to give a final concentration of 2.2 μM and 20 mM respectively. The reaction was initiated by addition of 20 μl/well of assay buffer (final concentration of 50 mM HEPES, 0.1 mM sodium orthovanadate, 0.01% BSA, 0.1 mM DTT, 0.05% Triton X-100, pH 7.4) containing active human recombinant c-Src kinase (Upstate, Dundee Technology Park, Dundee, UK, DD2 1SW, Catalogue No 14-117). Plates were then incubated at room temperature for 20 minutes before the kinase reaction was stopped by addition of 20 μl/well of 0.5M EDTA.

The plates were washed three times with 100 μl/well of PBS-Tween20 and then 40 μl of a PBS-Tween20 and 0.5% BSA solution containing 4G10-HRP anti-phosphotyrosine antibody (Upstate, Catalogue No 16-105) added to each well. The plates were incubated for 1 hour at room temperature before being washed three times with 100 μl/well of PBS-Tween20. Plates were developed with 40 μl/well TMB substrate solution in DMSO (Sigma-Aldrich Company Ltd, Catalogue No. T2885) for up to one hour at room temperature. The reaction was then stopped with the addition of 20 μl/well 2M sulphuric acid and the absorbances determined at 450 nm using a plate reading spectrophotometer. The minimum value was subtracted from all values, and the signal plotted against compound concentration to generate IC₅₀ data.

Compounds of the invention were active in the above assays, for instance, generally showing IC₅₀ values of less than 100 μM in Assay A and Assay B. Preferred compounds of the invention generally showing IC₅₀ values of less than 30 μM in Assay A and Assay B. For instance, Compound 59 of the Examples showed an IC₅₀ of 0.46 μM in assay A, an IC₅₀ is of 1.25 μM in assay B, an IC₅₀ of 0.33 μM in assay C. Further illustrative IC₅₀ values obtained using Assay B for a selection of the compounds exemplified in the present application are shown in Table A below.

TABLE A
Mean IC₅₀ values obtained using Assay B

| Compound No. | Mean IC₅₀ (μM) |
|---|---|
| 219 | 0.14 |
| 227 | 0.19 |
| 241 | 0.13 |
| 258 | 1.08 |
| 293 | 0.23 |
| 309 | 2.53 |
| 318 | 0.05 |
| 326 | 0.51 |

Compounds of the invention were also found active in a KinaseProfile™ assay for EphA2 kinase activity operated by Upstate of Charlotteville, Va. 22903, USA. For instance, the compound of Example 1 above showed an IC₅₀ of 15 nM in this assay.

As a result of their activity in screens described above, the compounds of the present invention are expected to be useful in the treatment of diseases or medical conditions mediated alone or in part by EphB4 enzyme activity, i.e. the compounds may be used to produce an EphB4 inhibitory effect in a warm-blooded animal in need of such treatment. Thus, the compounds of the present invention provide a method for treating the proliferation of malignant cells characterised by inhibition of the EphB4 enzyme, i.e. the compounds may be used to produce an anti-proliferative effect mediated alone or in part by the inhibition of EphB4.

In addition, certain compounds of the invention may also be active against the EphA2 or Src kinase enzymes, i.e. the compounds may also be used to produce an EphA2 and Src kinase inhibitory effect in a warm-blooded animal in need of such treatment. Thus, to the compounds of the present invention provide a method for treating the proliferation of malignant cells characterised by inhibition of EphB4, EphA2 or Src enzymes, i.e. the compounds may be used to produce an anti-proliferative effect mediated alone or in part by the inhibition of EphB4, EphA2 or Src kinase.

According to a further aspect of the invention, there is provided the use of a compound of formula (IH)

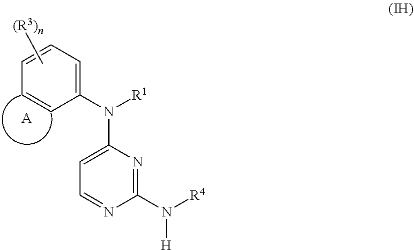

where R¹ is selected from hydrogen, C₁₋₆alkyl, C₂₋₆alkenyl, or C₂₋₆alkynyl, wherein the alkyl, alkenyl and alkynyl groups are optionally substituted by one or more substituent groups selected from cyano, nitro, —OR², —NR^{2a}R^{2b}, —C(O)NR^{2a}R^{2b}, or —N(R^{2a})C(O)R², halo or haloC₁₋₄alkyl, where R², R^{2a} and R^{2b} are selected from hydrogen or C₁₋₆alkyl such as methyl, or R^{2a} and R^{2b} together with the nitrogen atom to which they are attached may form a 5 or 6-membered heterocyclic ring, which optionally contains an additional heteroatom selected from N, O or S;
ring A is fused 5 or 6-membered carbocyclic or heterocyclic ring, which is saturated or unsaturated, and is optionally substituted on any available carbon atom by one or more substituent groups selected from halo, cyano, hydroxy, C₁₋₆alkyl, C₁₋₆alkoxy, —S(O)_{z}—C₁₋₆alkyl (where z is 0, 1 or 2), or —NR^{a}R^{b} (where R^{a} and R^{b} are each independently selected from hydrogen, C₁₋₄alkyl, or C₁₋₄alkylcarbonyl), and where any nitrogen atoms in the ring are optionally substituted by a C₁₋₆alkyl or C₁₋₆alkylcarbonyl;
n is 0, 1, 2 or 3
and each group R³ is independently selected from halo, trifluoromethyl, cyano, nitro or a group of sub-formula (i):

—X¹—R¹¹ (i)
where X¹ is selected from a direct bond or O, S, SO, SO₂, OSO₂, NR¹³, CO, CH(OR¹³), CONR¹³, N(R¹³)CO, SO₂N(R¹³), N(R¹³)SO₂, C(R¹³)₂O, C(R¹³)₂S, C(R¹³)₂N(R¹³) and N(R¹³)C(R¹³)₂, wherein R¹³ is hydrogen or C₁₋₆alkyl and R¹¹ is selected from hydrogen, C₁₋₆ alkyl, C₂₋₈alkenyl, C₂₋₈alkynyl, C₃₋₈cycloalkyl, aryl or heterocyclyl, C₃₋₈cycloalkylC₁₋₆ alkyl, arylC₁₋₆ alkyl or heterocyclylC₁₋₆alkyl, any of which may be optionally substituted with one or more groups selected from halo, trifluoromethyl, cyano, nitro, hydroxy, amino, carboxy, carbamoyl, C₁₋₆alkoxy, C₂₋₆alkenyoxyl, C₂₋₆alkynyloxy, C₁₋₆alkylthio, C₁₋₆alkylsulphinyl, C₁₋₆alkylsulphonyl, C₁₋₆alkylamino, di-(C₁₋₆alkyl)amino, C₁₋₆alkoxycarbonyl, N—C₁₋₆alkylcarbamoyl, N,N-di-(C₁₋₆alkyl)carbamoyl, C₂₋₆alkanoyl, C₂₋₆alkanoyloxy, C₂₋₆alkanoylamino, N-C₁₋₆alkyl-C₂₋₆alkanoylamino, C₃₋₆alkenoylamino, N—C₁₋₆alkyl-C₃₋₆alkenoylamino, C₃₋₆alkynoylamino, N—C₁₋₆alkyl-C₃₋₆alkynoylamino, N—C₁₋₆alkylsulphamoyl, N,N-di-(C₁₋₆alkyl)sulphamoyl, C₁₋₆alkanesulphonylamino and N—C₁₋₆alkyl-C₁₋₆alkanesulphonylamino, and any heterocyclyl group within R¹¹ optionally bears 1 or 2 oxo or thioxo substituents; and
R⁴ is a group of sub-formula (iii)

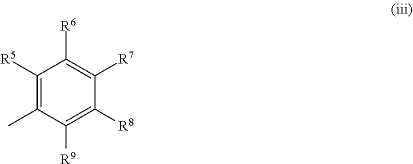

where R⁵, R⁶, R⁷, R⁸ and R⁹ are each independently selected from:
(i) hydrogen, halo, trifluoromethyl, trifluoromethoxy, cyano, nitro, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by halo, hydroxy, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino, and any nitrogen atoms present in a heterocyclyl group may, depending upon valency considerations, be substituted by a group selected from hydrogen, C₁₋₆alkyl or C₁₋₆alkylcarbonyl, and where any sulphur atoms may be optionally oxidised to a sulphur oxide;
(ii) a group of sub-formula (iv):

—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, —N(R¹⁶)C(O)N(R¹⁶)—, —N(R¹⁶)C(O)O—, SON(R¹⁶), N(R¹⁶)SO, SO₂N(R¹⁶), N(R¹⁶)SO₂, C(R¹⁶)₂O, C(R¹⁶)₂S and N(R¹⁶)C(R¹⁶)₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, aryl-C₁₋₆alkyl, or a 4- to 8-membered mono or bicyclic heterocyclyl ring (including 5 or 6 membered heteroaryl rings) or 4- to 8-membered mono or bicyclic heterocyclyl-C₁₋₆alkyl groups (including 5 or 6 membered heteroaryl-C₁₋₆alkyl groups) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
iii) a group of sub-formula (v):

—X³—R¹⁵—Z (v)
where X³ is a direct bond or is selected from O, NR¹⁷, S, SO, SO₂, OSO₂, CO, C(O)O, OC(O), CH(OR¹⁷), CON(R¹⁷), N(R¹⁷)CO, —N(R¹⁷)C(O)N(R¹⁷)—, —N(R¹⁷)C(O)O—, SO₂N(R¹⁷), N(R¹⁷)SO₂, C(R¹⁷)₂O, C(R¹⁷)₂S and N(R¹⁷)C(R¹⁷)₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, C₃₋₁₂ carbocyclyl or heterocyclyl (including heteroaryl) which optionally bears 1 or 2 substituents, which may be the same or different, selected from halo, C₁₋₆alkyl, C₂₋₈alkenyl, C₂₋₈alkynyl and C₁₋₆alkoxy and wherein any heterocyclyl group within Z optionally bears 1 or 2 oxo substituents,
or Z is a group of sub-formula (vi)

—X⁴—R¹⁸ (vi)
where X⁴ is selected from O, NR¹⁹, S, SO, SO₂, OSO₂, CO, C(O)O, OC(O), CH(OR¹⁹), CON(R¹⁹), N(R¹⁹)CO, SO₂N(R¹⁹), —N(R¹⁹)C(O)N(R¹⁹)—, —N(R¹⁹)C(O)O—N(R¹⁹)SO₂, C(R¹⁹)₂O, C(R¹⁹)₂S and N(R¹⁹)C(R¹⁹)₂, where each R¹⁹ is independently selected from hydrogen or C₁₋₆alkyl; and R¹⁸ is selected from hydrogen, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl) which optionally bears 1 or 2 substituents, which may be the same or different, selected from halo, C₁₋₆alkyl, C₂₋₈alkenyl, C₂₋₈alkynyl and C₁₋₆alkoxy, and wherein any heterocyclyl group within R¹⁸ optionally bears 1 or 2 oxo substituents;
or
(iv) R⁵ and R⁶, R⁶ and R⁷, R⁷ and R⁸ or R⁸ and R⁹ are joined together to form a fused 5, 6 or 7-membered ring, wherein said ring is unsaturated or partially or fully saturated and is optionally substituted on any available carbon atom by halo, C₁₋₆alkyl, hydroxyC₁₋₆alkyl, amino, N—C₁₋₆alkylamino, or N,N-diC₁₋₆alkylamino, and said ring may contain one or more heteroatoms selected from oxygen, sulphur or nitrogen, where sulphur atoms may be optionally oxidised to a sulphur oxide, where any CH₂ groups may be substituted by a C(O) group, and where nitrogen atoms, depending upon valency considerations, may be substituted by a group R²¹, where R²¹ is selected from hydrogen, C₁₋₆alkyl or C₁₋₆alkylcarbonyl;
or a pharmaceutically acceptable salt thereof,
   in the manufacture of a medicament for use in the treatment of cancer.

According to another aspect of the present invention there is provided a compound of the formula (I), (IA), (IB), (IC), (ID), (IE), (IF), (IG) or (IH), or a pharmaceutically acceptable salt thereof, as defined hereinbefore for use in a method of treatment of the human or animal body by therapy.

Thus according to a further aspect of the invention there is provided a compound of the formula (I), (IA), (IB), (IC), (ID), (IE), (IF), (IG) or (IH), or a pharmaceutically acceptable salt thereof, as defined hereinbefore for use as a medicament.

According to a further aspect of the invention there is provided a compound of the formula (I), (IA), (IB), (IC), (ID), (IE), (IF), (IG) or (IH), or a pharmaceutically acceptable salt thereof, as defined hereinbefore for use in the production of an EphB4 inhibitory effect in a warm blooded animal such as man.

According to a further aspect of the invention there is provided the use of a compound of the formula (I), (IA), (IB), (IC), (ID), (IE), (IF), (IG) or (IH), or a pharmaceutically acceptable salt thereof, as defined hereinbefore in the manufacture of a medicament for use in the production of an EphB4 inhibitory effect in a warm-blooded animal such as man.

According to a further aspect of the invention there is provided the use of a compound of the formula (I), (IA), (IB), (IC), (ID), (IF), (IF), (IG) or (IH), or a pharmaceutically acceptable salt thereof, as defined hereinbefore in the manufacture of a medicament for use in the production of an EphB4, EphA2 and Src kinase inhibitory effect in a warm-blooded animal such as man.

According to a further feature of this aspect of the invention there is provided a method for producing an EphB4 inhibitory effect in a warm-blooded animal, such as man, in need of such treatment which comprises administering to said animal an effective amount of a compound of the formula (I), (IA), (IB), (IC), (ID), (IE), (IF), (IG) or (IH), or a pharmaceutically acceptable salt thereof, as defined hereinbefore.

According to a further feature of this aspect of the invention there is provided a method for producing an EphB4, EphA2 and Src kinase inhibitory effect inhibitory effect in a warm-blooded animal, such as man, in need of such treatment which comprises administering to said animal an effective amount of a compound of the formula (I), (IA), (IB), (IC), (ID), (IE), (IF), (IG) or (IH), or a pharmaceutically acceptable salt thereof, as defined hereinbefore.

According to a further aspect of the invention there is provided a compound of the formula (I), (IA), (IB), (IC), (ID), (IE), (IF), (IG) or (IH), or a pharmaceutically acceptable salt thereof, as defined hereinbefore for use in the production of an anti-angiogenic effect in a warm-blooded animal such as man.

According to a further aspect of the invention there is provided the use of a compound of the formula (I), (IA), (IB), (IC), (ID), (IE), (IF), (IG) or (IH), or a pharmaceutically acceptable salt thereof, as defined hereinbefore in the manufacture of a medicament for use in the production of an anti-angiogenic effect in a warm-blooded animal such as man.

According to a further feature of this aspect of the invention there is provided a method for producing an anti-angiogenic effect in a warm-blooded animal, such as man, in need of such treatment which comprises administering to said animal an effective amount of a compound of the formula (I), (IA), (IB), (IC), (ID), (IE), (IF), (IG) or (IH), or a pharmaceutically acceptable salt thereof, as defined hereinbefore.

According to a further feature of the invention there is provided a compound of the formula (I), (IA), (IB), (IC), (ID), (IE), (IF), (IG) or (IH), or a pharmaceutically acceptable salt thereof, as defined hereinbefore in the manufacture of a medicament for use in the treatment of cancer.

According to an additional feature of this aspect of the invention there is provided a compound of the formula (I), (IA), (IB), (IC), (ID), (IE), (IF), (IG) or (IH), or a pharmaceutically acceptable salt thereof, as defined hereinbefore, for use in the treatment of cancer.

According to an additional feature of this aspect of the invention there is provided a method of treating cancer in a warm-blooded animal, such as man, in need of such treatment which comprises administering to said animal an effective amount of a compound of the formula (I), (IA), (IB), (IC), (ID), (IE), (IF), (IG) or (IH), or a pharmaceutically acceptable salt thereof, as defined hereinbefore.

In a further aspect of the present invention there is provided the use of a compound of the formula (I), (IA), (IB), (IC), (ID), (IE), (IF), (IG) or (IH), or a pharmaceutically acceptable salt thereof, as defined hereinbefore, in the manufacture of a medicament for use in the treatment of solid tumour disease, in particular neuroblastomas, breast, liver, lung and colon cancer or leukemias.

In a further aspect of the present invention there is provided a method of treating neuroblastomas, breast, liver, lung and colon cancer or leukemias in a warm-blooded animal, such as man, in need of such treatment which comprises administering to said animal an effective amount of a compound of the formula (I), (IA), (IB), (IC), (ID), (IE), (IF), (IG) or (IH), or a pharmaceutically acceptable salt thereof, as defined hereinbefore.

The anti-cancer treatment defined hereinbefore may be applied as a sole therapy or may involve, in addition to the compound of the invention, conventional surgery or radiotherapy or chemotherapy. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the anti-angiogenic treatment defined hereinbefore may be: surgery, radiotherapy or chemotherapy. Such chemotherapy may include one or more of the following categories of anti-tumour agents:
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as famesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications W097/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

According to this aspect of the invention there is provided a pharmaceutical composition comprising a compound of the formula (I) as defined hereinbefore and an additional anti-tumour substance as defined hereinbefore for the conjoint treatment of cancer.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

In addition to their use in therapeutic medicine, the compounds of formula (I), (IA),(IB) or (IC) and their pharmaceutically acceptable salts thereof, are also useful as pharmacological tools in the development and standardisation of in vitro and in vivo test systems for the evaluation of the effects of inhibitors of anti-angiogenic activity in laboratory animals such as cats, dogs, rabbits, monkeys, rats and mice, as part of the search for new therapeutic agents.

The invention will now be illustrated in the following Examples in which, generally:

### FOR EXAMPLES 1 TO 9

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulphate, work-up procedures were carried out using traditional layer separating techniques or an ALLEXIS (MTM) automated liquid handler, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz, a Bruker Dpx400 operating at 400 MHz or a Bruker Advance operating at 500 MHz. The following abbreviations have been used: s, singlet; d, doublet; t, triplet; q, quartet; in, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88)) and acetonitrile;

(x) the following analytical HPLC methods were used; in general, reversed-phase silica was used with a flow rate of about 1 ml per minute and detection was by Electrospray Mass Spectrometry and by UV absorbance at a wavelength of 254 nm; for each method Solvent A was water and Solvent B was acetonitrile; the following columns and solvent mixtures were used:

Preparative HPLC was performed on C18 reversed-phase silica, on a Phenomenex “Gemini” preparative reversed-phase column (5 microns silica, 110A, 21.1 mm diameter, 100 mm length) using decreasingly polar mixtures as eluent, for example decreasingly polar mixtures of water (containing 0.1% formic acid or 0.1% ammonia) as solvent A and acetonitrile as solvent B; either of the following preparative HPLC methods were used:

Method A: a solvent gradient over 9.5 minutes, at 25 mls per minute, from a 85:15 mixture of solvents A and B respectively to a 5:95 mixture of solvents A and B.

Method B: a solvent gradient over 9.5 minutes, at 25 mls per minute, from a 60:40 mixture of solvents A and B respectively to a 5:95 mixture of solvents A and B.

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

### FOR EXAMPLES 10 TO 28

(i) temperatures are given in degrees Celsius (° C.); operations were carried out at room or ambient temperature, that is, at a temperature in the range of 18 to 25° C.;

(ii) organic solutions were dried over anhydrous magnesium sulfate or anhydrous sodium sulfate; evaporation of solvent was carried out using a rotary evaporator under reduced pressure (600 to 4000 Pascals; 4.5 to 30 mmHg) with a bath temperature of up to 60° C.;

(iii) chromatography means flash chromatography on silica gel; thin layer chromatography (TLC) was carried out on silica gel plates;

(iv) in general, the course of reactions was followed by TLC and/or analytical LC-MS, and reaction times are given for illustration only. The retention times (t_{R}) were measured on a LC/MS Waters 2790/ZMD Micromass system equipped with a Waters Symmetry column (C18, 3.5 μM, 4.6×50 mm); detection UV 254 nM and MS; elution: flow rate 2.5 ml/min, linear gradient from 95% water-5% methanol containing 5% formic acid to 40% water-55% acetonitrile-5% methanol containing 5% formic acid over 3 minutes; then linear gradient to 95% acetonitrile-5% methanol containing 5% formic acid over 1 minute;

(v) final products had satisfactory proton nuclear magnetic resonance (NMR) spectra and/or mass spectral data;

(vi) yields are given for illustration only and are not necessarily those which can be obtained by diligent process development; preparations were repeated if more material was required;

(vii) when given, NMR data is in the form of delta values for major diagnostic protons, given in parts per million (ppm) relative to tetramethylsilane (TMS) as an internal standard, determined at 500 MHz using perdeuterio dimethyl sulfoxide (DMSO-d₆) as solvent unless otherwise indicated; the following abbreviations have been used: s, singlet; d, doublet; t, triplet; q, quartet; m, multiplet; br, broad;

(viii) chemical symbols have their usual meanings; SI units and symbols are used;

(ix) solvent ratios are given in volume:volume (v/v) terms; and

(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺ which refers to the protonated mass ion; reference to M⁺ is to the mass ion generated by loss of an electron; and reference to M−H⁺ is to the mass ion generated by loss of a proton;

(xi) unless stated otherwise compounds containing an asymmetrically substituted carbon and/or sulfur atom have not been resolved;

(xii) where a synthesis is described as being analogous to that described in a previous example the amounts used are the millimolar ratio equivalents to those used in the previous example;

(xiii) all microwave reactions were carried out in a Personal Chemistry EMRYS™ Optimizer EXP microwave synthesisor;

(xiv) preparative high performance liquid chromatography (HPLC) was performed on a Waters instrument using the following conditions:

Column: 30 mm×15 cm Xterra Waters, C18, 5 mm

Solvent A: Water with 1% acetic acid or 2 g/l ammonium carbonate

Solvent B: Acetonitrile

Flow rate: 40 ml/min

Run time: 15 minutes with a 10 minute gradient from 5-95% B

Wavelength: 254 nm

Injection volume 2.0-4.0 ml;

In addition, the following abbreviations have been used, where necessary:
DMSO dimethylsulphoxide
NMP 1-methyl-2-pyrrolidinone
DMA N,N-dimethylacetamide
DCM Dichloromethane
THF tetrahydrofuran;
DMF N,N-dimethylformamide;
DTAD di-tert-butyl azodicarboxylate;
DIPEA di-isopropylethylamine;
IPA isopropyl alcohol;
Ether diethyl ether; and
TFA trifluoroacetic acid.

### EXAMPLE 1

### Step 1

### 2-Chloro-N-(5-chloro-1,3-benzodioxol-4-yl)pyrimidin-4-amine

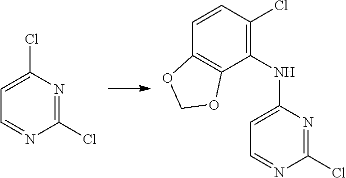

Sodium hydride (13.4 g, 60% dispersion in mineral oil) was added portion-wise to (5-chloro-1,3-benzodioxol-4-yl)amine (11.5 g, prepared as described in WO2001094341) in DMA (100 ml) at 0° C. 2,4-Dichloropyrimidine (10 g) was added and the reaction warmed to room temperature and stirred overnight. The reaction was quenched cautiously with water, the solution filtered and concentrated and the residue dissolved in DCM, washed with water and brine, dried and concentrated to give the title compound as a dark brown oil that was used without further purification (16 g, 85%); NMR Spectrum (300 MHz, DMSO) 6.10 (s, 2H), 6.58 (d, 1H), 6.94 (d, 1H), 7.05 (d, 1H), 8.15 (d, 1H), 9.76 (s, 1H); Mass Spectrum M⁺ 284.4.

### Step 2

### N˜4˜-(5-Chloro-1,3-benzodioxol-4-yl)-N˜2˜-(3,4,5-trimethoxyphenyl)pyrimidine-2,4-diamine (Compound No. 1)

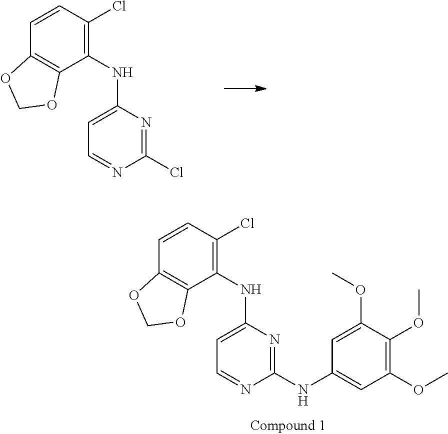

3,4,5-Trimethoxyaniline (103 mg) and 2-chloro-N-(5-chloro-1,3-benzodioxol-4-yl)pyrimidin-4-amine (200 mg) were dissolved in n-butanol (1 ml) and DMA (1 ml) and a solution of HCl in diethyl ether (0.7 ml, 1M) added. The reaction was heated at 120° C. for 3 hours then cooled to room temperature and concentrated in vacuo. The residue was purified by reverse phase chromatography to give the title compound as a solid (69 mg, 23%); NMR Spectrum (300 MHz, DMSO) 3.58 (s, 9H), 5.98 (s, 2H), 8.85 (s, 1H), 6.10 (d, 1H), 6.87 (d, 1H), 7.02 (d, 1H), 7.05 (s, 2H), 7.99 (d, 1H); Mass Spectrum MH⁺ 431.38.

### EXAMPLE 2

The procedure described above in Example 1 was repeated using the appropriate aniline (which were sourced commercially or prepared as described in the Method section below). Thus were obtained the compounds described below in Table 1.

TABLE 1

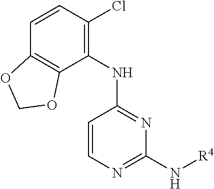

| | | | Molecular | NMR Spectrum | Aniline |
|---|---|---|---|---|---|
| | | | Ion | (400 MHz, d6- | (Meth- |
| No. | Name | R⁴ | (Observed) | DMSO) | od) |
| 2 | N~4~-(5-chloro- 1,3-benzodioxol- 4-yl)-N~2~-(2- chlorophenyl) pyrimidine-2,4- diamine | 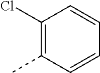 | 375.52 (M+) | | |
| 3 | N~4~-(5-chloro- 1,3-benzodioxol- 4-yl)-N~2~-1H- indazol-6- ylpyrimidine-2,4- diamine | 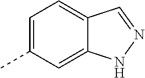 | 381.57 (MH+) | | |
| 4 | N~4~-(5-chloro- 1,3-benzodioxol- 4-yl)-N~2~- phenylpyrimidine- 2,4-diamine | 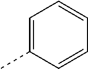 | 341.4 (MH+) | 6.00 (s, 2H), 6.15 (d, 1H), 6.82 (t, 1H), 6.93 (d, 1H), 7.09 (m, 3H), 7.60 (d, 2H), 8.00 (d, 2H), 8.97 (s, 1H), 9.08 (s, 1H) | |
| 5 | N~4~-(5-chloro- 1,3-benzodioxol- 4-yl)-N~2~-(2- fluorophenyl) pyrimidine-2,4- diamine | 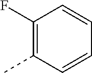 | 359.39 (MH+) | 6.00 (s, 2H), 6.14 (d, 1H), 6.91 (d, 1H), 6.98 (m, 2H), 7.04 (d, 1H), 7.13 (m, 1H), 7.86 (m, 1H), 7.99 (d, 1H), 8.30 (s, 1H), 9.00 (s, 1H) | |
| 6 | N~4~-(5-chloro- 1,3-benzodioxol- 4-yl)-N~2~-(3- fluorophenyl) pyrimidine-2,4- diamine | 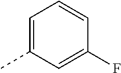 | 359.4 (MH+) | 6.03 (s, 2H), 6.20 (d, 1H), 6.61 (m, 1H), 6.92 (d, 1H), 7.05 (d, 1H), 7.12 (m, 1H), 7.32 (d, 1H), 7.64 (d, 1H), 8.03 (d, 1H), 9.07 (s, 1H), 9.33 (s, 1H) | |
| 7 | N~4~-(5-chloro- 1,3-benzodioxol- 4-yl)-N~2~-(4- fluorophenyl) pyrimidine-2,4- diamine | 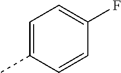 | 359.4 (MH+) | 6.03 (s, 2H), 6.16 (d, 1H), 6.94 (m, 7.07 (d, 1H), 7.61 (dd, 2H), 8.00 (d, 1H), 8.98 (s, 1H), 9.11 (s, 1H) | |
| 8 | N~4~-(5-chloro- 1,3-benzodioxol- 4-yl)-N~2~-(3- ethynylphenyl) pyrimidine-2,4- diamine | 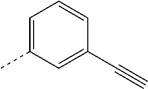 | 365.42 (MH+) | 4.03 (s, 1H), 6.02 (s, 2H), 6.19 (d, 1H), 6.94 (m, 2H), 7.08 (m, 2H), 7.68 (d, 1H), 7.76 (s, 1H), 8.03 (d, 1H), 9.02 (s, 1H), 9.21 (s, 1H) | |
| 9 | 3-({4-[(5-chloro- 1,3-benzodioxol- 4-yl)amino] pyrimidin-2-yl} amino) benzonitrile | 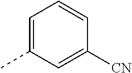 | 366.42 (MH+) | 6.04 (s, 2H), 6.23 (d, 1H), 6.92 (d, 1H), 7.08 (d, 1H), 7.70 (m, 2H), 7.86 (d, 1H), 8.09 (m, 2H), 9.13 (s, 1H), 9.50 (s, 1H) | |
| 10 | 4-({4-[(5-chloro- 1,3-benzodioxol- 4- yl)amino]pyrimidin- 2-yl}amino) benzonitrile | 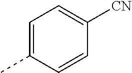 | 366.42 (MH+) | 6.04 (s, 2H), 6.28 (d, 1H), 6.99 (d, 1H), 7.09 (d, 1H), 7.52 (d, 2H), 7.72 (d, 2H), 8.09 (d, 1H), 9.19 (s, 1H), 9.70 (s, 1H) | |
| 11 | [3-({4-[(5-chloro- 1,3-benzodioxol- 4- yl)amino]pyrimidin- 2-yl}amino) phenyl] methanol | 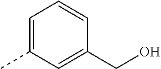 | 371.44 (MH+) | 4.36 (d, 2H), 5.01 (t, 1H), 6.01 (s, 2H), 6.13 (d, 1H), 6.82 (d, 1H), 6.92 (d, 1H), 7.04 (m, 2H), 7.50 (m, 2H), 7.99 (d, 1H), 8.94 (s, 1H), 9.05 (s, 1H) | |
| 12 | N~4~-(5-chloro- 1,3-benzodioxol- 4-yl)-N~2~-(4- methoxyphenyl) pyrimidine-2,4- diamine | 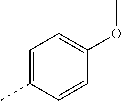 | 371.45 (MH+) | 3.70 (s, 3H), 6.01 (s, 2H), 6.09 (d, 1H), 6.70 (d, 2H), 6.93 (d, 1H), 7.04 (d, 1H), 7.50 (d, 2H), 7.95 (d, 1H), 8.86 (s, 1H), 8.90 (s, 1H) | |
| 13 | N~4~-(5-chloro- 1,3-benzodioxol- 4-yl)-N~2~-(3- chlorophenyl) pyrimidine-2,4- diamine | 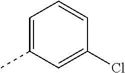 | 375.4 (M+) | 6.03 (s, 2H), 6.22 (d, 1H), 6.86 (dd, 1H), 7.92 (d, 1H), 7.06 (d, 1H), 7.13 (dd, 1H), 7.50 (d, 1H), 7.82 (m, 1H), 8.04 (d, 1H), 9.06 (s, 1H), 9.32 (s, 1H) | |
| 14 | N~4~-(5-chloro- 1,3-benzodioxol- 4-yl)-N~2~-(4- chlorophenyl) pyrimidine-2,4- diamine | 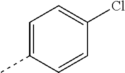 | 375.41 (M+) | 6.04 (s, 2H), 6.19 (d, 1H), 6.97 (d, 1H), 7.09 (m, 3H), 7.64 (d, 2H), 8.03 (d, 1H), 9.04 (s, 1H), 9.27 (s, 1H) | |
| 15 | N~4~-(5-chloro- 1,3-benzodioxol- 4-yl)-N~2~-(2,4- difluorophenyl) pyrimidine-2,4- diamine | 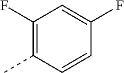 | 377.42 (MH+) | 6.06 (s, 2H), 6.16 (d, 1H), 7.91 (m, 2H), 7.08 (d, 1H), 7.22 (m, 1H), 7.78 (m, 1H), 8.00 (d, 1H), 8.46 (s, 1H), 8.98 (s, 1H) | |
| 16 | N~4~-(5-chloro- 1,3-benzodioxol- 4-yl)-N~2~-(3,5- difluorophenyl) pyrimidine-2,4- diamine | 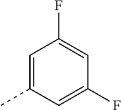 | 377.42 (MH+) | 6.03 (s, 2H), 6.26 (d, 1H), 6.58 (m, 1H), 6.92 (d, 1H), 7.05 (d, 1H), 7.87 (d, 2H), 8.06 (d, 1H), 9.15 (s, 1H), 9.55 (s, 1H) | |
| 17 | N~4~-(5-chloro- 1,3-benzodioxol- 4-yl)-N~2~-1H- indol-5-yl pyrimidine-2,4- diamine | 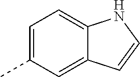 | 380.44 (MH+) | 6.01 (s, 2H), 6.10 (m, 1H), 6.20 (s, 1H), 6.96 (d, 1H), 7.07 (d, 1H), 7.17 (m, 3H), 7.90 (s, 1H), 7.97 (d, 1H), 8.81 (s, 1H), 8.87 (s, 1H), 10.78 (s, 1H) | |
| 18 | [4-({4-[(5-chloro- 1,3-benzodioxol- 4-yl)amino] pyrimidin-2- yl}amino)phenyl] acetonitrile | 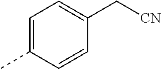 | 380.45 (MH+) | 3.90 (s, 1H), 6.04 (s, 2H), 6.17 (d, 1H), 6.92 (d, 1H), 7.08 (m, 3H), 7.62 (d, 2H), 8.01 (d, 1H), 9.01 (s, 1H), 9.17 (s, 1H) | |
| 19 | N~4~-(5-chloro- 1,3-benzodioxol- 4-yl)-N~2~-1H- indol-4-yl pyrimidine-2,4- diamine | 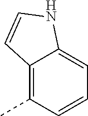 | 380.46 (MH+) | 6.02 (s, 2H), 6.15 (d, 1H), 6.80 (m, 2H), 7.96 (m, 2H), 7.05 (d, 1H), 7.20 (m, 1H), 7.64 (d, 1H), 8.02 (d, 1H), 8.54 (s, 1H), 8.93 (s, 1H), 10.94 (s, 1H) | |
| 20 | 3-({4-[(5-chloro- 1,3-benzodioxol- 4-yl)amino] pyrimidin-2- yl}amino) benzamide | 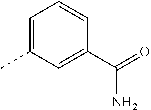 | 384.44 (MH+) | 6.03 (s, 2H), 6.21 (d, 1H), 6.98 (d, 1H), 7.09 (d, 2H), 7.66 (m, 5H), 8.05 (d, 1H), 9.08 (s, 1H), 9.39 (s, 1H) | |
| 21 | 4-({4-[(5-chloro- 1,3-benzodioxol- 4-yl)amino] pyrimidin-2- yl}amino) benzamide | 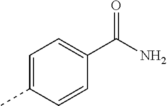 | 384.45 (MH+) | 6.03 (s, 2H), 6.21 (d, 1H), 6.98 (d, 1H), 7.09 (d, 2H), 7.65 (m, 5H), 8.05 (d, 1H), 9.08 (s, 1H), 9.38 (s, 1H) | |
| 22 | N~4~-(5-chloro- 1,3-benzodioxol- 4-yl)-N~2~-1H- indol-6- ylpyrimidine-2,4- diamine | 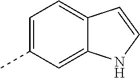 | 380.46 (MH+) | | |
| 23 | 3-({4-[(5-chloro- 1,3-benzodioxol- 4- yl)amino]pyrimidin- 2-yl}amino)-N- (2-methoxyethyl) benzamide | 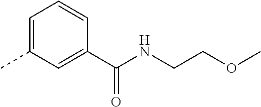 | 442.45 (MH+) | | |
| 24 | N~4~-(5-chloro- 1,3-benzodioxol- 4-yl)-N~2~-[4- (pyridin-2- ylmethoxy) phenyl] pyrimidine-2,4- diamine | 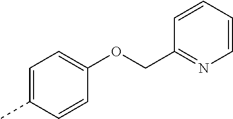 | | 5.19 (s, 2H), 6.03 (s, 2H), 6.17 (d, 1H), 6.90 (d, 1H), 7.00 (d, 1H), 7.16 (d, 1H), 7.37 (m, 1H), 7.43 (dd, 1H), 7.57 (d, 1H), 7.86 (m, 2H), 8.00 (d, 1H), 8.60 (d, 1H), 8.99 (s, 1H), 9.12 (s, 1H) | |
| 25 | 1-[4-({4-[(5- chloro-1,3- benzodioxol-4- yl)amino]pyrimidin- 2-yl}amino) phenyl]-N- methylmethane sulfonamide | 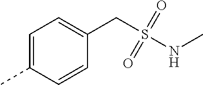 | 448.39 (MH+) | | |
| 26 | N~4~-(5-chloro- 1,3-benzodioxol- 4-yl)-N~2~-[3-(2- pyrrolidin-1- ylethoxy)phenyl] pyrimidine-2,4- diamine | 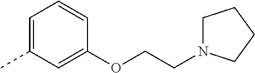 | 454.47 (MH+) | 1.70 (m, 4H), 2.53 (m, 4H, obscured by solvent), 2.77 (t, 2H), 3.95 (t, 2H), 6.02 (s, 2H), 6.15 (d, 1H), 6.43 (dd, 1H), 6.90 (d, 1H), 7.00 (t, 1H), 7.05 (d, 1H), 7.26 (m, 2H), 8.00 (d, 1H), 8.94 (s, 1H), 9.00 (s, 1H) | |
| 27 | N~4~-(5-chloro- 1,3-benzodioxol- 4-yl)-N~2~-(3- chloro-4- morpholin-4- ylphenyl) pyrimidine-2,4- diamine | 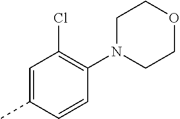 | 460.4 (M+) | 2.88 (m, 4H), 3.74 (m, 4H), 6.00 (s, 2H), 6.16 (d, 1H), 6.94 (m, 3H), 7.04 (d, 1H), 7.45 (dd, 1H), 7.82 (d, 1H), 8.01 (d, 1H), 8.99 (s, 1H), 9.13 (s, 1H) | |
| 28 | N~4~-(5-chloro- 1,3-benzodioxol- 4-yl)-N~2~-[4-(2- morpholin-4- ylethoxy) phenyl] pyrimidine-2,4- diamine | 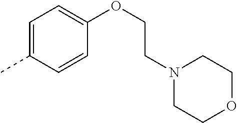 | 470.45 (MH+) | 2.47 (m, 4H), 2.67 (t, 2H), 3.60 (m, 4H), 4.02 (t, 2H), 6.03 (s, 2H), 6.10 (d, 1H), 6.70 (d, 2H), 6.93 (d, 1H), 7.06 (d, 1H), 7.50 (d, 2H), 7.96 (d, 1H), 8.87 (s, 1H), 8.89 (s, 1H) | |
| 29 | 4-({4-[(5-chloro- 1,3-benzodioxol- 4-yl)amino] pyrimidin-2- yl}amino)-N-(2- hydroxyethyl)-N- methyl benzene sulfonamide | 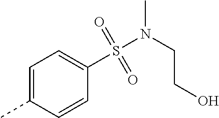 | 478.45 (MH+) | 2.68 (s, 3H), 2.95 (t, 2H), 3.53 (m, 2H), 4.74 (t, 1H), 6.04 (s, 2H), 6.26 (d, 1H), 6.97 (d, 1H), 7.08 (d, 1H), 7.47 (d, 2H), 7.83 (d, 2H), 8.07 (d, 1H), 9.15 (s, 1H), 9.65 (s, 1H) | |
| 30 | 4-({4-[(5-chloro- 1,3-benzodioxol- 4-yl)amino] pyrimidin-2- yl}amino)-N-[2- (diethylamino) ethyl]benzamide | 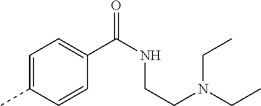 | 483.56 (MH+) | 1.00 (t, 6H), 2.53 (m, 6H, obscured by solvent), 3.30 (m, 2H, obscured by water), 6.03 (s, 2H), 6.21 (d, 1H), 6.96 (d, 1H), 7.07 (d, 1H), 7.60 (d, 2H), 7.67 (d, 2H), 8.05 (m, 2H), 9.07 (s, 1H), 9.38 (s, 1H) | |
| 31 | N~4~-(5-chloro- 1,3-benzodioxol- 4-yl)-N~2~-{4-[2- (4- methylpiperazin- 1-yl)ethoxy] phenyl} pyrimidine-2,4- diamine | 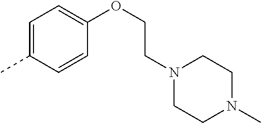 | 481.64 (M - H+) | 2.17 (s, 3H), 2.33 (m, 4H), 2.48 (m, 4H), 2.65 (t, 2H), 3.98 (t, 2H), 6.03 (s, 2H), 6.10 (d, 1H), 6.68 (d, 2H), 6.93 (d, 1H), 7.04 (d, 1H), 7.48 (d, 2H), 7.96 (d, 1H), 8.86 (s, 1H), 8.90 (s, 1H) | |
| 32 | N~4~-(5-chloro- 1,3-benzodioxol- 4-yl)-N~2~-{4- [(3-fluorobenzyl) oxy]-3-methoxy phenyl} pyrimidine-2,4- diamine | 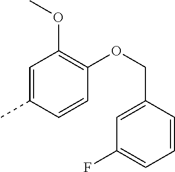 | 495.44 (MH+) | 3.62 (s, 3H), 5.01 (s, 2H), 5.97 (s, 2H), 6.08 (d, 1H), 6.75 (d, 1H), 6.90 (d, 1H), 7.03 (d, 1H), 7.16 (m, 2H), 7.27 (m, 2H), 7.43 (m, 1H), 7.96 (d, 1H), 8.86 (s, 1H), 8.89 (s, 1H) | |
| 33 | N~4~-(5-chloro- 1,3-benzodioxol- 4-yl)-N~2~-{4- [(2-fluorobenzyl) oxy]-3-methoxy phenyl} pyrimidine-2,4- diamine | 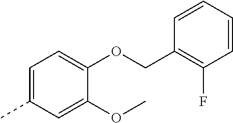 | 495.43 (MH+) | 3.62 (s, 3H), 5.01 (s, 2H), 5.97 (s, 2H), 6.08 (d, 1H), 6.75 (d, 1H), 6.90 (d, 1H), 7.03 (d, 1H), 7.16 (m, 2H), 7.27 (m, 2H), 7.43 (m, 1H), 7.96 (d, 1H), 8.86 (s, 1H), 8.89 (s, 1H) | |
| 34 | 4-({4-[(5-chloro- 1,3-benzodioxol- 4- yl)amino]pyrimidin- 2-yl}amino)-N- (2-methoxyethyl) benzene sulfonamide | 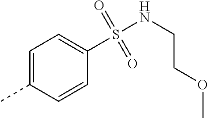 | 478.4 (MH+) | | |
| 35 | N-[4-({4-[(5- chloro-1,3- benzodioxol-4- yl)amino]pyrimidin- 2-yl}amino) phenyl]-N-methyl acetamide | 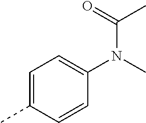 | 412.5 (MH+) | | |
| 36 | N-[5-({4-[(5- chloro-1,3- benzodioxol-4- yl)amino]pyrimidin- 2-yl}amino)-2- methylphenyl] acetamide | 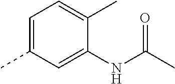 | 412.49 (MH+) | 2.04 (s, 3H), 2.08 (s, 3H), 6.03 (s, 2H), 6.12 (dd, 1H), 6.90 (m, 2H), 7.05 (d, 1H), 7.46 (m, 2H), 7.98 (d, 1H), 8.90 (s, 1H), 8.97 (s, 1H), 9.17 (s, 1H) | |
| 37 | N-[4-({4-[(5- chloro-1,3- benzodioxol-4- yl)amino]pyrimidin- 2-yl}amino) benzyl] acetamide | 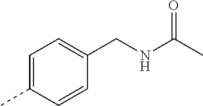 | 412.5 (MH+) | | |
| 38 | N~4~-(5-chloro- 1,3-benzodioxol- 4-yl)-N~2~-[3- (methylsulfonyl) phenyl} pyrimidine-2,4- diamine | 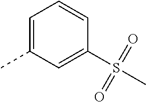 | 419.45 (MH+) | 3.12 (s, 3H), 6.05 (s, 2H), 6.21 (d, 1H), 6.94 (d, 1H), 7.06 (d, 1H), 7.37 (m, 2H), 8.10 (m, 3H), 9.06 (s, 1H), 9.47 (s, 1H) | |
| 39 | 4-({4-[(5-chloro- 1,3-benzodioxol- 4-yl)amino] pyrimidin-2- yl}amino) benzene sulfonamide | 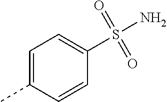 | 420.46 (MH+) | 6.05 (s, 2H), 6.22 (d, 1H), 6.96 (d, 1H), 7.10 (m, 3H), 7.54 (d, 2H), 7.86 (d, 2H), 8.07 (d, 1H), 9.13 (s, 1H), 9.54 (s, 1H) | |
| 40 | 3-({4-[(5-chloro- 1,3-benzodioxol- 4- yl)amino]pyrimidin- 2-yl}amino) benzene sulfonamide | 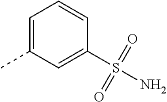 | 420.45 (MH+) | 6.06 (s, 2H), 6.17 (d, 1H), 6.93 (d, 1H), 7.07 (d, 1H), 7.27 (m, 4H), 7.98 (d, 1H), 8.04 (d, 1H), 8.09 (s, 1H), 9.02 (s, 1H), 9.42 (s, 1H) | |
| 41 | N~4~-(5-chloro- 1,3-benzodioxol- 4-yl)-N~2~-[4- (trifluoromethoxy) phenyl] pyrimidine-2,4- diamine | 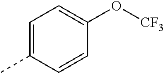 | 425.44 (MH+) | 6.02 (s, 2H), 6.19 (d, 1H), 6.94 (d, 1H), 7.07 (m, 3H), 7.70 (d, 2H), 8.03 (d, 1H), 9.04 (s, 1H), 9.30 (s, 1H) | |
| 42 | N~4~-(5-chloro- 1,3-benzodioxol- 4-yl)-N~2~-(4- morpholin-4- ylphenyl) pyrimidine-2,4- diamine | 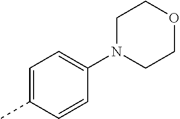 | 426.47 (MH+) | 3.00 (m, 4H), 3.75 (m, 4H), 6.00 (s, 2H), 6.07 (d, 1H), 6.73 (d, 2H), 6.92 (d, 1H), 7.03 (d, 1H), 7.46 (d, 2H), 7.94 (d, 1H), 8.80 (s, 1H), 8.88 (s, 1H) | |
| 43 | N~4~-(5-chloro- 1,3-benzodioxol- 4-yl)-N~2~-(2- morpholin-4- ylphenyl) pyrimidine-2,4- diamine | 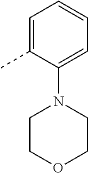 | 426.47 (MH+) | 2.80 (m, 4H), 3.78 (m, 4H), 6.03 (s, 2H), 6.18 (d, 1H), 6.90 (m, 2H), 6.96 (d, 1H), 7.07 (d, 1H), 7.20 (m, 1H), 8.03 (m, 2H), 8.18 (m, 1H), 9.13 (s, 1H) | |
| 44 | N~4~-(5-chloro- 1,3-benzodioxol- 4-yl)-N~2~-(3- morpholin-4- ylphenyl) pyrimidine-2,4- diamine | 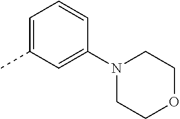 | 426.46 (MH+) | | |
| 45 | N~4~-(5-chloro- 1,3-benzodioxol- 4-yl)-N~2~-[4-(2- ethoxyethoxy) phenyl] pyrimidine-2,4- diamine | 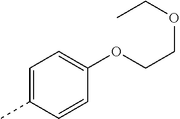 | 429.42 (MH+) | 1.15 (t, 3H), 3.53 (q, 4H), 3.68 (m, 2H), 4.00 (m, 2H), 6.03 (s, 2H), 6.10 (d, 1H), 6.70 (d, 2H), 6.93 (d, 1H), 7.04 (d, 1H), 7.50 (d, 2H), 7.96 (d, 1H), 9.88 (s, 1H), 9.90 (s, 1H) | |
| 46 | N~4~-(5-chloro- 1,3-benzodioxol- 4-yl)-N~2~- (2,3,4-trimethoxy phenyl) pyrimidine-2,4- diamine | 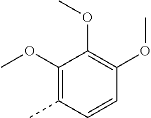 | 431.41 (MH+) | 3.75 (s, 6H), 3.81 (s, 3H), 6.02 (s, 2H), 6.10 (d, 1H), 6.53 (d, 1H), 6.93 (d, 1H), 7.05 (d, 1H), 7.50 (s, 1H), 7.64 (d, 1H), 7.96 (d, 1H), 8.98 (s, 1H) | |
| 47 | N-[3-({4-[(5- chloro-1,3- benzodioxol-4- yl)amino]pyrimidin- 2-yl}amino) phenyl]methane sulfonamide | 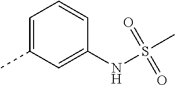 | 434.37 (MH+) | 2.98 (s, 3H), 6.04 (s, 2H), 6.13 (d, 1H), 6.71 (d, 1H), 6.92 (d, 1H), 7.05 (m, 2H), 7.42 (s, 1H), 7.57 (d, 1H), 8.00 (d, 1H), 8.90 (s, 1H), 9.11 (s, 1H) | |
| 48 | N~4~-(5-chloro- 1,3-benzodioxol- 4-yl)-N~2~-[3- (dimethylamino) phenyl] pyrimidine-2,4- diamine | 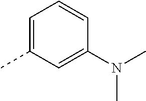 | 384.51 (MH+) | | |
| 49 | 2-[4-({4-[(5- chloro-1,3- benzodioxol-4- yl)amino]pyrimidin- 2-yl}amino) phenyl]ethanol | 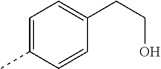 | 385.49 (MH+) | 2.52 (t, 2H), 3.55 (q, 2H), 4.55 (t, 1H), 6.02 (s, 2H), 6.13 (d, 1H), 6.95 (m, 3H), 7.05 (d, 1H), 7.51 (d, 2H), 7.98 (d, 1H), 8.93 (s, 1H), 9.97 (s, 1H) | |
| 50 | N~4~-(5-chloro- 1,3-benzodioxol- 4-yl)-N~2~-(3- chloro-4- fluorophenyl) pyrimidine-2,4- diamine | 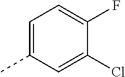 | 393.43 (M+) | 6.05 (s, 2H), 6.22 (d, 1H), 6.94 (d, 1H), 7,07 (d, 1H), 7.16 (t, 1H), 7.49 (m, 1H), 7.96 (m, 1H), 8.04 (d, 1H), 9.08 (s, 1H), 9.32 (s, 1H) | |
| 51 | N~4~-(5-chloro- 1,3-benzodioxol- 4-yl)-N~2~-(4- chloro-2- fluorophenyl) pyrimidine-2,4- diamine | 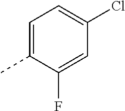 | 393.46 (M+) | 6.03 (s, 2H), 6.18 (d, 1H), 6.93 (d, 1H), 7.00 (d, 1H), 7.05 (d, 1H), 7.34 (dd, 1H), 7.89 (t, 1H), 8.00 (d, 1H), 8.51 (s, 1H), 9.03 (s, 1H) | |
| 52 | N~4~-(5-chloro- 1,3-benzodioxol- 4-yl)-N~2~-(3- chloro-2- fluorophenyl) pyrimidine-2,4- diamine | 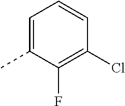 | 393.46 (M+) | 6.02 (s, 2H), 6,19 (d, 1H), 6.90 (d, 1H), 6.96 (m, 1H), 7.03 (d, 1H), 7.15 (m, 1H), 7.81 (m, 1H), 8.00 (d, 1H), 8.65 (s, 1H), 9.04 (s, 1H) | |
| 53 | N~4~-(5-chloro- 1,3-benzodioxol- 4-yl)-N~2~-(5- chloro-2- fluorophenyl) pyrimidine-2,4- diamine | 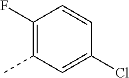 | 393.46 (M+) | 6.03 (s, 2H), 6.24 (d, 1H), 6.90 (d, 1H), 6.98 (m, 1H), 7.05 (d, 1H), 7.20 (dd, 1H), 8.04 (m, 2H), 8.47 (s, 1H), 9.11 (s, 1H) | |
| 54 | N~4~-(5-chloro- 1,3-benzodioxol- 4-yl)-N~2~-(4- chloro-3- fluorophenyl) pyrimidine-2,4- diamine | 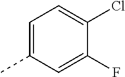 | 393.46 (M+) | 6.05 (s, 2H), 6.23 (d, 1H), 6.95 (d, 1H), 7.07 (d, 1H), 7.30 (m, 2H), 7.88 (d, 1H), 8.05 (d, 1H), 9.14 (s, 1H), 9.51 (s, 1H) | |
| 55 | 5-({4-[(5-chloro- 1,3-benzodioxol- 4- yl)amino]pyrimidin- 2-yl}amino)- 1,3-dihydro-2H- indol-2-one | 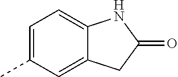 | 396.51 (MH+) | 6.02 (s, 2H), 6.12 (d, 1H), 6,59 (d, 1H), 6.95 (d, 1H), 7.06 (d, 1H), 7.28 (d, 1H), 7.57 (s, 1H), 7.97 (d, 1H), 8.93 (s, 1H), 8.95 (s, 1H), 10.12 (s, 1H) | |
| 56 | N-[4-({4-[(5- chloro-1,3- benzodioxol-4- yl)amino]pyrimidin- 2-yl}amino) phenyl]acetamide | 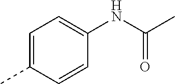 | 398.53 (MH+) | 6.03 (s, 2H), 6.12 (d, 1H), 6.92 (d, 1H), 7.06 (d, 1H), 7.20 (d, 2H), 7.62 (d, 2H), 7.98 (d, 1H), 8.94 (s, 1H), 9.00 (s, 1H), 9.70 (s, 1H) | |
| 57 | 3-({4-[(5-chloro- 1,3-benzodioxol- 4- yl)amino]pyrimidin- 2-yl}amino)-N- methyl benzamide | 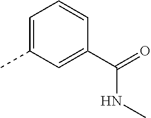 | 398.53 (MH+) | | |
| 58 | 4-({4-[(5-chloro- 1,3-benzodioxol- 4- yl)amino]pyrimidin- 2-yl}amino)-N- methyl benzamide | 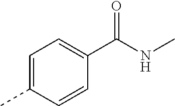 | 398.49 (MH+) | 2.78 (d, 3H), 6.04 (s, 2H), 6.22 (d, 1H), 6.97 (d, 1H), 7.08 (d, 1H), 7.60 (d, 2H), 7.68 (d, 2H), 8.05 (d, 1H), 8.16 (m, 1H), 9.07 (s, 1H), 9.38 (s, 1H) | |
| 59 | N~2~-1,3- benzothiazol-6-yl- N~4~-(5-chloro- 1,3-benzodioxol- 4-yl)pyrimidine- 2,4-diamine | 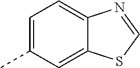 | 398.44 (MH+) | 6.10 (s, 2H), 6.30 (d, 1H), 7.09 (d, 1H), 7.19 (d, 1H), 7.63 (dd, 1H), 7.92 (d, 1H), 8.15 (d, 1H), 8.63 (s, 1H), 9.19 (m, 2H), 9.56 (s, 1H) | |
| 60 | N~4~-(5-chloro- 1,3-benzodioxol- 4-yl)-N~2~-(2,5- dimethoxy phenyl) pyrimidine-2,4- diamine | 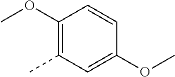 | 401.48 (MH+) | 3.59 (s, 3H), 3.81 (s, 3H), 6.02 (s, 2H), 6.17 (d, 1H), 6.44 (dd, 1H), 6.90 (m, 2H), 7.04 (d, 1H), 7.43 (s, 1H), 7.88 (m, 1H), 8.02 (d, 1H), 9.07 (s, 1H) | |
| 61 | N~4~-(5-chloro- 1,3-benzodioxol- 4-yl)-N~2~-(2,4- dimethoxy phenyl) pyrimidine-2,4- diamine | 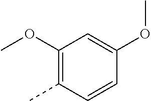 | 401.47 (MH+) | 3.74 (s, 3H), 3.82 (s, 3H), 6.01 (s, 2H), 6.09 (d, 1H), 6.30 (dd, 1H), 6.59 (m, 1H), 6.93 (d, 1H), 7.06 (d, 1H), 7.31 (s, 1H), 7.85 (d, 1H), 7.96 (d, 1H), 8.96 (s, 1H) | |
| 62 | N~4~-(5-chloro- 1,3-benzodioxol- 4-yl)-N~2~-(3,5- dimethoxy phenyl) pyrimidine-2,4- diamine | 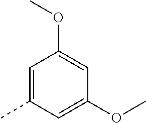 | 401.48 (MH+) | 3.63 (s, 6H), 6.02 (s, 2H), 6.13 (d, 1H), 6.88 (d, 1H), 7.95 (m, 2H), 7.03 (d, 1H), 8.01 (d, 1H), 8.94 (s, 3H), 8.98 (s, 1H) | |
| 63 | N~4~-(5-chloro- 1,3-benzodioxol- 4-yl)-N~2~-(3,4- dimethoxy phenyl) pyrimidine-2,4- diamine | 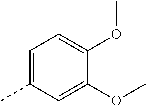 | 401.48 (MH+) | 3.59 (s, 3H), 3.69 (s, 3H), 6.00 (s, 2H), 6.09 (d, 1H), 6.70 (d, 1H), 6.90 (d, 1H), 7.04 (d, 1H), 7.18 (dd, 1H), 7.30 (m, 1H), 7.96 (d, 1H), 8.81 (s, 1H), 8.89 (s, 1H) | |
| 64 | N~4~-(5-chloro- 1,3-benzodioxol- 4-yl)-N~2~-(5- chloro-2- methoxyphenyl) pyrimidine-2,4- diamine | 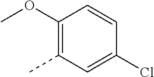 | 405.43 (M+) | 3.87 (s, 3H), 6.03 (s, 2H), 6.24 (d, 1H), 6.80 (m, 2H), 6.98 (d, 1H), 7.06 (d, 1H), 7.53 (s, 1H), 8.04(d, 1H), 8.20 (m, 1H), 9.18 (s, 1H) | |
| 65 | N~4~-(5-chloro- 1,3-benzodioxol- 4-yl)-N~2~-(2- chloro-5- methoxyphenyl) pyrimidine-2,4- diamine | 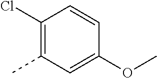 | 405.43 (M+) | 3.63 (s, 3H), 6.00 (s, 2H), 6.19 (d, 1H), 6.60 (dd, 1H), 6.90 (d, 1H), 7.02 (d, 1H), 7.32 (d, 1H), 7.70 (s, 1H), 7.79 (m, 1H), 8.03 (d, 1H), 9.10 (s, 1H) | |
| 66 | N~4~-(5-chloro- 1,3-benzodioxol- 4-yl)-N~2~-(3- chloro-2- methoxyphenyl) pyrimidine-2,4- diamine | 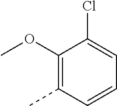 | 405.43 (M+) | 3.88 (s, 3H), 6.11 (s, 2H), 6.28 (d, 1H), 6.94 (m, 1H), 7.03 (d, 1H), 7.08 (d, 1H), 7.16 (d, 1H), 7.86 (s, 1H), 8.11 (d, 1H), 8.16 (d, 1H), 9.24 (s, 1H) | |
| 67 | N~4~-(5-chloro- 1,3-benzodioxol- 4-yl)-N~2~-[3- (1,3-oxazol-5- yl)phenyl] pyrimidine-2,4- diamine | 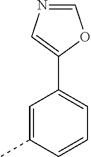 | 408.46 (M+) | 5.98 (s, 2H), 6.18 (d, 1H), 6.85 (d, 1H), 7.03 (d, 1H), 7.20 (m, 2H), 7.47 (s, 1H), 7.70 (m, 1H), 7.94 (s, 1H), 8.03 (d, 1H), 8.40 (s, 1H), 9.00 (s, 1H), 9.23 (s, 1H) | |
| 80 | N~4~-(5-chloro- 1,3-benzodioxol- 4-yl)-N~2~-(3- methylphenyl) pyrimidine-2,4- diamine | 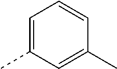 | 355.19 (M+) | | |
| 81 | N~4~-(5-chloro- 1,3-benzodioxol- 4-yl)-N~2~-(3- methoxyphenyl) pyrimidine-2,4- diamine | 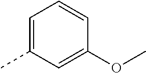 | 371.42 (M+) | | |
| 82 | N~4~-(5-chloro- 1,3-benzodioxol- 4-yl)-N~2~- quinolin-6-yl pyrimidine-2,4- diamine | 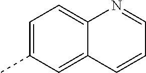 | 392.09 (M+) | | |
| 83 | N-[3-({4-[(5- chloro-1,3- benzodioxol-4- yl)amino]pyrimidin- 2-yl}}amino) phenyl] acetamide | 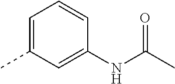 | 398.09 (M+) | | |
| 84 | N~4~-(5-chloro- 1,3-benzodioxol- 4-yl)-N~2~-(2,3- dihydro-1,4- benzodioxin-6- yl)pyrimidine-2,4- diamine | 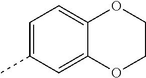 | 399.10 (M+) | | |
| 85 | N~4~-(5-chloro- 1,3-benzodioxol- 4-yl)-N~2~-[3- (difluoro methoxy)phenyl] pyrimidine-2,4- diamine | 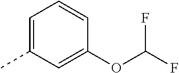 | 407.05 (M+) | | |
| 86 | N~4~-(5-chloro- 1,3-benzodioxol- 4-yl)-N~2~-[3- (trifluoromethyl) phenyl] pyrimidine-2,4- diamine | 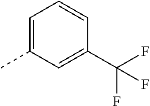 | 408.90 (M+) | | |
| 87 | 6-({4-[(5-chloro- 1,3-benzodioxol- 4- yl)amino]pyrimidin- 2-yl}amino)- 2H-chromen-2- one | 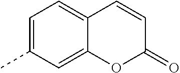 | 408.94 (M+) | | |
| 88 | N-(4-({4-[(5- chloro-1,3- benzodioxol-4- yl)amino]pyrimidin- 2-yl}amino)-2- methyl phenyl] acetamide | 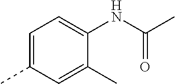 | 411.95 (M+) | | |
| 89 | 4-({4-[(5-chloro- 1,3-benzodioxol- 4- yl)amino]pyrimidin- 2-yl}amino)- N,N-dimethyl benzamide | 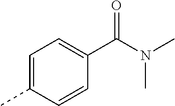 | 411.96 (M+) | | |
| 90 | 3-({4-[(5-chloro- 1,3-benzodioxol- 4- yl)amino]pyrimidin- 2-yl}amino)- N-ethyl benzamide | 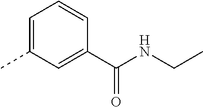 | 411.98 (M+) | | |
| 91 | N~4~-(5-chloro- 1,3-benzodioxol- 4-yl)-N~2~-(2- methyl-1,3- benzothiazol-5- yl)pyrimidine-2,4- diamine | 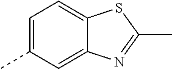 | 411.93 (M+) | | |
| 92 | N~4~-(5-chloro- 1,3-benzodioxol- 4-yl)-N~2~(2- methyl-1,3- benzothiazol-6- yl)pyrimidine-2,4- diamine | 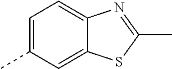 | 411.93 (M+) | | |
| 93 | N~2~-(1-acetyl- 2,3-dihydro-1H- indol-5-yl)-N~4~- (5-chloro-1,3- benzodioxol-4- yl)pyrimidine-2,4- diamine | 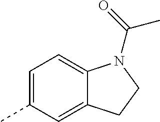 | 423.97 (M+) | | |
| 94 | N~2~-(1-acetyl- 2,3-dihydro-1H- indol-6-yl)-N~4~- (5-chloro-1,3- benzodioxol-4- yl)pyrimidine-2,4- diamine | 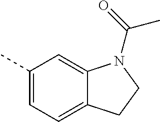 | 423.95 (M+) | | |
| 95 | 3-({4-[(5-chloro- 1,3-benzodioxol- 4- yl)amino]pyrimidin- 2-yl}amino)-N- isopropyl benzamide | 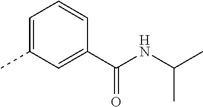 | 425.95 (M+) | | |
| 96 | N-[3-({4-[(5- chloro-1,3- benzodioxol-4- yl)amino]pyrimidin- 2-yl}amino)-4- methoxy phenyl] acetamide | 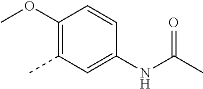 | 427.93 (M+) | | |
| 97 | 2-{[3-({4-[(5- chloro-1,3- benzodioxol-4- yl)amino]pyrimidin- 2-yl}amino) phenyl]sulfonyl} ethanol | 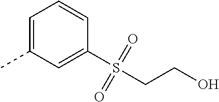 | 449.39 (M+) | | |
| 98 | N~4~-(5-chloro- 1,3-benzodioxol- 4-yl)-N~2~-[2- methoxy-5- (methylsulfonyl) phenyl] pyrimidine-2,4- diamine | 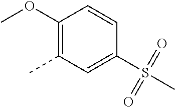 | 449.38 (M+) | | |
| 99 | N~4~-(5-chloro- 1,3-benzodioxol- 4-yl)-N~2~-{4-[3- (diethylamino) propoxy)phenyl} pyrimidine-2,4- diamine | 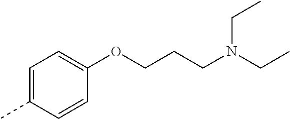 | 470.50 (M+) | | |
| 100 | 4-({4-[(5-chloro- 1,3-benzodioxol- 4- yl)amino]pyrimidin- 2-yl}amino)-N- [2- (dimethylamino) ethyl]-2-fluoro benzamide | 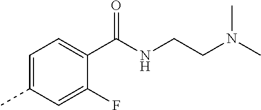 | 473.47 (M+) | | |
| 101 | 3-({4-[(5-chloro- 1,3-benzodioxol- 4- yl)amino]pyrimidin- 2-yl}amino)-N- methylbenzene sulfonamide | 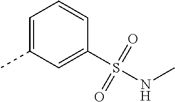 | 434.38 (M+) | (300 MHz) 2.70 (s, 3H), 6.04 (s, 2H), 6.18 (d, 1H), 6.92 (d, 1H), 7.05 (d, 1H), 7.25 (d, 1H), 7.29 (d, 1H), 7.31-8.00 (m, 1H), 8.03-8.05 m, (3H), 9.02 (s, 1H), 9.42 (s, 1H) | |
| 102 | 3-({4-[(5-chloro- 1,3-benzodioxol- 4- yl)amino]pyrimidin- 2-yl}amino)- N,N-dimethyl benzene sulfonamide | 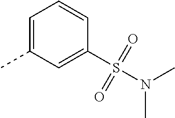 | 448.39 (M+) | (300 MHz) 2.60 (s, 6H), 6.04 (s, 2H), 6.21 (d, 1H), 6.92 (d, 1H), 7.05 (d, 1H), 7.18 (d, 1H), 7.32 (t, 1H), 7.94 (s, 1H), 8.04 (d, 1H), 8.15 (d, 1H), 9.04 (s, 1H), 9.42 (s, 1H) | |
| 103 | 4-({4-[(5-chloro- 1,3-benzodioxol- 4- yl)amino]pyrimidin- 2-yl}amino) phenylmethane sulfonate | 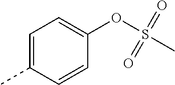 | 435.36 (M+) | (300 MHz) 3.30 (3H, under water), 6.02 (s, 2H), 6.17 (d, 1H), 6.93 (d, 1H), 7.04 (s, 1H, 7.04-7.08 (m, 2H), 7.68 (d, 2H), 8.01 (d, 1H), 9.03 (s, 1H), 9.28 (s, 1H) | |
| 104 | 3-({4-[(5-chloro- 1,3-benzodioxol- 4- yl)amino]pyrimidin- 2-yl}amino) phenylmethane sulfonate | 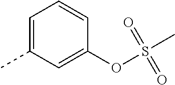 | 435.46 (M+) | (300 MHz) 3.37 (3H, under water), 6.02 (s, 2H), 6.18 (d, 1H), 6.80-6.83 (m, 1H), 6.91 (d, 1H), 7.05 (d, 1H), 7.18 (t, 1H), 7.61- 7.65 (d, 2H), 8.03 (d, 1H), 9.04 (s, 1H), 9.33 (s, 1H) | |
| 105 | N~4~-(5-chloro- 1,3-benzodioxol- 4-yl)-N~2~4- (methylsulfonyl) phenyl] pyrimidine-2,4- diamine | 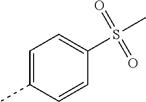 | 419.37 (M+) | (300 MHz) 3.10 (s, 3H), 6.04 (s, 2H), 6.26 (d, 1H), 6.97 (d, 1H), 7.08 (d, 1H), 7.60 (d, 2H), 7.84 (d, 2H), 8.07 (d, 1H), 9.16 (s, 1H), 9.69 (s, 1H) | |
| 106 | 3-( {4-[(5-chloro- 1,3-benzodioxol- 4- yl)amino]pyrimidin- 2-yl}amino) phenol | 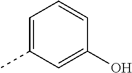 | 357.40 (M+) | | |
| 107 | N~4~-(5-chloro- 1,3-benzodioxol- 4-yl)-N~2~-1H- indazol-4-yl pyrimidine-2,4- diamine | 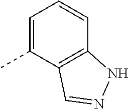 | 381.08 (M+) | | |
| 108 | N~2~-1H-1,2,3- benzotriazol-5-yl- N~4~-(5-chloro- 1,3-benzodioxol- 4-yl)pyrimidine- 2,4-diamine | 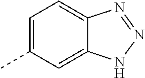 | 382.08 (M+) | | |
| 109 | N~4~-(5-chloro- 1,3-benzodioxol- 4-yl)-N~2~-(1,3- dihydro-2- benzofuran-5- yl)pyrimidine-2,4- diamine | 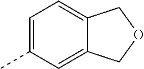 | 383.10 (M+) | | |
| 110 | N~4~-(5-chloro- 1,3-benzodioxol- 4-yl)-N~2~-[4- (dimethylamino) phenyl] pyrimidine-2,4- diamine | 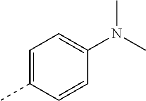 | 384.12 (M+) | | |
| 111 | N~2~-1,3- benzodioxol-5-yl- N~4~-(5-chloro- 1,3-benzodioxol- 4-yl)pyrimidine- 2,4-diamine | 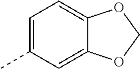 | 385.08 (M+) | | |
| 112 | N~4~-(5-chloro- 1,3-benzodioxol- 4-yl)-N~2~-(4- ethoxyphenyl) pyrimidine-2,4- diamine | 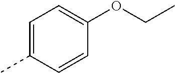 | 385.12 (M+) | | |
| 113 | N~4~-(5-chloro- 1,3-benzodioxol- 4-yl)-N~2~-(3- ethoxyphenyl) pyrimidine-2,4- diamine | 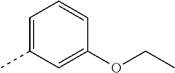 | 385.12 (M+) | | |
| 114 | N~4~-(5-chloro- 1,3-benzadioxol- 4-yl)-N~2~-[3- (difluoromethyl) phenyl] pyrimidine-2,4- diamine | 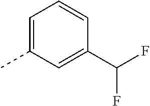 | 391.09 (M+) | | |
| 115 | N~4~-(5-chloro- 1,3-benzodioxol- 4-yl)-N~2~- quinoxalin-6- ylpyrimidine-2,4- diamine | 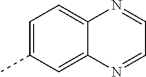 | 393.09 (M+) | | |
| 116 | N~4~-(5-chloro- 1,3-benzodioxol- 4-yl)-N~2~-(2- methyl-1H-indol- 5-yl) pyrimidine- 2,4-diamine | 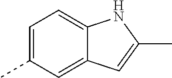 | 394.13 (M+) | | |
| 117 | N~4~-(5-chloro- 1,3-benzodioxol- 4-yl)-N~2~-(2- methyl-1H- benzimidazol-6- yl)pyrimidine-2,4- diamine | 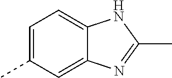 | 393.10 (M+) | | |
| 118 | N~2~-1- benzothien-5-yl- N~4~-(5-chloro- 1,3-benzodioxol- 4-yl)pyrimidine- 2,4-diamine | 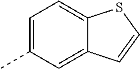 | 397.08 (M+) | | |
| 119 | N~4~-(5-chloro- 1,3-benzodioxol- 4-yl)-N~2~-[4- (1H-1,2,4-triazol- 1-yl)phenyl] pyrimidine-2,4- diamine | 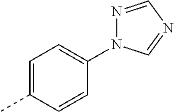 | 408.10 (M+) | | |
| 120 | [5-({4-{(5-chloro- 1,3-benzodioxol- 4- yl)amino]pyrimidin- 2-yl}amino)- 1H-benzimidazol- 2-yl]methanol | 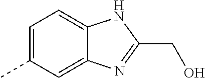 | 411.12 (M+) | | |
| 121 | N~4~-(5-chloro- 1,3-benzodioxol- 4-yl)-N~2~-[4- (diethylamino) phenyl] pyrimidine-2,4- diamine | 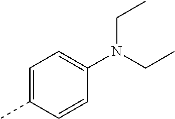 | 412.18 (M+) | | |
| 122 | N~4~-(5-chloro- 1,3-benzodioxol- 4-yl)-N~2~-(3,4- dihydro-2H-1,5- benzodioxepin-7- yl)pyrimidine-2,4- diamine | 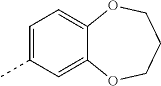 | 413.13 (M+) | | |
| 123 | N~6~-{4-[(5- chloro-1,3- benzodioxol-4- yl)amino]pyrimidin- 2-yl}-1,3- benzothiazole- 2,6-diamine | 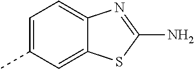 | 411.05 (M - H+) | | |
| 124 | N~4~-(5-chloro- 1,3-benzodioxol- 4-yl)-N~2~-(4- piperidin-1- ylphenyl) pyrimidine-2,4- diamine | 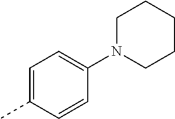 | 424.18 (M+) | | |
| 125 | 5-[[4-[(5- chlorobenzo[1,3] dioxol-4- yl)amino]pyrimidin- 2-yl]amino] benzene-1,3- dicarboxamide | 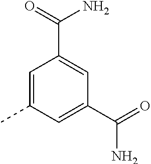 | 427.41 (M+) | 6.04 (s, 211), 6.10 (d, 1H), 6.85 (d, 1H), 7.00 (d, 1H), 7.30 (br s, 2H), 7.73 (br s, 2H), 7.78 (s, 1H), 8.02 (d, 1H), 8.20 (s, 2H), 8.93 (s, 1H), 9.18 (s, 1H) | |
| 126 | [3-[[4-[(5- chlorobenzo[1,3] dioxol-4- yl)amino]pyrimidin- 2-yl]amino]-5- morpholin-4-yl- phenyl]methanol | 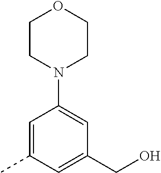 | 456.38 (M+) | 2.96 (m, 4H), 3.70 (m, 4H), 4.28 (d, 2H), 4.93 (t, 1H), 5.97 (s, 2H), 6.10 (d, 1H), 6.48 (s, 1H), 6.89 (d, 1H), 7.05 (m, 3H), 7.97 (d, 1H), 8.80 (s, 111), 8.88 (s, 1H) | 10 |
| 127 | 3-[[4-[(5- chlorobenzo[1,3] dioxol-4- yl)amino]pyrimidin- 2-yl]amino]-5- morpholin-4-yl- benzamide | 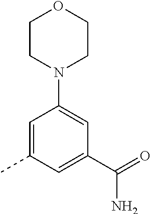 | 469.40 (M+) | 3.00 (m, 4H), 3.72 (m, 4H), 5.97 (s, 2H), 6.09 (d, 1H), 6.85 (d, 1H), 6.93 (s, 1H), 7.00 (d, 1H), 7.14 (s, 1H), 7.46 (s, 1H), 7.53 (s, 1H), 7.67 (s, 1H), 8.00 (d, 1H), 8.87 (s, 1H), 8.91 (s, 1H) | 8h |
| 128 | N-[3-[[4-[(5- chlorobenzo[1,3] dioxol-4- yl)amino]pyrimidin- 2-yl]amino]-5- morpholin-4-yl- phenyl]methane sulfonamide | 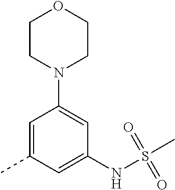 | 518.35 (M+) | 2.93 (m, 4H), 3.00 (s, 3H), 3.70 m, 4H), 6.00 (s, 2H), 6.10 (d, 1H), 6.31 (m, 1H), 6.87 (d, 1H), 7.04 (m, 2H), 7.15 (s, 1H), 7.98 (d, 1H), 8.80 (s, 1H), 8.88 (s, 1H), 9.32 (br s, | 14 |
| 129 | N-[3-[[4-[(5- chlorobenzo[1,3] dioxol-4- yl)amino]pyrimidin- 2-yl]amino]-5- methylsulfonyl- phenyl]methane sulfonamide | 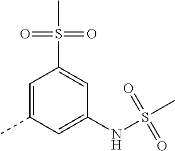 | 512.31 (M+) | 3.07 (s, 3H), 3.11 (s, 3H), 6.06 (s, 2H), 6.18 (d, 1H), 6.88 (d, 1H), 7.02 (d, 1H), 7.20 (m, 1H), 7.90 (m, 1H), 8.05 (m, 2H), 8.83 (s, 1H), 9.45 (s, 1H), 10.00 (br s, 1H) | 9 |
| 130 | N′-(5- chlorobenzo[1,3] dioxol-4-yl)-N- (3,5-dimorpholin- 4- ylphenyl)pyrimidine- 2,4-diamine | 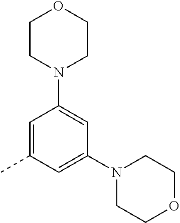 | 511.50 (M+) | 2.95 (m, 8H), 3.70 (m, 8H), 5.96 (s, 2H), 6.07 (m, 2H), 6.81 (d, 2H), 6.86 (d, 1H), 7.02 (d, 1H), 7.97 (d, 1H), 8.65 (s, 1H), 8.84 (s, 1H) | 8f |
| 131 | N′-(5- chlorobenzo[1,3] dioxol-4-yl)-N-(3- methylsulfonyl-5- morpholin-4-yl- phenyl) pyrimidine-2,4- diamine | 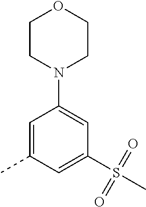 | 504.4 (M+) | 3.07 (m, 4H), 3.09 (s, 3H), 3.72 (m, 4H), 6.00 (s, 2H), 6.15 (d, 1H), 6.90 (m, 2H), 7.02 (d, 1H), 7.64 (s, 1H), 7.72 (s, 1H), 8.04 (d, 1H), 8.97 (s, 1H), 9.21 (s, 1H) | 8b |
| 132 | N′-(5- chlorobenzo[1,3] dioxol-4-yl)-N-(3- fluoro-5- morpholin-4-yl- phenyl) pyrimidine-2,4- diamine | 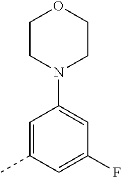 | 444.44 (M+) | 3.00 (m, 4H), 3.70 (m, 4H), 5.99 (s, 2H), 6.15 (d, 1H), 6.24 (m, 1H), 6.86 (m, 2H), 7.02 (d, 1H), 7.19 (d, 1H), 8.00 (d, 1H), 8.99 (s, 1H), 9.05 (s, 1H) | 8a |
| 133 | 3-[[4-[(5- chlorobenzo[1,3] dioxol-4- yl)amino]pyrimidin- 2-yl]amino]-5- methanesulfon- amido- benzenesulfonamide | 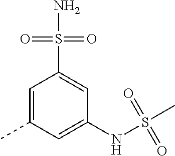 | 513.35 (M+) | 5.10 (s + m, 3H), 6.88 (d, 1H), 7.03 (d, 1H), 7.20 (m, 1H), 7.30 (s, 2H), 7.80 (m, 1H), 8.02 (d, 1H), 8.09 (m, 1H), 8.86 (s, 1H), 9.40 (s, 1H), 9.88 (br s, 1H) | 9a |
| 134 | 3-[[4-[(5- chlorobenzo[1,3] dioxol-4- yl)amino]pyrimidin- 2-yl]amino]-5- (hydroxymethyl) benzene sulfonamide | 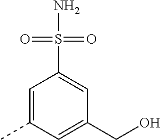 | 450.37 (M+) | 4.40 (d, 2H), 5.26 (t, 1H), 6.03 (s, 2H), 6.13 (d, 1H), 6.90 (d, 1H), 7.03 (d, 1H), 7.22 (s, 2H), 7.36 (s, 1H), 7.85 (s, 1H), 8.00 (m, 2H), 8.96 (s, 1H), 9.37 (s, 1H) | 16 |
| 135 | 3-[[4-[(5- chlorobenzo[1,3] dioxol-4- yl)amino]pyrimidin- 2-yl]amino]-5- methylsulfonyl- benzamide | 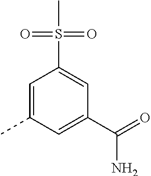 | 462.36 (M+) | 3.16 (s, 3H), 6.04 (s, 2H), 6.18 (d, 1H), 6.87 (d, 1H), 7.01 (d, 1H), 7.46 (s, 1H), 7.83 (s, 1H), 8.00 (s, 1H), 8.06 (d, 1H), 8.40 (s, 1H), 8.46 (s, 1H), 9.01 (s, 1H), 9.51 (s, 1H) | 8m |
| 136 | N-[3-[[4-[(5- chlorobenzo[1,3] dioxol-4- yl)amino]pyrimidin- 2-yl]amino]-5- methanesulfonamido- phenyl] methanesulfonamide | 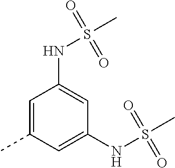 | 527.34 (M+) | 3.00 (s, 6H), 6.06 (s + m, 3H), 6.67 (m, 1H), 6.86 (d, 1H), 7.02 (d, 1H), 7.39 (m, 2H), 7.98 (d, 1H), 8.75 (s, 1H), 9.08 (s, 1H), 9.55 (br s, 2H) | 8i |
| 137 | 3-[[4-[(5- chlorobenzo[1,3] dioxol-4- yl)amino]pyrimidin- 2-yl}amino]-5- methanesulfonamido- benzamide | 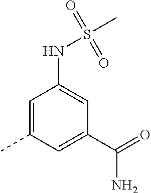 | 477.38 (M+) | 3.02 (s, 3H), 6.05 (s, 2H), 6.09 (d, 1H), 6.85 (d, 1H), 7.01 (d, 1H), 7.14 (m, 1H), 7.23 (br s, 1H), 7.65 (br s, 1H), 7.70 (m, 1H), 7.84 (s, 1H), 8.00 (d, 1H), 8.84 (s, 1H), 9.12 (s, 1H), 9.59 (br s, 1H) | 8j |
| 138 | [3-[[4-[(5- chlorobenzo[1,3] dioxol-4- yl)amino]pyrimidin- 2-yl]amino]-5- (hydroxymethyl) phenyl]methanol | 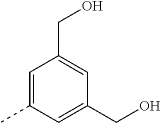 | 401.43 (M+) | 4.30 (d, 4H), 4.96 (t, 2H), 5.97 (s, 2H), 6.10 (d, 1H), 6.87 (m, 2H), 7.04 (d, 1H), 7.39 (s, 2H), 7.98 (d, 1H), 8.90 (s, 1H), 8.98 (s, 1H) | 8l |
| 139 | N′-(5- chlorobenzo[1,3] dioxol-4-yl)-N-[3- (4- methylpiperazin- 1-yl)-5- methylsulfonyl- phenyl]pyrimidine- 2,4-diamine | 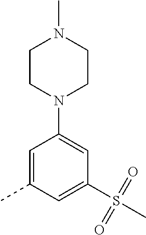 | 517.48 (M+) | | 8d |
| 140 | N′-(5- chlorobenzo[1,3] dioxol-4-yl)-N-(3- fluoro-5- methylsulfonyl- phenyl)pyrimidine- 2,4-diamine | 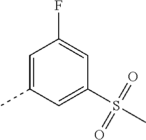 | 437.42 (M+) | 3.18 (s, 3H), 6.02 (s, 2H), 6.25 (d, 1H), 6.92 (d, 1H), 7.04 (d, 1H), 7.16 (m, 1H), 7.82 (s, 1H), 8.06 (d, 1H), 8.12 (m, 1H), 9.16 (s, 1H), 9.74 (s, 1H) | 8 |
| 141 | 3-[[4-[(5- chlorobenzo[1,3] dioxol-4- yl)amino]pyrimidin- 2-yl]amino]-5- (hydroxymethyl) benzamide | 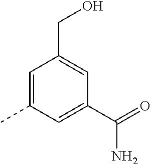 | 414.36 (M+) | | 8k |
| 142 | N-[3-[[4-[(5- chlorobenzo[1,3] dioxol-4- yl)amino]pyrimidin- 2-yl]amino]-5- (hydroxymethyl) phenyl]methane sulfonamide | 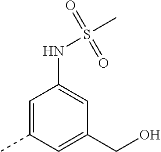 | 462.31 (M+) | 2.96 (s, 3H), 4.29 (m, 2H), 5.04 (t, 1H), 6.02 (s, 2H), 6.10 (d, 1H), 6.75 (m, 1H), 6.87 (d, 1H), 7.03 (d, 1H), 7.31 (m, 1H), 7.43 (s, 1H), 7.98 (d, 1H), 8.84 (s, 1H), 9.03 (s, 1H), 9.45 (br s, 1H) | 15a |

### EXAMPLE 3

### Step 1

### 2-[(3,4,5-Trimethoxyphenyl)amino]pyrimidin-4(3H)-one

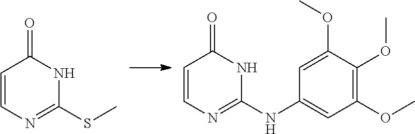

3,4,5-Trimethoxyaniline (6.82 g) and 2-(methylthio)pyrimidin-4(3H)-one (5.26 g) were suspended in diglyme (50 ml) and heated at 165° C. for 18 hours under nitrogen to give a red solution. The reaction was cooled to room temperature then poured into 500 ml diethyl ether with stirring to give an oily precipitate that was filtered and re-dissolved in water (250 ml). A solid precipitate formed which was stirred for 30 minutes then filtered to give the title compound as a cream solid (3.80 g, 37%); NMR Spectrum (300 MHz, DMSO) 3.63 (s, 3H), 3.76 (s, 6H), 5.80 (d, 1H), 6.95 (s, 2H), 7.76 (d, 1H); Mass Spectrum MH⁺ 278.5.

### Step 2

### 4-Chloro-N-(3,4,5-trimethoxyphenyl)pyrimidin-2-amine

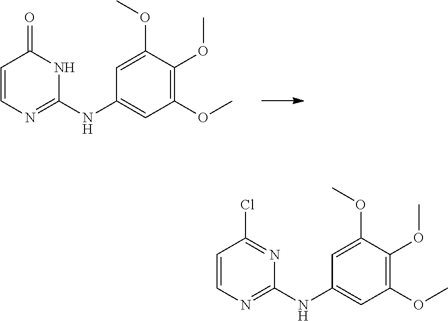

2-[(3,4,5-Trimethoxyphenyl)amino]pyrimidin-4(3H)-one (4.7 g) was suspended in acetonitrile (100 ml). Phosphorus oxychloride (10 ml) was added dropwise to give a dark solution. The reaction was heated at 85° C. for 2.5 hours then further phosphorus oxychloride (2 ml) added and the reaction heated overnight. The reaction was cooled to room temperature and concentrated in vacuo. The residue was dissolved in DCM (150 ml) and to ice water (100 ml) added. The mixture was stirred while adding saturated sodium bicarbonate solution to give pH=8. The organic layer was separated, washed with brine (25 ml), dried and concentrated to give a yellow solid. This was triturated with iso-hexane and filtered to give the title compound as a yellow solid (4.07 g, 81%); NMR Spectrum (300 MHz, DMSO) 3.63 (s, 3H), 3.76 (s, 6H), 6.94 (d, 1H), 7.13 (s, 2H), 8.43 (d, 1H), 9.87 (s, 1H); Mass Spectrum MH⁺ 296.5.

### Step 3

### N˜4˜-(1H-Indazol-7-yl)-N˜2˜-(3,4,5-trimethoxyphenyl)pyrimidine-2,4-diamine (Compound No. 68)

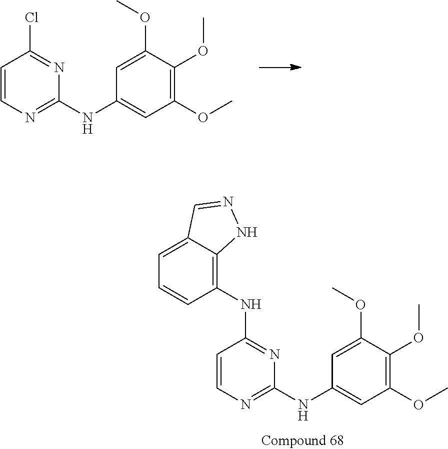

7-Aminoindazole (33 mg) and 4-chloro-N-(3,4,5-trimethoxyphenyl)pyrimidin-2-amine (70 mg) were dissolved in NMP (1 ml) and a solution of HCl in dioxane (0.07 ml, 4M) added. The reaction was heated at 130° C. for 5 hours then cooled to room temperature and concentrated in vacuo. The residue was purified by reverse phase chromatography to give the title compound as a solid (44 mg, 47%); NMR Spectrum (300 MHz, DMSO) 3.58 (s, 6H), 3.61 (s, 3H), 6.18 (d, 1H), 7.09 (m, 3H), 7.54 (d, 1H), 7.71 (d, 1H), 8.05 (d, 1H), 8.10 (s, 1H), 8.95 (s, 1H), 9.11 (s, 1H), 12.82 (s, 1H); Mass Spectrum M⁺ 392.4.

### EXAMPLE 4

The procedure described in example 3 above was repeated using the appropriate aniline. Thus were obtained the compounds described below in Table 2.

TABLE 2

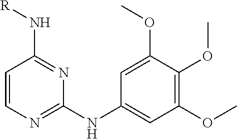

| | | | Molecular | NMR Spectrum |
|---|---|---|---|---|
| | | | Ion | (400 MHz, |
| No. | Name | R | (Observed) | d6-DMSO) |
| 69 | N′-(l-methylindol- 4-yl)-N-(3,4,5- trimethoxyphenyl)- pyrimidine-2,4- diamine | 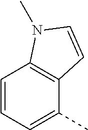 | 406.6 (MH+) | |
| 70 | N′-(5-bromobenzo [1,3]dioxol-4-yl)- N-(3,4,5- trimethoxyphenyl)- pyrimidine-2,4- diamine | 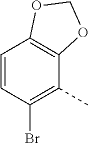 | 477.4 (M+) | 3.58 (s, 9H), 6.20 (d, 1H), 7.02 (s, 2H), 7.67 (d, 1H), 8.00 (d, 1H), 8.05 (d, 1H), 8.71 (s, 1H), 8.83 (s, 1H), 8.98 (s, 1H), 9.56 (s, 1H) |
| 71 | N′-benzo[l,3] dioxol-4-yl-N- (3,4,5-trimethoxy- phenyl)-pyrimidine- 2,4-diamine | 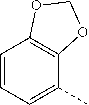 | 397.5 (MH+) | 3.53 (s, 3H), 3.59 (s, 6H), 6.15 (d,1H), 7.03 (s, 2H), 7.40 (s, 1H), 7.60 (s, 1H), 8.01 (d, 1H), 8.97 (s, 1H), 9.29 (s, 1H) |
| 72 | N′-(5-fluorobenzo [l,3]dioxol-4-yl)-N- (3,4,5-trimethoxy- phenyl)-pyrimidine- 2,4-diamine | 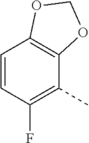 | 415.5 (MH+) | |
| 73 | N′-(2,2- difluorobenzo[l,3] dioxol-4-yl)-N- (3,4,5- trimethoxyphenyl)- pyrimidine-2,4- diamine | 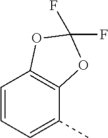 | 433.5 (MH+) | 3.60 (s, 9H), 6.34 (d, 1H), 7.06 (s, 2H), 7.14 (d, 1H), 7.78 (d, 1H), 8.08 (d, 1H), 9.01 (s, 1H), 9.43 (s, 1H) |
| 74 | 1-[7-[2-(3,4,5- trimethoxyphenyl) aminopyrimidin-4- yl]amino-2,3- dihydroindol-1- yl]ethanone | 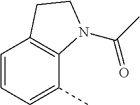 | 436.5 (MH+) | 3.10 (t, 2H), 3.61 (s, 3H), 3.72 (s, 6H), 4.15 (t, 2H), 6.17 (d, 1H), 7.06 (d, 1H), 7.17 (m, 3H),7.55 (d, 1H), 8.03 (d, 1H), 9.03 (s, 1H), 9.40 (s, 1H) |
| 75 | N′-(1H-indol-4- yl)-N-(3,4,5- trimethoxyphenyl)- pyrimidine-2,4- diamine | 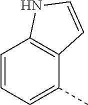 | 392.5 (MH+) | 3.63 (s, 9H), 6.31 (d, 1H), 6.61 (m, 1H), 7.03 (t, 1H), 7.15 (m, 3H), 7.29 (m, 1H), 7.64 (d, 1H), 8.00 (d, 1H), 8.88 (s, 1H), 8.96 (s, 1H), 11.10 (s, 1H) |
| 150 | N′-(3-chloro-4-yl)- N-(3,4,5- trimethoxyphenyl) pyrimidine-2,4- diamine | 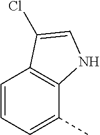 | 426.2 (MH+) | 3.48 (s, 9H), 6.13 (d, 1H), 7.08 (m, 3H), 7.30 (d, 1H), 7.46 (s, 1H), 7.52 (d, 1H), 7.95 (s, 1H), 8.01 (d, 1H), 8.90 (s, 1H), 9.00 (s, 1H) |
| 151 | N′-(1H-indazol-4- yl)-N-(3,4,5- trimethoxyphenyl) pyrimidine-2,4- diamine | 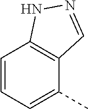 | 393.51 (MH+) | |
| 152 | N′-(7,10- dioxabicyclo[4.4.0] deca-2,4,11-trien-2- yl)-N-(3,4,5- trimethoxyphenyl) pyrimidine-2,4- diamine | 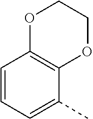 | 411.5 (MH+) | 3.60 (s, 3H), 3.65 (s, 6H), 4.28 (m, 4H), 6.31 (d, 1H), 7.62 (dd, 1H), 6.75 (t, 1H), 7.12 (s, 2H), 7.55 (d, 1H), 7.98 (d, 1H), 8.52 (s, 1H), 8.88 (s, 1H) |
| 153 | N′-isoquinolin-5-yl- N-(3,4,5- trimethoxyphenyl) pyrimidine-2,4- diamine | 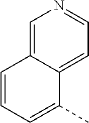 | 404.13 (MH+) | |
| 154 | N′-quinolin-5-yl-N- (3,4,5- trimethoxyphenyl) pyrimidine-2,4- diamine | 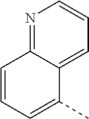 | 404.51 (MH+) | 3.50 (s, 6H), 3.58 (s, 3H), 6.26 (d, 1H), 7.02 (s, 2H), 7.55 (dd, 1H), 7.75 (m, 1H), 7.90 (m, 2H), 8.04 (d, 1H), 8.47 (d, 1H), 8.82 (m, 2H), 9.34 (s, 1H) |

### EXAMPLE 5

### 3-({4-[(5-Chloro-1,3-benzodioxol-4-yl)amino]pyrimidin-2-yl}amino)benzoic acid

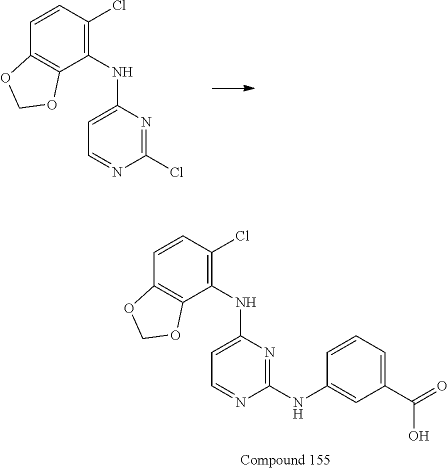

3-Aminobenzoic acid (6.1 g) and 2-chloro-N-(5-chloro-1,3-benzodioxol-4-yl)pyrimidin-4-amine (9 g) were dissolved in DMA (120 ml) and a solution of HCl in dioxane (11.1 ml, 4M) added. The reaction was heated at 96° C. for 4 hours then cooled to room temperature and DIPEA (5 ml) added. The solution was concentrated in vacuo. Water was added and the resulting solid filtered and triturated with methanol and dried in vacuo to give the title compound as an off-white solid (9.8 g, 80%); NMR Spectrum (300 MHz, DMSO) 6.00 (s, 2H), 6.17 (d, 1H), 6.90 (d, 1H), 7.03 (d, 1H), 7.18 (t, 1H), 7.41 (d, 1H), 8.04 (m, 3H), 9.00 (s, 1H), 9.28 (s, 1H), 12.72 (br s, 1H); Mass Spectrum MH⁺ 385.36.

### EXAMPLE 6

### Step 1

### 3-({4-[(5-Chloro-1,3-benzodioxol-4-yl)amino]pyrimidin-2-yl}amino)benzoyl chloride

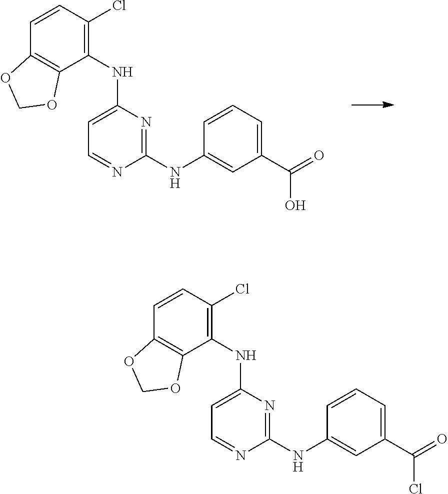

3-({4-[(5-Chloro-1,3-benzodioxol-4-yl)amino]pyrimidin-2-yl}amino)benzoic acid (3.61 g, Example 5) was added to thionyl chloride (35 ml) at 0° C. One drop of DMF was added to and the solution stirred at 0° C. for 2 hours, then concentrated in vacuo and azeotroped with toluene to give the title compound as a yellow solid (3.7 g, 98%) which was used without further purification.

### Step 2

### 3-({4-[(5-Chloro-1,3-benzodioxol-4-yl)amino]pyrimidin-2-yl}amino)-N,N-dimethylbenzamide

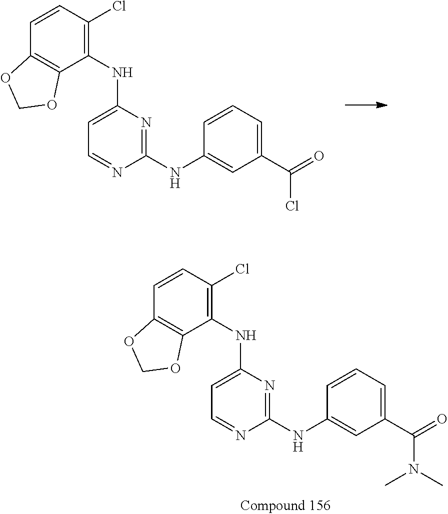

3-({4-[(5-Chloro-1,3-benzodioxol-4-yl)amino]pyrimidin-2-yl}amino)benzoyl chloride (100 mg) was dissolved in dry THF (5 ml) and a dimethylamine (5 ml, 2M in THF) added and the reaction stirred at room temperature for 2 hours. The solution was concentrated in vacuo and the residue purified by reverse phase chromatography to give the title compound as a solid (61 mg, 52%); NMR Spectrum (300 MHz, DMSO) 2.84 (s, 3H), 2.97 (s, 3H), 6.00 (s, 2H), 6.16 (d, 1H), 6.79 (d, 1H), 6.90 (d, 1H), 7.02 (d, 1H), 7.13 (t, 1H), 7.58 (s, 1H), 7.69 (d, 1H), 8.00 (d, 1H), 8.98 (s, 1H), 9.20 (s, 1H); Mass Spectrum MH⁺ 412.42.

### EXAMPLE 7

The procedure described in Example 6 above was repeated using the appropriate aniline. Thus were obtained the compounds described below in Table 3.

TABLE 3

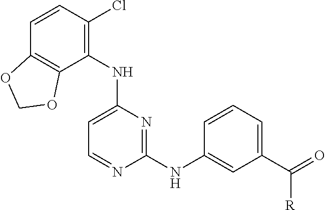

| | | | Molecular | NMR Spectrum |
|---|---|---|---|---|
| | | | Ion | (400 MHz, |
| No | Name | R | (Observed) | d6-DMSO) |
| 157 | 3-({4-[(5-chloro-l,3- benzodioxol-4- yl)amino]pyrimidin-2- yl}amino)-N-(3- hydroxy-2,2- dimethylpropyl) benzamide | 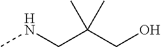 | 470.44 (MH+) | 0.84 (s, 6H), 3.14 (m, 2H), 3.32 (m, 2H), 4.62 (t, 1H), 6.0 l (s, 2H), 6.15 (s, 1H), 6.90 (m, 1H), 7.03 (m, 1H), 7.15 (m, 1H), 7.26 (m, 1H), 7.87 (s, 1H), 7.94 (d, 1H), 8.00 (d, 1H), 8.18 (s, 1H), 8.98 (s, 1H), 9.14 (s, 1H) |
| 158 | 3-({4-[(5-chloro-l,3- benzodioxol-4- yl)amino]pyrimidin-2- yl}amino)-N-[2- (methylsulfonyl)ethyl] benzamide | 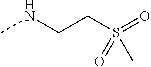 | 493.48 (MH+) | |
| 160 | 3-({4-l(5-chloro-l,3- benzodioxol-4- yl)amino]pyrimidin-2- yl}amino)-N-[2-(3- methyl-1H-1,2,4- triazol-5-yl)ethyl] benzamide | 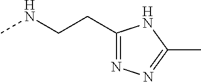 | 494.99 (M+) | |
| 161 | 3-({4-[(5-chloro-l,3- benzodioxol-4- yl)amino]pyrimidin-2- yl}amino)-N-(2- piperidin-1- ylethyl)benzamide | 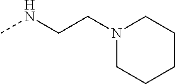 | 494.99 (M+) | 1.38 (m, 2H), 1.50 (m, 4H), 2.40 (m, 6H), 3.30 (m, 2H), 6.01 (s, 2H), 6.14 (s, 1H), 6.90 (m, 1H), 7.03 (m, 1H), 7.12 (m, 1H), 7.25 (m, 1H), 7.86 (s, 1H), 7.91 (d, 1H), 8.00 (m, 1H), 8.12 (s, 1H), 8.97(s, 1H), 9.16 (s, 1H) |
| 162 | 3-({4-[(5-chloro-l,3- benzodioxol-4- yl)amino]pyrimidin-2- yl}amino)-N-[(1R,2R)- 2-(hydroxymethyl) cyclohexyl]benzamide | 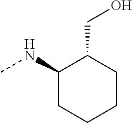 | 495.97 (M+) | 1.09 (m, 2H), 1.20 (m, 1H), 1.40 (m, 1H), 1.63 (m, 2H), 1.78 (m, 1H), 2.35 (m, 2H), 3.52 (m, 1H), 4.21 (t, 1H), 5.97 (s, 2H), 6.08 (s, 1H), 6.82 (m, 1H), 6.95 (m, 1H),7.05 (m, 1H), 7.19 (m, 1H), 7.75 (d, 1H), 7.88 (m, 1H), 7.93 (m, 1H), 7.99 (m, 1H), 8.88 (s, 1H), 9.06 (s, 1H) |
| 163 | 3-({4-[(5-chloro-l,3- benzodioxol-4- yl)amino]pyrimidin-2- yl}amino)-N-[3- (dimethylamino)-2,2- dimethylpropyl] benzamide | 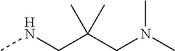 | 497.00 (M+) | 0.90 (s, 6H), 2.19 (m, 2H), 2.28 (s, 6H), 3.18 (s, 2H), 6.01 (s, 2H), 6.15 (s, 1H), 6.90 (m, 1H), 7.03 (m, 1H), 7.15 (m, 1H), 7.22 (m, 1H), 7.85 (m, 1H), 7.94 (d, 1H), 8.00 (s, 1H), 8.31 (s, 1H), 8.96 (s, 1H), 9.18 (s, 1H) |
| 164 | 3-({4-[(5-chloro-l,3- benzodioxol-4- yl)amino]pyrimidin-2- yl}amino)-N-(3- hydroxypropyl) benzamide | 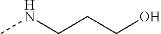 | 442.51 (MH+) | 1.69 (m, 2H), 3.30 (m, 2H), 3.48 (m, 2H), 6.01 (s, 2H), 6.15 (s, 1H), 6.90 (m, 1H), 7.03 (m, 1H), 7.12 (m, 1H), 7.25 (m, 1H), 7.85 (s, 1H), 7.92 (m, 1H), 8.01 (m, 1H), 8.20 (s, 1H), 8.96 (s, 1H), 9.15 (s, 1H) |
| 165 | 3-({4-[(5-chloro-l,3- benzodioxol-4- yl)amino]pyrimidin-2- yl}amino)-N-[(1S)-2- hydroxy-1-methylethyl] benzamide | 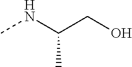 | 442.51 (MH+) | |
| 166 | 3-({4-[(5-chloro-l,3- benzodioxol-4- yl)amino]pyrimidin-2- yl}amino)-N-[(2R)-2- hydroxypropyl]benzamide | 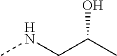 | 442.52 (MH+) | 1.07 (d, 3H), 3.19 (m, 2H), 3.78 (m, 1H), 6.01 (s, 2H), 6.15 (s, 1H), 6.90 (m, 1H), 7.03 (m, 1H), 7.12 (m, 1H), 7.28 (m, 1H), 7.88 (s, 1H), 7.92 (m, 1H), 8.01 (m, 1H), 8.12 (s, 1H), 8.96 (s, 1H), 9.15 (s, 1H) |
| 167 | N-(2-carbamoylethyl)- 3-[[4-[(5-chlorobenzo [l,3]dioxol-4-yl) amino]pyrimidin-2- yl]amino]benzamide | 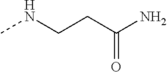 | 455.48 (MH+) | 2.35 (m, 2H), 3.29 (m, 2H), 6.01 (s, 2H), 6.15 (s, 1H), 6.80 (s, 1H), 6.90 (m, 1H), 7.03 (m, 1H), 7.13 (m, 1H), 7.26 (m, 1H), 7.35 (s, 1H), 7.88 (s, 1H), 7.92 (m, 1H), 8.01 (m, 1H), 8.24 (s, 1H), 8.96 (s, 1H), 9.17 (s, 1H) |
| 168 | 3-({4-[(5-chloro-1,3- benzodioxol-4- yl)amino]pyrimidin-2- yl}amino)-N-[2-(1H- imidazol-4-yl)ethyl] benzamide | 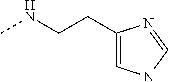 | 476.21 (M − H+) | |
| 169 | 3-({4-[(5-chloro-1,3- benzodioxol-4- yl)amino]pyrimidin-2- yl}amino)-N-[(5- methylisoxazol-3- yl)methyl]benzamide | 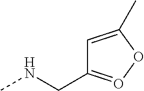 | 479.23 (MH+) | 2.38 (s, 3H), 4.43 (m, 2H), 6.01 (s, 2H), 6.15 (m, 2H), 6.90 (m, 1H), 7.03 (m, 1H), 7.14 (m, 1H), 7.30 (m, 1H), 7.92 (m, 2H), 8.01 (m, 1H), 8.84 (s, 1H), 8.96 (s, 1H), 9.20 (s, 1H) |
| 170 | 3-({4-[(5-chloro-1,3- benzodioxol-4- yl)amino]pyrimidin-2- yl}amino)-N-(2-pyrrolidin- 1-ylethy])benzamide | 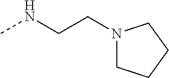 | 479.20 (M − H+) | 1.70 (m, 4H), 2.53 (m, 4H), 2.60 (m, 2H), 3.28 (m, 2H), 6.01 (s, 2H), 6.14 (s, 1H), 6.90 (m, 1H), 7.01 (m, 1H), 7.12 (m, 1H), 7.27 (m, 1H), 7.86 (s, 1H), 7.91 (m, 1H), 8.00 (m, 1H), 8.20 (s, 1H), 8.96 (s, 1H), 9.16 (s, 1H) |
| 171 | 3-[[4-[(5-chlorobenzo[1,3] dioxol-4-yl)amino] pyrimidin-2-yl]amino]- N-(propan-2-yl carbamoylmethyl) benzamide | 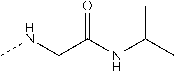 | 483.13 (M+) | 1.07 (d, 6H), 3.80 (m, 2H), 3.87 (m, 1H), 6.01 (s, 2H), 6.15 (s, 1H), 6.90 (m, 1H), 7.02 (m, 1H), 7.15 (m, 1H), 7.32 (m, 1H), 7.70 (d, 1H), 7.92 (m, 2H), 8.01 (m, 1H), 8.34 (s, 1H), 8.96 (s, 1H), 9.18 (s, 1H) |
| 172 | 3-({4-[(5-chloro-l,3- benzodioxol-4-yl) amino]pyrimidin-2- yl}amino)-N-[2- (diethylamino)ethyl] bcnzamide | 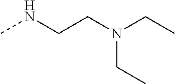 | 481.17 (M − H+) | 0.99 (m, 6H), 2.54 (m, 6H), 3.28 (m, 2H), 6.01 (s, 2H), 6.15 (s, 1H), 6.90 (m, 1H), 7.03 (m, 1H), 7.14 (m, 1H), 7.25 (m, 1H), 7.87 (s, 1H), 7.90 (m, 1H), 8.00 (m, 1H), 8.10 (s, 1H), 8.96 (s, 1H), 9.17 (s, 1H) |
| 173 | {(2S)-1-[3-({4-[(5- chloro-1,3-benzodioxol- 4-yl)amino]pyrimidin-2- yl}amino)benzoyl]piperidin- 2-yl}methanol | 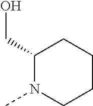 | 482.44 (M+) | |
| 174 | 1-[3-({4-[(5-chloro-1,3- benzodioxol-4-yl)amino] pyrimidin-2-yl}amino) benzoyl]-4-methylpiperidin- 4-ol | 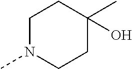 | 482.44 (M+) | 1.18 (s, 3H), 1.47 (m, 4H), 3.29 (m, 4H), 6.02 (s, 2H), 6.16 (s, 1H), 6.80 (m, 1H), 6.90 (m, 1H), 7.02 (m, 1H), 7.12 (m, 1H), 7.55 (s, 1H), 7.75 (m, 1H), 8.01 (m, 1H), 8.98 (s, 1H), 9.19 (s, 1H) |
| 175 | 3-[[4-[(5-chlorobenzo[1,3] dioxol-4-yl)amino]pyrimidin- 2-yl]amino]-N- (dimethylcarbamoylmethyl)- N-methyl-benzamide | 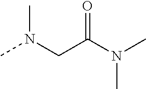 | 483.46 (M+) | |
| 176 | 3-({4-[(5-chloro-1,3- benzodioxol-4- yl)amino]pyrimidin-2- yl}amino)N-methyl- N-(pyridin-3-ylmethyl) benzamide | 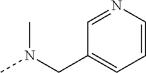 | 489.42 (M+) | |
| 177 | 3-({4-[(5-chloro-1,3- benzodioxol-4- yl)amino]pyrimidin-2- yl}amino)-N-methyl- N-(pyridin-4-ylmethyl) benzamide | 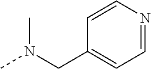 | 489.45 (M+) | |
| 178 | 3-({4-[(5-chloro-1,3- benzodioxol-4- yl)amino]pyrimidin-2- yl}amino)-N-methyl- N-[(1-methyl-1H- pyrazol-4-yl)methyl] benzamide | 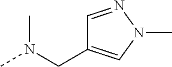 | 492.43 (M+) | |
| 179 | N~2~-{3-[(4- acetylpiperazin-1- yl)carbonyl]phenyl}- N~4~-(5-chloro-1,3- benzodioxol-4- yl)pyrimidine-2,4- diamine | 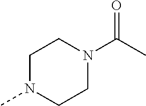 | 495.42 (M+) | 2.03 (s, 1H), 3.29 (m, 4H), 3.49 (m, 4H), 6.01 (s, 2H), 6.17 (s, 1H), 6.85 (m, 1H), 6.90 (m, 1H), 7.02 (m, 1H), 7.15 (m, 1H), 7.62 (s, 1H), 7.75 (m, 1H), 8.02 (m, 1H), 8.99 (s, 1H), 9.22 (s, 1H) |
| 181 | 3-({4-[(5-chloro-1,3- benzodioxol-4- yl)amino]pyrimidin-2- yl}amino)-N-methyl- N-(1-methylpiperidin- 4-yl)benzamide | 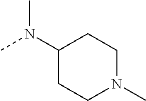 | 495.52 (M+) | |
| 182 | N~4~-(5-chloro-1,3- benzodioxol-4-yl)- N~2~{3-[(4-isopropyl- piperazin-1-yl)carbonyl] phenyl}pyrimidine-2,4- diamine | 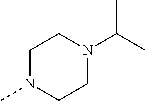 | 495.49 (M+) | 1.00 (d, 6H), 2.46 (m, 4H), 2.70 (m, 1H), 3.33 (m, 4H), 6.01 (s, 2H), 6.17 (s, 1H), 6.81 (m, 1H), 6.90 (m, 1H), 7.02 (m, 1H), 7.13 (m, 1H), 7.58 (s, 1H), 7.76 (m, 1H), 8.01 (m, 1H), 8.99 (s, 1H), 9.20 (s, 1H) |
| 183 | N~4~-(5-chloro-1,3- benzodioxol-4-yl)- N~2~-(3-{[4- (dimethylamino)piperidin- 1-yl]carbonyl}phenyl) pyrimidine-2,4-diamine | 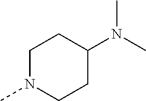 | 495.48 (M+) | |
| 184 | 3-({4-[(5-chloro-1,3- benzodioxol-4-yl)amino] pyrimidin-2-yl}amino)-N- (2-hydroxyethyl)-N- methylbenzamide | 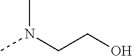 | 442.45 (M+) | 2.94 (s, 3H), 3.28 (m, 2H), 3.49 (m, 2H), 6.01 (s, 2H), 6.16 (s, 1H), 6.81 (m, 1H), 6.90 (m, 1H),7.03 (m, 1H), 7.12 (m, 1H), 7.55 (s, 1H), 7.72 (m, 1H), 8.00 (m, 1H), 8.98 (s, 1H), 9.19 (s, 1H) |
| 185 | N~4~-(5-chloro-1,3- benzodioxol-4-yl)- [N~2~-[3-(morpholin-4- ylcarbonyl)phenyl] pyrimidine-2,4-diamine | 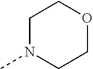 | 454.44 (M+) | 3.33 (m, 4H), 3.61 (m, 4H), 6.02 (s, 2H), 6.18 (s, 1H), 6.84 (m, 1H), 6.90 (m, 1H), 7.02 (m, 1H), 7.14 (m, 1H), 7.51 (s, 1H), 7.76 (m, 1H), 8.02 (m, 1H), 9.00 (s, 1H), 9.22 (s, 1H) |
| 186 | (3R)-1-[3-({4-[(5-chloro- 1,3-benzodioxol-4-yl) amino]pyrimidin-2-yl} amino)benzoyl]pyrrolidin- 3-ol | 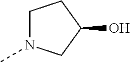 | 454.43 (M+) | |
| 187 | (3S)-1-[3-({4-[(5-chloro- 1,3-benzodioxol-4-yl) amino]pyrimidin-2-y1} amino)benzoyl]pyrrolidin- 3-ol | 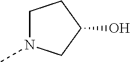 | 454.42 (M+) | |
| 188 | 1-[3-({4-[(5-chloro-1,3- benzodioxol-4-yl)amino] pyrimidin-2-yl}amino) bcnzoyl]-3-melhylazetidin- 3-ol | 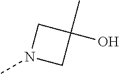 | 454.43 (M+) | 1.39 (s, 1H), 3.38 (m, 2H), 4.08 (m, 2H), 6.01 (s, 2H), 6.17 (s, 1H), 6.90 (m, 1H), 7.03 (m, 1H), 7.07 (m, 1H), 7.13 (m, 1H), 7.75 (s, 1H), 7.90 (m, 1H), 8.01 (m, 1H), 8.99 (s, 1H), 9.22 (s, 1H) |
| 189 | 3-({4-[(5-chloro-1,3- benzodioxol-4-yl) amino]pyrimidin-2-yl} amino)-N-ethyl-N-(2- hydroxyethyl)benzamide | 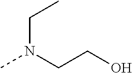 | 456.45 (M+) | |
| 190 | 4-[3-({4-[(5-chloro-1,3- benzodioxol-4-yl)amino] pyrimidin-2-yl}amino) benzoyl]piperazin-2-one | 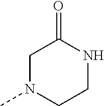 | 467.42 (M+) | |
| 191 | N~4~-(5-chloro-1,3- benzodioxol-4-yl)- N~2~-{3-[(4- methylpiperazin-1- yl)carbonyl]phenyl} pyrimidine-2,4-diamine | 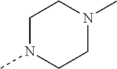 | 467.45 (M+) | 2.20 (s, 3H), 2.32 (m, 4H), 3.33 (m, 4H), 6.01 (s, 2H), 6.16 (s, 1H), 6.80 (m, 1H), 6.90 (m, 1H), 7.03 (m, 1H), 7.12 (m, 1H), 7.56 (s, 1H), 7.75 (m, 1H), 8.01 (m, 1H), 8.98 (s, 1H), 9.20 (s, 1H) |
| 192 | {(2S)-1-[3-({4-[(5- chloro-1,3- benzodioxol-4- yl)amino]pyrimidin-2- yl}amino)benzoyl] pyrrolidin-2-yl}methanol | 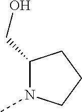 | 468.44 (M+) | |
| 193 | 1-[3-({4-[(5-chloro- 1,3-benzodioxol-4- yl)amino]pyrimidin-2- yl}amino)benzoyl] piperidin-4-ol | 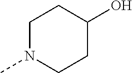 | 468.43 (M+) | 1.43 (m, 2H), 1.75 (m, 2H), 3.32 (m, 4H), 3.74 (m, 1H), 6.02 (s, 2H), 6.l6 (s, 1H), 6.80 (m, 1H), 6.90 (m, 1H), 7.03 (m, 1H), 7.12 (m, 1H), 7.55 (s, 1H), 7.75 (m, 1H), 8.01 (m, 1H), 8.98 (s, 1H), 9.20 (s, 1H) |
| 194 | {(2R)-1-[3-({4-[(5- chloro-1,3- benzodioxol-4- yl)amino]pyrimidin-2- yl}amino)benzoyl] pyrrolidin-2-yl}methanol | 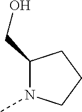 | 468.43 (M+) | |
| 195 | N~4~-(5-chloro-1,3- benzodioxol-4-yl)- N~2~-[3-(1,4- oxazepan-4- ylcarbonyl)phenyl] pyrimidine-2,4-diamine | 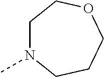 | 468.44 (M+) | 1.71 (m, 1H), 1.88 (m, 1H), 3.33 (M, 4H), 3.70 (m, 4H), 6.01 (s, 2H), 6.16 (s, 1H), 6.80 (m, 1H), 6.90 (m, 1H), 7.03 (m, 1H), 7.13 (m, 1H), 7.59 (s, 1H), 7.72 (m, 1H), 8.02 (m, 1H), 8.99 (s, 1H), 9.22 (s, 1H) |
| 196 | 1-[3-({4-[(5-chloro- 1,3-benzodioxol-4- yl)amino]pyrimidin-2- yl}amino)benzoyl]-1,4- diazepan-5-one | 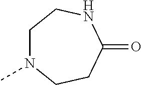 | 481.43 (M+) | |
| 197 | 4-[3-({4-[(5-chloro- 1,3-benzodioxol-4- yl)amino]pyrimidin-2- yl}amino)benzoyl]-1- methylpiperazin-2-one | 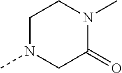 | 481.42 (M+) | |
| 198 | 3-({4-[(5-chloro-1,3- benzodioxol-4- yl)amino]pyrimidin-2- y1}amino)-N-methyl- N-(1-methylpyrrolidin- 3-yl)benzamide | 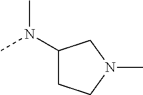 | 481.45 (M+) | |
| 199 | N~4~-(5-chloro-1,3- benzodioxol-4-yl)- N~2~-{3-[(4- ethylpiperazin-1- yl)carbonyl]phcnyl} pyrimidine-2,4-diamine | 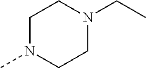 | 481.45 (M+) | 1.01 (t, 3H), 2.36 (m, 6H), 3.30 (m, 4H), 6.02 (s, 2H), 6.16 (s, 1H), 6.80 (m, 1H), 6.90 (m, 1H), 7.03 (m, 1H), 7.13 (m, 1H), 7.57 (s, 1H), 7.74 (m, 1H), 8.01 (m, 1H), 8.99 (s, 1H), 9.21 (S, 1H) |
| 200 | N~4~-(5-chloro-1,3- benzodioxol-4-yl)- N~2~-(3-{[(3R)-3- (dimethylamino) pyrrolidin-1- yl}carbonyl}phenyl) pyrimidine-2,4-diamine | 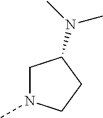 | 481.45 (M+) | |
| 201 | N~4~-(5-chloro-1,3- benzodioxol-4-yl)- N~2~-{3-[(3aR,6aS)- tetrahydro-5H- [1,3]dioxolo[4,5-c] pyrrol-5-ylcarbonyl] phenyl}pyrimidine-2,4- diamine | 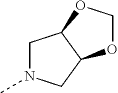 | 481.42 (M+) | 3.41 (m, 4H), 4.70 (m, 2H), 4.80 (s, 2H), 6.04 (s, 2H), 6.20 (s, 1H), 6.93 (m, 2H), 7.05 (m, 1H), 7.16 (m, 1H), 7.68 (s, 1H), 7.81 (m, 1H), 8.04 (m, 1H), 9.24 (s, 1H) |
| 202 | {1-[3-({4-[(5-chloro- 1,3-benzodioxol-4- yl)amino]pyrimidin-2- yl}amino)benzoyl] piperidin-4-yl}methanol | 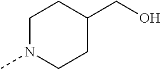 | 481.44 (M+) | |
| 204 | 3-({4-[(5-chloro-1,3- benzodioxol-4- yl)amino]pyrimidin-2- yl}amino)-N,N-bis(2- hydroxyethyl)benzamide | 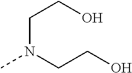 | 472.44 (M+) | |
| 205 | 3-({4-[(5-chloro-1,3- benzodioxol-4- yl)amino]pyrimidin-2- yl}amino)-N- cyclopropyl benzamide | 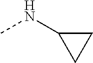 | 424.44 (M+) | |
| 206 | 3-({4-[(5-chloro-1,3- benzodioxol-4- yl)amino]pyrimidin-2- yl}amino)-N-prop-2- yn-1-ylbenzamide | 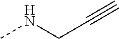 | | |

### EXAMPLE 8

### 7-[(2-{[3-(Methylsulfonyl)phenyl]amino}pyrimidin-4-yl)amino]-1,3-benzodioxole-5-carbonitrile

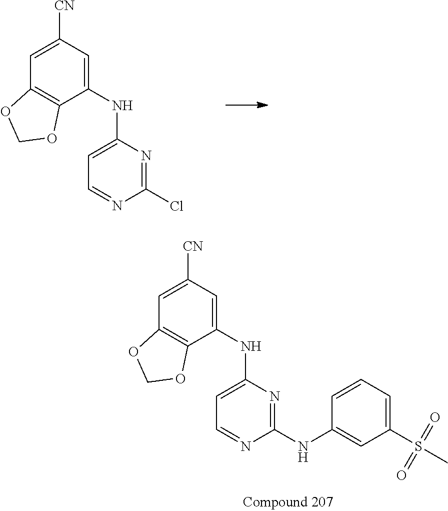

HCl (1 drop, 4N in dioxane) was added to a solution of 7-[(2-chloropyrimidin-4-yl)amino]benzo[1,3]dioxole-5-carbonitrile (83 mg-method 23) and 3-methylsulfonylaniline hydrochloride (69 mg) in iso-propanol (0.5 ml) and NMP (0.5 ml) and heated at 110° C. for 20 mins (microwave). The solution was cooled and added DIPEA before concentrating in vacuo. The residue was purified by reverse phase chromatography to give the title compound as a beige solid (21 mg, 17%); NMR Spectrum (300 MHz, DMSO) 3.16 (s, 3H), 6.20 (s, 2H), 6.43-6.46 (m, 1H), 7.23 (s, 1H), 7.42-7.49 (m, 2H), 7.99 (d, 1H), 8.12 (d, 1H), 8.17 (s, 1H), 8.20 (s, 1H), 9.37 (s, 1H), 9.63 (s, 1H); Mass Spectrum MH⁺ 410.33.

### EXAMPLE 9

The procedure described in Example 8 above was repeated using the appropriate chloropyrimidine and aniline. Thus were obtained the compounds described in Table 4 below:

TABLE 4

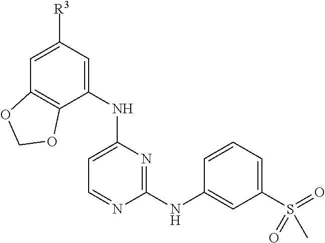

| | | | Molecular | NMR Spectrum | Starting |
|---|---|---|---|---|---|
| | | | Ion | (300 MHz, | Material |
| No. | Name | R³ | (Observed) | d6-DMSO) | (method) |
| 208 | N~4~-(6-bromo-1,3- benzodioxol-4-yl)- N~2~-[3- (methylsulfonyl) phenyl]pyrimidine- 2,4-diamine | 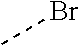 | 465.29 (MH+) | | 23a |
| 209 | N~4~-[6-(2- methoxyethyl)-1,3- benzodioxol-4-yl]- N~2~-[3- (methylsulfonyl) phenyl]pyrimidine- 2,4-diamine | 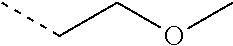 | 443.38 (MH+) | | 23b |
| 210 | [7-[[2-[(3- methylsulfonylphenyl) amino]pyrimidin-4- yl]amino]benzo[1,3] dioxol-5-yl]methanol | 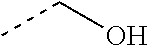 | 415.49 (MH+) | 3.30 (s, 3H), 4.42 (d, 2H), 5.11 (t, 1H), 6.00 (s, 2H), 6.25 (q, 1H), 6.74 (d, 1H), 7.07 (d, 1H), 7.37-7.46 (m, 2H), 8.05 (d, 1H), 8.16 (s, 1H), 8.20-8.24 (m, 1H), 9.08 (s, 1H), 9.46 (s, 1H) | 24 |

### EXAMPLE 10

### Starting Materials

### (1) 2-chloro-N-(5-chlorobenzo[1,3]dioxol-4-yl)-N-methyl-pyrimidin-4-amine

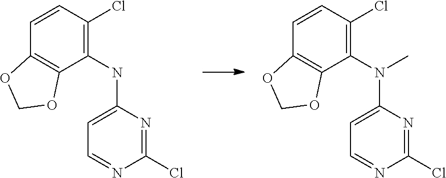

2-Chloro-N-(5-chloro-1,3-benzodioxol-4-yl)pyrimidin-4-amine (1.5 g, 5.30 mmol, see Example 1, Step 1) was dissolved in DMF (30 mL). Potassium carbonate (1.1 g, 8.0 mmol) was added, followed by iodomethane (0.36 mL, 5.8 mmol) and the mixture was stirred overnight. After evaporation under reduced pressure, the residue was dissolved in ethyl acetate, washed with water and brine, dried and evaporated to yield a brown oil (1.54 g, 98%) which solidified on standing; NMR Spectrum (500 MHz, DMSOd6 at 353° K.) 3.33 (s, 3H), 6.29 (s, 2H), 7.12 (bs, 1H), 7.00 (d, 1H), 7.10 (d, 1H), 8.12 (bs, 1H); Mass Spectrum MH⁺ 298.

### (2) (5-chlorobenzo[1,3]dioxol-4-yl)-(2-chloropyrimidin-4-yl)amino]acetonitrile

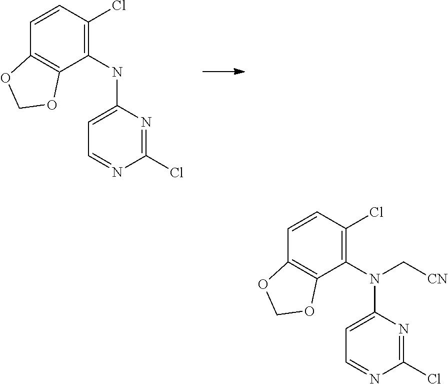

Following the same procedure as for (1) above, 2-chloro-N-(5-chloro-1,3-benzodioxol-4-yl)pyrimidin-4-amine (1.5 g, 5.30 mmol) was reacted with iodoacetonitrile (0.42 mL, 5.8 mmol) to yield a yellow solid (1.53 g, 89%) after trituration in ether/pentane; NMR Spectrum 4.95 (s, 2H), 6.18 (d, 2H), 6.42 (s, 1H), 7.11 (d, 1H), 7.16 (d, 1H), 8.26 (s, 1H); Mass Spectrum MH⁺ 323.

### (3) 2-chloro-N-(5-chlorobenzo[1,3]dioxol-4-yl)-N-(2-methoxyethyl)pyrimidin-4-amine

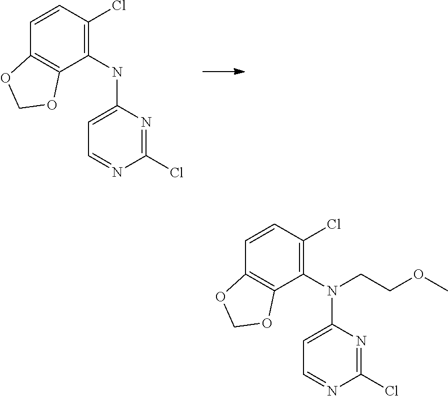

Following the same procedure as for (1) above, 2-chloro-N-(5-chloro-1,3-benzodioxol-4-yl)pyrimidin-4-amine (1.5 g, 5.30 mmol) was reacted with 2-bromoethyl methyl ether (0.55 mL, 5.8 mmol) to yield a brown oil (1.2 g, 67%); NMR Spectrum 3.21 (s, 3H), 3.53 (t, 2H), 3.83-3.92 (m, 1H), 4.09-4.19 (m, 1H), 6.13-6.21 (m, 3H), 7.08 (d, 1H), 7.15 (d, 1H), 8.10 (d, 1H); Mass Spectrum MH⁺ 342.

### (4) 2-[(5-chlorobenzo[1,3]dioxol-4-yl)-(2-chloropyrimidin-4-yl)amino]ethanol

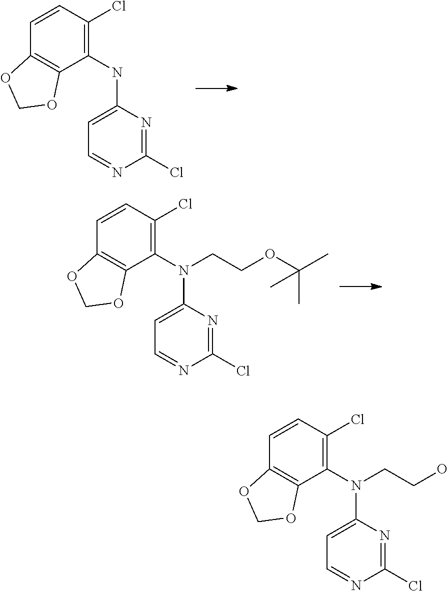

Following the same procedure as for (1) above, 2-chloro-N-(5-chloro-1,3-benzodioxol-4-yl)pyrimidin-4-amine (2.0 g, 7.07 mmol) was reacted with 2-bromoethyl t-butyl ether (1.92 mL, 10.6 mmol) to yield 2-chloro-N-(5-chlorobenzo[1,3]dioxol-4-yl)-N-[2-[(t-butyloxy]ethyl]pyrimidin-4-amine as a white solid (2.2 g, 81%) after chromatography on silica gel (EtOAc and petroleum ether, 1:9); NMR Spectrum 1.04 (s, 9H), 3.52 (t, 2H), 3.79-3.87 (m, 1H), 3.98-4.04 (m, 1H), 6.13 (s. 2H), 6.16 (d, 1H), 7.05 (d, 1H), 7.12 (d, 1H), 8.09 (d, 1H); Mass Spectrum MH⁺ 384.

2-chloro-N-(5-chlorobenzo[1,3]dioxol-4-yl)-N-[2-[(t-butyloxy]ethyl]pyrimidin-4-amine (2.1 g) was dissolved in a 1:1 mixture of methylene chloride and TFA (40 mL) and stirred at room temperature for 2 hours. The solvent was then removed and the residue dissolved in ether, washed with aqueous sodium bicarbonate and brine, dried, concentrated and purified by silica gel chromatography (EtOAc and petroleum ether, 3:7) to give the title compound as a white solid (1.06 g, 44%); NMR Spectrum (500 MHz, DMSOd6+TFAd at 297° K.) 3.59 (t, 2H), 3.70-3.78 (m, 1H), 3.99-4.08 (1H, m), 6.12-6.18 (m, 3H), 7.06 (d, 1H), 7.14 (d, 1H), 8.08 (d, 1H); Mass Spectrum MH⁺ 328.

### Final Compounds

### N′-(5-chlorobenzo[1,3]dioxol-4-yl)-N′-methyl-N-phenyl-pyrimidine-2,4-diamine

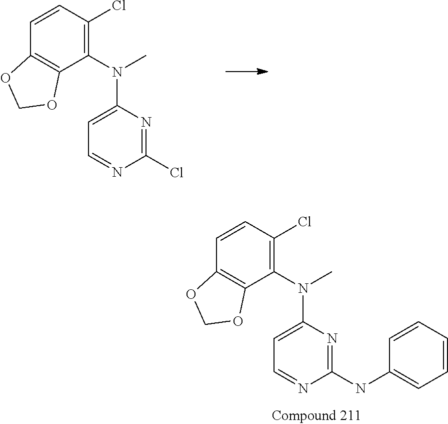

A mixture of 2-chloro-N-(5-chlorobenzo[1,3]dioxol-4-yl)-N-methyl-pyrimidin-4-amine (50 mg, 0.17 mmol), aniline (0.19 mmol), 4N HCl in dioxane (10 uL) and 1-pentanol (1 mL) was heated at 120° C. for 1 hour. The reaction mixture was cooled to room temperature, evaporated under reduced pressure and purified on a preparative HPLC-MS system (Column: C18, 5 microns, 19 mm diameter, 100 mm length; elution with a gradient of water and acetonitrile containing 2 g/l of ammonium carbonate); evaporation of the collected fractions gave the title compound (65 mg, 61%); NMR Spectrum (500 MHz, DMSOd6+TFAd) Major rotamer: 3.43 (s, 3H), 5.97 (d, 1H), 6.18 (s, 2H), 7.06-7.22 (m, 2H), 7.24 (t, 1H), 7.46 (t, 2H), 7.62 (d, 2H), 7.92 (d, 1H); Mass Spectrum MH⁺ 355.

The procedure described above was repeated using the appropriate aniline and 2-chloropyrimidine intermediate. Thus were obtained the compounds described in Table 5 below.

TABLE 5

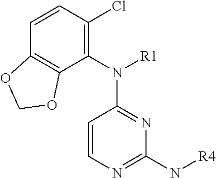

| | | | | | NMR |
|---|---|---|---|---|---|
| | | | | Molecular | Spectrum |
| | | | | Ion | (500 MHz, |
| No. | Name | R1 | R4 | (M + H⁺) | d6-DMSO) |
| 212 | 4-[[4-[(5- chlorobenzo[1,3]dioxol- 4-yl)-methyl- amino]pyrimidin-2- yl]amino] benzenesulfonamide | Me | 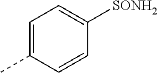 | 434 | (500 MHz, DMSOd6 + TFAd) Major rotamer: 3.46 (s, 3H), 6.04 (d, 1H), 6.18 (s, 2H), 7.08 (d, 1H), 7.16 (d, 1H), 7.83 (d, 2H), 7.90 (d, 2H), 8.00 (d, 1H) |
| 213 | N-[4-[[4-[(5- chlorobenzo[1,3]dioxol- 4-yl)-methyl- amino]pyrimidin-2- yl]amino]phenyl] acetamide | Me | 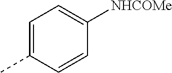 | 412 | (500 MHz, DMSOd6 + TFAd) Major rotamer: 1.95 (s, 3H), 3.31 (s, 3H), 5.82 (d, 1H), 6.07 (s, 2H), 6.98 (d, 1H), 7.05 (d, 1H), 7.40 (d, 2H), 7.57 (d, 2H), 7.78 (bs, 1H) |
| 214 | N′-(5- chlorobenzo[1,3]dioxol- 4-yl)-N′-methyl-N-(3,4,5- trimethoxyphenyl) pyrimidine-2,4-diamine | Me | 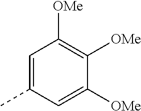 | 445 | (500 MHz, DMSOd6 at 353° K.) 3.36 (bs, 3H), 3.64 (s, 3H), 3.76 (s, 6H), 5.60 (bs, 1H), 6.12 (s, 2H), 7.00 (d, 1H), 7.10 (d, 1H), 7.21 (bs, 2H), 7.94 (bs, 1H), 8.94 (bs, 1H) |
| 215 | 3-[[4-[(5- chlorobenzo[1,3]dioxol- 4-yl)-methyl- amino]pyrimidin-2- yl]amino]benzonitrile | Me | 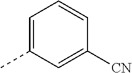 | 380 | (500 MHz, DMSOd6 + TFAd) Major rotamer: 3.43 (s, 3H), 6.04 (d, 1H), 6.17 (d, 2H), 7.08 (d, 1H), 7.13 (d, 1H), 7.61-7.68 (m, 2H), 7.91 (d, 1H), 7.98 (d, 1H), 8.13 (bs, 1H) |
| 216 | [3-[[4-[(5- chlorobenzo[1,3]dioxol- 4-yl)-methyl- amino]pyrimidin-2- yl]amino]phenyl] methanol | Me | 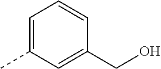 | 385 | (500 MHz, DMSOd6 + TFAd) Major rotamer: 3.34 (s, 3H), 4.54 (s, 2H), 5.94 (d, 1H), 6.15 (s, 2H), 6.97-7.17 (m, 3H), 7.36 (dd, 1H), 7.44 (d, 1H), 7.69 (bs, 1H), 7.91 (d, 1H) |
| 217 | 3-[[4-[(5- chlorobenzo[1,3]dioxol- 4-yl)-methyl- amino]pyrimidin-2- yl]amino] benzenesulfonamide | Me | 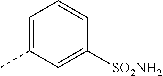 | 434 | (500 MHz, DMSOd6 + TFAd) Major rotamer: 3.47 (s, 3H), 6.02 (d, 1H), 6.18 (s, 2H), 7.09 (d, 1H), 7.16 (d, 1H), 7.63 (d, 1H), 7.67 (d, 1H), 7.71 (d, 1H), 8.01 (d, 1H), 8.44 (bs, 1H) |
| 218 | N′-(5- chlorobenzo[1,3]dioxol- 4-yl)-N′-methyl-N-(3- methylsulfonylphenyl) pyrimidine-2,4-diamine | Me | 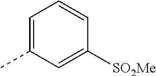 | 433 | (500 MHz, DMSOd6 at 363° K.) 3.14 (s, 3H), 3.41 (s, 3H), 6.03 (bs, 1H), 6.09 (s, 2H), 7.00 (d, 1H), 7.09 (d, 1H), 7.49 (d, 1H), 7.54 (d, 1H), 7.83 (d, 1H), 8.04 (bs, 1H), 8.36 (bs, 1H), 10.20 (bs, 1H) |
| 219 | N-[3-[[4-[(5-chlorobenzo [1,3]dioxol-4-yl)-methyl- amino]pyrimidin-2-yl] amino]phenyl]methane sulfonamide | Me | 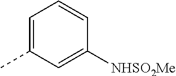 | 448 | (500 MHz, DMSOd6 + TFAd) Major rotamer: 3.00 (s, 3H), 3.46 (s, 3H), 5.97 (d, 1H), 6.17 (s, 2H), 7.02-7.10 (m, 2H), 7.14 (d, 1H), 7.32 (d, 1H), 7.38 (dd, 1H), 7.60 (bs, 1H), 7.93 (d, 1H) |
| 220 | 4-[[4-[(5-chlorobenzo[1,3] dioxol-4-yl)-(cyanomethyl) amino]pyrimidin-2-yl] amino]benzenesulfonamide | CH2CN | 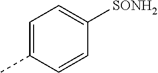 | 459 | (500 MHz, DMSOd6 + TFAd) Major rotamer: 5.03 (s, 2H), 6.14 (d, 1H), 6.19 (d, 2H), 6.24 (s, 2H), 7.12 (d, 1H), 6.19 (d, 1H), 7.89 (bs, 4H), 8.18 (d, 1H) |
| 221 | N-[4-[[4-[(5-chlorobenzo [1,3]dioxol-4-yl)- (cyanomethyl)amino] pyrimidin-2-yl]amino] phenyl]acetamide | CH2CN | 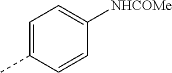 | 437 | (500 MHz, DMSOd6 + TFAd) Major rotamer: 2.05 (s, 3H), 4.95 (s, 2H), 6.07 (d, 1H), 7.12 (d, 1H), 7.18 (d, 1H), 7.55 (bs, 2H), 7.69 (d, 2H), 8.04 (bs, 1H) |
| 222 | 2-[(5-chlorobenzo(1,3] dioxol-4-yl)-2-[3,4,5- trimethoxyphenyl) amino]pyrimidin-4-yl] amino]acetonitrile | CH2CN | 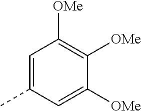 | 470 | (500 MHz, DMSOd6 at 353° K.) 3.65 (s, 3H), 3.77 (s, 6H), 4.86 (d, 2H), 4.95 (d, 1H), 5.77 (d, 1H), 6.11 (s, 2H), 7.02 (d, 1H), 7.10 (d, 1H), 7.11 (s, 2H), 8.05 (d, 1H), 8.90 (s, 1H) |
| 223 | 3-[[4-[(5- chlorobenzo[1,3]dioxol- 4-yl)-(cyanomethyl)amino] pyrimidin-2-yl]amino] bcnzonitrile | CH2CN | 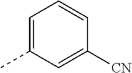 | 405 | (500 MHz, DMSOd6 + TFAd) Major rotamer: 4.99 (s, 2H), 6.14 (s, 1H), 6.19 (s, 2H), 7.12 (d, 1H), 7.17 (d, 1H), 7.57- 7.69 (m, 2H), 7.95 (s, 1H), 8.15 (d, 1H), 8.19 (s, 1H) |
| 224 | 2-[(5-chlorobenzo[1,3] dioxol-4-yl)-[2-[[3- (hydroxymethyl)phenyl] amino]pyrimidin-4- yl]amino]acetonitrile | CH2CN | 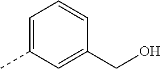 | 410 | (500 MHz, DMSOd6 + TFAd) Major rotamer: 4.56 (s, 2H), 4.97 (s, 2H), 6.10 (d, 1H), 6.18 (d, 2H), 7.09 (d, 1H), 7.15 (d, 1H), 7.21 (d, 1H), 7.39 (dd, 1H), 7.49 (d, 1H), 7.66 (s, 1H), 8.08 (d, 1H) |
| 225 | 3-[[4-[(5-chlorobenzo[1,3] dioxol-4-yl)-(cyanomethyl) amino]pyrimidin-2-yl] amino]benzenesulfonamide | CH2CN | 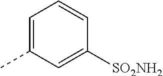 | 459 | (500 MHz, DMSOd6 + TFAd) Major rotamer. 5.01 (s, 2H), 6.15 (d, 1H), 6.19 (d, 2H), 7.12 (d, 1H), 7.18 (d, 1H), 7.63 (dd, 1H), 7.69 (d, 1H), 7.93 (d, 1H), 8.16 (d, 1H), 8.18 (s, 1H) |
| 226 | 2-[(5-chlorobenzo[1,3] dioxol-4-yl)-[2-[(3- methylsulfonylphenyl) amino]pyrimidin-4- yl]amino]acetonitrile | CH2CN | 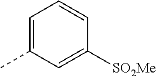 | 458 | (500 MHz, DMSOd6 + TFAd) Major rotamer: 3.75 (s, 3H), 5.02 (s, 2H), 6.15 (d, 1H), 6.19 (d, 2H), 7.12 (d, 1H), 7.18 (d, 1H), 7.70 (dd, 1H), 7.76 (d,1H), 7.94 (d, 1H), 8.17 (d, 1H), 8.47 (s, 1H), |
| 227 | N-[3-[[4-[(5-chlorobenzo [1,3]dioxol-(cyanomethyl) amino]pyrimidin-2- yl]amino]phenyl] methanesulfonamide | CH2CN | 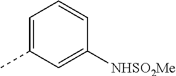 | 473 | (500 MHz,DMSOd6 + TFAd) Major rotamer: 3.02 (s, 3H), 5.07 (s, 2H), 6.12 (d, 1H), 6.20 (d, 2H), 7.12 (d, 1H), 7.14 (d, 1H),7.20 (d, 1H), 7.36 (d, 1H), 7.40 (dd, 1H), 7.60 (s, 1H), 8.11 (d, 1H) |
| 228 | N′-(5-chlorobenzo[1,3] dioxol-4-yl)-N′-(2- methoxyethyl)-N-phenyl- pyrimidine-2,4-diamine | CH2CH2OMe | 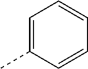 | 399 | (500 MHz, DMSOd6 + TFAd) Major rotamer 3.15 (s, 3H), 3.50-3.60 (m, 2H), 3.83-3.95 (m, 1H), 4.13-4.24 (m, 1H), 6.06 (d, 1H), 6.17 (d, 2H), 7.05-7.19 (m, 2H), 7.23 (t, 1H), 7.43 (dd, 1H),7.58 (d, 2H), 7.92 (d, 1H), 8.20 (d, 1H) |
| 229 | 4-[[4-[(5-chlorobenzo [1,3]dioxol-4-yl)-(2- methoxyethyl)amino] pyrimidin-2-yl]amino] benzenesulfonamide | CH2CH2OMe | 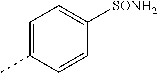 | 78 | (500 MHz, DMSOd6 + TFAd) Major rotamer: 3.20 (s, 3H), 3.55-3.63 (m, 2H), 3.92-4.02 (m, 1H), 4.15-4.28 (m, 1H), 6.00 (d, 1H), 6.19 (d, 2H), 7.10 (d, 1H), 7.12-7.21 (m, 1H), 7.82 (d, 2H), 7.89 (d, 2H), 8.02 (d, 1H) |
| 230 | N-[4-[[4-[(5-chlorobenzo [1,3]dioxol-4-yl)-(2- methoxyethyl)amino] pyrimidin-2-y1]amino] phenyl]acetamide | CH2CH2OMe | 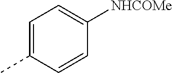 | 456 | (500 MHz, DMSOd6 + TFAd) Major rotamer: 2.04 (s, 3H), 3.16 (s, 3H), 3.50- 3.58 (m, 2H), 3.84- 3.95 (m, 1H), 4.13- 4.22 (m, 1H), 5.88 (d, 1H) 6.17 (d, 2H), 7.08 (d, 1H), 7.13 (d, 1H), 7.48 (d, 2H), 7.66 (d, 2H), 7.88 (bs, 1H) |
| 231 | N′-(5-chlorobenzo [1,3]dioxol-4-yl)-N′-(2- methoxyethyl)-N-(3,4,5- trimethoxyphenyl) pyrimidine-2,4-diamine | CH2CH2OMe | 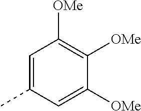 | 489 | (500 MHz, DMSOd6 at 353° K.): 3.18 (s, 3H), 3.54 (t, 2H), 3.64 (s, 3H), 3.75 (s, 6H), 3.90 (bs, 1H), 5.64 (bs, 1H), 6.08 (s, 2H), 6.95 (d, 1H), 7.06 (d, 1H), 7.10 (s, 2H), 7.91 (d, 1H), 8.67 (s, 1H) |
| 232 | 3-[[4-[(5-chlorobenzo [1,3]dioxol-4-yl)-(2- methoxyethyl)amino] pyrimidin-2-yl]amino] benzonitrile | CH2CH2OMe | 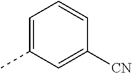 | 424 | (500 MHz, DMSOd6 + TFAd) Major rotamer: 3.25 (s, 3H), 3.58-3.66 (m, 2H), 3.97-4.06 (m, 1H), 4.22-4.31 (m, 1H), 6.05 (d, 1H), 6.23 (d, 2H), 7.14 (d, 1H), 7.21 (d, 1H), 7.68 (dd, 1H), 7.71 (d, 1H), 7.87 (ddd, 1H), 8.06 (d, 1H), 8.29 (s, 1H) |
| 233 | [3-[[4-[(5-chlorobenzo [1,3]dioxol-4-yl)-(2- methoxyethyl)amino] pyrimidin-2-yl]amino] phenyl]methanol | CH2CH2OMe | 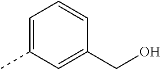 | 429 | (500 MHz, DMSOd6 + TFAd) Major rotamer: 3.15 (s, 3H), 3.51-3.60 (m, 2H), 3.91-3.99 (m, 1H), 4.18-4.26 (m, 1H), 4.54 (s, 2H), 5.91(d, 1H), 6.16 (d, 2H), 7.04-7.10 (m, 1H), 7.13 (d, 1H), 7.17 (d, 1H), 7.37 (d, 1H), 7.41 (d, 1H), 7.64 (s, 1H), 7.93 (d, 1H) |
| 234 | 3-[[4-[(5-chlorobenzo [1,3]dioxol-4-yl)-(2- methoxyethyl)amino] pyrimidin-2-yl]amino) benzenesulfonamide | CH2CH2OMe | 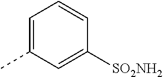 | 478 | (500 MHz, DMSOd6 + TFAd) Major retainer: 3.12 (s, 3H), 3.57 (t, 2H), 3.97- 4.04 (m, 1H), 4.19- 4.27 (m, 1H), 5.99 (d, 1H), 6.18 (d, 2H), 7.09 (d, 1H), 7.16 (d, 1H), 7.63 (dd, 1H), 7.69 (d, 1H), 7.78 (ddd, 1H), 8.08 (d, 1H), 8.23 (s, 1H) |
| 235 | N′-(5-chlorobenzo [1,3]dioxol-4-yl)-N′-(2- methoxyethyl)-N-(3- methylsulfonylphenyl) pyrimidine-2,4-diamine | CH2CH2OMe | 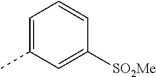 | 477 | 500 MHz, DMSOd6 + TFAd) Major rotamer: 3.11 (s, 3H), 3.22 (s, 3H), 3.54 (t, 2H), 3.97-4.04 (m, 1H), 4.20-4.28 (m, 1H), 5.98 (d, 1H), 6.15 (d, 2H), 7.05 (d, 1H), 7.12 (d, 1H), 7.69 (dd, 1H), 7.76 (d, 1H), 7.81 (dd, 1H), 8.00 (d, 1H), 8.42 (s, 1H) |
| 236 | N-[3-[[4-[(5-chlorobenzo [1,3]dioxol-4-yl)-(2- methoxyethyl)amino] pyrimidin-2- yl]amino]phenyl]methane sulfonamide | CH2CH2OMe | 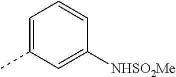 | 492 | 500 MHz, DMSOd6 + TFAd) Major rotamer: 3.02 (s, 3H), 3.12 (s, 3H), 3.50- 3.58 (m, 2H), 3.94- 4.02 (m, 1H), 4.19- 4.28 (m, 1H), 5.93 (d, 1H), 6.17 (d, 2H), 6.92 (d, 1H), 7.07 (d, 1H), 7.14 (d, 1H), 7.37 (s, 1H), 7.38 (d, 1H), 7.45 (s, 1H), 7.94 (d, 1H) |
| 237 | 2-[(2-anilinopyrimidin-4- yl)-(5-chlorobenzo[1,3] dioxol-[4-yl)amino]ethanol | CH2CH2OH | 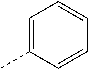 | 385 | 500 MHz, DMSOd6 + TFAd) Two rotamers are seen in the NMR spectrum (nearly 50/50): 3.61- 3.71 (m, 2H), 3.74- 3.86 (m, 1H), 4.01- 4.09 (0.5H), 4.10- 4.18 (0.5H), 5.92 (d, 1H), 6.06 (d, 1H), 6.17 (s, 1H), 6.95 (d, 0.5 H), 7.02 (dd, 0.5H), 7.06-7.20 (m, 4H), 7.45 (t, 1H), 7.760 (d, 1H), 7.93 (d, 0.5H), 8.22 (d, 0.5H) |
| 238 | 4-[[4-[(5-chlorobenzo [1,3]dioxol-4-yl)-(2- hydroxyethyl)amino] pyrimidin-2-yl]amino] benzenesulfonamide | CH2CH2OH | 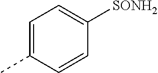 | 464 | 500 MHz, DMSOd6 + TFAd) Two rotamers are seen in the NMR spectrum (nearly 50/50): 3.65- 3.73 (m, 2H), 3.79- 3.91 (m, 1H), 4.02- 4.11 (m, 0.5H), 4.13- 4.22 (m, 0.5H), 5.99 (d, 0.5H), 6.11 (s, 1H), 6.18 (s, 1H), 7.02 (d, 0.5H), 7.09- 7.21 (m, 2H), 7.33 (d, 1H), 7.54 (d, 1H), 7.84 (dd, 1H), 7.90 (d, 1H), 8.02 (dd, 1H), 8.29 (dd, 1H) |
| 239 | N-[4-[[4-[(5-chlorobenzo [1,3]dioxol-4-yl)-(2- hydroxyethyl)amino] pyrimidin-2-yl]amino] phenyl]acetamide | CH2CH2OH | 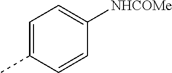 | 442 | 500 MHz, DMSOd6 + TFAd) Two rotamers are seen in the NMR spectrum (nearly 50/50): 2.01 (1.5H), 2.05 (1.5H), 3.60-3.70 (m, 2H), 3.72-3.86 (m, 1H), 4.00-4.08 (m, 0.5H), 4.09-4.17 (m, 0.5H), 5.88 (d, 1H), 6.08 (d, 1H), 6.16 (s, 1H), 6.92 (d, 0.5H), 7.03 (d, 0.5H), 7.06-7.10 (m, 1H), 7.11-7.17 (m, 1H), 7.34 (d, 1H), 7.50 (d, 1H), 7.67 (d, 1H), 7.88 (bs, 0.5H), 8.19 (d, 0.5H) |
| 240 | 2-[(5-chlorobenzo [1,3]dioxol-4-yl)- [2-[(3,4,5- trimethoxyphenyl)amino] pyrimidin-4-yl]amino] ethanol | CH2CH2OH | 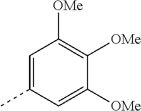 | 475 | 500 MHz, DMSOd6 at 353° K.) 3.64 (t, 2H), 3.68 (s, 3H), 3.78 (s, 6H), 3.83 (bs, 1H), 4.13 (bs, 1H), 6.09 (bs, 1H), 6.12 (s, 1H), 6.89 (bs, 2H), 7.01 (d, 1H), 7.09 (d, 1H), 7.94 (d, 1H), 10.03 (bs, 1H) |
| 241 | 3-[[4-[(5-chlorobenzo [1,3]dioxol-4-yl)-(2- hydroxyethyl)amino] pyrimidin-2-yl]amino] benzonitrile | CH2CH2OH | 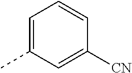 | 410 | 500 MHz, DMSOd6 + TFAd) Two rotamers are seen in the NMR spectrum (nearly 50/50): 3.63- 3.71 (m, 2H), 3.79- 3.88 (m, 1H), 3.97- 4.06 (m, 0.5H), 4,08- 4.17 (m, 0.5H), 5.99 (d, 0.5H), 6.07 (d, 1H), 6.18 (s, 1H), 7.07 (dd, 1H),7.10 (d, 0.5H), 7.14 (d, 0.5H), 7.16 (d, 0.5H), 7.31 (dd, 0.5H), 7.48 (dd, 1H), 7.54 (bs, 0.5H), 7.61-7.69 (m, 1H), 7.89 (dd, 0.5H), 7.99 (dd, 0.5H), 8.13 (d, 0.5H), 8.28 (dd, 0.5H) |
| 242 | 2-[(5-chlorobenzo [1,3]dioxol-4-yl)-2-[[3- (hydroxymethyl) phenyl]amino] pyrimidin-4-yl]amino] ethanol | CH2CH2OH | 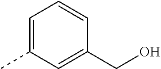 | 415 | 500 MHz, DMSOd6 + TFAd) Major rotamer: 3.67 (t, 2H), 3.78-3.87 (m, 1H), 4.11-4.20 (1H), 4.56 (s, 2H), 5.92 (d, 1H), 6.17 (s, 2H), 7.00 (s, 1H), 7.03-7.21 (m, 3H), 7.33-7.43 (m, m), 7.71 (s, 1H), 7.94 (d, 1H) |
| 243 | 3-[[4-[(5-chlorobenzo [1,3]dioxol-4-yl)-(2- hydroxyethyl)amino] pyrimidin-2-yl]amino] benzenesulfonamide | CH2CH2OH | 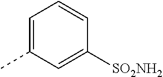 | 464 | 500 MHz, DMSOd6 + TFAd) Major rotamer: 3.66 (t, 2H), 3.82-3.91 (m, 1H), 4.12-4.23 (m, 1H), 5.98 (d, 1H), 6.18 (s, 2H), 7.06-7.19 (m, 2H), 7.63 (dd, 1H), 7.67 (d, 1H), 7.76 (d, 1H), 8.02 (d, 1H), 8.28 (bs, 1H) |
| 244 | 2-[(5-chlorobenzo [1,3]dioxol-4-yl)-[2-[(3- methylsulfonylphenyl) amino]pyrimidin-4- yl]amino]ethanol | CH2CH2OH | 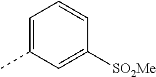 | 463 | 500 MHz, DMSOd6 at 353° K.) 3.19 (s, 3H), 3.71 (t, 2H), 3.86 (bs, 1H), 4.12 (bs, 1H), 6.13 (s, 2H), 7.05 (d, 1H), 7.14 (d, 1H), 7.52 (bs, 1H), 7.58 (s, 1H), 7.88 (s, 1H), 8.08 (s, 1H), 8.25 (bs, 1H), 10.30 (s, 1H) |
| 245 | N-[3-[[4-[(5-chlorobcnzo [1,3]dioxol-4-yl)-(2- hydroxyethyl)amino] pyrimidin-2-yl]amino] phcnyl]methane sulfonamide | CH2CH2OH | 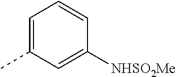 | 478 | 500 MHz, DMSOd6 + TFAd) 3.03 (s, 3H), 3.65 (t, 2H), 3.82- 3.91 (m, 1H), 4.12 4.21 (m, 1H), 5.93 (d, 1H), 6.17 (s, 2H), 6.93 (d, 1H), 6.86- 7.17 (m, 3H), 7.35- 7.42 (m, 2H), 7.47 (s, 1H), |

### EXAMPLE 11

### Starting Material

### 2-chloro-N-(5-fluorobenzo[1,3]dioxol-4-yl)pyrimidin-4-amine

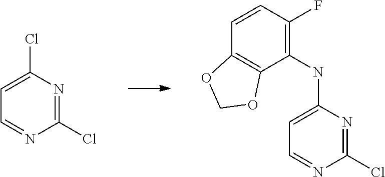

The title compound was prepared from 5-fluorobenzo[1,3]dioxol-4-amine following the procedure described in Example 1, Step 1, except that THF was used as a solvent (30% yield); NMR Spectrum 6.09 (s, 2H), 6.62 (br s, 1H), 6.79 (dd, 1H), 6.87 (dd, 1H), 8.16 (d, 1H), 9.77 (br s, 1H); Mass Spectrum MH⁺ 268.

### Final Compounds

The procedure described in Example 10 (Final compounds) was repeated using the appropriate aniline and 2-chloro-N-(5-fluorobenzo[1,3]dioxol-4-yl)pyrimidin-4-amine. Thus were obtained the compounds described in Table 6 below.

TABLE 6

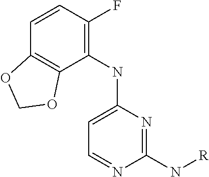

| | | | Molecular | NMR Spectrum |
|---|---|---|---|---|
| | | | Ion | (500 MHz, |
| No. | Name | R | (M + H⁺) | d6-DMSO) |
| 246 | N-(3,5-dimethoxyphenyl)-N′-(5- fluorobenzo[1,3]dioxol-4-yl) pyrimidine-2,4-diamine | 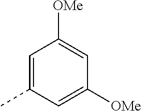 | 385 | 3.61 (s, 6H), 6.01 (s, 2H), 6.02 (t, 1H), 6.13 (d, 1H), 6.74 (dd, 1H), 6.81 (dd, 1H), 6.95 (d, 2H), 8.01 (d, 1H), 8.96 (s,1H), 9.00 (s, 1H). |
| 247 | 3-[[4-[(5-fluorobenzo[1,3]dioxol- 4-yl)amino]pyrimidin-2-yl]amino]- N,N-dimethyl-benzamide | 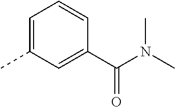 | 396 | 2.84 (s, 3H), 2.98 (s, 3H), 6.02 (s, 2H), 6.17 (d, 1H), 6.75 (dd, 1H), 6.78-6.90 (m, 2H), 7.14 (dd, 1H),7.62 (s, 1H), 7.70 (d, 1H), 8.02 (d, 1H), 9.02 (bs, 1H), 9.25 (s, 1H). |
| 248 | 4-[[4-[(5-fluorobenzo[1,3]dioxol- 4-yl)amino]pyrimidin-2-yl]amino]- N-methyl-benzamide | 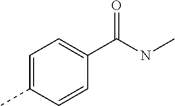 | 382 | 2.76 (d, 3H), 6.03 (s, 2H), 6.20 (d, 1H), 6.81 (dd, 1H), 6.89 (dd, 1H), 7.59 (d, 2H), 7.68 (d, 2H), 8.05 (d, 1H), 8.18 (q, 1H), 9.09 (s, 1H), 9.41 (s, 1H). |
| 249 | 4-[[4-[(5-fluorobenzo[1,3]dioxol- 4-yl)amino]pyrimidin-2- yl]amino]benzenesulfonamide | 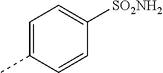 | 404 | 6.05 (s, 2H), 6.24 (d, 1H), 6.81 (dd, 1H), 6.90 (dd, 1H), 7.12 (s, 2H), 7.54 (d, 2H), 7.77 (d, 2H), 8.07 (d, 1H), 9.14 (s, 1H), 9.58 (s, 1H). |
| 250 | 3-[[4-[(5-fluorobenzo[1,3]dioxol- 4-yl)amino]pyrimidin-2- yl]amino]benzonitrile | 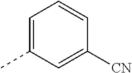 | 350 | 6.02 (s, 2H), 6.22 (d, 1H), 6.77-6.87 (m, 2H), 7.27 (d, 1H), 7.33 (dd, 1H), 7.86 (d, 1H), 8.06 (d, 1H), 8.11 (bs, 1H), 9.15 (s, 1H), 9.52 (s, 1H). |
| 251 | N′-(5-fluorobenzo[1,3]dioxol-4- yl)-N-(3-1,3-oxazol-5-ylphenyl) pyrimidine-2,4-diamine | 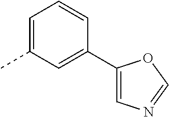 | 392 | 5.97 (s, 2H), 6.17 (d, 1H), 6.72-6.82 (m, 2H), 7.19-7.24 (m, 2H), 7.48 (s, 1H), 7.70 (dd, 1H), 7.96 (bs, 1H), 8.04 (d, 1H), 8.39 (s, 1H), 9.03 (bs, 1H), 9.27 (s, 1H). |
| 252 | 2-[4-[[4-[(5-fluorobenzo[1,3] dioxol-4-yl)amino]pyrimidin-2- yl]amino]phenyl]cthanol | 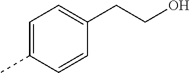 | 369 | 2.61 (t, 2H), 3.49-3.56 (m, 2H), 4.58 (t, 1H), 6.01 (s, 2H), 6.11 (d, 1H), 6.78 (dd, 1H), 6.86 (dd, 1H), 6.94 (d, 1H), 6.95 (s, 1H), 7.51 (d, 1H), 7.98 (d, 1H), 8.95 (s, 1H), 9.00 (s. 1H). |
| 253 | N-(3,4-dimethoxyphenyl)-N′-(5- fluorobenzo[1,3]dioxol-4-yl) pyrimidine-2,4-diamine | 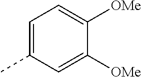 | 385 | 3.56 (s, 3H), 3.68 (s, 3H), 5.98 (s, 2H), 6.08 (d, 1H), 6.70 (d, 1H), 6.77 (dd, 1H), 6.83 (dd, 1H), 7.18 (dd, 1H), 7.29 (bs, 1H),7.97 (d, 1H), 8.87 (bs, 1H), 8.91 (bs, 1H). |
| 254 | N′-(5-fluorobenzo[1,3]dioxol-4- yl)-N-(3-methylsulfonylphenyl) pyrimidine-(2,4-diamine | 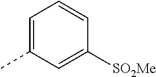 | 403 | 3.13 (s, 3H), 6.05 (s, 2H), 6.21 (d, 1H), 6.79 (dd, 1H), 6.86 (dd, 1H), 7.32-7.40 (m, 2H), 8.06 (d, 1H), 8.09-8.15 (m, 2H), 9.08 (bs, 1H), 9.51 (s, 1H). |
| 255 | 3-[[4-[(5-fluorobenzo[1,3]dioxol- 4-yl)amino]pyrimidin-2-yl] amino]-N-methyl-benzene- sulfonamide | 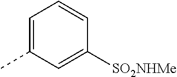 | 418 | 2.42 (s, 3H), 6.04 (s, 2H), 6.19 (d, 1H), 6.79 (dd, 1H), 6.86 (dd, 1H), 7.24 (d, 1H), 7.30 (dd, 1H), 7.37 (bs, 1H), 8.00 (s, 1H), 8.05 (d, 1H), 8.08 (d, 1H), 9.04 (bs, 1H), 9.46 (s, 1H). |
| 256 | [3-[[4-[(5-fluorobenzo[1,3]dioxol- 4-yl)amino]pyrimidin-2-yl]amino] phenyl]methanol | 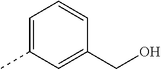 | 355 | 4.37 (s, 2H), 5.01 (bs, 1H), 6.00 (s, 2H), 6.13 (d, 1H), 6.78 (dd, 1H), 6.81-6.87 (m, 2H), 7.05 (dd, 1H), 7.48 (s, 1H), 7.93 (bs, 1H), 8.95 (bs, 1H), 9.08 (s, 1H). |
| 257 | N′-(5-fluorobenzo[1,3]dioxol-4- yl)-N-[3-(2-pyrrolidin-l- ylethoxy)phenyl]pyrimidine- 2,4-diamine | 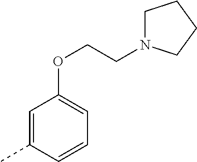 | 438 | 1.63-1.75 (m, 4H), 2.48- 2.52 (m partially hidden by DMSO-d5, 4H), 2.75 (t, 2H), 3.93 (t, 2H), 6.01 (s, 2H), 6.14 (d, 1H), 6.43 (dd, 1H), 6.77 (dd, 1H), 6.82 (dd, 1H), 6.99 (dd, 1H), 7.24 (d, 1H), 7.27 (s, 1H), 8.01 (d, 1H), 8.98 (s, 1H), 9.04 (s, 1H). |

### EXAMPLE 12

### Starting Material

### N-benzo[1,3]dioxol-4-yl-2-chloro-pyrimidin-4-amine

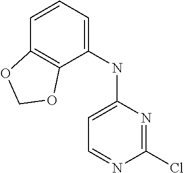

A mixture of 2,4-dichloropyrimidine (4.0 g, 27 mmol), 4-aminobenzodioxole (3.7 g, 27 mmol) and diethylisopropylamine (5.1 ml, 29.7 mmol) in DMF (25 ml) was stirred at 50° C. for 18 hours, then at 80° C. for 9 hours. After concentration under vacuum, the residue was partitioned between water and ethyl acetate and the precipitate was filtered, washed with water then ether and dried under vacuum. The organic layer from the filtrate was dried, evaporated and the residue purified on silica gel (10 to 50% EtOAc in petroleum ether) to give a solid, which was combined with the precipitate to provide 3.35 g of the title compound (50% yield); NMR Spectrum 6.05 (s, 2H), 6.68 (br s, 1H), 6.81 (d, 1H), 6.87 (t, 1H), 7.05 (br s, 1H), 8.15 (d, 1H), 9.83 (br s, 1H); Mass Spectrum MH⁺ 250.

### Final Compounds

The procedure described in Example 10 (Final compounds) was repeated using N-benzo[1,3]dioxol-4-yl-2-chloro-pyrimidin-4-amine and the appropriate aniline. Thus were obtained the compounds described in Table 7 below.

TABLE 7

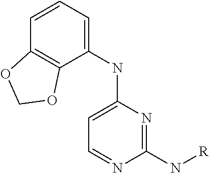

| | | | Molecular | NMR Spectrum |
|---|---|---|---|---|
| | | | Ion | (500 MHz, |
| No. | Name | R | (M + H⁺) | d6-DMSO) |
| 258 | N′-benzo[1,3]dioxol-4-yl- N-(3,5-dimethoxyphenyl) pyrimidine-2,4-diamine | 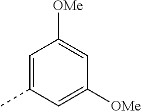 | 367 | 3.65 (s, 6H), 5.98 (s, 2H), 6.31 (s, 1H), 6.49 (bs, 1H), 6.69 (d, 2H), 6.82-6.91 (m, 2H), 7.06 (d, 1H), 8.01 (d, 1H), 10.57 (bs, 1H), 10.72 (bs, 1H) |
| 259 | N-[5-[[4-(benzo[1,3] dioxol-4-ylamino)pyrimidin- 2-yl]amino]-2-methyl- phenyl]acetamide | 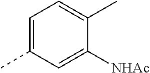 | 378 | 2.07 (s, 3H), 2.17 (s, 3H), 6.02 (s, 2H), 6.49 (bs, 1H), 6.84-6.93 (m, 2H), 7.03- 7.16 (m, 2H), 7.28 (bs, 1H), 7.53 (s, 1H), 7.99 (d, 1H), 9.30 (s, 1H), 10.56 (bs, 1H), 10.74 (bs, 1H). |
| 260 | 3-[[4-(benzo[1,3]dioxol- 4-ylamino)pyrimidin-2- yl]amino]-N,N-dimethyl- benzamide | 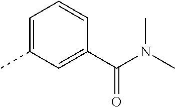 | 378 | 2.86 (s, 3H), 2.96 (s, 3H), 6.01 (s, 2H), 6.26 (d, 1H), 6.75 (d, 1H), 6.82 (dd, 1H), 6.86 (d, 1H), 7.21 (d, 1H), 7.24 (d, 1H), 7.75 (d, 1H), 7.77 (d, 1H), 8.02 (d, 1H), 9.08 (s, 1H), 9.26 (s, 1H). |
| 261 | 4-[[4-(benzo[1,3]dioxol- 4-ylamino)pyrimidin-2- yl]amino]benzamide | 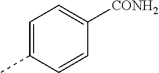 | 350 | — |
| 262 | 4-[[4-(benzo[1,3]dioxol- 4-ylamino)pyrimidin-2- yl]amino]-N-methyl- benzamide | 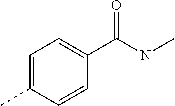 | 364 | 2.79 (d, 3H), 6.02 (s, 2H), 6.51 (d, 1H), 6.73 (d, 1H), 6.93 (d, 1H), 7.04 (dd, 1H), 7.59 (d, 2H), 7.75 (d, 2H), 8.07 (d, 1H), 8.27 (q, 1H), 10.67 (s, 1H), 10.73 (s, 1H). |
| 263 | 4-[[4-(benzo[1,3]dioxol- 4-ylamino)pyrimidin-2- yl]amino]benzene- sulfonamide | 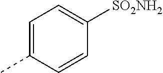 | 386 | 6.03 (s, 2H), 6.55 (d, 1H), 6.72 (d, 2H), 6.88 (dd, 1H), 6.94 (d, 1H), 7.03 (dd, 1H), 7.23 (bs, 2H), 7.53 (d, 2H), 8.11 (d, 1H), 10.84 (s, 1H), 11.08 (s, 1H). |
| 264 | 3-[[4-(benzo[1,3]dioxol- 4-ylamino)pyrimidin-2- yl]amino]benzonitrile | 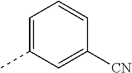 | 332 | 5.99 (s, 2H), 6.51 (d, 1H), 6.87 (dd, 1H), 6.92 (dd, 1H), 7.05 (dd, 1H), 7.48 (dd, 1H), 7.53 (ddd, 1H), 7.77 (ddd, 1H), 8.03 (bs, 1H), 8.08 (d, 1H), 10.57 (bs, 1H), 10.78 (bs, 1H). |
| 265 | 3-[[4-(benzo[1,3]dioxol- 4-ylamino)pyrimidin-2- yl]amino]-N-methyl- bcnzamidc | 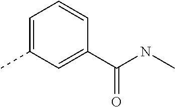 | 364 | 2.76 (d, 3H), 6.01 (s, 2H), 6.27 (d, 1H), 6.73 (dd, 1H), 6.80 (dd, 1H), 7.24 (dd, 1H), 7.29 (ddd, 1H), 7.34 (d, 1H), 7.93 (ddd, 1H), 8.01 (bs, 1H), 8.02 (d, 1H), 8.25 (q, 1H), 9.05 (s, 1H), 9.23 (s, 1H). |
| 266 | N′-benzo[1,3]dioxol-4-yl- N-(3-dimethylaminophenyl) pyrimidine-2,4-diamine | 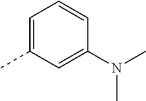 | 350 | 2.96 (s, 6H), 6.05 (s, 2H), 6.66 (dd, 1H), 6.68 (d, 1H), 6.74 (dd, 1H), 6.79 (dd, 1H), 6.80 (dd, 1H), 6.88 (dd, 1H), 7.03 (bs, 1H), 7.08 (d, 1H), 7.27 (dd, 1H), 8.14 (d, 1H), 9.74 (bs, 1H). |
| 267 | 2-[4-[[4-(benzo[1,3]dioxol- 4-ylamino)pyrimidin-2- yl]amino]phenyl]ethanol | 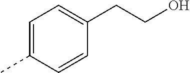 | 351 | 2.64 (t, 2H), 3.55 (td, 2H), 4.60 (t, 1H), 6.00 (s, 2H), 6.20 (d, 1H), 6.75 (dd, 1H), 6.83 (dd, 1H), 7.01 (d, 2H), 7.27 (d, 1H), 7.59 (d, 2H), 7.98 (d, 1H), 9.00 (s, 1H), 9.01 (s, 1H). |
| 268 | 1-[4-[[4-(benzo[1,3]dioxol- 4-ylamino)pyrimidin-2- yl]amino]phenyl]-N- methyl-methanesulfonamide | 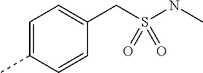 | 414 | 2.56 (d, 3H), 4.23 (s, 2H), 6.03 (s, 2H), 6.25 (d, 1H), 6.78 (dd, 1H), 6.84 (q, 1H), 6.86 (dd, 1H), 7.18 (d, 2H), 7.26 (d, 1H), 7.73 (d, 2H), 8.02 (d, 1H), 9.09 (s, 1H), 9.21 (s, 1H). |
| 269 | N′-benzo[1,3]dioxol- 4-yl-N-(3,4-dimethoxy- phenyl)pyrimidine-2,4- diamine | 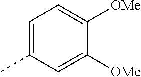 | 367 | (500 MHz, DMSOd6 at 323° K.) 3.63 (s, 3H), 3.77 s, 3H), 5.97 (s, 2H), 6.44 (d, 1H), 6.81-6.88 (m, 2H), 6.91 (d, 1H), 6.97 (dd, 1H), 7.05-7.12 (m, 2H), 7.94 (d, 1H), 10.26 (bs, 1H), 10.47 (bs, 1H). |
| 270 | 5-[[4-(benzo[1,3]dioxol- 4-ylamino)pyrimidin-2- yl]amino]-1,3-dihydroindol- 2-one | 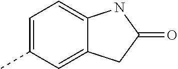 | 362 | 3.32 (s, 2H), 5.99 (s, 2H), 6.17 (d, 1H), 6.63 (d, 1H), 6.77 (dd, 1H), 6.84 (dd, 1H), 7.20 (d, 1H), 7.37 (dd, 1H), 7.67 (s, 1H), 7.96 (d, 1H), 8.96 (s, 1H), 9.00 (s, 1H), 10.16 (s, 1H) |
| 271 | N′-benzo[1,3]dioxol-4-yl- N-(3-methylsulfonylphenyl) pyrimidine-2,4-diamine | 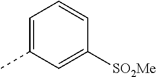 | 385 | 3.15 (s, 3H), 6.03 (s, 2H), 6.31 (d, 1H), 6.76 (dd, 1H), 6.86 (dd, 1H), 7.29 (d, 1H), 7.41 (ddd, 1H), 7.44 (dd, 1H), 8.06 (d, 1H), 8.18 (ddd, 1H), 8.22 (bs, 1H), 9.14 (s, 1H), 9.52 (s, 1H). |
| 272 | 3-[[4-(benzo[1,3]dioxol-4- ylamino)pyrimidin-2-yl] amino]-N-methyl- benzenesulfonamide | 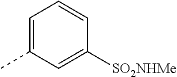 | 400 | (500 MHz, DMSOd6 at 323° K.) 2.44 (d, 3H), 6.00 (s, 2H), 6.49 (d, 1H), 6.84-6.91 (m, 2H), 7.06 (dd, 1H), 7.40 (bs, 1H), 7.46 (dd, 1H), 7.51 (ddd, 1H), 7.80 (bs, 1H), 7.96 (d, 1H), 8.06 (d, 1H), 10.44 (bs, 1H), 10.60 (bs, 1H), |
| 273 | [3-[[4-(benzo[1,3]dioxol-4- ylamino)pyrimidin-2-yl] amino]phenyl]methanol | 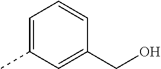 | 337 | 4.39 (d, 2H), 5.07 (t, 1H), 6.00 (s, 2H), 6.23 (d, 1H), 6.74 (dd, 1H), 6.84 (dd, 1H), 6.86 (d, 1H), 7.12 (dd, 1H), 731 (d, 1H), 7.60 (d, 1H), 7.61 (s, 1H), 8.00 (d, 1H), 9.03 (s, 1H), 9.08 (s, 1H). |
| 274 | N′-benzo[1,3]dioxol-4- yl-N-(1H-indol-4-yl) pyrimidine-2,4-diamine | 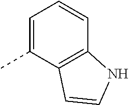 | 346 | (500 MHz, DMSOd6 at 323° K.) 6.00 (s, 2H), 6.49 (bs, 1H), 6.67 (s, 1H), 6.80- 6.91 (m, 2H), 6.94-7.10 (m, 2H), 7.29 (d, 1H), 7.33- 7.41 (m, 2H), 8.00 (d, 1H), 10.42 (s, 1H), 10.63 (s, 1H), 11.22 (s, 1H). |
| 275 | N-[4-[[4-(benzo[1,3]dioxol- 4-ylamino)pyrimidin-2-yl] amino]phenyl]acetamide | 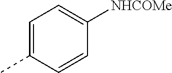 | 364 | 2.00 (s, 3H), 6.01 (s, 2H), 6.20 (d, 1H), 6.75 (dd, 1H), 6.83 (dd, 1H), 7.25 (d, 1H), 7.37 (d, 2H), 7.60 (d, 2H), 7.98 (d, 1H), 9.02 (s, 1H), 9.03 (s, 1H), 9.75 (s, 1H). |

### EXAMPLE 13

### Starting Material

### N-benzo[1,3]dioxol-4-yl-2-chloro-N-methyl-pyrimidin-4-amine

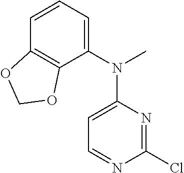

The title compound was prepared following the same procedure as for Example 10 (starting material (1)) except that caesium carbonate was used as a base (68% yield); NMR Spectrum 3.36 (s, 3H), 6.06 (s, 2H), 6.41 (br s, 1H), 6.88-6.91 (m, 1H), 6.94-6.98 (m, 2H), 8.07 (d, 1H); Mass Spectrum MH⁺ 264.

### Final Compounds

The procedure described in Example 10 (Final compounds) was repeated using N-benzo[1,3]dioxol-4-yl-2-chloro-N-methyl-pyrimidin-4-amine and the appropriate aniline. Thus were obtained the compounds described in Table 8 below.

TABLE 8

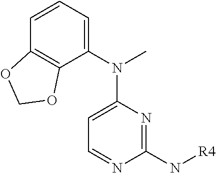

| | | | Molecular | NMR Spectrum |
|---|---|---|---|---|
| | | | Ion | (500 MHz, |
| No. | Name | R4 | (M + H⁺) | d6-DMSO) |
| 276 | N′-benzo[1,3]dioxol-4-yl- N-(3,5-dimethoxyphenyl)- N′-methyl-pyrimidine-2,4- diamine | 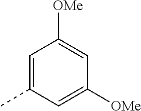 | 381 | 3.41 (s, 3H), 3.69 (s, 6H), 5.85 (s, 1H), 6.04 (s, 2H), 6.07 (t, 1H), 6.88 (dd, 1H), 6.91-6.97 (m, 2H), 7.07 (d, 2H), 7.93 (d, 1H), 9.14 (s, 1H). |
| 277 | N-[5-[[4-(benzo[1,3]dioxol- 4-yl-methyl-amino) pyrimidin- 2-yl]amino]-2-methyl- phenyl]acetamidc | 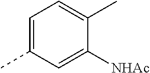 | 392 | 2.03 (s, 3H), 2.10 (s, 3H), 3.37 (s, partially hidden by H20, 3H), 5.86 (d, 1H), 6.03 (s, 2H), 6.84-6.89 (m, 1H), 6.93 (d, 1H), 6.94 (s, 1H), 6.99 (d, 1H), 7.44 (dd, 1H), 7.73 (bs, 1H), 7.92 (d, 1H), 9.10 (s, 1H), 9.23 (s, 1H). |
| 278 | 3-[[4-(benzo[1,3]dioxol- 4-yl-methyl-amino) pyrimidin-2-yl]amino]- N,N-dimethyl-benzamide | 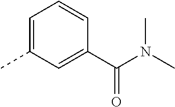 | 392 | 2.90 (s, 3H), 2.98 (s, 3H), 3.38 (s, 3H), 5.94 (d, 1H), 6.02 (s, 2H), 6.84-6.90 (m, 2H), 6.91- 6.98 (m, 2H), 7.22 (dd, 1H), 7.75 (s, 1H), 7.76 (d, 1H), 7.97 (d, 1H), 9.33 (s, 1H). |
| 279 | 4-[[4-(benzo[1,3]dioxol- 4-yl-methyl-amino) pyrimidin-2-yl]amino] benzamide | 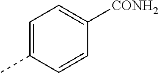 | 364 | 3.40 (s, 3H), 6.00 (bs, 1H), 6.03 (s, 2H), 6.89 (dd, 1H), 6.95-6.99 (m, 2H), 7.11 (bs, 1H), 7.68- 7.79 (s, 5H), 8.01 (d, 1H), 9.48 (s, 1H). |
| 280 | 4-[[4-(benzo[1,3]dioxol- 4-yl-methyl-amino) pyrimidin-2-yl]amino] benzenesulfonamide | 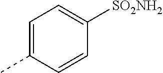 | 400 | 3.40 (s, 3H), 6.03 (s, 2H), 6.05 (bs, 1H), 6.89 (dd, 1H), 6.95-6.99 (m, 2H), 7.13 (bs, 2H), 7.61 (d, 2H), 7.81 (d, 2H), 8.03 (d, 1H), 9.62 (s, 1H). |
| 281 | 3-[[4-(benzo[1,3]dioxol- 4-yl-methyl-amino) pyrimidin-2-yl]amino] benzonitrile | 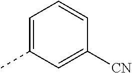 | 346 | 3.39 (s, 3H), 6.01 (bs, 1H), 6.02 (s, 2H), 6.89 (dd, 1H), 6.93-6.98 (m, 2H), 7.30 (d, 1H), 7.38 (dd, 1H), 7.94 (d, 1H), 8.02 (d, 1H), 3.18 (bs, 1H), 9.59 (s, 1H). |
| 282 | 3-[[4-(benzo[1,3]dioxol- 4-yl-methyl-amino) pyrimidin-2-yl]amino] benzamide | 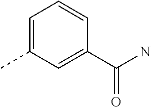 | 364 | 3.42 (s, 3H), 5.91 (d, 1H), 6.05 (s, 2H), 6.86- 6.92 (m, 1H), 6.96 (d, 2H), 7.20-7.31 (m, 2H), 7.38 (d, 1H), 7.80-7.88 (m, 2H), 7.97 (d, 1H), 8.28 (bs, 1H), 9.32 (s, 1H). |
| 283 | 3-[[4-(benzo[1,3]dioxol-4-yl- methyl-amino)pyrimidin-2- yl]amino]-N-methyl-benzamide | 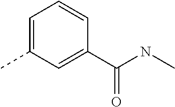 | 378 | 2.77 (d, 3H), 3.41 (s, 3H), 5.90 (d, 1H), 6.03 (s, 2H), 6.86-6.90 (m, 1H), 6.92-6.97 (m, 2H), 7.24 (dd, 1H), 7.30 (d, 1H), 7.80 (dd, 1H), 7.95 (d, 1H), 8.26 (bs, 1H), 8.29 (q, 1H), 9.33 (s, 1H). |
| 284 | N′-benzo[1,3]dioxol-4-yl-N-(3- dimethylaminophenyl)-N′- methyl-pyrimidine-2,4-diamine | 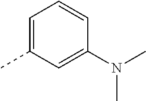 | 364 | 2.86 (s, 6H), 3.40 (s, 3H), 5.83 (d, 1H), 6.03 (s, 2H), 6.30 (dd, 1H), 6.87 (dd, 1H), 6.91-6.96 (m, 2H), 6.99 (dd, 1H), 7.08 (d, 1H), 7.21 (s, 1H), 7.92 (d, 1H), 8.94 (s, 1H). |
| 285 | N′-benzo[1,3]dioxol-4-yl-N′- methyl-N-(3-methylsulfonyl- phenyl)pyrimidine-2,4-diamine | 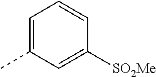 | 399 | 3.17 (s, 3H), 3.43 (s, 3H), 5.93 (d, 1H), 6.05 (s, 2H), 6.89 (dd, 1H), 6.92-6.99 (m, 2H), 7.42 (d, 1H), 7.47 (dd, 1H), 7.89 (d, 1H), 7.98 (d, 1H), 8.62 (bs, 1H), 9.67 (s, 1H). |
| 286 | 3-[[4-(benzo[1,3]dioxol-4-yl- methyl-amino)pyrimidin-2- yl]amino]benzenesulfonamide | 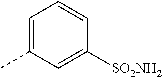 | 400 | 3.42 (s, 3H), 5.91 (d, 1H), 6.05 (s, 2H), 6.86- 6.91 (m, 1H), 6.93-6.99 (m, 2H), 7.26 (s, 2H), 7.32-7.41 (m, 2H), 7.79 (d, 1H), 7.97 (d, 1H), 8.50 (bs, 1H), 9.57 (s, 1H). |
| 287 | [3-[[4-(benzo[1,3]dioxol-4-yl- methyl-amino)pyrimidin-2- yl]amino]phenyl]methanol | 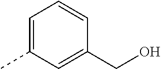 | 351 | 3.39 (s, 3H), 4.42 (d, 2H), 5.10 (t, 1H), 5.89 (d, 1H), 6.02 (s, 2H), 6.84 (d, 1H), 6.86-6.90 (m, 1H), 6.92-6.97 (m, 2H), 7.13 (dd, 1H), 7.55 (d, 1H), 7.72 (bs, 1H), 7.95 (d, 1H), 9.15 (s, 1H). |
| 288 | N-[3-[[4-(benzo[1,3]dioxol-4-yl- methyl-amino)pyrimidin-2- yl]amino]phenyl] methanesulfonamide | 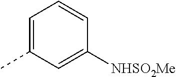 | 414 | 3.40 (s, 3H), 5.87 (d, 1H), 6.04 (s, 2H), 6.74 (dd, 1H), 6.84-6.90 (m, 1H), 6.92-6.97 (m, 2H), 7.13 (dd, 1H), 7.47 (dd, 1H), 7.66 (s, 1H), 7.93 (d, 1H), 9.24 (s, 1H), 9.59 (s, 1H). |
| 289 | N′-benzo[1,3]dioxol-4-yl-N′- methyl-N-[3-(2-pyrrolidin-1- ylethoxy)phenyl]pyrimidine- 2,4-diamine | 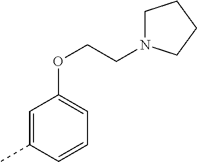 | 434 | 1.62-1.73 (m, 4H), 2.51- 2.63 (m, partially hidden by H20, 4H) 2.77 (t, 2H), 3.39 (s, 3H), 4.00 (t, 2H), 5.89 (d, 1H), 6.03 (s, 2H), 6.46 (dd, 1H), 6.85-6.90 (m, 1H), 6.92-6.97 (m, 2H), 7.07 (dd, 1H), 7.27 (dd, 1H), 7.49 (bs, 1H), 7.95 (d, 1H, 9.15 (s, 1H). |
| 290 | N′-benzo[1,3]dioxol-4-yl-N- methyl-N-(4-methylsulfonyl- phenyl)pyrimidine-2,4-diamine | 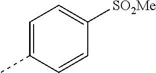 | 399 | 3.13 (s, 3H), 3.41 (s, 3H), 6.03 (s, 2H), 6.07 (bs, 1H), 6.90 (dd, 1H), 6.94-7.01 (m, 2H), 7.69 (d, 2H), 7.91 (d, 2H), 8.04 (d, 1H), 9.78 (s, 1H). |
| 291 | N-[4-[[4-(benzo[1,3]dioxol-4-yl- methyl-amino)pyrimidin-2- yl]amino]phenyl]acetamide | 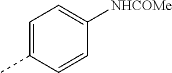 | 378 | 2.00 (s, 3H), 3.38 (s, partially hidden by H20, 3H), 5.91 (d, 1H), 6.01 (s, 2H), 6.84-6.89 (m, 1H), 6.90-6.97 (m, 2H), 7.37 (d, 2H), 7.56 (d, 2H), 7.94 (d, 1H), 9.08 (s, 1H), 9.75 (s, 1H). |

### EXAMPLE 14

### Starting Material

### N-(2-methylsulfonylpyrimidin-4-yl)-1H-indazol-4-amine

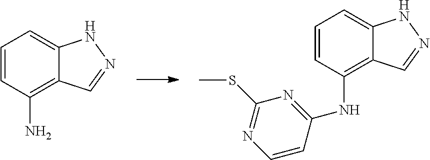

A mixture of 4-chloro-2-methylthiopyrimidine (2.75 ml, 23.7 mmol) and 4-aminoindazole (3.0 g, 22.5 mmol) and hydrogen chloride (1 drop, 4N in dioxane) in n-butanol (45 ml) was heated at 80° C. for 4 hours. Diethyl ether was added and the resulting precipitate was filtered and rinsed with ether. This solid was taken in water, the pH adjusted to 7 by to addition of aqueous sodium bicarbonate and the solid was filtered and rinsed with water, ether and dried under vacuum to yield 5.7 g (93%) of a pale yellow solid. NMR Spectrum: (500 MHz, DMSO) 2.46 (s, 3H), 6.69 (d, 1H), 7.23 (d, 1H), 7.31 (t, 1H), 7.71 (d, 1H), 8.16 (d, 1H), 8.23 (s, 1H), 9.71 (s, 1H), 13.1 (br s, 1H); Mass spectrum: MH⁺ 258.

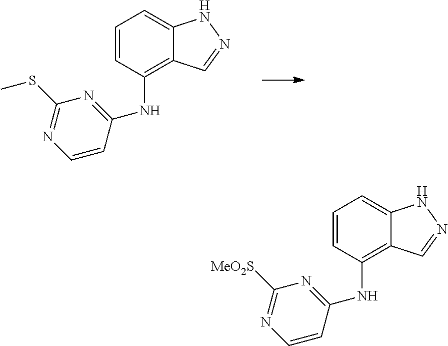

m-Chloroperbenzoic acid (6.81 g, 27.7 mmol) was added to an ice-cooled solution of N-(2-methylsulfanylpyrimidin-4-yl)-1H-indazol-4-amine (3 g, 11.6 mmol) in DMF (80 ml). The mixture was then stirred at room temperature for 3 hours. The mixture was concentrated, diluted in DCM, washed with sodium bicarbonate and brine, and dried over MgSO₄. After evaporation of the solvents, the residue was triturated in EtOAc/ether and dried to give N-(2-methylsulfonylpyrimidin-4-yl)-1H-indazol-4-amine (2.4 g, 71%) as a solid. NMR Spectrum: (500 MHz, DMSO) 3.31 (s, 3H), 7.13 (d, 1H), 7.32-7.38 (m, 2H), 7.66 (br s, 1H), 8.23 (s, 1H), 8.47 (d, 1H), 10.3 (br s, 1H), 13.1 (br s, 1H); Mass spectrum: MH⁺ 290.

### Final Compounds

### N-(3,5-dimethoxyphenyl)-N′-(1H-indazol-4-yl)pyrimidine-2,4-diamine

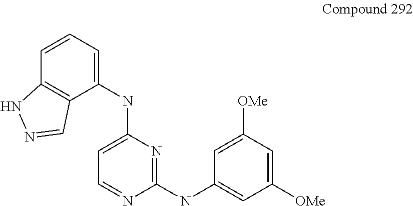

A 4N HCl solution in dioxane (0.1 ml) was added to a mixture of N-(2-methylsulfonylpyrimidin-4-yl)-1H-indazol-4-amine (87 mg, 0.3 mmol) and 3,5-dimethoxyaniline (1 eq.) in 2-pentanol (0.9 ml). The mixture was irradiated in a Personal Chemistry EMRYS™ Optimizer EXP microwave synthesisor at 170° C. for 10 minutes. The reaction mixture was cooled to room temperature and purified on a preparative HPLC-MS system (Column: C18, 5 microns, 19 mm diameter, 100 mm length; elution with a gradient of water and acetonitrile containing 2 g/l of ammonium carbonate); evaporation of the collected fractions gave the title compound (65 mg, 61%); NMR Spectrum: 3.66 (s, 6H), 6.09 (t, 1H), 6.47 (d, 1H), 7.04 (d, 2H), 7.20 (d, 1H), 7.28 (dd, 1H), 7.97 (d, 1H), 8.10 (s, 1H), 8.30 (s, 1H), 9.13 (s, 1H), 9.39 (s, 1H), 13.06 (s, 1H); Mass Spectrum MH⁺ 363.

The procedure described above was repeated using the appropriate aniline. Thus were obtained the compounds described in Table 9 below.

TABLE 9

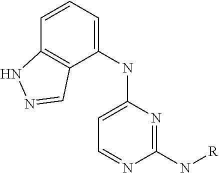

| | | | Molecular | NMR Spectrum |
|---|---|---|---|---|
| | | | Ion | (500 MHz, |
| No. | Name | R | (M + H⁺) | d6-DMSO) |
| 293 | N-[5-[[4-(1H-indazol-4- ylamino)pyrimidin-2-yl] amino]-2-methyl-phcnyl] acetamide | 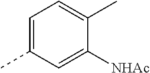 | 374 | 2.03 (s, 3H), 2.12 (s, 3H), 6.45 (d, 1H), 7.04 (d, 1H), 7.18 (d, 1H), 7.29 (dd, 1H), 7.48 (dd, 1H), 7.74 (d, 1H), 8.01 (d, 1H), 8.07 (d, 1H), 8.33 (s, 1H), 9.14 (s, 1H), 9.28 (s, 1H), 9.35 (s, 1H), 13.05 (s, 1H) |
| 294 | 3-[[4-(1H-indazol-4- ylamino)pyrimidin-2- yl]amino]-N,N-dimethyl- benzamide | 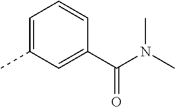 | 374 | 2.87 (bs, 3H), 2.97 (bs, 3H), 6.48 (d, 1H), 7.27 (d, 1H), 7.33 (dd, 1H),7.65 1H), 7.92 (d, 1H), 8.14 (d, 1H), 8.26 (s, 1H), 8.74 (d, 1H), 9.16 (s, 1H), 9.51 (s, 1H), 9.55 (s, 1H), 13.11 (s, 1H) |
| 295 | 3-[[4-(1H-indazol-4- ylamino)pyrimidin-2- yl]amino]benzamide | 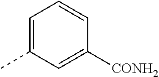 | 346 | 6.49 (d, 1H), 7.18 (d, 1H), 7.23-7.33 (m, 3H), 7.41 (d, 1H), 7.84 (s, 1H), 7.93 (dd, 1H), 7.99 (d, 1H), 8.11 (d, 1H), 8.12 (s, 1H), 8.32 (s, 1H), 9.31 (s, 1H), 9.39 (s, 1H), 13.05 (s, 1H) |
| 296 | 3-[[4-(1H-indazol-4- ylamino)pyrimidin-2- yl]amino]-N-methyl- benzamide | 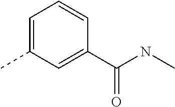 | 360 | 2.76 (d, 3H), 6.49 (d, 1H), 7.18 (d, 1H), 7.24 (dd, 1H), 7.29 (dd, 1H), 7.34 (ddd, 1H), 7.90 (dd, 1H), 7.98 (d, 1H), 8.10 (d, 1H), 8.12 (s, 1H), 8.29 (q, 1H), 8.32 (s, 1H), 9.33 (s, 1H), 9.39 (s, 1H), 13.05 (s, 1H) |
| 297 | N′-(1H-indazol-4-yl)- N-(3-1,3-oxazol-5- ylphenyl)pyrimidine- 2,4-diamine | 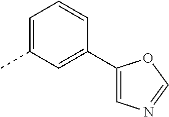 | 370 | 6.48 (d, 1H), 7.11- 7.20 (m, 2H), 7.29 (d, 1H), 7.33 (dd, 1H), 7.50 (s, 1H), 7.74 (ddd, 1H), 7.93 (d, 1H), 8.12 (d, 1H), 8.13 (s, 1H), 8.30 (s, 1H), 8.39 (s, 1H), 9.37 (s, 1H), 9.43 (s, 1H), 13.05 (s, 1H) |
| 298 | N-(3-dimethylaminophenyl)- N′-(1H-indazol-4-yl) pyrimidine-2,4-diamine | 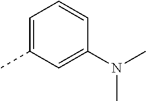 | 346 | 2.82 (s, 6H), 6.33 (dd, 1H), 6.44 (d, 1H), 7.03 (dd, 1H), 7.09 (dd, 1H), 7.16- 7.21 (m, 2H), 7.26 (dd, 1H), 8.01 (d, 1H), 8.07 (d, 1H), 8.31 (s, 1H), 8.95 (s, 1H), 9.33 (s, 1H), 13.04 (s, 1H) |
| 299 | 2-[4-[[4-(1H-indazol-4- ylamino)pyrimidin-2- yl]amino]phenyl]ethanol | 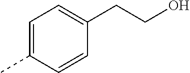 | 347 | 2.66 (t, 2H), 3.57 (dt, 2H), 4.62 (t, 1H), 6.43 (d, 1H), 7.07 (d, 2H), 7.20 (d, 1H), 7.29 (dd, 1H), 7.62 (d, 2H), 7.95 (d, 1H), 8.07 (d, 1H), 8.30 bs, 1H), 9.09 (s, 1H), 9.36 (s, 1H), 13.05 (s, 1H) |
| 300 | N-(3,4-dimethoxyphenyl)- N′-(1H-indazol-4-yl) pyrimidine-2,4-diamine | 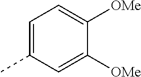 | 363 | 3.62 (s, 3H), 3.72 (s, 3H), 6.42 (d, 1H), 6.83 (d, 1H), 7.18 (d, 1H), 7.22-7.29 (m, 2H), 7.37 (d, 1H), 7.95 (d, 1H), 8.06 (d, 1H), 8.30 (s, 1H), 8.98 (s, 1H), 9.33 (s, 1H), 13.05 (s, 1H) |
| 301 | N′-(1H-indazol-4-yl)-N- (1H-indol-5-yl)pyrimidine- 2,4-diamine | 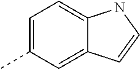 | 343 | 6.31 (dd, 1H), 6.39 (d, 1H), 7.17 (d, 1H), 7.21 (dd, 1H), 7.25- 7.32 (m, 3H), 7.98 (s, 1H), 8.03 (d, 1H), 8.05 (d, 1H), 8.33 (s, 1H), 8.92 (s, 1H), 9.28 (s, 1H), 10.89 (s, 1H), 13.04 (s, 1H) |
| 302 | 5-[[4-(1H-indazol-4- ylamino)pyrimidin-2- yl]amino]-1,3- dihydroindol-2-one | 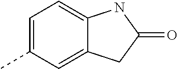 | 358 | 3.39 (s, 2H), 6.39 (d, 1H), 6.68 (d, 1H), 7.21 (d, 1H), 7.28 (dd, 1H), 7.39 (dd, 1H), 7.68 (s, 1H), 7.86 (d, 1H), 8.04 (d, 1H), 8.27 (s, 1H), 9.03 (s, 1H), 9.35 (s, 1H), 10.20 (s, 1H), 13.06 (s, 1H) |
| 303 | 3-[[4-(1H-indazol-4- ylamino)pyrimidin-2- yl]amino]phenyl]methanol | 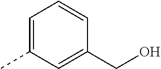 | 333 | 4.43 (d, 2H), 5.11 (t, 1H), 6.46 (d, 1H), 6.90 (d, 1H), 7.18 (dd, 1H), 7.20 (d, 1H), 7.30 (dd, 1H), 7.61 (d, 1H), 7.69 (s, 1H), 7.97 (d, 1H), 8.09 (d, 1H), 8.31 (s, 1H), 9.18 (s, 1H), 9.37 (s, 1H), 10.06 (s, 1H) |
| 304 | N-[3-[[4-(1H-indazol-4- ylamino)pyrimidin-2- yl]amino]phenyl] methanesulfonamide | 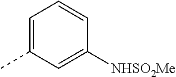 | 396 | 2.99 (s, 3H), 6.48 (d, 1H), 6.78 (ddd, 1H), 7.17 (dd, 1H), 7.19 (d, 1H), 7.31 (dd, 1H), 7.58 (s, 1H), 7.61 (d, 1H), 8.01 (d, 1H), 8.09 (d, 1H), 8.32 (s, 1H), 9.25 (s, 1H), 9.35 (s, 1H), 9.61 (bs, 1H), 13.06 (s, 1H) |

### EXAMPLE 15

### N′-(5-chlorobenzo[1,3]dioxol-4-yl)-N′-(2-dimethylaminoethyl)-N-(3-methylsulfonylphenyl)pyrimidine-2,4-diamine

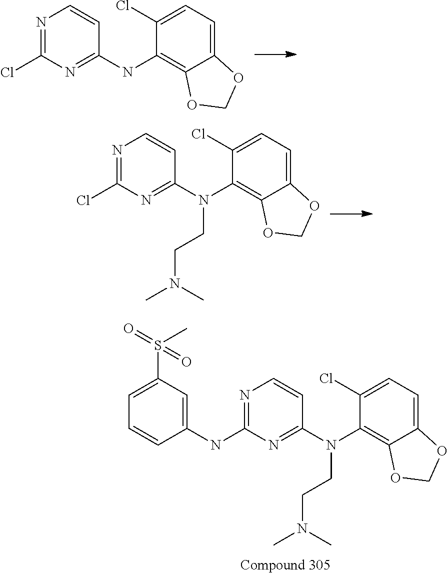

2-Chloro-N-(5-chloro-1,3-benzodioxol-4-yl)pyrimidin-4-amine (2.0 g, 7.07 mmol) was dissolved in DMF (20 mL). Sodium hydride (60%, 680 mg, 17 mmol) was added, followed by 2-dimethylaminoethyl chloride (hydrochloride, 1.22 g, 8.5 mmol) and the mixture was heated at 50° C. overnight. After evaporation under reduced pressure, the residue was purified on silica gel chromatography (0-5% MeOH in methylene chloride) to yield N-(5-chlorobenzo[1,3]dioxol-4-yl)-N-(2-chloropyrimidin-4-yl)-N′,N′-dimethyl-ethane-1,2-diamine as a colorless oil (8.45 mg, 33%); NMR Spectrum (500 MHz, DMSOd6+TFAd) 2.91 (d, 6H), 3.34 (t, 2H), 3.88-3.93 (m, 1H), 4.44-4.47 (m, 1H), 6.18-6.25 (m, 3H), 7.12 (d, 1H), 7.19 (d, 1H), 8.17 (bs, 1H); Mass Spectrum MH⁺ 355.

The procedure described in Example 10 (Final compounds) was repeated using N-(5-chlorobenzo[1,3]dioxol-4-yl)-N-(2-chloropyrimidin-4-yl)-N′,N′-dimethyl-ethane-1,2-diamine (20 mg, 0.06 mmol) and 3-methylsulfonylaniline hydrochloride (13 mg, 0.06 mmol) except that the mixture was heated for 3 hours. Yield: 10 mg, 36%; NMR Spectrum (500 MHz, DMSOd6+TFAd) 2.69 (s, 6H), 3.27-3.29 (m, 2H), 3.29 (s, 3H), 3.91-3.95 (m, 1H), 4.45-4.50 (m, 1H), 6.02 (d, 1H), 6.23 (d, 2H), 7.16 (d, 1H), 7.23 (d, 1H), 7.73-7.82 (m, 2H), 7.91 (d, 1H), 8.10 (d, 1H), 8.21 (s, 1H); Mass Spectrum MH⁺ 490.

### EXAMPLE 16

### N′-(6-chlorobenzofuran-7-yl)-N-(3-methylsulfonylphenyl)pyrimidine-2,4-diamine

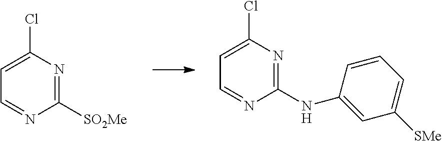

Sodium hydride (13.4 g, 60% dispersion in mineral oil) was added portion-wise to a ice-cooled solution of 3-methylthioformanilide (6.7 g, 40 mmol) [prepared by heating 3-methylthioaniline (13.9 g) in formic acid (50 ml) for 2 h at reflux, evaporation of the solvent, partitioning with ethyl acetate/aq. sodium bicarbonate and chromatography on silica gel (10% EtOAc in DCM)] in THF (200 ml). The mixture was stirred at room temperature for 10 minutes, then cooled at 0° C. 4-Chloro-2-methylsulfonylpyrimidine (8.49, 44.1 mmol, L. Xu et al, J. Org. Chem. 2003, 68, 5388) was added portionwise to the mixture. The reaction was warmed to room temperature, stirred for one hour and quenched cautiously with water. The mixture was extracted with EtOAc. The organic layer was washed with water and brine, dried over MgSO₄ and concentrated. The residue was triturated in 20 ml of diethyl ether to give N-(4-chloropyrimidin-2-yl)-N-(3-methylsulfanylphenyl)formamide as a solid (9 g). Aqueous 2N sodium hydroxide (20 ml, 40 mmol) was added to a solution of this solid (9 g) in THF-methanol (50 ml:50 ml). After 15 minute stirring at room temperature, the mixture was evaporated under vacuum. The residue was diluted with EtOAc, washed with water and brine, dried and concentrated to give 4-chloro-N-(3-methylsulfanylphenyl)pyrimidin-2-amine (7.1 g, 71%). NMR Spectrum (500 MHz, DMSO) 2.51 (s, 3H), 6.76 (d, 1H), 6.98 (m, 1H), 7.29 (m, 2H), 7.64 (s, 1H), 8.29 (d, 1H); Mass Spectrum MH⁺ 252

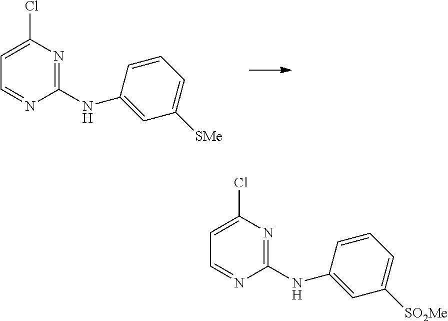

m-Chloroperbenzoic acid (13.6 g, 70% strength, 55 mmol) was added portionwise to an ice-cooled solution of 4-chloro-N-(3-methylsulfanylphenyl)pyrimidin-2-amine (6.6 g, 26.3 mmol) in DCM (250 ml). The mixture was stirred at room temperature for 1 hour. The mixture was washed with aqueous sodium dithionate, aqueous sodium bicarbonate, then brine. After evaporation of the solvent, the residue was purified by chromatography on to silica gel (15% EtOAc in DCM) to give 4-chloro-N-(3-methylsulfonylphenyl)pyrimidin-2-amine (6 g, 80%) as a white solid. NMR Spectrum (500 MHz, DMSO) 3.20 (s, 3H), 7.07 (d, 1H), 7.58 (m, 2H), 7.99 (m, 1H), 8.39 (s, 1H), 8.52 (d, 1H), 10.44 (s, 1H); Mass Spectrum MH⁺ 284

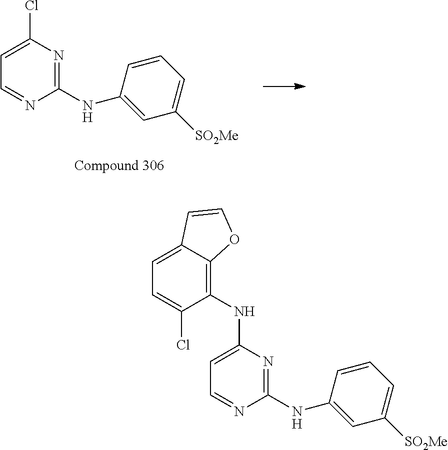

4-Chloro-N-(3-methylsulfonylphenyl)pyrimidin-2-amine (200 mg, 0.69 mmol) and 6-chlorobenzofuran-7-amine (127 mg, 0.76 mmol, Plé P. et al., J. Med. Chem. 2004, 47, 871) were dissolved in isopropanol (3 ml). 1M HCl in diethyl ether (1 drop) added. The reaction was heated at 90° C. for 1 hour then cooled to room temperature and concentrated in vacuo. The residue was directly injected on an HPLC column (C18, 5 microns, 19 mm diameter, 100 mm length) of a preparative HPLC-MS system eluting with a mixture of water and acetonitrile containing 2 g/l of ammonium carbonate (gradient). After evaporation of the solvents, the mixture was repurified by chromatography on silica gel (eluting with 20% to 30% EtOAc in DCM) to give the title compound as a white solid (85 mg, 30%); NMR Spectrum (500 MHz, DMSO) 3.09 (s, 3H), 6.24 (m, 1H), 7.05 (s, 1H), 7.11 (m, 1H), 7.29 (d, 1H), 7.45 (d, 1H), 7.63 (d, 1H), 7.71 (m, 1H), 7.98 (m, 1H), 8.02 (s, 1H), 8.07 (d, 1H), 9.36 (s, 1H), 9.45 (s, 1H); Mass Spectrum MH⁺ 415.

### EXAMPLE 17

The procedure described above was repeated using the appropriate aniline. Thus were obtained the compounds described in Table 10 below

TABLE 10

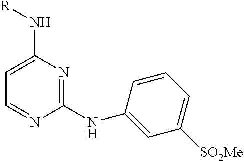

| | | | Molecular | |
|---|---|---|---|---|
| | | | Ion | NMR Spectrum (500 |
| No. | Name | R | (Observed) | MHz, d6-DMSO) |
| 307 (Note 1) | N′-(2,3- dihydrobenzofuran-7-yl)- N-(3- methylsulfonylphenyl) pyrimidine-2,4-diamine | 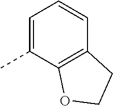 | 383 (MH⁺) | 3.14 (s, 3H), 3.24 (t, 2H), 4.56 (t, 2H), 6.32 (m, 1H), 6.84 (t, 1H), 7.02 (d, 1H), 7.40 (m, 2H), 7.66 (m, 1H), 8.01 (d, 1H), 8.17 (d, 1H), 8.22 (s, 1H), 8.86 (s, 1H), 9.49 (s, 1H) |
| 308 (Note 2) | N′-(benzofuran-7-yl)-N- methylsulfonylphenyl) pyrimidine-2,4-diamine | 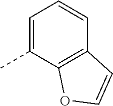 | 381 (MH⁺) | 3.13 (s, 3H), 6.42 (m, 1H), 7.01 (d, 1H), 7.25 (m, 1H), 7.36 (m, 2H), 7.42 (m, 1H), 7.86 (m, 1H), 8.10-8.02 (m, 3H), 8.19 (s, 1H), 9.49 (s, 1H), 9.55 (s, 1H) |
| 309 | N′-(1H-indazol-4-yl)-N- (3- methylsulfonylphenyl) pyrimidine-2,4-diamine | 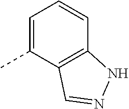 | 381 (MH⁺) | 3.16 (s, 3H), 6.53 (d, 1H), 7.21 (d, 1H), 7.32 (t, 1H), 7.43 (d, 1H), 7.48 (t, 1H), 7.94 (m, 1H), 8.15 (m, 2H), 8.29 (s, 2H), 9.46 (s, 1H), 9.61 (s, 1H) |
| 311 (Note 3) | N′-(3-chloro-1H-indol-7- yl)-N-(3- methylsulfonylphenyl) pyrimidine-2,4-diamine | 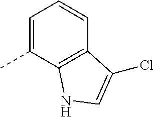 | 414 (MH⁺) | 3.13 (s, 3H), 6.20 (d, 1H), 7.13 (t, 1H), 7.33 (m, 3H), 7.45 (m, 2H), 7.97 (s, 1H), 8.06 (d, 1H), 8.17 (s, 1H), 9.17 (s, 1H), 9.49 (s, 1H), 11.17 (s, 1H) |
| 312 (Note 4) | N′-(6- methoxybenzo[1,3]dioxol- 4-yl)-N-(3- methylsulfonylphenyl) pyrimidine-2,4-diamine | 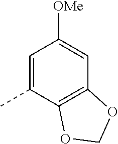 | 415 (MH⁺) | 3.14 (s, 3H), 3.67 (s, 3H), 5.97 (s, 2H), 6.29 (d, 1H), 6.46 (d, 1H), 6.76 (s, 1H), 7.41 (m, 2H), 8.05 (d, 1H), 8.21 (m, 2H), 9.13 (s, 1H), 9.52 (s, 1H) |
| 313 (Note 5) | 4-[[2-[(3- methylsulfonylphenyl) amino]pyrimidin-4- yl]amino]benzo[1,3]dioxole- 5-carboxamide | 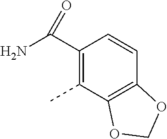 | 428 (MH⁺) | 3.14 (s, 3H), 6.05 (s, 2H), 6.28 (d, 1H), 6.86 (d, 1H), 7.26 (d, 1H), 7.40 (m, 3H), 7.81 (s br, 1H), 8.09 (m, 2H), 8.21 (s br, 1H), 9.42 (s, 1H), 9.54 (s, 1H) |
| 314 (Note 6) | N′-isoquinolin-5-yl-N-(3- methylsulfonylphenyl) pyrimidine-2,4-diamine | 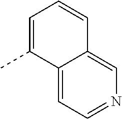 | 392 (MH⁺) | 3.11 (s, 3H), 6.41 (d, 1H), 7.30 (m, 1H), 7.36 (d, 1H), 7.73 (t, 1H), 7.96 (m, 3H), 8.13 (m, 3H), 8.53 (d, 1H), 9.36 (s, 1H), 9.51 (s, 1H) |

Note 1:
2,3-dihydrobenzofuran-7-amine (Birch A. et al. J. Med. Chem., 1999, 42, 3342)
Note 2:
Benzofuran-7-amine (Plé P et al., J Med. Chem, 2004, 47, 871)
Note 3:
3-Chloro-1H-indol-7-amine (Plé P et al., J Med. Chem, 2004, 47, 871)
Note 4:
6-Methoxybenzo[1,3]dioxol-4-amine (Astrazeneca, PCT Appl. WO2002016352)
Note 5:
4-Aminobenzo[1,3]dioxole-5-carboxamide (Dallacker F., Annalen, 1960, 633, 14)
Note 6:
5-Aminoisoquinoline and 4-chloro-N-(3-methylsulfonylphenyl)pyrimidin-2-amine were reacted using Buchwald type conditions (procedure described in Example 24, Step 2, except that the mixture was irradiated in the microwave at 130° C. for 15 minutes)

### EXAMPLE 18

### N′-benzooxazol-7-yl-N-(3-methylsulfonylphenyl)pyrimidine-2,4-diamine

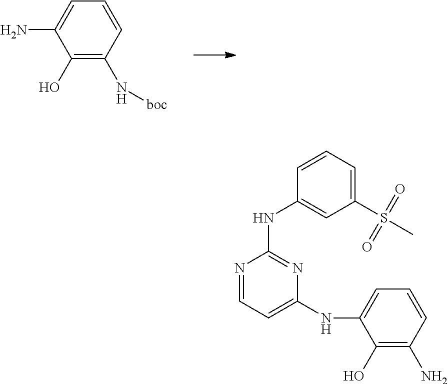

The procedure described in Example 16 was repeated using tert-butyl N-(3-amino-2-hydroxy-phenyl)carbamate [365 mg, 1.6 mmol; obtained from 2,6-dinitrophenol by hydrogenation with 10% palladium over charcoal in ethanol to obtain the 2,6-diaminophenol (quantitative) and treatment of di-tert-butyldicarbonate (3.2 g, 1 eq.) in THF (50 ml) and chromatography on silica gel (eluant: 4% EtOAc in DCM)] as the aniline. After cooling, the crude mixture was concentrated and treated with 50% TFA in DCM (10 ml) for 1 hour at room temperature. After evaporation of the solvents, the residue was directly injected on an HPLC column (C18, 5 microns, 19 mm diameter, 100 mm length) of a preparative HPLC-MS system eluting with a mixture of water and acetonitrile containing 2 g/l of ammonium carbonate (gradient) to give 2-amino-6-[[2-[(3-methylsulfonylphenyl)amino]pyrimidin-4-yl]amino]phenol (290 mg, 53%). NMR Spectrum: (500 MHz, DMSO) 3.13 (s, 3H), 4.7 (m, 2H), 6.25 (d, 1H), 6.49 (m, 1H), 6.61 (t, 1H), 6.71 (d, 1H), 7.42 (m, 2H), 7.97 (m, 1H), 8.16 (d, 1H), 8.26 (s, 1H), 8.71 (s, 1H), 9.53 (s, 1H); Mass spectrum: MH⁺ 372.

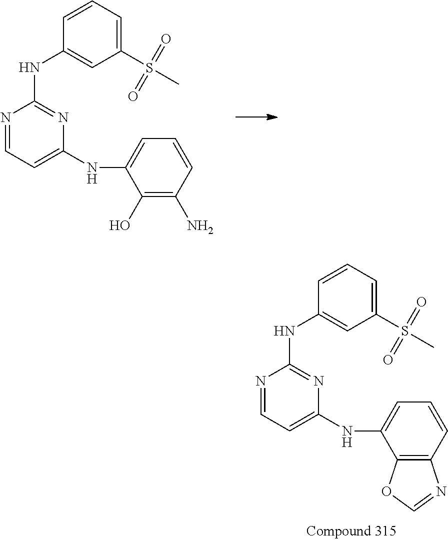

A mixture of 2-amino-6-[[2-[(3-methylsulfonylphenyl)amino]pyrimidin-4-yl]amino]phenol (260 mg, 0.70 mmol), trimethylorthoformate (0.614 ml, 5.6 mmol) and p-toluenesulfonic acid (5 mg) was heated at 95° C. for 30 minutes. After evaporation of the solvent, the residue was purified by chromatography on silica gel (eluant: 60% EtOAc in DCM) to give the title compound (60 mg, 22%) as white solid. NMR Spectrum: (500 MHz, DMSO) 3.14 (s, 3H), 6.45 (d, 1H), 7.41-7.33 (m, 3H), 7.55 (d, 1H), 7.91 (d, 1H), 8.00 (d, 1H), 8.13 (d, 1H), 8.19 (s, 1H), 8.75 (s, 1H), 9.57 (s, 1H), 9.69 (s, 1H); Mass spectrum: MH⁺ 382.

### EXAMPLE 19

### N′-benzooxazol-4-yl-N-(3-methylsulfonylphenyl)pyrimidine-2,4-diamine

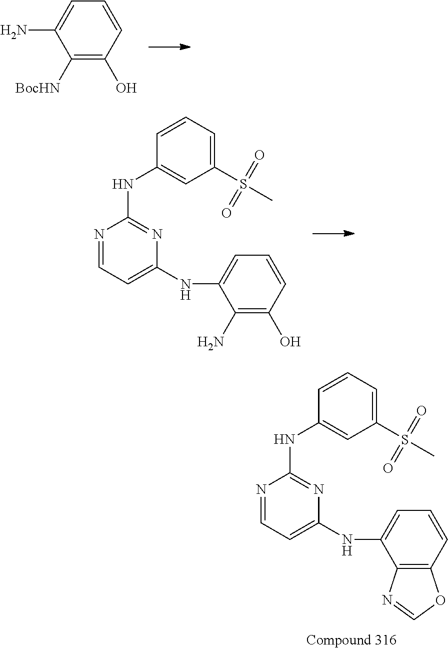

The procedure described in Example 18 was repeated using tert-butyl N-(2-amino-6-hydroxy-phenyl)carbamate (365 mg, 1.63 mmol, Astrazeneca, PCT Int. App. WO 2003053960 p 59 Ex. 3 starting material) as the aniline:

2-amino-3-[[2-[(3-methylsulfonylphenyl)amino]pyrimidin-4-yl]amino]phenol (260 mg, 47%), brown solid; NMR Spectrum: (500 MHz, DMSO) 3.14 (s, 3H), 4.30 (m, 2H), 6.05 (d, 1H), 6.48 (m, 1H), 6.62 (m, 1H), 6.73 (d, 1H), 7.39 (m, 2H), 7.96 (d, 1H), 8.20 (m, 2H), 8.51 (s, 1H), 9.30 (m, 1H), 9.43 (s, 1H); Mass spectrum: MH⁺ 372.

N′-benzooxazol-4-yl-N-(3-methylsulfonylphenyl)pyrimidine-2,4-diamine (90 mg, 36%), white solid; NMR Spectrum: (500 MHz, DMSO) 3.16 (s, 3H), 6.68 (d, 1H), 7.50-7.39 (m, 4H), 8.14 (d, 2H), 8.32 (m, 2H), 8.76 (s, 1H), 9.63 (s, 1H), 9.67 (s, 1H); Mass spectrum: MH⁺ 382.

### EXAMPLE 20

### 3-[4-(1H-indazol-4-yl-methyl-amino)pyrimidin-2-yl]-N,N-dimethyl-benzamide

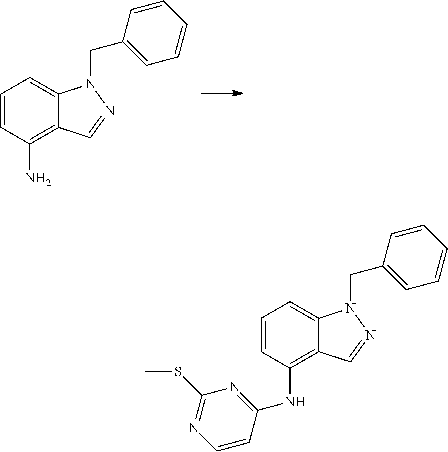

A mixture of 4-chloro-2-methylthiopyrimidine (2.4 ml, 20.7 mmol) and 1-benzylindazol-4-amine (4.15 g, 18.6 mmol, Kampe W. et al, Ger. Offen. DE2737630) and hydrogen chloride (1 drop, 4N in dioxane) in n-butanol (55 ml) was heated at reflux for 3 hours. After cooling and evaporation of the solvents, the residue was stirred with water. The pH was adjusted to 7 by addition of aqueous sodium bicarbonate and the mixture was extracted with DCM. The organic layer was washed with water and brine, and dried over MgSO₄. After evaporation of the solvents, the residue was purified by chromatography on silica gel (eluant: 10% to 70% EtOAc in petroleum ether) to give 1-benzyl-N-(2-methylsulfanylpyrimidin-4-yl)indazol-4-amine (5.6 g, 78%) as an orange solid. NMR Spectrum: (500 MHz, DMSO) 2.36 (s, 3H), 5.65 (s, 2H), 6.69 (d, 1H), 7.40-7.20 (m, 7H), 7.76 (d, 1H), 8.17 (d, 1H), 8.28 (s, 1H), 9.73 (s, 1H); Mass spectrum: MH⁺ 348.

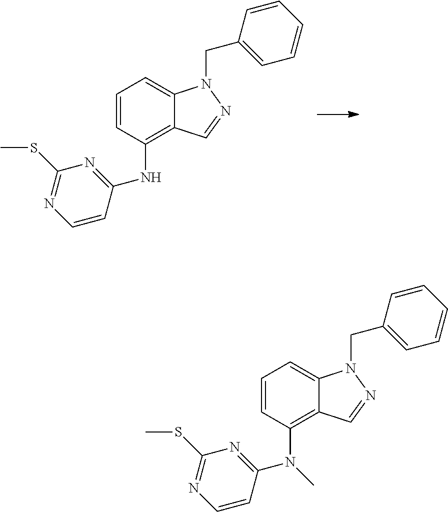

Methyl iodide (1 ml, 16.1 mmol) was added to a mixture of 1-benzyl-N-(2-methylsulfanylpyrimidin-4-yl)indazol-4-amine (5.6 g, 16.1 mmol) and cesium carbonate (10.5 g, 32.3 mmol) in acetonitrile (60 ml). The mixture was stirred at room temperature for 18 hours. The mixture was diluted with acetonitrile and the solids were filtered off. After evaporation of the solvents, the residue was dissolved in DCM, filtered and purified by chromatography on silica gel (eluant: 10 to 40% EtOAc in petroleum ether) to give 1-benzyl-N-methyl-N-(2-methylsulfanylpyrimidin-4-yl)indazol-4-amine (5 g, 86%) as a solid. NMR Spectrum: (500 MHz, DMSO) 2.41 (s, 3H), 3.52 (s, 3H), 5.70 (s, 2H), 5.95 (d, 1H), 7.13 (d, 1H), 7.34-7.25 (m, 5H), 7.47 (t, 1H), 7.75 (d, 1H), 7.91 (d, 1H), 7.96 (s, 1H).

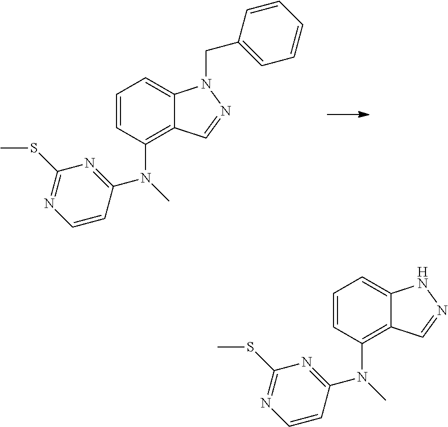

Potassium tert-butoxide (97 ml, 97 mmol, 1M solution in THF) was added to a mixture of 1-benzyl-N-methyl-N-(2-methylsulfanylpyrimidin-4-yl)indazol-4-amine (5 g, 13.85 mmol) in DMSO (9.9 ml)-THF (20 ml) at room temperature. Oxygen was bubbled through the solution for 20 minutes while maintaining the temperature below 30° C. with a cooling bath. The mixture was quenched with saturated aqueous ammonium chloride and extracted with EtOAc. The organic layer was washed with water and brine, and dried over MgSO₄. After evaporation of the solvents, the residue was purified by chromatography on silica gel (eluant: 10% to 70% EtOAc in petroleum ether) to give N-methyl-N-(2-methylsulfanylpyrimidin-4-yl)-1H-indazol-4-amine (3.1 g, 83%) as a white solid. NMR Spectrum: (500 MHz, DMSO) 2.44 (s, 3H), 3.52 (s, 3H), 5.91 (d, 1H), 7.09 (d, 1H), 7.44 (t, 1H), 7.56 (d, 1H), 7.91 (m, 2H), 13.34 (s br, 1H).

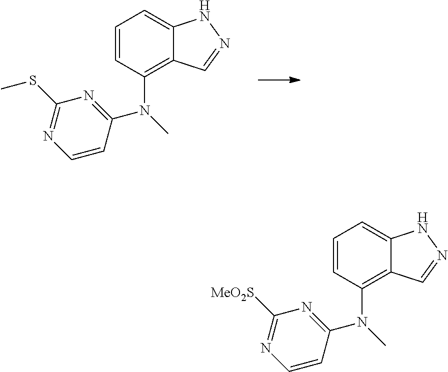

m-Chloroperbenzoic acid (6.81 g, 27.7 mmol) was added to an ice-cooled solution of N-methyl-N-(2-methylsulfanylpyrimidin-4-yl)-1H-indazol-4-amine (3 g, 11.1 mmol) in DMF (80 ml). The mixture was then stirred at room temperature for 4 hours. The mixture was concentrated, diluted in DCM, washed with sodium bicarbonate and brine, and dried over MgSO₄. After evaporation of the solvents, the residue was triturated in ether and dried to give N-methyl-N-(2-methylsulfonylpyrimidin-4-yl)-1H-indazol-4-amine (2.4 g, 72%) as a white solid. NMR Spectrum: (500 MHz, DMSO) 3.30 (s, 3H), 3.59 (s, 3H), 6.39 (m, 1H), 7.18 (d, 1H), 7.48 (t, 1H), 7.63 (d, 1H), 7.99 (s, 1H), 8.23 (d, 1H).

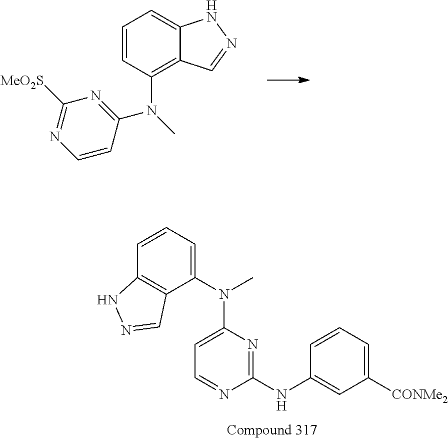

A mixture of N-methyl-N-(2-methylsulfonylpyrimidin-4-yl)-1H-indazol-4-amine (200 mg, 0.66 mmol), 3-amino-N,N-dimethyl-benzamide (130 mg, 0.79 mmol) and hydrogen chloride (4N in dioxane, 0.165 ml, 0.66 mmol) in 2-pentanol (3 ml) was irradiated in a Personal Chemistry EMRYS™ Optimizer EXP microwave synthesisor at 150° C. for 15 minutes. After cooling and evaporation of the solvents, the residue was dissolved in DMF (1.5 ml) and concentrated aqueous ammonia (50 μl) was added. The mixture was injected on an HPLC column (C18, 5 microns, 19 mm diameter, 100 mm length) of a preparative HPLC-MS system eluting with a mixture of water and acetonitrile containing 2 g/l of ammonium carbonate (gradient). Evaporation of the fractions gave the title compound (69 mg, 27%).

NMR Spectrum: (500 MHz, DMSO) 2.90-2.97 (br s, 3H), 2.97 (br s, 3H), 3.55 (s, 3H), 5.74 (d, 1H), 6.86 (d, 1H), 7.10 (d, 1H), 7.22 (dd, 1H), 7.45 (dd, 1H), 7.55 (d, 1H), 7.75 (d, 1H), 7.80 (d, 1H), 7.88 (d, 1H), 7.92 (s, 1H), 9.34 (s, 1H), 13.31 (br s, 1H); Mass spectrum: MH⁺ 388

### EXAMPLE 21

The procedure described in Example 20 above was repeated using the appropriate aniline in the last step. Thus were obtained the compounds described in Table 11 below.

TABLE 11

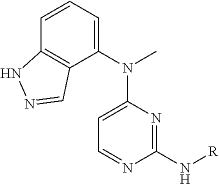

| | | | Molecular | |
|---|---|---|---|---|
| | | | Ion | NMR Spectrum (500 |
| No. | Name | R | (Observed) | MHz, d6-DMSO) |
| 318 | N-methyl-N-[2-(3- methylsulfonylphenyl) pyrimidin-4-yl]-1H- indazol-4-amine | 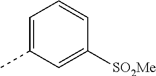 | 395 (MH⁺) | 3.17 (s, 3H), 3.59 (s, 3H), 5.73 (d, 1H), 7.12 (d, 1H), 7.39-7.49 (m, 3H), 7.57 (d, 1H), 7.86 (d, 1H), 7.89 (d, 1H), 7.93 (s, 1H), 8.69 (s, 1H), 9.69 (s, 1H), 13.33 (br s, 1H) |
| 319 | 3-[4-(1H-indazol-4-yl- methyl-amino)pyrimidin- 2-yl]benzenesulfonamide | 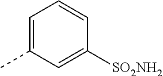 | 396 (MH⁺) | 3.58 (s, 3H), 5.70 (d, 1H), 7.11 (d, 1H), 7.26 (br s, 2H), 7.37-7.39 (m, 2H), 7.45 (dd, 1H), 7.56 (d, 1H), 7.77 (d, 1H), 7.86 (d, 1H), 7.92 (d. 1H), 8.56 (s, 1H), 9.58 (s, 1H), 13.32 (br s, 1H) |
| 320 | [3-[4-(1H-indazol-4-yl- methyl-amino)pyrimidin- 2-yl]phenyl]methanol | 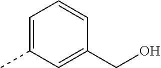 | 347 (MH⁺) | 3.56 (s, 3H), 4.42 (d, 2H), 5.09 (t, 1H), 5.70 (d, 1H), 6.84 (d, 1H), 7.10 (d, 1H), 7.12 (dd, 1H), 7.44 (dd, 1H), 7.52-7.58 (m, 2H), 7.76 (s, 1H), 7.85 (d, 1H), 7.91 (s, 1H), 9.17 (s, 1H), 13.31 (br s, 1H) |
| 321 | N-[3-[4-(1H-indazol-4- yl-methyl- amino)pyrimidin-2- yl]phenyl] methanesulfonamide | 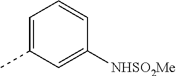 | 410 (MH⁺) | 2.98 (s, 3H), 3.56 (s, 3H), 5.67 (d, 1H), 6.74 (dd, 1H), 7.10 (d, 1H), 7.13 (dd, 1H), 7.44 (dd, 1H), 7.46 (d, 1H), 7.55 (d, 1H), 7.72 (s, 1H), 7.83 (d, 1H), 7.91 (s, 1H), 9.23 (s, 1H), 9.57 (bs, 1H), 13.32 (br s, 1H) |
| 322 | N-(3,5- dimorpholinophenyl)-N′- (1H-indazol-4-yl)-N′- methyl-pyrimidine-2,4- diamine | 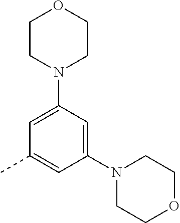 | 487 (MH⁺) | 3.04 (m, 8H), 3.56 (s, 3H), 3.71 (m, 8H), 5.62 (d, 1H), 6.11 (s, 1H), 7.01 (s, 2H), 7.09 (d, 1H), 7.43 (d, 1H), 7.54 (d, 1H), 7.81 (d, 1H), 7.93 (s, 1H), 8.88 (s, 1H), 13.31 (br s, 1H) |

### EXAMPLE 22

### N-(3,5-dimorpholin-4-ylphenyl)-N′-(1H-indazol-4-yl)pyrimidine-2,4-diamine

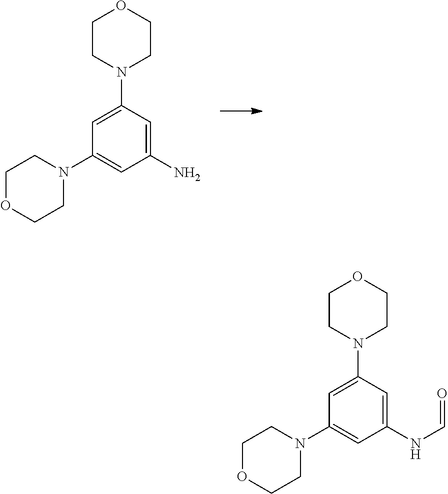

A mixture of 3,5-dimorpholin-4-ylaniline (500 mg, 1.90 mmol) in formic acid (8 ml) was heated at reflux for 2 hours. After cooling, the mixture was concentrated and the residue was dissolved in EtOAc, washed with aqueous saturated sodium bicarbonate and dried over MgSO4. After evaporation of the solvents, the residue was purified by chromatography on silica gel (eluant: 1% to 4% methanol in DCM) to give 3,5-dimorpholin-4-ylformanilide (400 mg, 58%) as a solid. Mass spectrum: MH⁺ 292.

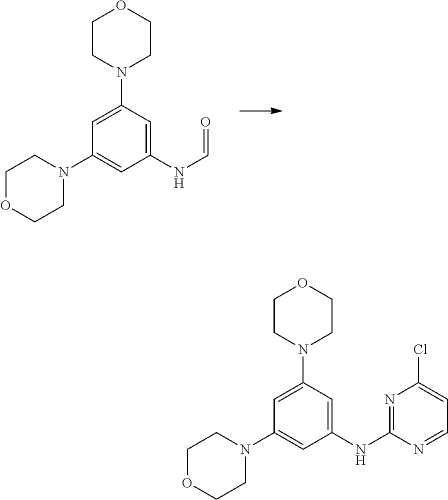

3,5-Dimorpholin-4-ylformanilide (400 mg, 1.37 mmol) and 4-chloro-2-methylsulfonylpyrimidine (291 mg, 1.51 mmol) were reacted according to procedure in Example 16, step 1, to give 4-chloro-N-(3,5-dimorpholin-4-ylphenyl)pyrimidin-2-amine (314 mg, 61%) as a solid. NMR Spectrum: (500 MHz, DMSO) 3.06 (m, 8H), 3.72 (m, 8H), 6.19 (s, 1H), 6.91 (m, 3H), 8.41 (d, 1H), 9.74 (s, 1H); Mass spectrum: MH⁺ 376.

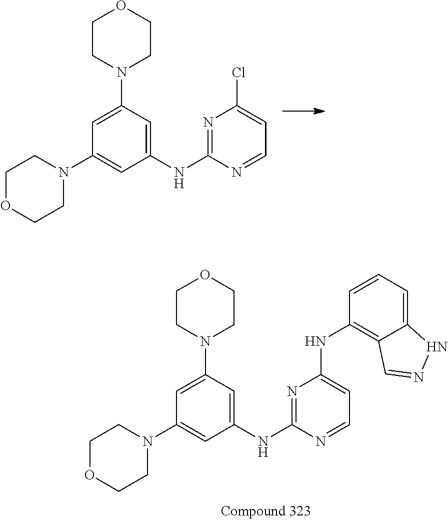

4-chloro-N-(3,5-dimorpholin-4-ylphenyl)pyrimidin-2-amine (100 mg, 0.27 mmol) and 4-aminoindazole (39 mg, 0.29 mmol) were reacted according to the procedure in Example 16, step 3, to give the title compound (70 mg, 56%) as a solid. NMR Spectrum: (500 MHz, DMSO) 2.96 (m, 8H), 3.66 (m, 8H), 6.09 (s, 1H), 6.42 (d, 1H), 6.88 (s, 2H), 7.18 (d, 1H), 7.25 (t, 1H), 7.91 (m, 1H), 8.07 (d, 1H), 8.27 (s, 1H), 8.87 (s, 1H), 9.33 (s, 1H), 13.03 (m, 1H); Mass spectrum: MH⁺ 473.

### EXAMPLE 23

The procedure described above in Example 22, step 3 was repeated using the appropriate aniline. Thus were obtained the compounds described in Table 12 below.

TABLE 12

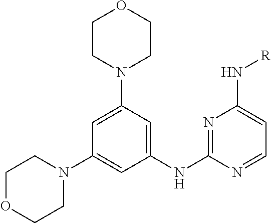

| | | | Molecular | |
|---|---|---|---|---|
| | | | Ion | NMR Spectrum (500 |
| No. | Name | R | (Observed) | MHz, d6-DMSO) |
| 325 (Note 1) | N′-(3-chloro-1H- indazol-4-yl)-N-(3,5- dimorpholinophenyl) pyrimidine-2,4- diamine | 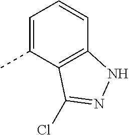 | 507 (MH⁺) | 2.90 (m, 8H), 3.65 (m, 8H), 6.02 (s, 1H), 6.18 (d, 1H), 6.83 (s, 2H), 7.39-7.28 (m, 3H), 8.01 (d, 1H), 8.74 (s, 1H), 8.91 (s, 1H), 13.3 (m, 1H) |
| 326 (Note 2) | N-(3,5- dimorpholinophenyl)- N′-(3-methyl-1H- indazol-4- yl)pyrimidine-2,4- diamine | 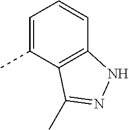 | 487 (MH⁺) | 2.47 (s, 3H), 2.90 (m, 8H), 3.65 (m, 8H), 6.02 (s, 1H), 6.06 (d, 1H), 6.86 (s, 2H), 7.07 (m, 1H), 7.28 (m, 2H), 7.97 (d, 1H), 8.71 (s, 1H), 8.91 (s, 1H), 12.66 (m, 1H) |

Note 1:
3-chloro-1H-indazol-4-amine was made as follows: 3-Chloro-4-nitro-1H-indazole (500 mg, 2.54 mmol; M. Benchidmi et al., J. Het. Chem., 1979, 16, 1599) in ethanol (20 ml) was hydrogenated at atmospheric pressure in the presence of platinum(IV) oxide (50 mg) at room temperature for 1 hour. After filtration of the catalyst, the mixture was concentrated and purified by chromatography on silica gel (eluant: 0% to 6% EtOAc in DCM) to give 3-chloro-1H-indazol-4-amine (242 mg, 57%) as an orange solid. NMR Spectrum: (500
MHz, DMSO) 5.57 (s, 2H), 6.21 (d, 1H), 6.61 (d, 1H), 7.05 (t, 1H), 12.84 (m, 1H); Mass spectrum: MH⁺ 168.
Note 2:
3-methyl-1H-indazol-4 -amine was made as follows: A solution of dimethylzinc (2.07 ml, 4.14 mmol, 2M in toluene was added dropwise to a mixture of 3-bromo-4-nitro-1H-indazole (500 mg, 2.07 mmol; M. Benchidmi et al., J. Het. Chem., 1979, 16, 1599) and [1,1′-bis(diphenylphosphino)ferrocene]dichloropalladium(II) (43 mg, 0.062 mmol) in 1,4-dioxane (8 ml) under argon. The mixture was heated under reflux for 2 hours. After cooling, methanol (0.5 ml) was added, followed by 2N hydrochloric acid (3 ml) and DCM (10 ml).
This mixture was stirred for 30 minutes. The organic layer was collected, washed with saturated aqueous sodium bicarbonate, water and brine, and dried over MgSO4. This solution was concentrated under vacuum to give 3-methyl-4-nitro-1H-indazole (235 mg, 64%) as a solid used without purification in the next step. NMR Spectrum: (500 MHz, DMSO) 2.61 (s, 3H), 7.52 (m, 1H), 7.93 (m, 2H), 13.54 (m, 1H); Mass spectrum: MH⁺ 178.
3-Methyl-4-nitro-1H-indazole (100 mg, 0.56 mmol) in ethanol (10 ml) was hydrogenated at atmospheric pressure in the presence of platinum(IV) oxide (10 mg) at room temperature for 1 hour. After filtration of the catalyst, the mixture was concentrated to give 3-methyl-1H-indazol-4-amine (90 mg, 75%) as a yellow solid. NMR Spectrum: (500 MHz, DMSO) 2.58 (s, 3H), 5.26 (s, 2H), 6.12 (d, 1H), 6.55 (d, 1H), 6.92 (t, 1H), 12.14 (m, 1H); Mass spectrum: MH⁺ 148.

### EXAMPLE 24

### N′-benzooxazol-7-yl-N-(3,5-dimorpholin-4-ylphenyl)pyrimidine-2,4-diamine

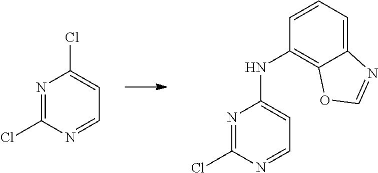

A mixture of benzooxazol-7-amine (135 mg, 1.01 mmol, Astrazeneca, PCT Appl. WO2003053960), 2.4-dichloropyrimidine (150 mg, 1.01 mmol), DBU (0.197 ml, 1.32 mmol), 4,5-bis(diphenylphosphino)-9,9-dimethylxanthene (58 mg, 0.1 mmol, also named xantphos) and tris(dibenzylideneacetone)dipalladium(0) (58 mg, 0.1 mmol, also named Pd2dba3) in dioxane (3 ml) under argon was irradiated in a Personal Chemistry EMRYS™ Optimizer EXP microwave synthesisor at 110° C. for 10 minutes. After cooling and evaporation of the solvents, the residue was dissolved in DCM and purified by chromatography on silica gel (eluant: 30% to 60% EtOAc in petroleum ether) to give N-(2-chloropyrimidin-4-yl)benzooxazol-7-amine (88 mg, 35%) as a beige solid. Mass spectrum: MH⁺ 247

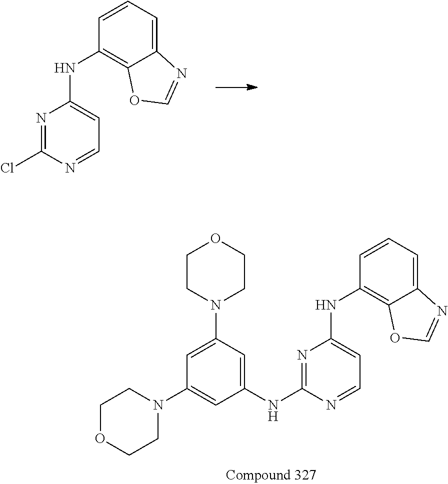

A mixture of N-(2-chloropyrimidin-4-yl)benzooxazol-7-amine (75 mg, 0.3 mmol), 3,5-dimorpholin-4-ylaniline (79 mg, 0.3 mmol), DBU (60 μl, 0.4 mmol), xantphos (17 mg, 0.03 mmol) and Pd2dba3 (17 mg, 0.03 mmol) in dioxane (2 ml) under argon was irradiated in a Personal Chemistry EMRYS™ Optimizer EXP microwave synthesisor at 150° C. for 20 minutes. After cooling, concentrated aqueous ammonia (2 drops) was added and the mixture was injected on an HPLC column (C18, 5 microns, 19 mm diameter, 100 mm length) of a preparative HPLC-MS system eluting with a mixture of water and acetonitrile containing 2 g/l of ammonium carbonate (gradient). Evaporation of the fractions gave the title compound (20 mg, 14%). NMR Spectrum: (500 MHz, DMSO) 2.92 (m, 8H), 3.65 (m, 8H), 6.06 (s, 1H), 6.352 (d, 1H), 6.82 (s, 2H), 7.33 (t, 1H), 7.50 (d, 1H), 7.98 (d br, 1H), 8.06 (d, 1H), 8.74 (s, 1H), 8.85 (s, 1H), 9.55 (s, 1H); Mass spectrum: MH⁺ 474.

### EXAMPLE 25

### N′-benzooxazol-7-yl-N-(3,5-dimorpholinophenyl)-N′-methyl-pyrimidine-2,4-diamine (Compound 328)

N-(2-chloropyrimidin-4-yl)benzooxazol-7-amine (600 mg, 2.44 mmol) was reacted with methyl iodide according to the procedure of Example 10 (starting material (1)) to give N-(2-chloropyrimidin-4-yl)-N-methyl-benzooxazol-7-amine (363 mg, 57%) as a gum. NMR Spectrum: (500 MHz, DMSO) 3.50 (s, 3H), 6.43 (m, 1H), 7.53 (m, 2H), 7.84 (m, 1H), 8.10 (m, 1H), 8.79 (s, 1H).

N-(2-chloropyrimidin-4-yl)-N-methyl-benzooxazol-7-amine (180 mg, 0.69 mmol) was reacted with 3,5-dimorpholin-4-ylaniline according to the procedure in Example 24, Step 2 to give the title compound (15 mg, 4%) as a white solid; NMR Spectrum (500 MHz, DMSO) 2.99-3.05 (m, 8H), 3.53 (s, 3H), 3.67-3.73 (m, 8H), 5.75 (d, 1H), 6.10 (t, 1H), 6.93 (d, 2H), 7.45 (d, 1H), 7.48 (d, 1H), 7.50 (s, 1H), 7.78 (dd, 1H), 7.92 (d, 1H), 8.90 (bs, 1H); Mass spectrum: MH⁺ 488.

### EXAMPLE 26

### N-(3,5-dimorpholin-4-ylphenyl)-N′-methyl-N′-(3-methyl-1H-indazol-4-yl)pyrimidine-2,4-diamine (Compound 329)

Iodine (9.31 g, 36.8 mmol) and potassium hydroxide (3.81 g, 68.1 mmol) were added to a solution of 4-nitro-1H-indazole (3 g, 18.4 mmol) in DMF (40 ml) at room temperature. The mixture was stirred at room temperature for 2.5 hours, and poured in 10% aqueous sodium hydrogensulfite (200 ml). The precipitate was filtered, washed with water and dried over phosphorus pentoxide to give 3-iodo-4-nitro-1H-indazole (5 g, 94%) as a light yellow solid.

NMR Spectrum: (500 MHz, DMSO) 7.60 (t, 1H), 7.86 (d, 1H), 8.00 (d, 1H), 14.3 (m, 1H); Mass spectrum: M−H⁻ 288

Potassium tert-butoxide (23.5 ml, 1M in THF, 23.5 mmol) was added dropwise to an ice-cooled solution of 3-iodo-4-nitro-1H-indazole (4.85 g, 16.8 mmol) in THF (30 ml) under argon. The mixture was stirred at 0° C. for 1 hour. 4-Methoxybenzyl chloride (2.5 ml, 18.5 mmol) and tetrabutylammonium iodide (63 mg, 0.17 mmol) were added and the mixture was stirred at 70° C. for 2.5 hours. The mixture was cooled and concentrated under vacuum. The residue was dissolved in ethyl acetate, washed with water and brine and dried over MgSO4. After evaporation of the solvents, the residue was purified by chromatography on silica gel (eluant: 10% to 40% EtOAc in petroleum ether) to give 3-iodo-1-[(4-methoxyphenyl)methyl]-4-nitro-indazole (4.4 g, 64%) as a solid. NMR Spectrum: (500 MHz, DMSO) 3.71 (s, 3H), 5.71 (s, 2H), 6.89 (d, 2H), 7.25 (d, 2H), 7.64 (t, 1H), 7.87 (d, 1H), 8.27 (d, 1H).

A solution of dimethylzinc (9.05 ml, 18.1 mmol, 2M in toluene) was added dropwise to a mixture of 3-iodo-1-[(4-methoxyphenyl)methyl]-4-nitro-indazole (3.7 g, 9.05 mmol) and [1,1′-bis(diphenylphosphino)ferrocene]dichloropalladium(II) (189 mg, 0.27 mmol) in 1,4-dioxane (30 ml) under argon. The mixture was heated at 100° C. for 1.5 hour. After cooling, methanol (3 ml) was added, followed by 2N hydrochloric acid until the pH was acidic. This mixture was stirred for 10 minutes, extracted with EtOAc, washed with saturated aqueous sodium bicarbonate, water and brine, and dried over MgSO4. After evaporation of the solvents, the residue was purified by chromatography on silica gel (eluant: 10% to 25% EtOAc in petroleum ether) to give 1-[(4-methoxyphenyl)methyl]-3-methyl-4-nitro-indazole (1.07 g, 40%) as a yellow solid. NMR Spectrum: (500 MHz, DMSO) 2.59 (s, 3H), 3.70 (s, 3H), 5.61 (s, 2H), 6.87 (d, 2H), 7.22 (d, 2H), 7.56 (t, 1H), 7.92 (d, 1H), 8.19 (d, 1H).

1-[(4-Methoxyphenyl)methyl]-3-methyl-4-nitro-indazole (1 g, 3.37 mmol) in ethanol (30 ml) was hydrogenated at atmospheric pressure in the presence of platinum(IV) oxide (80 mg) at room temperature for 1 hour. After filtration of the catalyst, the mixture was concentrated to give 1-[(4-methoxyphenyl)methyl]-3-methylindazol-4-amine (900 mg, 100%) as a yellow gum. NMR Spectrum: (500 MHz, DMSO) 2.58 (s, 3H), 3.68 (s, 3H), 5.30 (s, 2H), 5.33 (s, 2H), 6.15 (d, 1H), 6.66 (d, 1H), 6.84 (d, 2H), 6.95 (t, 1H), 7.13 (d, 2H); Mass spectrum: MH⁺ 268.

A mixture of 4-chloro-2-methylthiopyrimidine (0.4 ml, 3.45 mmol), 1-[(4-methoxyphenyl)methyl]-3-methylindazol-4-amine (0.83 g, 3.11 mmol) and hydrogen chloride (1 drop, 7N in dioxane) in n-butanol (10 ml) was heated at 80° C. for 2 hours. After cooling and evaporation of the solvents, water was added. The pH was adjusted to 8 by addition of aqueous ammonia and the mixture was extracted with EtOAc. The precipitate which had formed at the interface was filtered, washed with water and ether and dried to give a solid. The organic layer was washed with water and brine, and dried over MgSO₄. After evaporation of the solvents, the residue was triturated with ether. The two batches were combined to give 1-[(4-methoxyphenyl)methyl]-3-methyl-N-(2-methylsulfanylpyrimidin-4-yl)indazol-4-amine (1 g, 74%) as a white solid. NMR Spectrum: (500 MHz, DMSO) 2.31 (s, 3H), 2.41 (s, 3H), 3.70 (s, 3H), 5.47 (s, 2H), 6.28 (d, 1H), 6.86 (d, 2H), 7.02 (d, 1H), 7.19 (d, 2H), 7.33 (t, 1H), 7.50 (d, 1H), 8.05 (d, 1H), 9.41 (s, 1H); Mass spectrum: MH⁺ 392.

Methyl iodide (0.17 ml, 2.69 mmol) was added to a mixture of 1-[(4-methoxyphenyl)methyl]-3-methyl-N-(2-methylsulfanylpyrimidin-4-yl)indazol-4-amine (1 g, 2.56 mmol) and cesium carbonate (1.25 g, 3.84 mmol) in acetonitrile (6 ml). The mixture was stirred at room temperature for 18 hours. The mixture was diluted with acetonitrile and the solids were filtered off. After evaporation of the solvents, the residue was dissolved in DCM, filtered and purified by chromatography on silica gel (eluant: 10 to 40% EtOAc in petroleum ether) to give 1-[(4-methoxyphenyl)methyl]-N,3-dimethyl-N-(2-methylsulfanylpyrimidin-4-yl)indazol-4-amine (0.7 g, 67%) as a solid. Mass spectrum: MH⁺ 406.

m-Chloroperbenzoic acid (909 mg, 3.7 mmol) was added to an ice-cooled solution of 1-[(4-methoxyphenyl)methyl]-N,3-dimethyl-N-(2-methylsulfanylpyrimidin-4-yl)indazol-4-amine (600 mg, 1.48 mmol) in DMF (17 ml). The mixture was then stirred at room temperature for 1.5 hour. 10% Aqueous sodium metabisulfite was added. The mixture was concentrated, diluted in DCM, washed with sodium bicarbonate and brine, and dried over MgSO₄. After evaporation of the solvents, the residue was purified by chromatography on silica gel (eluant: 10 to 50% EtOAc in petroleum ether) to give 1-[(4-methoxyphenyl)methyl]-N,3-dimethyl-N-(2-methylsulfonylpyrimidin-4-yl)indazol-4-amine (0.6 g, 92%) as a solid. NMR Spectrum: (500 MHz, DMSO) 2.17 (s, 3H), 3.39 (s, 3H), 3.51 (s, 3H), 3.71 (s, 3H), 5.53 (m, 2H), 6.04 (d, 1H), 6.89 (d, 2H), 7.12 (d, 1H), 7.28 (d, 2H), 7.50 (t, 1H), 7.82 (d, 1H), 8.15 (d, 1H).

A mixture of 1-[(4-methoxyphenyl)methyl]-N,3-dimethyl-N-(2-methylsulfonylpyrimidin-4-yl)indazol-4-amine (200 mg, 0.46 mmol), 3,5-dimorpholin-4-ylaniline (127 mg, 0.46 mmol) and hydrogen chloride (4N in dioxane, 7 drops) in 2-pentanol (4 ml) was irradiated in a Personal Chemistry EMRYS™ Optimizer EXP microwave synthesisor at 150° C. for 30 minutes. After cooling and evaporation of the solvents, water was added. The pH was adjusted to pH 7 by addition of aqueous ammonia. The mixture was extracted with EtOAc. The organic layer was washed with sodium bicarbonate and brine, and dried over MgSO₄. After evaporation of the solvents, the residue was purified by chromatography on silica gel (eluant: 0 to 5% methanol in EtOAc-DCM (1:4)) to give N-(3,5-dimorpholin-4-ylphenyl)-N′-[1-[(4-methoxyphenyl)methyl]-3-methyl-indazol-4-yl]-N′-methyl-pyrimidine-2,4-diamine (71 mg, 25%); Mass spectrum: MH⁺ 621.

A mixture of N-(3,5-dimorpholin-4-ylphenyl)-N′-[1-[(4-methoxyphenyl)methyl]-3-methyl-indazol-4-yl]-N-methyl-pyrimidine-2,4-diamine (100 mg, 0.16 mmol) and anisole (1 drop) in TFA (1 ml) was irradiated in a Personal Chemistry EMRYS™ Optimizer EXP microwave synthesisor at 130° C. for 40 minutes. After cooling and evaporation of the solvents, the residue was dissolved in DCM. A few drops of 6N ammonia in methanol followed by water (0.5 ml) was added. The organic layer was collected and purified by chromatography on silica gel (eluant: 0 to 5% methanol in DCM). Trituration of the resulting solid in ether gave the title compound (54 mg, 67%) as a white solid. NMR Spectrum: (500 MHz, DMSO) 2.20 (s, 3H), 3.07 (m, 8H), 3.56 (s, 3H), 3.72 (m, 8H), 5.23 (br s, 1H), 6.12 (br s, 1H), 7.02 (m, 3H), 7.41 (m, 1H), 7.51 (m, 1H), 7.75 (br s, 1H), 8.89 (br s, 1H), 12.9 (m, 1H); Mass spectrum: MH⁺ 501.

### EXAMPLE 27

### N′-methyl-N′-(3-methyl-1H-indazol-4-yl)-N-(3-methylsulfonylphenyl)pyrimidine-2,4-diamine (Compound 330)

The last 2 steps from procedure in Example 26 were repeated using 1-[(4-methoxyphenyl)methyl]-N,3-dimethyl-N-(2-methylsulfonylpyrimidin-4-yl)indazol-4-amine (286 mg, 0.65 mmol) and 3-methylsulfonylaniline hydrochloride (142 mg, 0.65 mmol) to give the title compound (61 mg, 23% over 2 steps). NMR Spectrum: (500 MHz, DMSO) 2.20 (s, 3H), 3.17 (s, 3H), 3.53 (s, 3H), 5.34 (br s, 1H), 7.03 (br d, 1H), 7.43 (m, 2H), 7.54 (m, 2H), 7.91-7.82 (m, 2H), 8.82 (br s, 1H), 9.70 (br s, 1H), 12.9 (m, 1H); Mass spectrum: MH⁺ 409.

### EXAMPLE 28

4-Chloro-N-(3,5-dimorpholin-4-ylphenyl)pyrimidin-2-amine (70 mg, 0.19 mmol) and the corresponding aniline (0.22 mmol) were dissolved in pentanol (1 ml). 4M HCl in dioxane (0.1 ml) was added. The reaction was heated at 100° C. for 15 hours then cooled to room temperature and concentrated in vacuo. The residue was dissolved in DMF (1 ml) and directly injected on an HPLC column (C18, 5 microns, 19 mm diameter, 100 mm length) of a preparative HPLC-MS system eluting with a mixture of water and acetonitrile containing 2 g/l of ammonium carbonate (gradient). Evaporation of the solvents gave the title compound as a solid.

The examples in the Table 13 below were made according to the procedure above.

TABLE 13

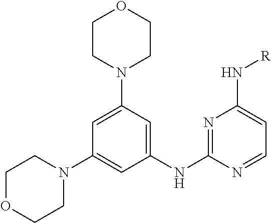

| | | | Molecular | |
|---|---|---|---|---|
| | | | Ion | NMR Spectrum (500 |
| No. | Name | R | (Observed) | MHz, d6-DMSO) |
| 331 | N-(3,5-dimorpholin-4- ylphenyl)-N′-quinolin- 5-yl-pyrimidine-2,4- diamine | 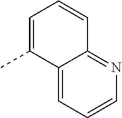 | 484 (MH⁺) | 2.79-2.96 (m, 8H), 3.55-3.69 (m, 8H), 6.03 (s, 1H), 6.28 (d, 2H), 6.82 (s, 1H), 7.57 (dd, 1H), 7.74 (dd, 1H), 7.85 (d, 1H), 7.93 (d, 1H), 8.05 (d, 1H), 8.52 (d, 1H), 8.78 (s, 1H), 8.93 (dd, 1H), 9.36 (s, 1H) |
| 332 | N′-(2,2- difluorobenzo[1,3] dioxol-4-yl)-N-(3,5- dimorpholin-4- ylphenyl)pyrimidine- 2,4-diamine | 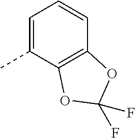 | 513 (MH⁺) | 2.88-3.02 (m, 8H), 3.62-3.76 (m, 8H), 6.09 (s, 1H), 6.33 (d, 1H), 6.82 (s, 2H), 7.09-7.17 (m, 2H), 7.82 (d, 1H), 8.08 (d, 1H), 8.90 (s, 1H), 9.45 (s, 1H) |
| 333 | N-(3,5-dimorpholin-4- ylphenyl)-N′-(1H- indol-4-yl)pyrimidine- 2,4-diamine | 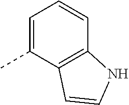 | 472 (MH⁺) | 2.87-3.06 (m, 8H), 3.59-3.75 (m, 8H), 6.07 (s, 1H), 6.32 (d, 1H), 6.63 (t, 1H), 6.91 (d, 2H), 7.03 (dd, 1H), 7.15 (d, 1H), 7.29 (dd, 1H), 7.66 (d, 1H), 7.99 (d, 1H), 8.75 (s, 1H), 8.94 (s, 1H), 11.12 (s, 1H) |
| 334 | N-(3,5-dimorpholin-4- ylphenyl)-N′-(2,5- dioxabicyclo[4.4.0]dec a-6,8,10-trien-10- yl)pyrimidine-2,4- diamine | 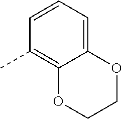 | 491 (MH⁺) | 2.90-3.06 (m, 8H), 3.62-3.76 (m, 8H), 4.23-4.33 (m, 4H), 6.08 (t, 1H), 6.32 (d, 1H), 6.61 (dd, 1H), 6.76 (dd, 1H), 6.88 (d, 2H), 7.58 (d, 1H), 7.97 (d, 1H), 8.52 (s, 1H), 8.76 (s, 1H) |
| 335 | N′-(1H-benzotriazol-4- yl)-N-(3,5- dimorpholin-4- ylphenyl)pyrimidine- 2,4-diamine | 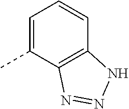 | 474 (MH⁺) | 2.88-3.09 (m, 8H), 3.71-3.76 (m, 8H), 6.11 (s, 1H), 6.60 (bs, 1H), 6.87 (s, 2H), 7.38 (dd, 1H), 7.44 (bs, 1H), 8.10 (d, 1H), 8.25 (bs, 1H), 8.93 (s, 1H), 9.81 (bs, 1H) |
| 336 (Note 1) | N′-(3-chloro-1H-indol- 7-yl)-N-(3,5- dimorpholin-4- ylphenyl)pyrimidine- 2,4-diamine | 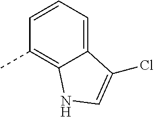 | 506 (MH⁺) | 2.82-3.04 (m, 8H), 3.56-3.78 (m, 8H), 6.06 (d, 1H), 6.12 (d, 1H), 6.85 (s, 2H), 7.10 (dd, 1H), 7.29 (d, 1H), 7.48 (d, 1H), 7.55 (d, 1H), 8.01 (d, 1H), 8.78 (s, 1H), 8.98 (s, 1H), 11.11 (s, 1H) |
| 337 | N-(3,5-dimorpholin-4- ylphenyl)-N′-(1H- indazol-7- yl)pyrimidine-2,4- diamine | 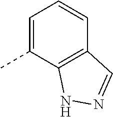 | 473 (MH⁺) | 2.84-3.02 (m, 8H), 3.57-3.76 (m, 8H), 6.06 (s, 1H), 6.16 (d, 1H), 6.85 (s, 2H), 7.09 (dd, 1H), 7.53 (d, 1H), 7.75 (d, 1H), 8.04 (d, 1H), 8.09 (s, 1H), 8.82 (s, 1H), 9.09 (s, 1H), 12.81 (s, 1H) |

Note 1:
3-chloro-1H-indol-7-amine (AstraZeneca, PCT Appl. WO200234744)

### Method Section

### Method 1

### 1-Fluoro-3-methylsulfonyl-5-nitro-benzene

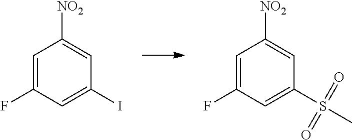

A mixture of 1-fluoro-3-iodo-5-nitro-benzene (1.95 g), copper (I) iodide (2.23 g) and sodium methansulfinate (0.75 g, 85%) in DMF (25 ml) was heated at 110° C. overnight, then poured into a mixture of ethyl acetate and water and filtered. The organic layer was separated, dried and concentrated in vacuo and the residue triturated with methanol to give the title compound as a brown solid (0.6 g, 37%); NMR Spectrum (300 MHz, DMSO) 3.40 (s, 3H), 8.31 (m, 1H), 8.52 (m, 2H); Mass Spectrum M⁺ 219.0.

The procedure described above was repeated using the appropriate iodobenzene. Thus was obtained the example described below:

| | | | Molecular | NMR Spectrum |
|---|---|---|---|---|
| | | | Ion | (300 MHz, |
| Method | Name | Structure | (Observed) | d6-DMSO) |
| 1a | 1-methylsulfonyl- 3,5-dinitro-benzene | 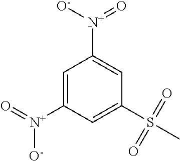 | 247.39 (MH+) | 3.50 (s, 3H), 9.00 (m, 2H), 9.09 (m, 1H) |

### Method 2

### 4-(3-Methylsulfonyl-5-nitro-phenyl)morpholine

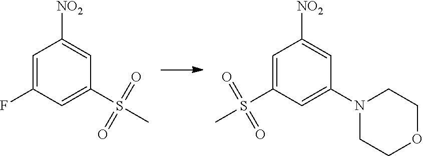

Morpholine (0.77 ml) and 1-fluoro-3-methylsulfonyl-5-nitro-benzene (0.35 g-Method 1) in DMSO (15 ml) were heated at 100° C. for 6 hrs. The solution was cooled, poured into water and the resulting precipitate filtered and dried to give the title compound as an orange solid (0.39 g, 85%); NMR Spectrum (300 MHz, DMSO) 3.25-3.42 (m+s, 7H), 3.76 (m, 4H), 7.75 (m, 1H), 7.94 (m, 2H).

The procedure described above was repeated using the appropriate fluorobenzene. Thus were obtained the examples described below:

| | | | Molecular | |
|---|---|---|---|---|
| | | | Ion | NMR Spectrum (300 |
| Method | Name | Structure | (Observed) | MHz, d6-DMSO) |
| 2a | 1-methyl-4-(3- methylsulfonyl-5- nitro-phenyl) piperazine | 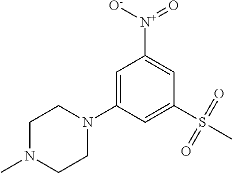 | 300.44 (MH+) | |
| 2b | ethyl 3-morpholin- 4-yl-5-nitro- benzoate | 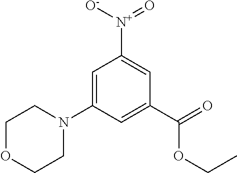 | 281.47 (MH+) | 1.35 (t, 3H), 3.30 (m, 4H), 3.77 (m, 4H), 4.37 (q, 2H), 7.80 (m, 1H), 7.90 (m, 1H), 8.00 (s, 1H) |

### Method 3

### 4-(3-Fluoro-5-nitro-phenyl)morpholine and 4-(3-morpholin-4-yl-5-nitro-phenyl)morpholine

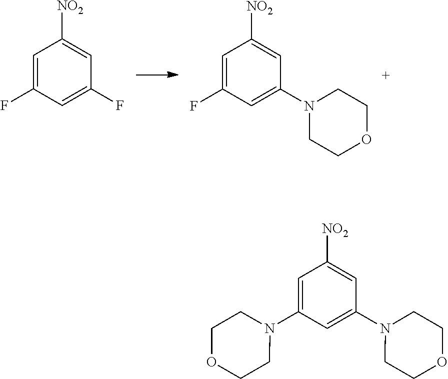

Morpholine (12 ml) and 1,3-difluoro-5-nitro-benzene (4 g) in DMSO (50 ml) were heated at 100° C. for 4 days. The solution was cooled, poured into water and the resulting precipitate filtered and dried. This was purified by chromatography using 25% to 60% ethyl acetate in iso-hexane as eluent to give firstly 4-(3-fluoro-5-nitro-phenyl)morpholine as a yellow solid (2.86 g, 50%); NMR Spectrum (300 MHz, DMSO) 3.30 (m, 4H), 3.72 (m, 4H), 7.24 (m, 1H), 7.38 (m, 1H), 7.52 (m, 1H); followed by 4-(3-morpholin-4-yl-5-nitro-phenyl)morpholine as an orange solid (2.11 g, 29%); Mass Spectrum MH⁺ 294.50.

### Method 4

### 3,5-Dinitrobenzenesulfonamide

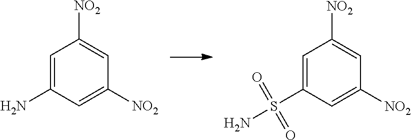

Thionyl chloride (20 ml) was added dropwise to water (70 ml) at 0° C. with vigorous stirring. The solution was stirred for 1 hour at 5° C. and at 18° C. for 50 minutes. Copper (I) chloride (0.16 g) was added to give a pale green solution that was stirred at room temperature for 5 minutes then cooled to −10° C. Separately, 3,5-dinitroaniline (5 g) was added to c. HCl (25 ml) and stirred for 1 hour at room temperature. The solution was cooled to −10° C. and treated dropwise with a solution of sodium nitrite (2.26 g) in water (20 ml). The resulting dark orange solution was stirred at −10° C. for 10 minutes then added at −5° C. to the solution of copper (I) chloride from the first step over 5 minutes. The reaction was stirred at −5° C. for 1 hour then filtered to give a pale pink solid that was dried in vacuo. This solid was added portionwise to a solution of ammonia in methanol (7N, 200 ml) at 0° C. and the reaction stirred for 2 hrs then concentrated in vacuo. The resulting solid was triturated with methanol, then water and filtered and dried to give the title compound as a beige solid (2.88 g, 43%); NMR Spectrum (300 MHz, DMSO) 7.86 (br s, 2H), 8.80 (m, 2H), 8.90 (m, 1H); Mass Spectrum M⁺ 246.39.

### Method 5

### 3-Morpholin-4-yl-5-nitro-benzoic acid

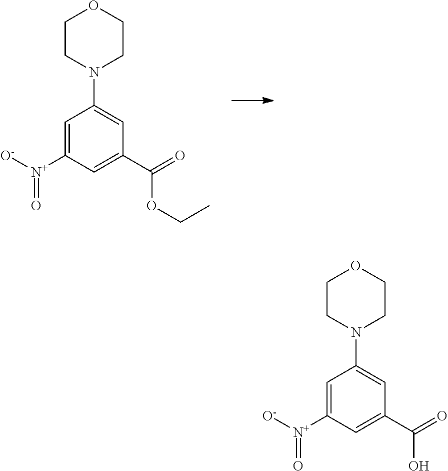

Sodium hydroxide (1.8 ml, 2N) was added to a solution of ethyl 3-morpholin-4-yl-5-nitro-benzoate (337 mg-Method 2b) in methanol (10 ml) and THF (10 ml) and stirred for 3 hours at room temperature. Water (3 ml) was added, followed by aq. HCl (1.6 ml, 2N) and the resulting precipitate filtered and dried to give the title compound as a yellow solid (0.23 g, 76%); Mass Spectrum MH⁺ 253.44.

### Method 6

### 3-Methylsulfonyl-5-nitro-benzoic acid

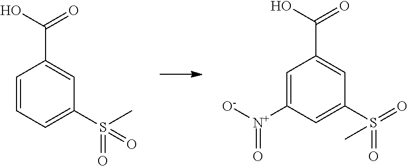

3-Methylsulfonylbenzoic acid (0.75 g) was added to c.sulphuric acid (1.2 ml) and heated to 80° C. Fuming nitric acid (0.6 ml) was added dropwise maintaining the temperature at 80-85° C., and the reaction stirred at this temperature for 2 hours. The reaction was cooled to room temperature and poured onto 20 ml ice-water to give a white solid which was filtered, washed with water and dried in vacuo to give the title compound as a white solid (0.69 g, 75%); NMR Spectrum (300 MHz, DMSO) 3.44 (s, 3H), 8.75 (t, 1H), 8.84-8.87 (m, 2H).

### Method 7

### 3-Morpholin-4-yl-5-nitro-benzamide

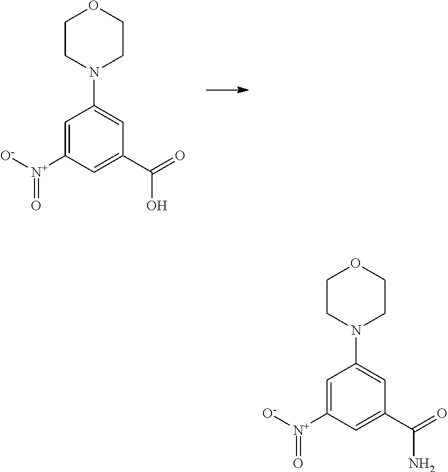

HATU (0.45 g) was added to a solution of 3-morpholin-4-yl-5-nitro-benzoic acid (0.23 g-Method 5), ammonium chloride (146 mg) and DIPEA (0.21 ml) in DMF (1 ml) and the reaction stirred overnight. The solution was concentrated in vacuo and partitioned between ethyl acetate and saturated aqueous sodium bicarbonate solution. The organic layer was dried and concentrated to give the title compound as a yellow solid (0.2 g, 87%); Mass Spectrum MH⁺ 252.47.

The procedure described above was repeated using the appropriate acid. Thus were obtained the compounds described below:

| | | | Molecular | |
|---|---|---|---|---|
| | | | Ion | NMR Spectrum (300 |
| Method | Name | Structure | (Observed) | MHz, d6-DMSO) |
| 7a | 3-amino-5- nitro-benzamide | 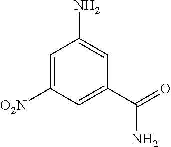 | 180.44 (M+) | 5.96 (d, 2H), 7.44 (t, 2H), 7.50 (t, 1H), 7.80 (t, 1H), 8.09 (br s, 1H) |
| 7b | methyl 3- carbamoyl-5- nitro-benzoate | 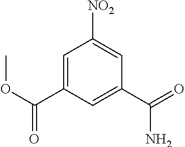 | 223.46 (M+) | 3.88 (s, 3H), 7.73 (br s, 1H), 8.44 (br s, 1H), 8.64 (m, 1H), 8.72 (m, 1H), 8.85 (m, 1H) |
| 7c | 3- methylsulfonyl- 5-nitro- benzamide | 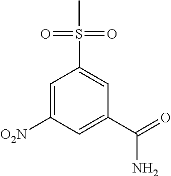 | 243.43 (M+) | 3.40 (s, 3H), 7.93 (br s, 1H), 8.57 (br s, 1H), 8.77-8.80 (m, 2H), 8.97 (t, 1H) |

### Method 8

### 3-Fluoro-5-methylsulfonyl-aniline

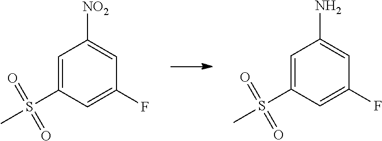

A mixture of 1-fluoro-3-methylsulfonyl-5-nitro-benzene (0.2 g-method 1) and 10% Pd/C (50 mg) in ethanol (20 ml) was stirred under a hydrogen atmosphere overnight. The solution was filtered and concentrated to give the title compound as a pale brown oil (0.18 g, 100%); Mass Spectrum M⁺ 189.03.

The procedure described above was repeated using the appropriate nitrobenzene. Thus were obtained the examples described below:

| | | | Molecular | NMR Spectrum | Starting |
|---|---|---|---|---|---|
| | | | Ion | (300 MHz, | material |
| Method | Name | Structure | (Observed) | d6-DMSO) | (Method) |
| 8a | 3-fluoro-5- morpholin- 4-yl-aniline | 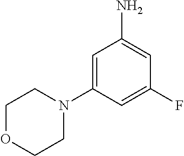 | 197.51 (MH+) | | 3 |
| 8b | 3- methyl- sulfonyl- 5- morpholin- 4-yl-aniline | 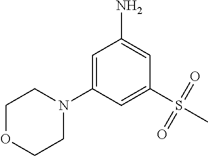 | 247.57 (MH+) | | 2 |
| 8c | 5- methyl- sulfonyl- benzene-1,3- diamine | 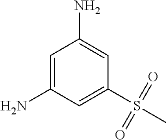 | 187.46 (MH+) | | 1a |
| 8d | 3-(4- methyl- piperazin- 1-yl)-5- methyl- sulfonyl- aniline | 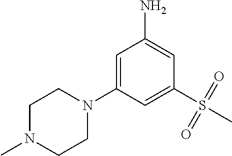 | 270.48 (MH+) | | 2a |
| 8e | 3,5- diamino- benzene- sulfonamide | 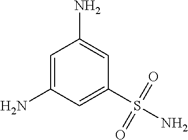 | 188.45 (MH+) | | 4 |
| 8f | 3,5- dimorpholin- 4- ylaniline | 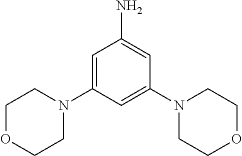 | 264.52 (MH+) | 2.98 (t, 8H), 3.69 (t, 8H), 4.71 (d, 2H), 5.70-5.74 (m, 2H), 5.75 (s, 1H) | 3 |
| 8g | ethyl 3- amino-5- morpholin- 4-yl- benzoate | 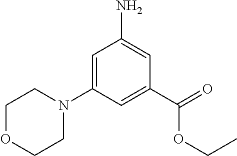 | 251.47 (MH+) | 1.28 (t, 3H), 3.06 (m, 4H), 3.73 (m, 4H), 4.25 (q, 2H), 5.20 (br s, 2H), 6.40 (m, 1H), 6.70 (m, 2H) | 2b |
| 8h | 3-amino-5- morpholin- 4-yl- benzamide | 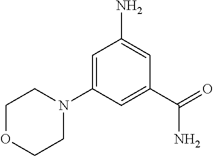 | 222.54 (MH+) | 3.05 (t, 4H), 3.72 (t, 4H), 5.00 (d, 1H), 6.27 (t, 1H), 6.56 (s, 1H), 6.62 (s, 1H), 7.01 (br s, 1H), 7.64 (br s, 1H) | 7 |
| 8i | 3- (hydroxy- methyl)- 5- methyl- benzene- sulfonamide | 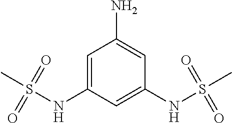 | 279.04 (M+) | 2.95 (s, 6H), 5.27 (br s, 2H), 6.20 (m, 2H), 6.30 (m, 1H), 9.43 (br s, 2H) | |
| 8j | 3-amino-5- methane- sulfonamido- benzamide | 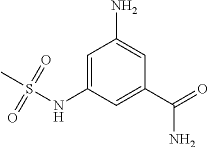 | 228.48 (M+) | 2.97 (s, 3H), 5.30 (s, 2H), 6.58 (t, 1H), 6.74-6.78 (m, 2H), 7.11 (s, 1H), 7.66 (s, 1H), 9.48 (s, 1H) | 9b |
| 8k | 3-amino-5- (hydroxy- methyl) benzamide | 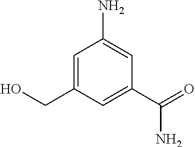 | 166.07 (M+) | | 10a |
| 8l | [3-amino-5- (hydroxy- methyl) phenyl] methanol | 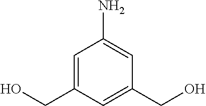 | 154.47 (MH+) | 3.25 (1H, under water), 4.29-4.37 (m, 4H), 4.93 (t, 3H), 6.41 (s, 3H) | |
| 8m | 3-amino-5- methyl- sulfonyl- benzamide | 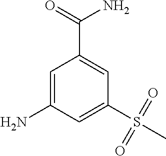 | 215.47 (MH+) | 3.15 (s, 3H), 5.82 (d, 2H), 7.19 (t, 1H), 7.31-7.36 (m, 2H), 7.47 (s, 1H), 7.98 (br s, 1H) | 7c |

### Method 9

### N-(3-Amino-5-methylsulfonyl-phenyl)methanesulfonamide

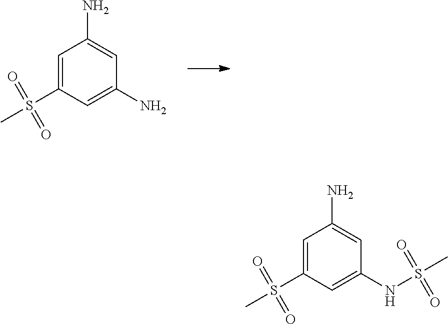

Methanesulfonyl chloride (62 μl) was added to a solution of 5-methylsulfonylbenzene-1,3-diamine (0.15 g-Method 8c) and pyridine (0.33 ml) in DCM (15 ml) and the reaction stirred for two hours at room temperature. The solution was washed with water, dried and concentrated and the residue purified by chromatography to give the title compound as a brown oil (45 mg, 21%); Mass Spectrum MH⁺ 265.36.

The procedure described above was repeated using the appropriate aniline. Thus were obtained the compounds described below:

| | | | Molecular | NMR Spectrum | Starting |
|---|---|---|---|---|---|
| | | | Ion | (300 MHz, | material |
| Method | Name | Structure | (Observed) | d6-DMSO) | (Method) |
| 9a | 3-amino-5- methane- sulfonamido- benzene- sulfonamide | 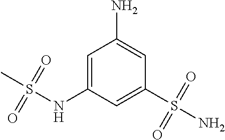 | 266.39 (MH+) | | 8e |
| 9b | 3- methane- sulfonamido- 5-nitro- benzamide | 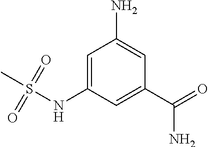 | 258.42 (M+) | 3.14 (s, 3H), 7.70 (br s, 1H), 8.08 (s, 1H), 8.17 (s, 1H), 8.35 (br s, 1H), 8.43 (s, 1H), 10.46 (s, 1H) | |

### Method 10

### (3-Amino-5-morpholin-4-yl-phenyl)methanol

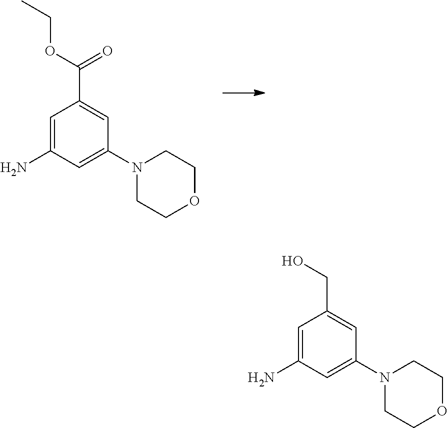

Lithium aluminum hydride (0.48 ml, 1M in THF) was added dropwise to ethyl 3-amino-5-morpholin-4-yl-benzoate (0.1 g-Method 8g) in THF (3 ml) and the mixture stirred overnight at room temperature. Water (0.1 ml) was added, followed by aqueous sodium hydroxide (0.1 ml, 1M), then magnesium sulfate (1 g) and diethyl ether (10 ml) added. The mixture was stirred at room temperature for 20 minutes then filtered and washed with ether. The filtrate was concentrated in vacuo and the residue purified by chromatography using 0 to 10% methanol in DCM as eluent to give the title compound as an orange solid (80 mg, 96%); NMR Spectrum (300 MHz, DMSO) 2.99 (m, 4H), 3.70 (m, 4H), 4.30 (m, 2H), 4.85 (br s, 2H), 6.02 (m, 1H), 6.07 (s, 1H), 6.11 (s, 1H); Mass Spectrum MH⁺ 209.52.

The procedure described above was repeated using the appropriate ester. Thus was obtained the compound described below:

| | | | Molecular | | Starting |
|---|---|---|---|---|---|
| | | | Ion | NMR Spectrum (300 | material |
| Method | Name | Structure | (Observed) | MHz, d6-DMSO) | (Method) |
| 10a | 3- (hydroxymethyl)- 5-nitro- benzamide | 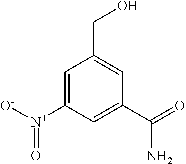 | 195.44 (M+) | 4.69 (m, 2H), 5.60 (t, 1H), 7.65 (br s, 1H), 8.27 (m, 1H), 8.34 (m, 2H), 8.58 (m, 1H) | 7b |

### Method 11

### Ethyl 3-methanesulfonamido-5-morpholin-4-yl-benzoate

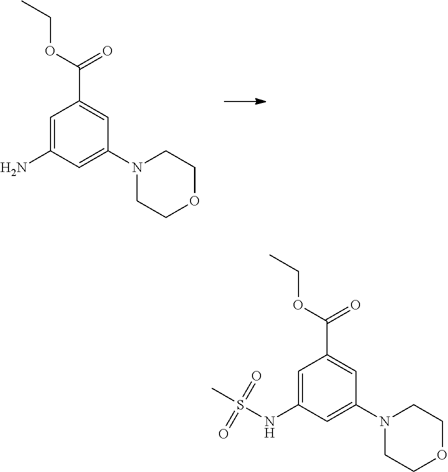

Methanesulfonyl chloride (127 μl) was added to a solution of ethyl 3-amino-5-morpholin-4-yl-benzoate (0.344 g-Method 8g) and pyridine (0.54 ml) in THF (3 ml) and the reaction stirred overnight at room temperature. The solution was concentrated in vacuo and the residue partitioned between 1M HCl and diethyl ether. The organic layer was concentrated and triturated with diethyl ether and iso-hexane to give the title compound as a yellow solid (404 mg, 89%); NMR Spectrum (300 MHz, DMSO) 1.22 (t, 3H), 3.03 (m, 4H), 3.65 (m, 4H), 4.21 (q, 2H), 6.91 (m, 1H), 7.14 (m, 1H ), 7.20 (m, 1H), 9.68 (s, 1H); Mass Spectrum MH⁺ 329.49.

The procedure described above was repeated using the appropriate aniline. Thus was obtained the example described below:

| | | | Molecular | |
|---|---|---|---|---|
| | | | Ion | NMR Spectrum (300 |
| Method | Name | Structure | (Observed) | MHz, d6-DMSO) |
| 11a | methyl 3-amino- 5- methane- sulfonamido- benzoate | 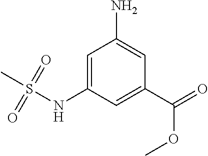 | 254.41 (MH+) | 2.94 (s, 3H), 3.80 (s, 3H), 5.50 (br s, 2H), 6.69 (m, 1H), 6.93 (m, 1H), 6.98 (m, 1H), 9.60 (br s, 1H) |

### Method 12

### 3-Methanesulfonamido-5-morpholin-4-yl-benzoic acid

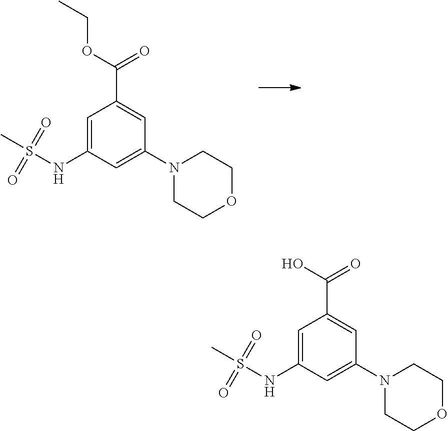

Lithium hydroxide (71 mg) and ethyl 3-methanesulfonamido-5-morpholin-4-yl-benzoate (404 mg-method 11) in THF (3 ml) and water (0.1 ml) were stirred for 48 hours then concentrated in vacuo. The residue was dissolved in water (5 ml) and pH adjusted to 5. The resulting precipitate was filtered and dried to give the title compound as a yellow solid (0.26 g, 71%); Mass Spectrum MH⁺ 301.47.

### Method 13

### Tert-Butyl N-(3-methanesulfonamido-5-morpholin-4-yl-phenyl)carbamate

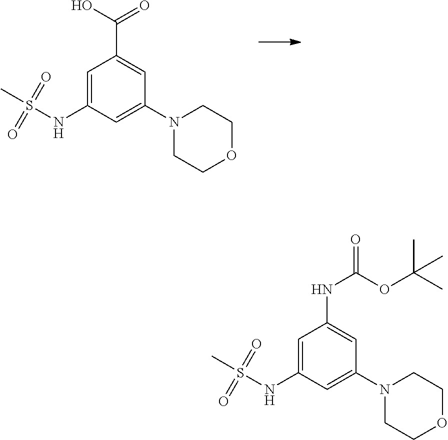

Diphenylphosphoryl azide (0.224 ml) was added to a solution of 3-methanesulfonamido-5-morpholin-4-yl-benzoic acid (0.26 g-method 12) and DIPEA (0.18 ml) in tert-butanol (10 ml) and the reaction heated at 80° C. for 5 hours. The reaction was concentrated in vacuo and the residue purified by chromatography using 0 to 100% ethyl acetate in iso-hexane then 5% methanol in DCM as eluent to give the title compound as a white foam (150 mg, 35%); Mass Spectrum MH⁺ 372.49.

### Method 14

### N-(3-Amino-5-morpholin-4-yl-phenyl)methanesulfonamide

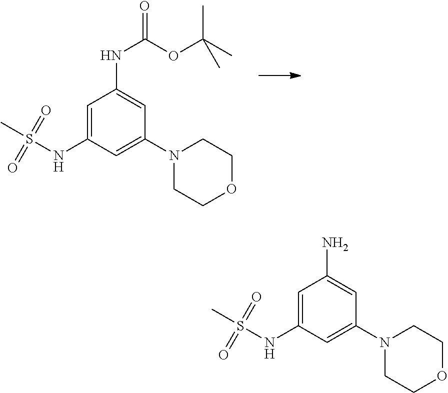

Tert-butyl N-(3-methanesulfonamido-5-morpholin-4-yl-phenyl)carbamate (0.15 g-method 13) and c. HCl (3 ml) in methanol (5 ml) were heated at 70° C. for 5 hours then cooled and concentrated in vacuo. The residue was partitioned between ethyl acetate and saturated aqueous sodium bicarbonate and combined organic layers dried and concentrated and the residue purified by chromatography using ethyl acetate as eluent followed by trituration with ethyl acetate-diethyl ether-iso-hexane to give the title compound as a white solid (24 mg, 22%); NMR Spectrum (300 MHz, DMSO) 2.92 (s, 3H), 2.96 (m, 4H), 3.70 (m, 4H), 5.90 (m, 1H), 6.00 (m, 2H), 9.20 (br s, 1H); Mass Spectrum MH⁺ 272.46.

### Method 15

### 2-Chloro-5-(hydroxymethyl)-3-nitro-benzenesulfonamide

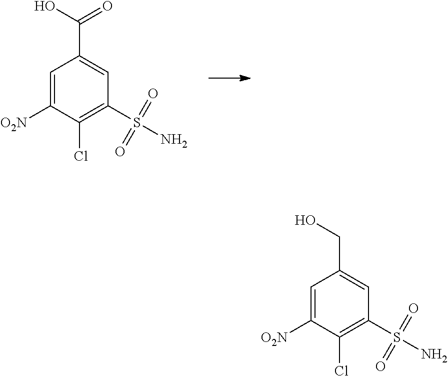

Borane in THF (12 ml, 1M) was added dropwise to 4-chloro-3-nitro-5-sulfamoyl-benzoic acid (1.6 g) in THF (30 ml) and the reaction stirred overnight at room temperature. Methanol was added dropwise and the reaction mixture stirred for 20 minutes at room temperature. The reaction mixture was concentrated in vacuo and the residue partitioned between water and ethyl acetate, dried and concentrated. The residue was purified by chromatography using 0 to 100% ethyl acetate in iso-hexane then 5% methanol in DCM as eluent to give the title compound as a yellow solid (2.5 g, >100%); NMR Spectrum (300 MHz, DMSO) 4.52 (m, 2H), 5.60 (t, 1H), 7.82 (br s, 2H), 8.04 (s, 1H), 8.14 (s, 1H); Mass Spectrum MH⁺ 265.33.

The procedure described above was repeated using, in this case, the appropriate methyl benzoate ester (rather than benzoic acid). Thus was obtained the compound described below:

| | | | Molecular | NMR Spectrum | |
|---|---|---|---|---|---|
| | | | Ion | (300 MHz, | Starting |
| Method | Name | Structure | (Observed) | d6-DMSO) | Material |
| 15a | N-[3-amino-5- (hydroxymethyl) phenyl] methane- sulfonamide | 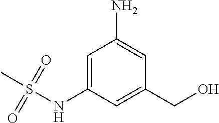 | | 2.91 (s, 3H), 4.29 (d, 2H), 4.98 (t, 1H), 5.10 (br s, 2H), 6.29 (s, 1H), 6.34 (m, 2H), 9.30 (s, 1H) | |

### Method 16

### 3-(Hydroxymethyl)-5-methyl-benzenesulfonamide

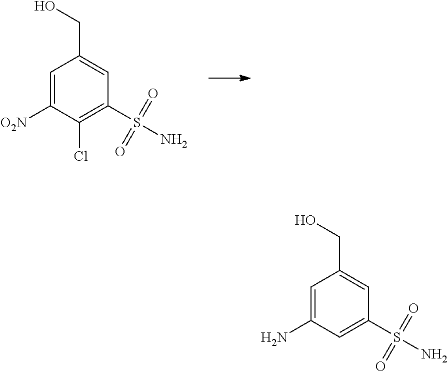

A mixture of 2-chloro-5-(hydroxymethyl)-3-nitro-benzenesulfonamide (2.5 g-method 15) and 10% Pd/C (200 mg) in ethanol (200 ml) was stirred under a hydrogen atmosphere at 50° C. overnight. The solution was cooled, filtered and concentrated, and the residue purified by chromatography using 0 to 25% methanol in DCM as eluent to give the title compound as a white solid (509 mg, 51%); NMR Spectrum (300 MHz, DMSO) 4.40 (m, 2H), 5.19 (t, 1H), 5.44 (br s, 2H), 6.68 (s, 1H), 6.90 (m, 1H), 6.94 (s, 1H), 7.09 (br s, 2H); Mass Spectrum MH⁺ 203.

### Method 17

### 7-Aminobenzo[1,3]dioxole-5-carbonitrile

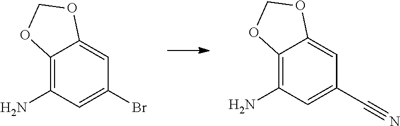

Zinc powder (125 mg), zinc cyanide (560 mg), tris(dibenzylideneacetone)dipalladium(0) (290 mg) and 1,1′-bis(diphenylphosphino)ferrocene (350 mg) were added to a solution of 6-bromobenzo[1,3]dioxol-4-amine (1 g, prepared as described in WO2004005284) and DIPEA (0.69 ml) in DMF (30 ml) and the reaction heated at 110° C. overnight. The solution was concentrated in vacuo and the residue partitioned between ethyl acetate and saturated aqueous sodium bicarbonate and filtered. Combined organic extracts were dried and concentrated to give a brown oil that was purified by chromatography using ethyl acetate:iso-hexane (80% to 50%) as eluent to give the title compound as a yellow solid (548 mg, 73%); NMR Spectrum (300 MHz, DMSO) 5.42 (br s, 2H), 6.06 (s, 2H), 6.66 (s, 2H); Mass Spectrum MH⁺ 161.

### Method 18

### Tert-butyl N-(6-formylbenzo[1,3]dioxol-4-yl)carbamate

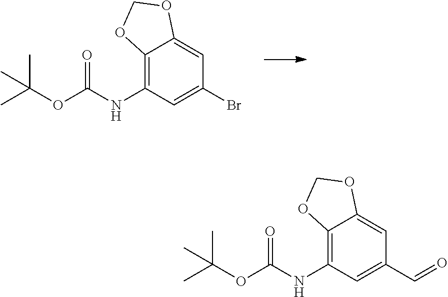

n-Butyl Lithium (9.96 ml, 2.5M in hexanes) was added dropwise to a solution of tert-butyl N-(6-bromobenzo[1,3]dioxol-4-yl)carbamate (3 g, prepared as described in WO2004005284) in THF (60 ml) at −78° C. and the mixture was stirred for 20 minutes. DMF (0.9 ml) was added and the solution allowed to warm to room temperature. Saturated aqueous sodium bicarbonate solution (75 ml) was added and the solution extracted with ethyl acetate, dried and concentrated. The residue was purified by chromatography using hexane to hexane-ethyl acetate (2:3) as eluent to give the title compound as a white solid (1.9 g, 76%); NMR Spectrum (300 MHz, DMSO) 1.45 (s, 9H), 6.18 (s, 2H), 7.17 (d, 1H), 7.68 (s, 1H), 9.14 (s, 1H), 9.78 (s, 1H); Mass Spectrum M⁺ 265.09.

### Method 19

### Tert-butyl N-[6-(hydroxymethyl)benzo[1,3]dioxol-4-yl]carbamate

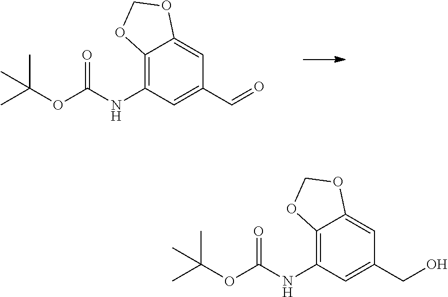

Sodium borohydride (306 mg) was added to a solution of tert-butyl N-(6-formylbenzo[1,3]dioxol-4-yl)carbamate (1.79 g-method 18) in methanol (50 ml) and the reaction stirred at room temperature for 2 hours then concentrated in vacuo. The residue was partitioned between water and ethyl acetate, dried and concentrated to give the title compound as a white foam (1.8 g, 100%); NMR Spectrum (300 MHz, DMSO) 1.43 (s, 9H), 4.35 (m, 2H), 5.08 (t, 1H), 5.96 (s, 2H), 6.62 (s, 1H), 6.89 (s, 1H), 8.75 (s, 1H).

### Method 20

### Tert-butyl N-[6-[(E/Z)-2-methoxyethenyl]benzo[1,3]dioxol-4-yl]carbamate

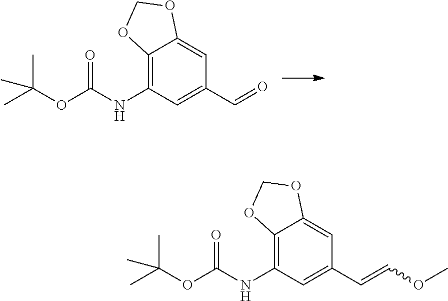

Potassium tert-butoxide (0.75 ml, 1M in THF) was added dropwise to a stirred suspension of the (methoxymethyl)triphenylphosphonium chloride (288 mg) in THF (3 ml) cooled in an ice bath. After stirring the red solution at room temperature for 30 minutes, tert-butyl N-(6-formylbenzo[1,3]dioxol-4-yl)carbamate (100 mg-method 18) in THF (3 ml) was added and the reaction stirred at room temperature for 12 hours. The reaction was partitioned between saturated aqueous ammonium chloride solution and diethyl ether. Combined organic extracts were washed with water, dried and concentrated and the residue purified by chromatography using iso-hexane to iso-hexane-10% ethyl acetate as eluent to give the title compound as a pale yellow oil (65 mg, 59%); NMR Spectrum (300 MHz, DMSO) 1.44 (d, 9H), 3.60 (s, 1.5H), 3.72 (s, 1.5H), 5.11 (d, 0.5H), 5.74 (d, 0.5H), 5.95 (d, 2H), 6.18 (d, 0.5H), 6.74-6.77 (d, 1H), 6.95-6.97 (m, 1H), 7.10 (d, 0.5H), 8.72 (d, 1H); Mass Spectrum M⁺ 292.43.

### Method 21

### Tert-butyl N-[6-(2-methoxyethyl)benzo[1,3]dioxol-4-yl]carbamate

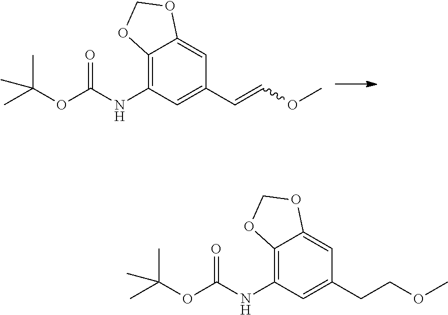

Tert-butyl N-[6-[(E)-2-methoxyethenyl]benzo[1,3]dioxol-4-yl]carbamate (0.9 g-method 20) and 10% Pd/C (90 mg) in ethanol (90 ml) were stirred under an atmosphere of hydrogen overnight. The solution was filtered and concentrated in vacuo to give the title compound as a pale brown oil (0.8 g, 88%); NMR Spectrum (300 MHz, DMSO) 1.44 (s, 9H), 2.68 (t, 2H), 3.30 (s, 3H), 3.40-3.47 (m, 2H), 5.95 (s, 2H), 6.58-6.59 (m, 1H), 6.76 (s, 1H), 8.72 (s, 1H); Mass Spectrum M−H⁺ 294.5.

### Method 22

### 6-(2-Methoxyethyl)benzo[1,3]dioxol-4-amine

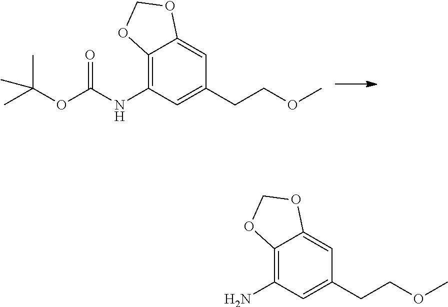

Tert-butyl N-[6-(2-methoxyethyl)benzo[1,3]dioxol-4-yl]carbamate (0.8 g-method 21) and c. HCl (0.25 ml) in methanol (10 ml) were heated at 70° C. for 2 hours then cooled and concentrated in vacuo. The residue was partitioned between ethyl acetate and saturated aqueous sodium bicarbonate and combined organic layers dried and concentrated and the residue purified by chromatography using 1:1 DCM-ethyl acetate as eluent to give the title compound as a pale brown oil (0.4 g, 76%); NMR Spectrum (300 MHz, DMSO) 3.29 (s, 3H), 3.44 (t, 2H), 4.79 (d, 2H), 5.84 (s, 2H), 6.07 (s, 1H), 6.11 (s, 1H); Mass Spectrum MH⁺ 196.49.

The procedure described above was repeated using the appropriate tert-butyl carbamate. Thus was obtained the compound described below:

| | | | Molecular | | Starting |
|---|---|---|---|---|---|
| | | | Ion | NMR Spectrum (300 | material |
| Method | Name | Structure | (Observed) | MHz, d6-DMSO) | (Method) |
| 22a | (7- aminobenzo [1,3]dioxol-5- yl)methanol | 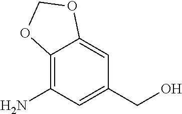 | 168.46 (MH+) | 4.27 (m, 2H), 4.83 (m, 1H), 4.92 (t, 1H), 5.86 (s, 2H), 6.13 (s, 1H), 6.24 (s, 1H) | 19 |

### Method 23

### 7-[(2-Chloropyrimidin-4-yl)amino]benzo[1,3]dioxole-5-carbonitrile

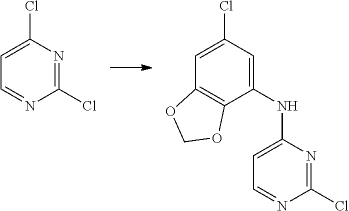

Sodium hydride (67 mg, 60% dispersion in mineral oil) was added to 7-aminobenzo[1,3]dioxole-5-carbonitrile (109 mg-method 17) in DMA (1 ml) at room temperature. 2,4-Dichloropyrimidine (100 mg) was added and the reaction stirred overnight. The reaction was quenched cautiously with water and the solution concentrated. The residue was triturated with water to give a solid which was filtered and dried in vacuo to give the title compound as a beige solid (83 mg, 45%); Mass Spectrum MH⁺ 275.37.

The procedure described above was repeated using the appropriate aniline. Thus were obtained the compounds described below:

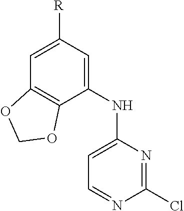

| | | | Molecular | Starting |
|---|---|---|---|---|
| | | | Ion | Material |
| Method | Name | R | (Observed) | (method) |
| 23a | N-(6- bromobenzo[1,3] dioxol-4- yl)-2-chloro- pyrimidin-4- amine | 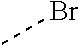 | 330.29 (MH+) | |
| 23b | 2-chloro-N-[6-(2- methoxyethyl) benzo[1,3] dioxol-4-yl] pyrimidin-4- amine | 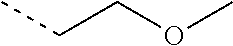 | 308.42 (MH+) | 22 |

### Method 24

### [7-[(2-Chloropyrimidin-4-yl)amino]benzo[1,3]dioxol-5-yl]methanol

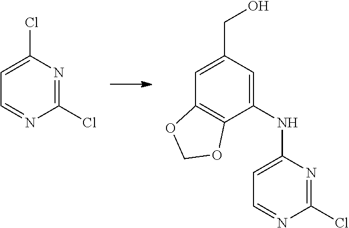

A mixture of DIPEA (0.07 ml), 2,4-dichloropyrimidine (50 mg) and (7-aminobenzo[1,3]dioxol-5-yl)methanol (56 mg-method 22a) in n-butanol (1 ml) was heated at 115° C. overnight then concentrated in vacuo. The residue was partitioned between ethyl acetate and water, and the organic solution concentrated. The residue was purified by chromatography using ethyl acetate as eluent to give the title compound as a white solid (37 mg, 39%); Mass Spectrum MH⁺ 280.42.

1. A compound of formula (I) 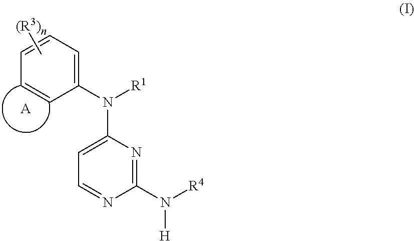
where R¹ is selected from hydrogen, C₁₋₆alkyl, C₂₋₆alkenyl, or C₂₋₆alkynyl, wherein the alkyl, alkenyl and alkynyl groups are optionally substituted by one or more substituent groups selected from cyano, nitro, —OR², —NR^{2a}R^{2b}, —C(O)NR^{2a}R^{2b}, or —N(R^{2a})C(O)R², halo or haloC₁₋₄alkyl, where R², R^{2a} and R^{2b} are selected from hydrogen or C₁₋₆alkyl such as methyl, or R^{2a} and R^{2b} together with the nitrogen atom to which they are attached may form a 5 or 6-membered heterocyclic ring, which optionally contains an additional heteroatom selected from N, O or S;
ring A is fused 5 or 6-membered carbocyclic or heterocyclic ring, which is saturated or unsaturated, and is optionally substituted on any available carbon atom by one or more substituent groups selected from halo, cyano, hydroxy, C₁₋₆alkyl, C₁₋₆alkoxy, —S(O)_{z}—C₁₋₆alkyl (where z is 0, 1 or 2), or —NR^{a}R^{b} (where R^{a} and R^{b} are each independently selected from hydrogen, C₁₋₄alkyl, or C₁₋₄alkylcarbonyl), and where any nitrogen atoms in the ring are optionally substituted by a C₁₋₆alkyl or C₁₋₆alkylcarbonyl;
n is 0, 1, 2 or 3
and each group R³ is independently selected from halo, trifluoromethyl, cyano, nitro or a group of sub-formula (i):
—X¹—R¹¹ (i)
where X¹ is selected from a direct bond or O, S, SO, SO₂, OSO₂, NR¹³, CO, CH(OR¹³), CONR¹³, N(R¹³)CO, SO₂N(R¹³), N(R¹³)SO₂, C(R¹³)₂O, C(R¹³)₂S, C(R¹³)₂N(R¹³) and N(R¹³)C(R¹³)₂, wherein R¹³ is hydrogen or C₁₋₆alkyl and R¹¹ is selected from hydrogen, C₁₋₆ alkyl, C₂₋₈alkenyl, C₂₋₈alkynyl, C₃₋₈cycloalkyl, aryl or heterocyclyl, C₃₋₈cycloalkylC₁₋₆ alkyl, arylC₁₋₆ alkyl or heterocyclylC₁₋₆alkyl, any of which may be optionally substituted with one or more groups selected from halo, trifluoromethyl, cyano, nitro, hydroxy, amino, carboxy, carbamoyl, C₁₋₆alkoxy, C₂₋₆alkenyoxyl, C₂₋₆alkynyloxy, C₁₋₆alkylthio, C₁₋₆alkylsulphinyl, C₁₋₆alkylsulphonyl, C₁₋₆alkylamino, di-(C₁₋₆alkyl)amino, C₁₋₆alkoxycarbonyl, N—C₁₋₆alkylcarbamoyl, N,N-di-(C₁₋₆alkyl)carbamoyl, C₂₋₆alkanoyl, C₂₋₆alkanoyloxy, C₂₋₆alkanoylamino, N—C₁₋₆alkyl-C₂₋₆alkanoylamino, C₃₋₆alkenoylamino, N—C₁₋₆alkyl-C₃₋₆alkenoylamino, C₃₋₆alkynoylamino, N—C₁₋₆alkyl-C₃₋₆alkynoylamino, N—C₁₋₆alkylsulphamoyl, N,N-di-(C₁₋₆alkyl)sulphamoyl, C₁₋₆alkanesulphonylamino and N—C₁₋₆alkyl-C₁₋₆alkanesulphonylamino, and any heterocyclyl group within R¹¹ optionally bears 1 or 2 oxo or thioxo substituents; and
R⁴ is a group of sub-formula (iii) 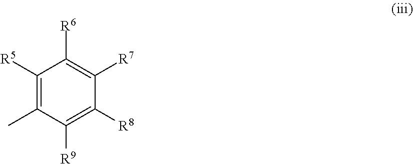
where R⁵, R⁶, R⁷, R⁸ and R⁹ are each independently selected from:
(i) hydrogen, halo, trifluoromethyl, trifluoromethoxy, cyano, nitro, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by halo, hydroxy, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino, and any nitrogen atoms present in a heterocyclyl group may, depending upon valency considerations, be substituted by a group selected from hydrogen, C₁₋₆alkyl or C₁₋₆alkylcarbonyl, and where any sulphur atoms may be optionally oxidised to a sulphur oxide;
a group of sub-formula (iv):
—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, —N(R¹⁶)C(O)N(R¹⁶)—, —N(R¹⁶)C(O)O—, SON(R¹⁶), N(R¹⁶)SO, SO₂N(R¹⁶), N(R¹⁶)SO₂, C(R¹⁶)₂O, C(R¹⁶)₂S and N(R¹⁶)C(R¹⁶)₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, aryl-C₁₋₆alkyl, or a 4- to 8-membered mono or bicyclic heterocyclyl ring (including 5 or 6 membered heteroaryl rings) or 4- to 8-membered mono or bicyclic heterocyclyl-C₁₋₆alkyl groups (including 5 or 6 membered heteroaryl-C₁₋₆alkyl groups) and wherein any aryl, C₃₋₁₂ carbocyclyl, aryl-C₁₋₆alkyl, heterocyclyl (including heteroaryl), heterocyclyl-C₁₋₆alkyl (including heteroaryl-C₁₋₆alkyl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
iii) a group of sub-formula (v):
—X³—R¹⁵—Z (v)
where X³ is a direct bond or is selected from O, NR¹⁷, S, SO, SO₂, OSO₂, CO, C(O)O, OC(O), CH(OR¹⁷), CON(R¹⁷), N(R¹⁷)CO, —N(R¹⁷)C(O)N(R¹⁷)—, —N(R¹⁷)C(O)O—, SO₂N(R¹⁷), N(R¹⁷)SO₂, C(R¹⁷)₂O, C(R¹⁷)₂S and N(R¹⁷)C(R¹⁷)₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, C₃₋₁₂ carbocyclyl or heterocyclyl (including heteroaryl) which optionally bears 1 or 2 substituents, which may be the same or different, selected from halo, C₁₋₆alkyl, C₂₋₈alkenyl, C₂₋₈alkynyl and C₁₋₆alkoxy and wherein any heterocyclyl group within Z optionally bears 1 or 2 oxo substituents, or Z is a group of sub-formula (vi)
—X⁴—R¹⁸ (vi)
where X⁴ is selected from O, NR¹⁹, S, SO, SO₂, OSO₂, CO, C(O)O, OC(O), CH(OR¹⁹), CON(R¹⁹), N(R¹⁹)CO, SO₂N(R¹⁹), —N(R¹⁹)C(O)N(R¹⁹)—, —N(R¹⁹)C(O)O—N(R¹⁹)SO₂, C(R¹⁹)₂O, C(R¹⁹)₂S and N(R¹⁹)C(R¹⁹)₂, where each R¹⁹ is independently selected from hydrogen or C₁₋₆alkyl; and R¹⁸ is selected from hydrogen, C₁₋₆ alkyl, C₂₋₈alkenyl, C₂₋₈alkynyl, aryl, C₃₋₁₂ carbocyclyl, aryl-C₁₋₆alkyl, heterocyclyl (including heteroaryl) or heterocyclyl-C₁₋₆alkyl (including heteroaryl-C₁₋₆alkyl) which optionally bears 1 or 2 substituents, which may be the same or different, selected from halo, C₁₋₆alkyl, C₂₋₈alkenyl, C₂₋₈alkynyl and C₁₋₆alkoxy, and wherein any heterocyclyl group within R¹⁸ optionally bears 1 or 2 oxo substituents; or
(iv) R⁵ and R⁶, R⁶ and R⁷, R⁷ and R⁸ or R⁸ and R⁹ are joined together to form a fused 5, 6 or 7-membered ring, wherein said ring is unsaturated or partially or fully saturated and is optionally substituted on any available carbon atom by halo, C₁₋₆alkyl, hydroxyC₁₋₆alkyl, amino, N—C₁₋₆alkylamino, or N,N-diC₁₋₆alkylamino, and said ring may contain one or more heteroatoms selected from oxygen, sulphur or nitrogen, where sulphur atoms may be optionally oxidised to a sulphur oxide, where any CH₂ groups may be substituted by a C(O) group, and where nitrogen atoms, depending upon valency considerations, may be substituted by a group R²¹, where R²¹ is selected from hydrogen, C₁₋₆alkyl or C₁₋₆alkylcarbonyl;
or a pharmaceutically acceptable salt thereof,
with the proviso that if Ring A, together with the phenyl ring to which attached, forms an indazol-4-yl group, then R¹ is not hydrogen.

2. A compound of formula (IA) 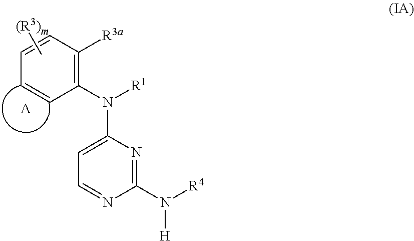
where A, R¹, R³ and R⁴ are as defined in claim 1, R^{3a} is a group R³ as defined in claim 1, and m is 0, 1 or 2.

3. A compound according to claim 2 wherein R^{3a} is halo.

4. A compound according to claim 2 where m is 0.

5. A compound according to claim 1 wherein Ring A is selected from —CR²²═CR²²—CR²²═CR²²—, —N═CR²²—CR²²═CR²²—, —CR²²═N—CR²²═CR²²—, —CR²²═CR²²—N═CR²²—, —CR²²═CR²²—CR²²═N—, —N═CR²²—N═CR²²—, —CR²²═N—CR²²═N—, —N═CR²²—CR²²═N—, —N═N—CR²²═CR²²—, —CR²²═CR²²—N═N—, —CR²²═CR²²—O—, —O—CR²²═CR²²—, —CR²²═CR²²—S—, —S—CR²²═CR²²—, —CR²²H—CR²²H—O—, —O—CR²²H—CR²²H—, —CR²²H—CR²²H—S—, —S—CR²²H—CR²²H—, —O—CR²²H—O—, —O—CF₂—O—, —O—CR²²H—CR²²H—O—, —S—CR²²H—S—, —S—CR²²H—CR²²H—S—, —CR²²═CR²²—NR²⁰—, —NR²⁰—CR²²═CR²²—, —CR²²H—CR²²H—NR²⁰—, —NR²⁰—CR²²H—CR²²H—, —N═CR²²—NR²⁰—, —NR²⁰—CR²²═N—, —NR²⁰—CR²²H—NR²⁰—, —OCR²²═N—, —N═CR²²—O—, —S—CR²²═N—, —N═CR²²—S—, —O—CR²²H—NR²⁰—, —NR²⁰—CR²²H—O—, —S—CR²²H—NR²⁰—, —NR²⁰—CR²²H—S—, —O—N═CR²²—, —CR²²═N—O—, —S—N═CR²²—, —CR²²═N—S—, —O—NR²⁰—CR²²H—, —CR²²H—NR²⁰—O—, —S—NR²⁰—CR²²H—, —CR²²H—NR²⁰—S—, —NR²⁰—N═CR²²—, —CR²²═N—NR²⁰—, —NR²⁰—NR²⁰—CR²²H—, —CR²²H—NR²⁰—NR²⁰—, —N═N—NR²⁰—, or —NR²⁰—N═N—,
where each R²⁰ is independently selected from hydrogen, C₁₋₄alkyl or C₁₋₄alkylcarbonyl, and where each R²² is independently selected from hydrogen, halo, cyano, hydroxy, C₁₋₄alkyl, C₁₋₄alkoxy, —S(O)_{z}—C₁₋₄alkyl (where z is 0, 1 or 2), or —NR^{a}R^{b} (where R^{a} and R^{b} are each independently selected from hydrogen, C₁₋₂alkyl, or C₁₋₂alkanoyl).

6. A compound according to claim 1, wherein Ring A is selected from Ring A is selected from —O—CR²²H—O—, —O—CF₂—O—, —OCR²²═N—, —N═CR²²—O—, —S—CR²²═N—, —N═CR²²—S—, —NR²⁰—N═CR²²—, or —CR²²═N—NR²⁰—, and each R²⁰ is independently selected from hydrogen, or C₁₋₂alkyl, and each R²² is independently selected from hydrogen, halo, or methyl.

7. A compound according to claim 1, wherein R¹ is hydrogen or a C₁₋₂alkyl group, which is optionally substituted with one or more substituents selected from cyano, —OR², —NR^{2a}R^{2b}, —C(O)NR^{2a}R^{2b}, or —N(R^{2a})C(O)R², halo or haloC₁₋₄alkyl, wherein R², R^{2a} and R^{2b} are selected from hydrogen or C₁₋₄alkyl.

8. A compound according to claim 1, wherein R¹ is methyl.

9. A compound according to claim 1, wherein each R³ group present is independently selected from halo, trifluoromethyl, cyano, nitro or a group of sub-formula (i):
—X¹—R¹¹ (i)
where X¹ is selected from a direct bond or O, CONR¹³, wherein R¹³ is hydrogen or C₁₋₆alkyl and R¹¹ is selected from hydrogen or C₁₋₄alkyl, which may be optionally substituted with one or more C₁₋₂alkoxy groups.

10. A compound according to claim 1, wherein n is 0 or 1.

11. A compound according to claim 1 wherein R⁴ is a group of sub-formula (iiib) 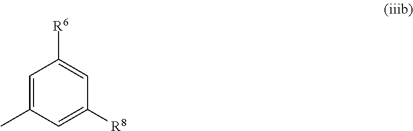
wherein at least one of R⁶ and R⁸ is a 5 or 6-membered nitrogen linked heterocyclic ring and the other is independently selected from:
(a) hydrogen, halo, trifluoromethyl, trifluoromethoxy, cyano, nitro, C₁₋₆ alkyl, C₂₋₈alkenyl, C₂₋₈alkynyl, aryl, heterocyclyl (including heteroaryl), and wherein any aryl or heterocyclyl (including heteroaryl) groups are optionally substituted on any available carbon atoms by halo, hydroxy, cyano, amino, C₁₋₆alkyl, hydroxyC₁₋₆alkyl, C₁₋₆alkoxy, and any nitrogen atoms present in the heterocyclyl moieties may, depending upon valency considerations, be substituted by a group selected from hydrogen, C₁₋₆alkyl or C₁₋₆alkylcarbonyl;
(b) a group of sub-formula (iv):
—X²—R¹⁴ (iv)
where X² is selected from O, NR¹⁶, S, SO, SO₂, OSO₂, CO, C(O)O, OC(O), CH(OR¹⁶), CON(R¹⁶), N(R¹⁶)CO, SON(R¹⁶), N(R¹⁶)SO, SO₂N(R¹⁶), and N(R¹⁶)SO₂, where each R¹⁶ is independently selected from hydrogen or C₁₋₆alkyl,
R¹⁴ is hydrogen, C₁₋₆ alkyl, trifluoromethyl, C₂₋₈alkenyl, C₂₋₈alkynyl, aryl, C₃₋₁₂ carbocyclyl, or a 4- to 8-membered mono or bicyclic heterocyclyl ring (including 5 or 6 membered heteroaryl rings) and wherein any aryl, C₃₋₁₂ carbocyclyl, heterocyclyl (including heteroaryl) groups are optionally substituted on any available carbon atoms by oxo, halo, cyano, amino, C₁₋₆alkyl, hydroxyC₁₋₆alkyl, C₁₋₆alkoxy, C₁₋₆alkylcarbonyl, N—C₁₋₆alkylamino, or N,N-diC₁₋₆alkylamino and any nitrogen atoms present in the heterocyclyl moieties may, depending upon valency considerations, be substituted by a group selected from hydrogen, C₁₋₆alkyl or C₁₋₆alkylcarbonyl, and where any sulphur atoms may be optionally oxidised to a sulphur oxide;
(c) a group of sub-formula (v) is
—X³—R¹⁵—Z (v)
where X³ is a direct bond or is selected from O, NR¹⁷, S, SO, SO₂, OSO₂, CO, C(O)O, OC(O), CON(R¹⁷), N(R¹⁷)CO, SO₂N(R¹⁷), and N(R¹⁷)SO₂, where each R¹⁷ is independently selected from hydrogen or C₁₋₆alkyl;
R¹⁵ is a C₁₋₆alkylene, C₂₋₆alkenylene or C₂₋₆alkynylene, arylene, C₃₋₁₂ carbocyclyl, heterocyclyl (including heteroaryl), any of which may be optionally substituted by one or more groups selected from halo, hydroxy, C₁₋₆alkyl, C₁₋₆alkoxy, cyano, amino, C₁₋₆alkylamino or di-(C₁₋₆alkyl)amino;
Z is halo, trifluoromethyl, cyano, nitro, aryl, or heterocyclyl (including heteroaryl) which optionally bears 1 or 2 substituents, which may be the same or different, selected from halo, C₁₋₆alkyl and C₁₋₆alkoxy and wherein any heterocyclyl group within Z optionally bears 1 or 2 oxo substituents, or Z is a group of sub-formula (vi)
—X⁴—R¹⁸ (vi)
where X⁴ is selected from O, NR¹⁹, S, SO, SO₂, OSO₂, CO, C(O)O, OC(O), CON(R¹⁹), N(R¹⁹)CO, SO₂N(R¹⁹), and N(R¹⁶)SO₂, where each R¹⁹ is independently selected from hydrogen or C₁₋₆alkyl; and R¹⁸ is selected from hydrogen, C₁₋₆ alkyl, aryl, or heterocyclyl (including heteroaryl) which optionally bears 1 or 2 substituents, which may be the same or different, selected from halo, C₁₋₆alkyl, and C₁₋₆alkoxy, and wherein any heterocyclyl group within R¹⁸ optionally bears 1 or 2 oxo substituents.

12. A compound according to claim 1, wherein R⁴ is a group of sub-formula (iiib) 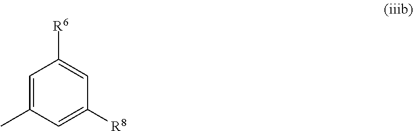
wherein at least one of R⁶ and R⁸ is morpholin-4yl and the other is independently selected from:
(a) hydrogen, halo, trifluoromethyl, cyano, C₁₋₄ alkyl, phenyl, a 5 or 6-membered heterocyclyl (including heteroaryl) comprising one or more heteroatoms selected from N, O or S,
and wherein any C₁₋₄ alkyl, aryl or heterocyclyl (including heteroaryl) groups are optionally substituted on any available carbon atoms by halo, hydroxy, cyano, amino, C₁₋₄alkyl, hydroxyC₁₋₄alkyl, C₁₋₄alkoxy, and any nitrogen atoms present in the heterocyclyl moieties may, depending upon valency considerations, be substituted by a group selected from hydrogen, C₁₋₄alkyl or C₁₋₄alkylcarbonyl; or
(b) a group of sub-formula (iv):
—X²—R¹⁴ (iv)
where X² is selected from O, NR¹⁶, S, SO, SO₂, OSO₂, CO, CON(R¹⁶), N(R¹⁶)CO, SON(R¹⁶), N(R¹⁶)SO, SO₂N(R¹⁶), and N(R¹⁶)SO₂, where each R¹⁶ is independently selected from hydrogen or C₁₋₄alkyl,
R¹⁴ is hydrogen, or C₁₋₄alkyl.

13. (canceled)

14. (canceled)

15. (canceled)

16. A compound according to claim 1, wherein R¹ is hydrogen or a C₁₋₂alkyl group, which is optionally substituted with one or more substituents selected from cyano, —OR², —NR^{2a}R^{2b}, where R², R^{2a} and R^{2b} are selected from hydrogen or C₁₋₂alkyl.

17. A compound according to claim 1, wherein R¹ is a C₁₋₂alkyl group, which is optionally substituted with one or more substituents selected from cyano, —OR², —NR^{2a}R^{2b}, where R², R^{2a} and R^{2b} are selected from hydrogen or C₁₋₂alkyl.

18. A compound according to claim 1, wherein R¹ is methyl.

19. A compound according to claim 1, wherein n is 0.

20. A compound according to claim 1, wherein R²² is hydrogen, halo, or C₁₋₂alkyl.

21. A compound according to claim 1, wherein R⁴ is as defined in claim 12.

22. A compound according to claim 1, wherein R⁴ is a group of sub-formula (iiib) 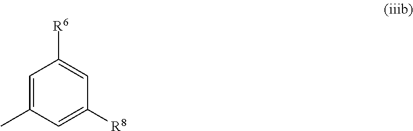
wherein both R⁶ and R⁸ are a 5 or 6-membered nitrogen linked heterocyclylic rings.

23. A compound according to claim 1, wherein R⁴ is a group of sub-formula (iiib) 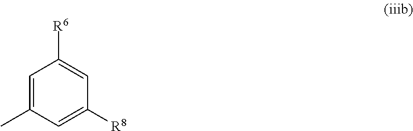
wherein both R⁶ and R⁸ are morpholin-4-yl.

24. A pharmaceutical composition comprising a compound according to claim 1, or a pharmaceutically acceptable salt thereof, in combination with a pharmaceutically acceptable carrier or diluent.

25. A process for preparing a compound of formula (I) by reacting a compound of formula (II): 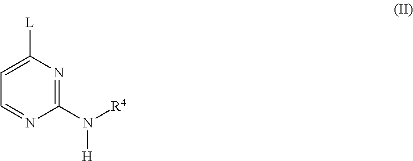
where R⁴ is as defined in claim 1 provided that any functional groups are optionally protected, and L is a leaving group, with a compound of formula (III) 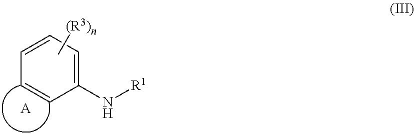
where A, R¹, R³ and n are as defined in claim 1, provided that any functional groups are optionally protected; or
by reaction a compound of formula (VII) 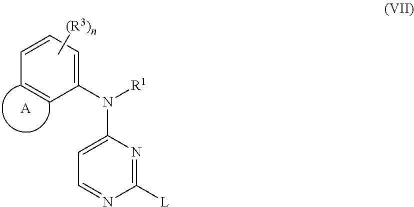
where A, R³ R¹ and n are as defined in claim 1 provided that any functional groups are optionally protected and L is a leaving group as defined in relation to formula (II), with a compound of formula (VI) as defined above;
and thereafter if desired or necessary carrying out one or more of the following steps:
(i) removing any protecting groups, or
(ii) converting a compound of formula (I) obtained into a different compound of formula (I);
(iii) forming a salt.

26. (canceled)

27. (canceled)

28. A method for producing an EphB4 inhibitory effect in a warm-blooded animal, such as man, in need of such treatment which comprises administering to said animal an effective amount of a compound according to claim 1, or a pharmaceutically acceptable salt thereof.

29. (canceled)

30. A method for producing an anti-angiogenic effect in a warm-blooded animal, such as man, in need of such treatment which comprises administering to said animal an effective amount of a compound according to claim 1, or a pharmaceutically acceptable salt thereof.

31. A method of treating cancer in a warm-blooded animal, such as man, in need of such treatment which comprises administering to said animal an effective amount of a compound according to claim 1, or a pharmaceutically acceptable salt thereof.

32. (canceled)

33. (canceled)
